SEQUENCE LISTING (238 sequences)

SEQ ID NO: 1 - protein, 6 aa, Artificial Sequence
  Synthesized
QDISNY

SEQ ID NO: 2 - protein, 9 aa, Artificial Sequence
  Synthesized
QQYDNLPPT

SEQ ID NO: 3 - protein, 8 aa, Artificial Sequence
  Synthesized
GFTFSYAW

SEQ ID NO: 4 - protein, 10 aa, Artificial Sequence
  Synthesized
IKSKTDGGTT

SEQ ID NO: 5 - protein, 15 aa, Artificial Sequence
  Synthesized
TTGWFTGTYGDYFDY

SEQ ID NO: 6 - protein, 107 aa, Artificial Sequence
  Synthesized
DIQMTQSPSSLSASVGDRVTITCQASQDISNYLNWYQQKPGKAPKLLIYDASNLQTGVPSRFSGSGSGTDFTFTISSLQPEDIATYYCQQYDNLPPTFGGGTKVEIK

SEQ ID NO: 7 - protein, 124 aa, Artificial Sequence
  Synthesized
EVQLVESGGGLVKPGGSLRLSCAASGFTFSYAWMTWVRQAPGKGLEWVGRIKSKTDGGTTDYAAPVKGRFTISRDDSKNTLFLQMNSLKTEDTAVYFCTTGWFTGTYGDYFDYWGQGTLVTVSS

SEQ ID NO: 8 - protein, 6 aa, Artificial Sequence
  Synthesized
QGISSY

SEQ ID NO: 9 - protein, 9 aa, Artificial Sequence
  Synthesized
QQLNSYPPT

SEQ ID NO: 10 - protein, 8 aa, Artificial Sequence
  Synthesized
GIIVSSNY

SEQ ID NO: 11 - protein, 7 aa, Artificial Sequence
  Synthesized
IYSGGST

SEQ ID NO: 12 - protein, 11 aa, Artificial Sequence
  Synthesized
ARDFREGAFDI

SEQ ID NO: 13 - protein, 107 aa, Artificial Sequence
  Synthesized
DIQLTQSPSFLSASVGDRVTITCRASQGISSYLAWYQQKPGKAPKLLIYAASTLQSGVPSRFSGSGSGTEFTLTISSLQPEDFATYYCQQLNSYPPTFGGGTKVEIK

SEQ ID NO: 14 - protein, 117 aa, Artificial Sequence
  Synthesized
EVQLVESGGGLVQPGGSLRLSCAASGIIVSSNYMSWVRQAPGKGLEWVSVIYSGGSTYYADSVKGRFTISRDNSKNTLYLQMSSLRAEDTAVYYCARDFREGAFDIWGQGTMVTVSS

SEQ ID NO: 15 - protein, 6 aa, Artificial Sequence
  Synthesized
KLGNKY

SEQ ID NO: 16 - protein, 9 aa, Artificial Sequence
  Synthesized
QAWGSSTVV

SEQ ID NO: 17 - protein, 8 aa, Artificial Sequence
  Synthesized
GGSISSYY

SEQ ID NO: 18 - protein, 7 aa, Artificial Sequence
  Synthesized
IYTSGST

SEQ ID NO: 19 - protein, 11 aa, Artificial Sequence
  Synthesized
ATDGGWYTFDH

SEQ ID NO: 20 - protein, 106 aa, Artificial Sequence
  Synthesized
SYELTQPPSVSVSPGQTASITCSGDKLGNKYACWYQQKPGQSPVLVIYQDNKRPSGIPERFSGSNSGNTATLTISGTQAMDEADYYCQAWGSSTVVFGGGTKLTVL

SEQ ID NO: 21 - protein, 117 aa, Artificial Sequence
  Synthesized
QVQLQESGPGLVKPSETLSLTCTVSGGSISSYYWNWIRQPAGKGLEWIGRIYTSGSTNYNPSLKSRVTMSVDTSKNQFSLKLSSVTAADTAVYYCATDGGWYTFDHWGQGTLVTVSS

SEQ ID NO: 22 - protein, 6 aa, Artificial Sequence
  Synthesized
QDISNY

SEQ ID NO: 23 - protein, 9 aa, Artificial Sequence
  Synthesized
QHYDNLPPT

SEQ ID NO: 24 - protein, 10 aa, Artificial Sequence
  Synthesized
GGSISSGGYY

SEQ ID NO: 25 - protein, 7 aa, Artificial Sequence
  Synthesized
IYYSGST

SEQ ID NO: 26 - protein, 11 aa, Artificial Sequence
  Synthesized
ARYPVWGAFDI

SEQ ID NO: 27 - protein, 107 aa, Artificial Sequence
  Synthesized
DIQMTQSPSSLSASVGDRVTITCQASQDISNYLNWYQQKPGKAPNLLIYDASNLETGVPSRFSGSGSGTDFTFTISSLQPEDFATYYCQHYDNLPPTFGPGTKVDIK

SEQ ID NO: 28 - protein, 119 aa, Artificial Sequence
  Synthesized
QVQLQESGPGLAKPSQTLSLTCTVSGGSISSGGYYWSWIRQHPGKGLEWIGYIYYSGSTYYNPSLKSRVTISVDTSKNQFSLKLSSVTAADTAVYYCARYPVWGAFDIWGQGTMVTVSS

SEQ ID NO: 29 - protein, 7 aa, Artificial Sequence
  Synthesized
QSVSSSY

SEQ ID NO: 30 - protein, 9 aa, Artificial Sequence
  Synthesized
QQYGSSPWT

SEQ ID NO: 31 - protein, 8 aa, Artificial Sequence
  Synthesized
GFTFSSSA

SEQ ID NO: 32 - protein, 8 aa, Artificial Sequence
  Synthesized
IVVGSGNT

SEQ ID NO: 33 - protein, 16 aa, Artificial Sequence
  Synthesized
AAAYCSGGSCSDGFDI

SEQ ID NO: 34 - protein, 108 aa, Artificial Sequence
  Synthesized
EIVLTQSPGTLSLSPGERATLSCRASQSVSSSYLAWYQQKPGQAPRLLICATSSRATGIPDRFSGSGSGTDFTLTIRRLEPEDFALYYCQQYGSSPWTFGQGTKVEIK

SEQ ID NO: 35 - protein, 123 aa, Artificial Sequence
  Synthesized
EVQLVQSGPEVKKPGTSVKVSCKASGFTFSSSAVQWVRQARGQRLEWIGWIVVGSGNTNYAQKFQERVTITRDMSTNTAYMELSSLRSEDTAVYYCAAAYCSGGSCSDGFDIWGQGTMVTVSS

SEQ ID NO: 36 - protein, 8 aa, Artificial Sequence
  Synthesized
SGSIASNY

SEQ ID NO: 37 - protein, 10 aa, Artificial Sequence
  Synthesized
QSYDISNHWV

SEQ ID NO: 38 - protein, 8 aa, Artificial Sequence
  Synthesized
GFTFSRYT

SEQ ID NO: 39 - protein, 8 aa, Artificial Sequence
  Synthesized
ISYDGSNK

SEQ ID NO: 40 - protein, 12 aa, Artificial Sequence
  Synthesized
ARVLWLRGMFDY

SEQ ID NO: 41 - protein, 111 aa, Artificial Sequence
  Synthesized
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDISNHWVFGGGTKLTVL

SEQ ID NO: 42 - protein, 119 aa, Artificial Sequence
  Synthesized
EVQLVESGGGVVQPGRSLRLSCAASGFTFSRYTMHWVRQAPGKGLEWVAFISYDGSNKYYADSVKGRFTISRDNSKNTLYLQMNSLRAEDTAVYYCARVLWLRGMFDYWGQGTLVTVSS

SEQ ID NO: 43 - protein, 6 aa, Artificial Sequence
  Synthesized
QDITNY

SEQ ID NO: 44 - protein, 9 aa, Artificial Sequence
  Synthesized
QQYDNLPLT

SEQ ID NO: 45 - protein, 8 aa, Artificial Sequence
  Synthesized
GFTFSSYA

SEQ ID NO: 46 - protein, 8 aa, Artificial Sequence
  Synthesized
ISYDGSNE

SEQ ID NO: 47 - protein, 18 aa, Artificial Sequence
  Synthesized
ARGDYYGSGSYPGKTFDY

SEQ ID NO: 48 - protein, 107 aa, Artificial Sequence
  Synthesized
DIQMTQSPSSLSASVGDRVTITCQASQDITNYLNWYQQKPGKAPKLLIYDASNLETGVPSRFSGSGSGTDFTFTISSLQPEDIATYYCQQYDNLPLTFGGGTKVEIK

SEQ ID NO: 49 - protein, 125 aa, Artificial Sequence
  Synthesized
EVQLVESGGGVVQPGRSLRLSCAASGFTFSSYAMFWVRQAPGKGLEWVAVISYDGSNEYYADSVKGRFTISRDNSKNTLYLQMNSLRAEDTAVYYCARGDYYGSGSYPGKTFDYWGQGTLVTVSS

SEQ ID NO: 50 - protein, 12 aa, Artificial Sequence
  Synthesized
QSVLYSSNNKDY

SEQ ID NO: 51 - protein, 9 aa, Artificial Sequence
  Synthesized
QQYYSTPYT

SEQ ID NO: 52 - protein, 8 aa, Artificial Sequence
  Synthesized
GGTFSSYA

SEQ ID NO: 53 - protein, 8 aa, Artificial Sequence
  Synthesized
IIPILGIA

SEQ ID NO: 54 - protein, 18 aa, Artificial Sequence
  Synthesized
ARGRLDSYSGSYYSWFDP

SEQ ID NO: 55 - protein, 113 aa, Artificial Sequence
  Synthesized
DIVMTQSPDSLAVSLGERATINCKSSQSVLYSSNNKDYLAWYQQKPGQPPNLLIYWASTRESGVPDRFSGSGSGTDFTLTISSLQAEDVAVYYCQQYYSTPYTFGQGTKVEIK

SEQ ID NO: 56 - protein, 125 aa, Artificial Sequence
  Synthesized
EVQLVQSGAEVKKPGSSVKVSCKASGGTFSSYAINWVRQAPGQGLEWMGRIIPILGIANYAQKFQGRVTITADKSTSTAYMELSSLRSEDTAVYYCARGRLDSYSGSYYSWFDPWGQGTLVTVSS

SEQ ID NO: 57 - protein, 9 aa, Artificial Sequence
  Synthesized
SSDVGSYNL

SEQ ID NO: 58 - protein, 10 aa, Artificial Sequence
  Synthesized
CSYAGSSTWV

SEQ ID NO: 59 - protein, 9 aa, Artificial Sequence
  Synthesized
GGSISSSNW

SEQ ID NO: 60 - protein, 7 aa, Artificial Sequence
  Synthesized
IYHSGNT

SEQ ID NO: 61 - protein, 15 aa, Artificial Sequence
  Synthesized
ATKYCSGGSCSYFGY

SEQ ID NO: 62 - protein, 110 aa, Artificial Sequence
  Synthesized
QSALTQPASVSGSPGQSITISCTGTSSDVGSYNLVSWYQQHPGKAPKLMIYEVSKRPSGVSNRFSGSKSGNTASLTISGLQAEDEADYYCCSYAGSSTWVFGGGTKLTVL

SEQ ID NO: 63 - protein, 122 aa, Artificial Sequence
  Synthesized
QVQLQESGPGLVKPSGTLSLTCAVSGGSISSSNWWSWVRQPPGKGLEWIGEIYHSGNTNYNPSLKSRVTISVDKSKNQFSLKLSSVTAADTAVYYCATKYCSGGSCSYFGYWGQGTLVTVSS

SEQ ID NO: 64 - protein, 7 aa, Artificial Sequence
  Synthesized
QSVSSSY

SEQ ID NO: 65 - protein, 8 aa, Artificial Sequence
  Synthesized
QQYGSSYT

SEQ ID NO: 66 - protein, 8 aa, Artificial Sequence
  Synthesized
GFTFSSCG

SEQ ID NO: 67 - protein, 8 aa, Artificial Sequence
  Synthesized
ISYDGSNK

SEQ ID NO: 68 - protein, 14 aa, Artificial Sequence
  Synthesized
AKGHSGSYRAPFDY

SEQ ID NO: 69 - protein, 107 aa, Artificial Sequence
  Synthesized
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSYTFGQGTKVEIK

SEQ ID NO: 70 - protein, 121 aa, Artificial Sequence
  Synthesized
EVQLVESGGGVVQPGRSLRLSCAASGFTFSSCGMHWVRQAPGKGLEWVAVISYDGSNKYYADSVKGRFTISRDNSKNTLYLQMNSLRAEDTAVYYCAKGHSGSYRAPFDYWGQGTLVTVSS

SEQ ID NO: 71 - protein, 6 aa, Artificial Sequence
  Synthesized
ALPKKY

SEQ ID NO: 72 - protein, 12 aa, Artificial Sequence
  Synthesized
YSTDSSDNHRRV

SEQ ID NO: 73 - protein, 8 aa, Artificial Sequence
  Synthesized
GFTFSTYG

SEQ ID NO: 74 - protein, 8 aa, Artificial Sequence
  Synthesized
IWYDGSNK

SEQ ID NO: 75 - protein, 14 aa, Artificial Sequence
  Synthesized
AREAYFGSGSSPDY

SEQ ID NO: 76 - protein, 109 aa, Artificial Sequence
  Synthesized
SYELTQPPSVSVSPGQTARITCSGDALPKKYAYWYQQKSGQAPVLVIYEDSKRPSGIPERFSGSSSGTMATLTISGAQVGDEADYYCYSTDSSDNHRRVFGGGTKLTVL

SEQ ID NO: 77 - protein, 121 aa, Artificial Sequence
  Synthesized
EVQLVESGGGVVQPGRSLRLSCAASGFTFSTYGMHWVRQAPGKGLEWVAVIWYDGSNKYYADSVKGRFTISRDNSKNTLYLQMNSLRAEDTAVYYCAREAYFGSGSSPDYWGQGTLVTVSS

SEQ ID NO: 78 - protein, 6 aa, Artificial Sequence
  Synthesized
ALPKKY

SEQ ID NO: 79 - protein, 12 aa, Artificial Sequence
  Synthesized
YSTDSGGNPQGV

SEQ ID NO: 80 - protein, 8 aa, Artificial Sequence
  Synthesized
GFTFSSYW

SEQ ID NO: 81 - protein, 8 aa, Artificial Sequence
  Synthesized
IKEDGSEK

SEQ ID NO: 82 - protein, 20 aa, Artificial Sequence
  Synthesized
AREGTYYYDSSAYYNGGLDY

SEQ ID NO: 83 - protein, 109 aa, Artificial Sequence
  Synthesized
SYELTQPPSVSVSPGQTARITCSGDALPKKYAYWFQQKSGQAPVLVIYEDSKRPSGIPERFSGSSSGTMATLTISGAQVEDEADYYCYSTDSSGNHRRLFGTGTKVTVL

SEQ ID NO: 84 - protein, 127 aa, Artificial Sequence
  Synthesized
EVQLVESGGGLVQPGGSLRLSCAASGFTFSSYWMSWVRQAPGKGLEWVANIKEDGSEKYYVDSVKGRFTISRDNAKNSLYLQMNSLRAEDTAVYYCAREGTYYYDSSAYYNGGLDYWGQGTLVTVSS

SEQ ID NO: 85 - protein, 6 aa, Artificial Sequence
  Synthesized
QDISNY

SEQ ID NO: 86 - protein, 9 aa, Artificial Sequence
  Synthesized
QQYDNIPLT

SEQ ID NO: 87 - protein, 8 aa, Artificial Sequence
  Synthesized
GFTFYNYG

SEQ ID NO: 88 - protein, 8 aa, Artificial Sequence
  Synthesized
ISYDGSNK

SEQ ID NO: 89 - protein, 21 aa, Artificial Sequence
  Synthesized
AKQGGGTYCGGGSCYRGYFDY

SEQ ID NO: 90 - protein, 107 aa, Artificial Sequence
  Synthesized
DIQMTQSPSSLSASVGDRVTITCQASQDISNYLNWYQQKPGKAPKLLIYDASNLETGVPSRFSGSGSGTDFTFIISSLQPEDIATYYCQQYDNIPLTFGGGTKVEIK

SEQ ID NO: 91 - protein, 128 aa, Artificial Sequence
  Synthesized
EVQLVESGGVVVQPGRSLRLSCAASGFTFYNYGMHWVRQAPGKGLEWVAVISYDGSNKYYADSVKGRFTISRDNSKNTLYLQMNSLRAEDTAVYYCAKQGGGTYCGGGSCYRGYFDYWGQGTLVTVSS

SEQ ID NO: 92 - protein, 6 aa, Artificial Sequence
  Synthesized
QSVSSY

SEQ ID NO: 93 - protein, 11 aa, Artificial Sequence
  Synthesized
QQRSNRPPRWT

SEQ ID NO: 94 - protein, 8 aa, Artificial Sequence
  Synthesized
GYTFSNYY

SEQ ID NO: 95 - protein, 8 aa, Artificial Sequence
  Synthesized
FNPSGGGT

SEQ ID NO: 96 - protein, 18 aa, Artificial Sequence
  Synthesized
ARDPRVPAVTNVNDAFDL

SEQ ID NO: 97 - protein, 109 aa, Artificial Sequence
  Synthesized
EIVLTQSPATLSLSPGERATLSCRASQSVSSYLAWYQHKPGQAPRLIIYNASNRATGIPARFSGSRSGTDFTLTISSLEPEDFAVYYCQQRSNRPPRWTFGQGTKVEIK

SEQ ID NO: 98 - protein, 125 aa, Artificial Sequence
  Synthesized
EVQLVQSGAEAKKPGASVNISCRTSGYTFSNYYIHWVRQAPGQGLEWMGIFNPSGGGTSYAQNFQGRLTMTSDTSTSTVFMELSSLGSEDTAVYYCARDPRVPAVTNVNDAFDLWGQGTMVTVSS

SEQ ID NO: 99 - protein, 12 aa, Artificial Sequence
  Synthesized
QSVLYSSNNKNY

SEQ ID NO: 100 - protein, 9 aa, Artificial Sequence
  Synthesized
QQYYSTPCS

SEQ ID NO: 101 - protein, 8 aa, Artificial Sequence
  Synthesized
EFTVSSNY

SEQ ID NO: 102 - protein, 7 aa, Artificial Sequence
  Synthesized
IYLGGST

SEQ ID NO: 103 - protein, 13 aa, Artificial Sequence
  Synthesized
ARSHLEVRGVFDN

SEQ ID NO: 104 - protein, 113 aa, Artificial Sequence
  Synthesized
DIVMTQSPDSLAVSLGERATVNCKSSQSVLYSSNNKNYLAWYQQKPGQPPKLLIYWASTRESGVPDRFSGSGSGTDFTLTISSLQAEDVAVYYCQQYYSTPCSFGQGTKVEIK

SEQ ID NO: 105 - protein, 119 aa, Artificial Sequence
  Synthesized
EVQLVETGGGLIQPGGSLRLSCAVSEFTVSSNYMSWVRQAPGEGLEWVSVIYLGGSTDYADSVKGRFTISRDNSKNTLYLQMNSLRAEDTAVYYCARSHLEVRGVFDNWGQGTLVTVSS

SEQ ID NO: 106 - protein, 6 aa, Artificial Sequence
  Synthesized
ALPKKY

SEQ ID NO: 107 - protein, 11 aa, Artificial Sequence
  Synthesized
YSTDSSVNGRV

SEQ ID NO: 108 - protein, 8 aa, Artificial Sequence
  Synthesized
GFTFSSYG

SEQ ID NO: 109 - protein, 8 aa, Artificial Sequence
  Synthesized
IWYDGGNK

SEQ ID NO: 110 - protein, 16 aa, Artificial Sequence
  Synthesized
AREGVYGDIGGAGLDY

SEQ ID NO: 111 - protein, 108 aa, Artificial Sequence
  Synthesized
SYELTQPPSVSVSPGQTARITCSGDALPKKYAYWYQQKSGQAPVLVIYEDSKRPSGIPERFSGSSSGTMATLTISGAQVEDEADYYCYSTDSSVNGRVFGTGTKVTVL

SEQ ID NO: 112 - protein, 123 aa, Artificial Sequence
  Synthesized
EVQLVESGGGVVQPGRSLRLSCAASGFTFSSYGMHWVRQAPGKGLEWVAVIWYDGGNKHYADSVKGRFTISRDNSKNTLYLQMDSLRAEDTAVYYCAREGVYGDIGGAGLDYWGQGTLVTVSS

SEQ ID NO: 113 - protein, 6 aa, Artificial Sequence
  Synthesized
QDISNY

SEQ ID NO: 114 - protein, 8 aa, Artificial Sequence
  Synthesized
QQYDNLLT

SEQ ID NO: 115 - protein, 8 aa, Artificial Sequence
  Synthesized
GFTFSSYG

SEQ ID NO: 116 - protein, 8 aa, Artificial Sequence
  Synthesized
ISYDGSNK

SEQ ID NO: 117 - protein, 20 aa, Artificial Sequence
  Synthesized
AKMGGVYCSAGNCYSGRLEY

SEQ ID NO: 118 - protein, 106 aa, Artificial Sequence
  Synthesized
DIQMTQSPSSLSASVGDRVTITCQASQDISNYLNWYQQKPGKAPKLLIYDASNLETGVPSRFSGSGSGTDFTFTISSLQPEDIATYYCQQYDNLLTFGPGTKVDIK

SEQ ID NO: 119 - protein, 127 aa, Artificial Sequence
  Synthesized
EVQLVESGGGVVQPGRSLRLSCAASGFTFSSYGMHWVRQAPGKGLEWVAVISYDGSNKYYADSVKGRFTISRDNSKNTLFLQMSSLRAEDTAVYYCAKMGGVYCSAGNCYSGRLEYWGLGTLVTVSS

SEQ ID NO: 120 - protein, 12 aa, Artificial Sequence
  Synthesized
QSISYFSNNKNY

SEQ ID NO: 121 - protein, 9 aa, Artificial Sequence
  Synthesized
QQYFTTPWT

SEQ ID NO: 122 - protein, 8 aa, Artificial Sequence
  Synthesized
GGSMNSNY

SEQ ID NO: 123 - protein, 7 aa, Artificial Sequence
  Synthesized
IYYRGST

SEQ ID NO: 124 - protein, 11 aa, Artificial Sequence
  Synthesized
ARETVNNWVDP

SEQ ID NO: 125 - protein, 113 aa, Artificial Sequence
  Synthesized
DIVMTQSPDSLTVSLGERATINCKSSQSISYFSNNKNYLAWYQQKPGQPPKLLIYWASTRESGVPDRFGGSGSGADFTLTISSLQAEDVAVYYCQQYFTTPWTFGQGTKVEIK

SEQ ID NO: 126 - protein, 117 aa, Artificial Sequence
  Synthesized
QVQLQESGPRLVRPLETLSLTCTVSGGSMNSNYWSWIRQPPGKRLEWIGYIYYRGSTNYNPSLKSRVTISVDTSKNQFSLNLTSVTAADTAIYYCARETVNNWVDPWGQGTLVTVSS

SEQ ID NO: 127 - protein, 8 aa, Artificial Sequence
  Synthesized
RGSIAGNY

SEQ ID NO: 128 - protein, 9 aa, Artificial Sequence
  Synthesized
QSFDSSNVV

SEQ ID NO: 129 - protein, 8 aa, Artificial Sequence
  Synthesized
GYSFTSYW

SEQ ID NO: 130 - protein, 8 aa, Artificial Sequence
  Synthesized
IYPGDSDT

SEQ ID NO: 131 - protein, 14 aa, Artificial Sequence
  Synthesized
ARREWGGSLGHIDY

SEQ ID NO: 132 - protein, 110 aa, Artificial Sequence
  Synthesized
NFMLTQPHSVSESPGKTVTISCTRSRGSIAGNYVQWYQQRPGSAPTTVIYEDNQRPSGVPDRFSGSIDSSSNSASLTISGLKTEDEAEYYCQSFDSSNVVFGGGTKVTVL

SEQ ID NO: 133 - protein, 121 aa, Artificial Sequence
  Synthesized
EVQLVQSGAEVKKPGESLKISCKGSGYSFTSYWIGWVRQMPGRGLEWMGIIYPGDSDTRYSPSFQGQVTISADKSISTAYLQWSSLKASDTAMYYCARREWGGSLGHIDYWGQGTLVTVSS

SEQ ID NO: 134 - protein, 6 aa, Artificial Sequence
  Synthesized
NIGSNS

SEQ ID NO: 135 - protein, 11 aa, Artificial Sequence
  Synthesized
QVWDSSSDPVV

SEQ ID NO: 136 - protein, 8 aa, Artificial Sequence
  Synthesized
GFTVSSNY

SEQ ID NO: 137 - protein, 7 aa, Artificial Sequence
  Synthesized
IYSGGST

SEQ ID NO: 138 - protein, 11 aa, Artificial Sequence
  Synthesized
ARDLQLYGMDV

SEQ ID NO: 139 - protein, 108 aa, Artificial Sequence
  Synthesized
SYELTQPPSVSVAPGQTARITCGGNNIGSNSVHWYQQKPGQAPVLVVYDDSDRPSGIPERFSGSNSGNTATLTISRVEAGDEADYHCQVWDSSSDPVVFGGGTKLTVL

SEQ ID NO: 140 - protein, 117 aa, Artificial Sequence
  Synthesized
EVQLVETGGGLIQPGGSLRLSCAASGFTVSSNYMTWVRQAPGKGLEWVSLIYSGGSTYYADSVKGRFTISRDNSKNTLYLQMNSLRAEDTAVYYCARDLQLYGMDVWGQGTTVTVSS

SEQ ID NO: 141 - protein, 6 aa, Artificial Sequence
  Synthesized
ALPKQY

SEQ ID NO: 142 - protein, 10 aa, Artificial Sequence
  Synthesized
QSADSSGTYV

SEQ ID NO: 143 - protein, 8 aa, Artificial Sequence
  Synthesized
GYIFTSYG

SEQ ID NO: 144 - protein, 8 aa, Artificial Sequence
  Synthesized
ISAYNGNT

SEQ ID NO: 145 - protein, 29 aa, Artificial Sequence
  Synthesized
ARVPGLVGYSSSWYDNEKNYYYYYYGMDV

SEQ ID NO: 146 - protein, 107 aa, Artificial Sequence
  Synthesized
SYELTQPPSVSVSPGQTARITCSGDALPKQYAYWYQQKPGQAPVLVIYKDSERPSGIPERFSGSSTGTTVTLTISGVQAEDEADYYCQSADSSGTYVFGTGTKVTVL

SEQ ID NO: 147 - protein, 136 aa, Artificial Sequence
  Synthesized
EVQLVQSGAEVKKPGASVKVSCKASGYIFTSYGISWVRQAPGQGLEWMGWISAYNGNTNYAQKLQGRVTMTTDTSTSTAYMELRSLRSDDTAVYYCARVPGLVGYSSSWYDNEKNYYYYYYGMDVWGQGTTVTVSS

SEQ ID NO: 148 - protein, 6 aa, Artificial Sequence
  Synthesized
QSVSTY

SEQ ID NO: 149 - protein, 9 aa, Artificial Sequence
  Synthesized
QHRSNWPLT

SEQ ID NO: 150 - protein, 8 aa, Artificial Sequence
  Synthesized
GFTFSSYA

SEQ ID NO: 151 - protein, 8 aa, Artificial Sequence
  Synthesized
ISGSGGST

SEQ ID NO: 152 - protein, 15 aa, Artificial Sequence
  Synthesized
AKADTAMAWYNWFDP

SEQ ID NO: 153 - protein, 107 aa, Artificial Sequence
  Synthesized
EIVLTQSPATLSLSPGERATLSCRASQSVSTYLAWYQQKPGQALRLLIYDASNRATGIPARFSGSGSGTDFTLTISSLEPEDFAVYYCQHRSNWPLTFGGGTKVEIK

SEQ ID NO: 154 - protein, 122 aa, Artificial Sequence
  Synthesized
EVQLLESGGGLVQPGGSLRLSCAASGFTFSSYAMSWVRQAPGKGLEWVSAISGSGGSTYYADSVKGRLTISRDNSKNTLYMQMNSLRAEDTAVYYCAKADTAMAWYNWFDPWGQGTLVTVSS

SEQ ID NO: 155 - protein, 6 aa, Artificial Sequence
  Synthesized
NIGRKS

SEQ ID NO: 156 - protein, 13 aa, Artificial Sequence
  Synthesized
QVWDNSSDQPNYV

SEQ ID NO: 157 - protein, 8 aa, Artificial Sequence
  Synthesized
GGSFSGYY

SEQ ID NO: 158 - protein, 7 aa, Artificial Sequence
  Synthesized
INHSGST

SEQ ID NO: 159 - protein, 12 aa, Artificial Sequence
  Synthesized
ARVWVRWWYFDL

SEQ ID NO: 160 - protein, 110 aa, Artificial Sequence
  Synthesized
SYELTQPPSVSVAPGQTARITCGGNNIGRKSVHWYQQKPGQAPVLVVYDDSDRPSGIPERFSGSNSGNTATLTLSRVEAGDEADYYCQVWDNSSDQPNYVFGTGTKVTVL

SEQ ID NO: 161 - protein, 118 aa, Artificial Sequence
  Synthesized
QVQLQQWGAGLLKPSETLSLTCAVYGGSFSGYYWSWIRQPPGKGLEWLGEINHSGSTNYNPSLKSRVTISVDTSKNQFSLKLSSVTAADTAVYYCARVWVRWWYFDLWGRGTLVTVSS

SEQ ID NO: 162 - protein, 13 aa, Artificial Sequence
  Synthesized
CQQRSNRPPRWTF

SEQ ID NO: 163 - protein, 11 aa, Artificial Sequence [sequence omitted]

SEQ ID NO: 164 - protein, 13 aa, Artificial Sequence
  Synthesized
CYSTDSSVNGRVF

SEQ ID NO: 165 - protein, 10 aa, Artificial Sequence [sequence omitted]

SEQ ID NO: 166 - protein, 11 aa, Artificial Sequence [sequence omitted]

SEQ ID NO: 167 - protein, 11 aa, Artificial Sequence
  Synthesized
CQSFDSSNVVF

SEQ ID NO: 168 - protein, 13 aa, Artificial Sequence [sequence omitted]

SEQ ID NO: 169 - protein, 12 aa, Artificial Sequence [sequence omitted]

SEQ ID NO: 170 - protein, 11 aa, Artificial Sequence [sequence omitted]

SEQ ID NO: 171 - protein, 15 aa, Artificial Sequence
  Synthesized
CQVWDNSSDQPNYVF

SEQ ID NO: 172 - protein, 17 aa, Artificial Sequence
  Synthesized
CTTGWFTGTYGDYFDYW

SEQ ID NO: 173 - protein, 13 aa, Artificial Sequence
  Synthesized
CARDFREGAFDIW

SEQ ID NO: 174 - protein, 13 aa, Artificial Sequence
  Synthesized
CATDGGWYTFDHW

SEQ ID NO: 175 - protein, 13 aa, Artificial Sequence
  Synthesized
CARYPVWGAFDIW

SEQ ID NO: 176 - protein, 18 aa, Artificial Sequence
  Synthesized
CAAAYCSGGSCSDGFDIW

SEQ ID NO: 177 - protein, 14 aa, Artificial Sequence
  Synthesized
CARVLWLRGMFDYW

SEQ ID NO: 178 - protein, 20 aa, Artificial Sequence
  Synthesized
CARGDYYGSGSYPGKTFDYW

SEQ ID NO: 179 - protein, 20 aa, Artificial Sequence
  Synthesized
CARGRLDSYSGSYYSWFDPW

SEQ ID NO: 180 - protein, 17 aa, Artificial Sequence
  Synthesized
CATKYCSGGSCSYFGYW

SEQ ID NO: 181 - protein, 16 aa, Artificial Sequence
  Synthesized
CAKGHSGSYRAPFDYW

SEQ ID NO: 182 - protein, 16 aa, Artificial Sequence
  Synthesized
CAREAYFGSGSSPDYW

SEQ ID NO: 183 - protein, 22 aa, Artificial Sequence
  Synthesized
CAREGTYYYDSSAYYNGGLDYW

SEQ ID NO: 184 - protein, 23 aa, Artificial Sequence
  Synthesized
CAKQGGGTYCGGGSCYRGYFDYW

SEQ ID NO: 185 - protein, 20 aa, Artificial Sequence
  Synthesized
CARDPRVPAVTNVNDAFDLW

SEQ ID NO: 186 - protein, 15 aa, Artificial Sequence
  Synthesized
CARSHLEVRGVFDNW

SEQ ID NO: 187 - protein, 18 aa, Artificial Sequence
  Synthesized
CAREGVYGDIGGAGLDYW

SEQ ID NO: 188 - protein, 22 aa, Artificial Sequence
  Synthesized
CAKMGGVYCSAGNCYSGRLEYW

SEQ ID NO: 189 - protein, 13 aa, Artificial Sequence
  Synthesized
CARETVNNWVDPW

SEQ ID NO: 190 - protein, 16 aa, Artificial Sequence
  Synthesized
CARREWGGSLGHIDYW

SEQ ID NO: 191 - protein, 13 aa, Artificial Sequence
  Synthesized
CARDLQLYGMDVW

SEQ ID NO: 192 - protein, 31 aa, Artificial Sequence
  Synthesized
CARVPGLVGYSSSWYDNEKNYYYYYYGMDVW

SEQ ID NO: 193 - protein, 17 aa, Artificial Sequence
  Synthesized
CAKADTAMAWYNWFDPW

SEQ ID NO: 194 - protein, 14 aa, Artificial Sequence
  Synthesized
CARVWVRWWYFDLW

SEQ ID NO: 195 - protein, 11 aa, Artificial Sequence [sequence omitted]

SEQ ID NO: 196 - protein, 11 aa, Artificial Sequence [sequence omitted]

SEQ ID NO: 197 - protein, 11 aa, Artificial Sequence [sequence omitted]

SEQ ID NO: 198 - protein, 11 aa, Artificial Sequence [sequence omitted]

SEQ ID NO: 199 - protein, 11 aa, Artificial Sequence [sequence omitted]

SEQ ID NO: 200 - protein, 12 aa, Artificial Sequence
  Synthesized
CQSYDISNHWVF

SEQ ID NO: 201 - protein, 11 aa, Artificial Sequence [sequence omitted]

SEQ ID NO: 202 - protein, 11 aa, Artificial Sequence [sequence omitted]

SEQ ID NO: 203 - protein, 12 aa, Artificial Sequence [sequence omitted]

SEQ ID NO: 204 - protein, 10 aa, Artificial Sequence [sequence omitted]

SEQ ID NO: 205 - protein, 14 aa, Artificial Sequence
  Synthesized
CYSTDSSDNHRRVF

SEQ ID NO: 206 - protein, 14 aa, Artificial Sequence
  Synthesized
CYSTDSSGNHRRLF

SEQ ID NO: 207 - protein, 26 aa, Artificial Sequence
  Synthesized
CQQYDNIPLTFAAVYCTTTCSDAFDI

SEQ ID NO: 208 - protein, 15 aa, Artificial Sequence
  Synthesized
AAPACGTSCSDAFDI

SEQ ID NO: 209 - protein, 16 aa, Artificial Sequence
  Synthesized
AAPHCIGGSCHDAFDI

SEQ ID NO: 210 - protein, 16 aa, Artificial Sequence
  Synethesized
AAPHCSSTSCFDAFDI

SEQ ID NO: 211 - protein, 16 aa, Artificial Sequence
  Synthesized
AAPHCNRTSCYDAFDL

SEQ ID NO: 212 - protein, 16 aa, Artificial Sequence
  Synthesized
AAVDCNSTSCYDAFDI

SEQ ID NO: 213 - protein, 16 aa, Artificial Sequence
  Synthesized
AAPHCNRTSCFDGFDI

SEQ ID NO: 214 - protein, 16 aa, Artificial Sequence
  Synthesized
AANHCSGGSCYDGFDI

SEQ ID NO: 215 - protein, 16 aa, Artificial Sequence
  Synthesized
AAPHCSSTICYDGFDI

SEQ ID NO: 216 - protein, 16 aa, Artificial Sequence
  Synthesized
AAPYCSNGVCHDGFDI

SEQ ID NO: 217 - protein, 16 aa, Artificial Sequence
  Synthesized
AAPYCSRTSCHDAFDI

SEQ ID NO: 218 - protein, 16 aa, Artificial Sequence
  Synthesized
AAPYCSSTSCHDGFDI

SEQ ID NO: 219 - protein, 16 aa, Artificial Sequence
  Synthesized
AAPYCSSTRCYDAFDI

SEQ ID NO: 220 - protein, 16 aa, Artificial Sequence
  Synthesized
AAPRCSGGSCYDGFDI

SEQ ID NO: 221 - protein, 16 aa, Artificial Sequence
  Synthesized
AAPHCSGGSCYDAFDI

SEQ ID NO: 222 - protein, 16 aa, Artificial Sequence
  Synthesized
AAPHCSGGSCLDAFDI

SEQ ID NO: 223 - protein, 16 aa, Artificial Sequence
  Synthesized
AAPYCSSISCNDGFDI

SEQ ID NO: 224 - protein, 16 aa, Artificial Sequence
  Synthesized
AAPYCSGGSCYDAFDI

SEQ ID NO: 225 - protein, 16 aa, Artificial Sequence
  Synthesized
AAPYCSGGSCNDAFDI

SEQ ID NO: 226 - protein, 16 aa, Artificial Sequence
  Synthesized
AAPYCSGGSCNDAFDI

SEQ ID NO: 227 - protein, 16 aa, Artificial Sequence
  Synthesized
AAPDCNRTTCRDGFDI

SEQ ID NO: 228 - protein, 10 aa, Artificial Sequence
  Synthesized
QHYGSSRGWT

SEQ ID NO: 229 - protein, 9 aa, Artificial Sequence
  Synthesized
QQYGSSPWM

SEQ ID NO: 230 - protein, 9 aa, Artificial Sequence
  Synthesized
QQYGRSPWT

SEQ ID NO: 231 - protein, 9 aa, Artificial Sequence
  Synthesized
QQYGNSPWT

SEQ ID NO: 232 - protein, 9 aa, Artificial Sequence
  Synthesized
QQYDISPWT

SEQ ID NO: 233 - protein, 18 aa, Artificial Sequence
  Synethesized
CAAAYCSGGSCSDGFDIW

SEQ ID NO: 234 - protein, 11 aa, Artificial Sequence [sequence omitted]

SEQ ID NO: 235 - protein, 5 aa, Artificial Sequence
  Synthesized
VGLTG

SEQ ID NO: 236 - DNA, 20 bp, Artificial Sequence
  Synthesized
gaccccaaaatcagcgaaat

SEQ ID NO: 237 - DNA, 24 bp, Artificial Sequence
  Synthesized
tctggttactgccagttgaatctg

SEQ ID NO: 238 - DNA, 24 bp, Artificial Sequence
  Synthesized
accccgcattacgtttggtggacc

(19) United States
(12) Patent Application Publication
(10) Pub. No.: US 2022/0289827 A1
(43) Pub. Date: Sep. 15, 2022

(54) NEUTRALIZING ANTIBODIES TO SARS-COV-2 AND ITS VARIANTS

(71) Applicant: Washington University, St. Louis, MO (US)

(72) Inventor: Ali Ellebedy, St. Louis, MO (US)

(21) Appl. No.: 17/692,838

(22) Filed: Mar. 11, 2022

Related U.S. Application Data
(60) Provisional application No. 63/159,961, filed on Mar. 11, 2021.
(60) Provisional application No. 63/164,961, filed on Mar. 23, 2021.

(51) Int. Cl. C07K 16/10; A61K 45/06; A61P 31/14

(52) U.S. Cl. CPC C07K 16/10; A61K 45/06; A61P 31/14

(57) ABSTRACT

The present invention relates to antibodies or antigen-binding fragments that are useful for treating coronavirus infections (e.g., COVID-19 caused by SARS-CoV-2). The present invention also relates to various pharmaceutical compositions and methods of treating coronavirus using the antibodies or antigen-binding fragments.

### CROSS REFERENCE TO RELATED APPLICATIONS

This application claims the benefit of U.S. Provisional Application No. 63/159,961, filed Mar. 11, 2021, and the benefit of U.S. Provisional Application No. 63/164,961, filed Mar. 23, 2021, the disclosures of which are hereby incorporated by reference in their entirety.

### FIELD OF THE TECHNOLOGY

The present disclosure relates to antibodies or antigen-binding fragments that are useful for treating infections caused by coronaviruses (e.g., SARS-CoV-2). The present invention also relates to various pharmaceutical compositions and methods of treating coronavirus infections (e.g., COVID-19) using the antibodies or antigen-binding fragments.

### REFERENCE TO SEQUENCE LISTING

This application contains a Sequence Listing that has been submitted in ASCII format via EFS-Web and is hereby incorporated by reference in its entirety. The ASCII copy, created on Mar. 11, 2022, is named Untitled_ST25.txt, and is 90,524 bytes in size.

### BACKGROUND

Several members of the family Coronaviridae typically affect the respiratory tract of mammals, including humans, and usually cause mild respiratory disease. In the past two decades, however, two highly pathogenic coronaviruses (CoVs), including severe acute respiratory syndrome coronavirus (SARS-CoV) and Middle East respiratory syndrome coronavirus (MERS-CoV), have crossed the species barrier and led to global epidemics with high morbidity and mortality. SARS-CoV first appeared in 2002 in the Guangdong province of China and then quickly spread as a global epidemic in more than 30 countries, infecting 8,098 people and causing 774 deaths. In 2012, MERS-CoV emerged in the Arabian Peninsula, and its subsequent spread to 27 countries was associated with 2,494 confirmed cases and 858 deaths. In December 2019, the third highly pathogenic human coronavirus (HCoV), 2019 novel coronavirus (2019-nCoV), as denoted by the World Health Organization (WHO), was discovered in Wuhan, Hubei province of China. 2019-nCoV, with 79.5 and 96% sequence identity to SARS-CoV and a bat coronavirus, SL-CoV-RaTG13, respectively, was then renamed SARS-CoV-2 by the Coronaviridae Study Group (CSG) of the International Committee on Taxonomy of Viruses (ICTV). Compared to SARS-CoV and MERS-CoV, SARS-CoV-2 appears to be more readily transmitted from human-to-human, spreading to multiple continents and leading to the WHO declaration of a global pandemic on Mar. 11, 2020.

There is a need for novel treatments for treating this novel and virulent infection. For example, specific antibodies that can target and neutralize SARS-CoV-2 (or other related SARS or MERS coronaviruses) could be used to treat or prevent active COVID-19 infections.

### BRIEF DESCRIPTION OF THE FIGURES

The application file contains at least one photograph executed in color. Copies of this patent application publication with color photographs will be provided by the Office upon request and payment of the necessary fee.

FIG. 1A, FIG. 1B, FIG. 1C and FIG. 1D shows plasmablast and antibody response to SARS-CoV-2 immunization. FIG. 1A shows the study design. Forty-one healthy adult volunteers (ages 28-73, 8 with a history of SARS-CoV-2 infection) were enrolled and received the BNT162b2 mRNA SARS-CoV-2 vaccine. Blood was collected before immunization, and at 3, 4, 5, 7 and 15 weeks after immunization. For 14 participants (ages 28-52, none with a history of SARS-CoV-2 infection), FNAs of ipsilateral axillary lymph nodes (LNs) were collected at 3, 4, 5, 7 and 15 weeks after immunization. FIG. 1B and FIG. 1C show ELISpot quantification of S-binding IgG- (FIG. 1B) and IgA- (FIG. 1C) secreting plasmablasts (PBs) in blood at baseline, and at 3, 4, 5 and 7 weeks after immunization in participants without (red) and with (black) a history of SARS-CoV-2 infection. FIG. 1D shows plasma IgG titres against SARS-CoV-2 S (left) and the RBD of S (right) measured by ELISA in participants without (red) and with (black) a history of SARS-CoV-2 infection at baseline, and at 3, 4, 5, 7 and 15 weeks after immunization. Dotted lines indicate limits of detection. Symbols at each time point in b-d represent one sample (n=41). Results are from one experiment performed in duplicate.

FIG. 2A and FIG. 2B show antibody response to SARS-CoV-2 immunization. FIG. 2A shows the plasma IgA (left) and IgM (right) titres against SARS-CoV-2 S measured by ELISA in participants without (red) and with (black) a history of SARS-CoV-2 infection at baseline, and 3, 4, 5, 7 and 15 weeks after immunization. FIG. 2B shows neutralizing activity of serum against WA1/2020 D614G (left), B.1.1.7 (middle) and a chimeric virus expressing B.1.351 S (right) in Vero-TMPRSS2 cells at baseline, 3, and 5 or 7 weeks after immunization in participants without (red) and with (black) a history of SARS-CoV-2 infection. P values from two-sided Mann-Whitney tests. Dotted lines indicate limits of detection. Horizontal lines indicate the geometric mean. Symbols at each time point represent one sample (n=41). Results are from one experiment performed in duplicate.

FIG. 3A, FIG. 3B, FIG. 3C, FIG. 3D, and FIG. 3E show germinal centre B cell response to SARS-CoV-2 immunization. FIG. 3A show representative colour Doppler ultrasound image of two draining lymph nodes (‘1’ and ‘2’) adjacent to the axillary vein ‘LAX V’ 5 weeks after immunization. FIG. 3B and FIG. 3C show representative flow cytometry plots of BCL6 and CD38 staining on IgDlowCD19⁺CD3-live singlet lymphocytes in FNA samples (FIG. 3B; LN1, top row; LN2, bottom row) and S staining on BCL6⁺CD38^{int} germinal centre B cells in tonsil and FNA samples (FIG. 3C) at the indicated times after immunization. FIG. 3D and FIG. 3E show kinetics of total (blue) and S+(white) germinal centre (GC) B cells as gated in b and c (FIG. 3D) and S-binding percent of germinal centre B cells (FIG. 3E) from FNA of draining lymph nodes. Symbols at each time point represent one FNA sample; square symbols denote the second lymph node sampled (n=14). Horizontal lines indicate the median.

FIG. 4A, FIG. 4B, and FIG. 4C show gating strategies for analysis of germinal centre response to SARS-CoV-2 immunization. FIG. 4A and FIG. 4B show sorting gating strategies for S-binding germinal centre B cells from FNAs (FIG. 4A) and total plasmablasts from PBMCs (FIG. 4C). FIG. 4B show representative plot of germinal centre B cells in tonsil.

FIG. 5A, FIG. 5B, FIG. 5C, and FIG. 5D show clonal analysis of germinal centre response to SARS-CoV-2 immunization. FIG. 5A show binding of monoclonal antibodies (mAbs) generated from germinal centre B cells to SARS-CoV-2 S, N-terminal domain (NTD) of S, RBD, or S proteins of betacoronavirus OC43 or HKU1, measured by ELISA. Results are from one experiment performed in duplicate. Baseline for area under the curve was set to the mean+three times the s.d. of background binding to bovine serum albumin. FIG. 5B shows clonal relationship of sequences from S-binding germinal centre-derived monoclonal antibodies (cyan) to sequences from bulk repertoire analysis of plasmablasts from PBMCs (red) and germinal centre B cells (blue) sorted 4 weeks after immunization. Each clone is visualized as a network in which each node represents a sequence and sequences are linked as a minimum spanning tree of the network. Symbol shape indicates sequence isotype: IgG (circle), IgA (star) and IgM (square); symbol size corresponds to sequence count. FIG. 5C and FIG. 5D show comparison of nucleotide mutation frequency in IGHV genes of naive B cells sorted from individuals vaccinated with influenza virus vaccine (grey) to clonal relatives of S-binding monoclonal antibodies among plasmablasts sorted from PBMCs and germinal centre B cells 4 weeks after immunization (green) in indicated participants (FIG. 5C) and between clonal relatives of S-binding monoclonal antibodies cross-reactive (purple) or not (teal) to seasonal coronavirus S proteins among plasmablasts sorted from PBMCs and germinal centre B cells 4 weeks after immunization (FIG. 5D). Horizontal lines and error bars indicate the median and interquartile range. Sequence counts were 2,553 (naive), 199 (participant 07), 6 (participant 20), 240 (participant 22), 54 (cross-reactive) and 391 (not cross-reactive). P values from two-sided Kruskal-Wallis test with Dunn's post-test between naive B cells and S-binding clones (FIG. 5C) or two-sided Mann-Whitney U test (FIG. 5D).

FIG. 6A and FIG. 6B show clonal analysis of germinal centre response to SARS-CoV-2 immunization. FIG. 6A shows a distance-to-nearest-neighbour plots for choosing a distance threshold for inferring clones via hierarchical clustering. After partitioning sequences based on common V and J genes and CDR3 length, the nucleotide Hamming distance of a CDR3 to its nearest nonidentical neighbour from the same participant within its partition was calculated and normalized by CDR3 length (blue histogram). For reference, the distance to the nearest nonidentical neighbour from other participants was calculated (green histogram). A clustering threshold of 0.15 (dashed black line) was chosen via manual inspection and kernel density estimate (dashed purple line) to separate the two modes of the within-participant distance distribution representing, respectively, sequences that were probably clonally related and unrelated. FIG. 6B shows clonal relationship of sequences from S-binding germinal centre-derived monoclonal antibodies (cyan) to sequences from bulk repertoire analysis of plasmablasts sorted from PBMCs (red) and germinal centre B cells (blue) 4 weeks after immunization. Each clone is visualized as a network in which each node represents a sequence and sequences are linked as a minimum spanning tree of the network. Symbol shape indicates sequence isotype: IgG (circle), IgA (star) and IgM (square); symbol size corresponds to sequence count.

FIG. 7A, FIG. 7B, FIG. 7C, and FIG. 7D show lymph node plasmablast response to SARS-CoV-2 immunization. FIG. 7A and FIG. 7C show representative flow cytometry plots showing gating of CD20^{low}CD38⁺CD71⁺BLIMP1⁺S⁺ plasmablasts from IgD^{low}CD19⁺CD3⁻ live singlet lymphocytes (FIG. 7A) and IgA and IgM staining on S⁺ plasmablasts (FIG. 7C) in FNA samples. FIG. 7B shows the kinetics of S⁺ plasmablasts gated as in a from FNA of draining lymph nodes. Symbols at each time point represent one FNA sample; square symbols denote second lymph node sampled (n=14). Horizontal lines indicate the median. FIG. 7D shows the percentages of IgM⁺ (teal), IgA⁺ (yellow) or IgM⁻IgA⁻ (purple) S⁺ plasmablasts gated as in c in FNA of draining lymph nodes 4 weeks after primary immunization. Each bar represents one sample (n=14).

FIG. 8A and FIG. 8B show mAb 2C08 potently neutralizes diverse SARS-CoV-2 strains. FIG. 8A and FIG. 8B show ELISA binding to recombinant RBD from (FIG. 8A) and neutralizing activity in Vero-TMPRSS2 cells against (FIG. 8A) indicated SARS-CoV-2 strains by the indicated mAbs. ELISA binding to D614G RBD previously reported. Baseline for area under the curve was set to the mean+three times the standard deviation of background binding to bovine serum albumin. Dotted lines indicate limit of detection. Bars indicate mean±SEM. Results are from one experiment performed in duplicate (panel A, D614G) or in singlet (panel A, B.1.1.7, B.1.351, and B.1.1.248), or two experiments performed in duplicate (panel B).

FIG. 9A, FIG. 9B, FIG. 9C and FIG. 9D show mAb 2C08 protects hamsters from SARS-CoV-2 challenge. FIG. 9A-FIG. 9D show percent weight change (FIG. 9A), lung viral RNA titer (FIG. 9B), lung infectious virus titer (FIG. 9C), and lung cytokine gene expression (FIG. 9D) of hamsters that received isotype (black) or 2C08 (grey) one day prior to intranasal challenge with 5×10⁵ PFU D614G (left) or B.1.351 (right) SARS-CoV-2. In (FIG. 9A), symbols indicate mean±SEM. In (FIG. 9B and FIG. 9C), bars indicate geometric mean±geometric SD, and each symbol represents one hamster. In (FIG. 9D), bars indicate mean±SD, and each symbol represents one hamster. Data are from one experiment, n=5 per condition. P-values from two-tailed Mann-Whitney tests (FIG. 9A-FIG. 9C) and unpaired two-tailed t-tests (FIG. 9D).

FIG. 10 shows mAb 2C08 protects hamsters from SARS-CoV-2 challenge. Lung viral RNA titer using 5′ UTR probe of hamsters that received isotype (black) or 2C08 (grey) one day prior to intranasal challenge with 105 TCID50 D614G (left) or B.1.351 (right) SARS-CoV-2 variants. Bars indicate geometric mean±geometric SD, and each symbol represents one hamster. Data are from one experiment, n=5 per condition. P-values from two-tailed Mann-Whitney tests.

FIG. 11A, FIG. 11B, and FIG. 11C shows mAb 2C08 recognizes a public epitope in SARS-CoV-2 RBD. FIG. 11A shows a plaque assay on Vero cells with no antibody (left) or 2C08 (right) in the overlay to isolate escape mutants (red arrow). Data are representative of three experiments. FIG. 11B shows the structure of RBD (from PDB 6M0J) with hACE2 footprint highlighted in magenta and amino acids whose substitution confers resistance to 2C08 in plaque assays highlighted in yellow. FIG. 11C shows a sequence alignment of 2C08 with RBD-binding mAbs from SARS-CoV-2 infected patients and vaccines that utilize the same immunoglobulin heavy and light chain variable region genes (see also Table 4). Stars indicate contact residues. (SEQ ID NOs: 34 and 35)

FIG. 12A and FIG. 12B show Escape mutant mapping of mAB 2C08. FIG. 12A shows 2C08 and a control anti-influenza virus mAb were tested for neutralizing activity against VSV-SARS-CoV-2. The concentration of 2C08 added in the overlay completely inhibited viral infection. Data are representative of two independent experiments. FIG. 12B shows 2C08 escape profile in currently circulating SARS-CoV-2 viruses isolated from humans. For each site of escape, we counted the sequences in GISAID with an amino acid change (829,521 total sequences at the time of the analysis). Variant circulating frequency is represented as a rainbow color map from red (less circulating with low frequency) to violet (most circulating with high frequency). A black cell indicates the variant has not yet been isolated from a patient. A rainbow cell with cross indicates the variant has been isolated from a patient, but not appear in those 2C08 mAb escape mutants.

FIG. 13A and FIG. 13B show mAb 2C08 recognizes a public epitope in SARS-CoV-2 RBD. FIG. 13A and FIG. 13B show structures of mAbs S2E12 (PDB 7K45) and 253H55L (PDB 7ND9) complexed with RBD and their heavy (pink) and light (red) chain CDR3 sequence alignments with 2C08. (SEQ ID NOs: 233-235)

### DETAILED DESCRIPTION

The present disclosure is based, at least in part, on the discovery of various antibodies and antigen-binding fragments thereof that show specificity to coronaviruses. Antibodies and antigen-binding fragments thereof described herein can neutralize the virus in vitro and in vivo. Disclosed herein are compositions, methods, and treatment plans for treating an individual who is at risk of having a respiratory viral infection, has mild symptoms of a respiratory viral infection, or has severe symptoms of a respiratory viral infection. A composition of the present disclosure comprising an antibody and/or antigen-binding fragment disclosed herein may be used to treat, prevent, or reduce the infectivity of a respiratory viral infection. A treatment plan may comprise administering a composition (e.g., a composition comprising an antibody and/or antigen-binding fragment of the disclosure) to an individual at risk of having a viral infection or who has a viral infection, thereby preventing or treating the viral infection. In some embodiments, a viral infection may be prevented by reducing the amount of virus capable of binding to a host cell or tissue. For example, a composition of the present disclosure may comprise an antibody and/or antigen-binding fragment of the disclosure and a viral infection may be prevented by disrupting interactions between a viral surface proteins and host cell proteins that activate or enhance insertion of the viral genetic material into the host cell. For example, interactions between a SARS-CoV-2 spike protein, and a host cell ACE-2 receptor.

### I. Definitions

The term “a” or “an” entity refers to one or more of that entity; for example, a “polypeptide subunit” is understood to represent one or more polypeptide subunits. As such, the terms “a” (or “an”), “one or more,” and “at least one” can be used interchangeably herein.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone).

Unless defined otherwise, technical and scientific terms used herein have the same meaning as commonly understood by one of ordinary skill in the art to which this disclosure is related.

Where applicable, units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. Nucleic acid sequences are written from 5′ to 3′, left to right.

The headings provided herein are not limitations of the various aspects and embodiments of the disclosure, which can be had by reference to the specification as a whole.

Terms defined immediately below are more fully defined by reference to the specification in its entirety.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

As used herein, the term “polypeptide” is intended to encompass a singular “polypeptide” as well as plural “polypeptides,” and refers to a molecule composed of amino acid monomers linearly linked by peptide bonds (also known as amide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, hydrophobic interactions, etc., to produce, e.g., a multimeric protein.

As used herein, the term “non-naturally occurring” polypeptide, or any grammatical variants thereof, is a conditional term that explicitly excludes, but only excludes, those forms of the polypeptide that are well-understood by persons of ordinary skill in the art as being “naturally-occurring,” or that are, or might be at any time, determined or interpreted by a judge or an administrative or judicial body to be, “naturally-occurring.”

Other polypeptides disclosed herein are fragments, derivatives, analogs, or variants of the foregoing polypeptides, and any combination thereof. The terms “fragment,” “variant,” “derivative” and “analog” when referring to polypeptide subunit or multimeric protein as disclosed herein can include any polypeptide or protein that retain at least some of the activities of the complete polypeptide or protein, but which is structurally different. Fragments of polypeptides include, for example, proteolytic fragments, as well as deletion fragments. Variants include fragments as described above, and also polypeptides with altered amino acid sequences due to amino acid substitutions, deletions, or insertions. Variants can occur spontaneously or be intentionally constructed. Intentionally constructed variants can be produced using art-known mutagenesis techniques. Variant polypeptides can comprise conservative or non-conservative amino acid substitutions, insertions, and/or deletions. Derivatives are polypeptides that have been altered so as to exhibit additional features not found on the native polypeptide, such as increased resistance to proteolytic degradation. Examples include fusion proteins. Variant polypeptides can also be referred to herein as “polypeptide analogs.” As used herein a “derivative” also refers to a subject polypeptide having one or more amino acids chemically derivatized by reaction of a functional side group. Also included as “derivatives” are those peptides that contain one or more standard or synthetic amino acid derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate protein activity are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

As used herein, the term “binding molecule” refers in its broadest sense to a molecule that specifically binds an antigenic determinant. As described further herein, a binding molecule can comprise one of more “binding domains.” As used herein, a “binding domain” is a two- or three-dimensional polypeptide structure that cans specifically bind a given antigenic determinant, or epitope. A non-limiting example of a binding molecule is an antibody or fragment thereof that comprises a binding domain that specifically binds an antigenic determinant or epitope. Another example of a binding molecule is a bispecific antibody comprising a first binding domain binding to a first epitope, and a second binding domain binding to a second epitope.

Disclosed herein are certain binding molecules, or antigen-binding fragments, variants and/or derivatives thereof. Unless specifically referring to full-sized antibodies such as naturally-occurring antibodies, the term “binding molecule” encompasses full-sized antibodies as well as antigen-binding fragments, variants, analogs, or derivatives of such antibodies, e.g., naturally-occurring antibody or immunoglobulin molecules or engineered antibody molecules or fragments that bind antigen in a manner similar to antibody molecules.

By “specifically binds,” it is meant that a binding molecule, e.g., an antibody or antigen-binding fragment thereof binds to an epitope via its antigen binding domain, and that the binding entails some recognition between the antigen binding domain and the epitope. According to this definition, a binding molecule is said to “specifically bind” to an epitope when it binds to that epitope, via its antigen-binding domain binds more readily than it would bind to a random, unrelated epitope.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition, or disorder or those in which the disease, condition or disorder is to be prevented.

The term “pharmaceutical composition” refers to a preparation that is in such form as to permit the biological activity of the active ingredient to be effective and does not contain components that are unacceptably toxic to a subject to which the composition would be administered. Such composition can be sterile.

An “effective amount” as disclosed herein is an amount sufficient to carry out a specifically stated purpose. An “effective amount” can be determined empirically and in a routine manner, in relation to the stated purpose.

Coronavirus is a family of positive-sense, single-stranded RNA viruses that are known to cause severe respiratory illness. Viruses currently known to infect human from the coronavirus family are from the alphacoronavirus and betacoronavirus genera. Additionally, it is believed that the gammacoronavirus and deltacoronavirus genera may infect humans in the future. Non-limiting examples of betacoronaviruses include Middle East respiratory syndrome coronavirus (MERS-CoV), Severe Acute Respiratory Syndrome coronavirus (SARS-CoV), Human coronavirus HKU1 (HKU1-CoV), Human coronavirus OC43 (OC43-CoV), Murine Hepatitis Virus (MHV-CoV), Bat SARS-like coronavirus WIV1 (WIVI-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV).

The viral genome is capped, polyadenylated, and covered with nucleocapsid proteins. The coronavirus virion includes a viral envelope containing type I fusion glycoproteins referred to as the spike (S) protein. Most coronaviruses have a common genome organization with the replicase gene included in the 5′-portion of the genome, and structural genes included in the 3′-portion of the genome.

Coronavirus Spike (S) protein: A class I fusion glycoprotein initially synthesized as a precursor protein. Individual precursor S polypeptides form a homotrimer and undergo glycosylation within the Golgi apparatus as well as processing to remove the signal peptide, and cleavage by a cellular protease to generate separate SI and S2 polypeptide chains, which remain associated as S1/S2 protomers within the homotrimer and is therefore a trimer of heterodimers. The S1 subunit is distal to the virus membrane and contains the receptor-binding domain (RBD) that mediates virus attachment to its host receptor. The S2 subunit contains fusion protein machinery, such as the fusion peptide, two heptadrepeat sequences (HR1 and HR2) and a central helix typical of fusion glycoproteins, a transmembrane domain, and the cytosolic tail domain.

Coronavirus Spike (S) protein prefusion conformation is a structural conformation adopted by the ectodomain of the coronavirus S protein following processing into a mature coronavirus S protein in the secretory system, and prior to triggering of the fusogenic event that leads to transition of coronavirus S to the post fusion conformation. The three-dimensional structure of an exemplary coronavirus S protein (HKU1-CoV) in a prefusion conformation is disclosed herein and provided in Kirchdoerfer et al., “Prefusion structure of a human coronavirus spike protein,” Nature, 531: 118-121, 2016 (incorporated by reference herein).

A coronavirus S ectodomain trimer “stabilized in a prefusion conformation” comprises one or more amino acid substitutions, deletions, or insertions compared to a native coronavirus S sequence that provide for increased retention of the prefusion conformation compared to coronavirus S ectodomain trimers formed from a corresponding native coronavirus S sequence. The “stabilization” of the prefusion conformation by the one or more amino acid substitutions, deletions, or insertions can be, for example, energetic stabilization (for example, reducing the energy of the prefusion conformation relative to the post fusion open conformation) and/or kinetic stabilization (for example, reducing the rate of transition from the prefusion conformation to the post fusion conformation). Additionally, stabilization of the coronavirus S ectodomain trimer in the prefusion conformation can include an increase in resistance to denaturation compared to a corresponding native coronavirus S sequence. Methods of determining if a coronavirus S ectodomain trimer is in the prefusion conformation are provided herein, and include (but are not limited to) negative-stain electron microscopy and antibody binding assays using a prefusion-conformation-specific antibody.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

In one example, a desired response is to inhibit or reduce or prevent CoV (such as SARS-CoV-2) infection. The CoV infection does not need to be completely eliminated or reduced or prevented for the method to be effective. For example, administration of an effective amount of the immunogen can induce an immune response that decreases the CoV infection (for example, as measured by infection of cells, or by number or percentage of subjects infected by the CoV) by a desired amount, for example by at least 50%, at least 60%, at least 70%, at least 80%, at least 90%, at least 95%, at least 98%, or even at least 100% (elimination or prevention of detectable CoV infection), as compared to a suitable control. Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on coronavirus S ectodomain, such as a SARS-CoV S ectodomain. Epitopes can be formed both from contiguous amino acids or noncontiguous amino acids juxtaposed by tertiary folding of a protein.

The term “antibody,” as used herein, is used in the broadest sense and encompasses various antibody and antibody-like structures, including but not limited to full-length monoclonal, polyclonal, and multispecific (e.g., bispecific, trispecific, etc.) antibodies, as well as heavy chain antibodies and antibody fragments provided they exhibit the desired antigen-binding activity. The domain(s) of an antibody that is involved in binding an antigen is referred to as a “variable region” or “variable domain,” and is described in further detail below. A single variable domain may be sufficient to confer antigen-binding specificity. Preferably, but not necessarily, antibodies useful in the discovery are produced recombinantly. Antibodies may or may not be glycosylated, though glycosylated antibodies may be preferred. An “isolated” antibody is one which has been separated from a component of its natural environment. In some embodiments, an antibody is purified to greater than 95% or 99% purity as determined by methods known in the art.

In addition to antibodies described herein, it may be possible to design an antibody mimetic or an aptamer using methods known in the art that functions substantially the same as an antibody of the invention. An “antibody mimetic” refers to a polypeptide or a protein that can specifically bind to an antigen but is not structurally related to an antibody. Antibody mimetics have a mass of about 3 kDa to about 20 kDa. Non-limiting examples of antibody mimetics are affibody molecules, affilins, affimers, alphabodies, anticalins, avimers, DARPins, and monobodies. Aptamers are a class of small nucleic acid ligands that are composed of RNA or single-stranded DNA oligonucleotides and have high specificity and affinity for their targets. Aptamers interact with and bind to their targets through structural recognition, a process similar to that of an antigen-antibody reaction. Aptamers have a lower molecular weight than antibodies, typically about 8-25 kDa.

The terms “full length antibody” and “intact antibody” may be used interchangeably, and refer to an antibody having a structure substantially similar to a native antibody structure or having heavy chains that contain an Fc region as defined herein. The basic structural unit of a native antibody comprises a tetramer. Each tetramer is composed of two identical pairs of polypeptide chains, each pair having one “light” chain (about 25 kDa) and one “heavy” chain (about 50-70 kDa). Light chains are classified as gamma, mu, alpha, and lambda. Heavy chains are classified as gamma, mu, alpha, delta, or epsilon, and define the antibody's isotype as IgG, IgM, IgA, IgD and IgE, respectively. The amino-terminal portion of each light and heavy chain includes a variable region of about 100 to 110 or more amino acid sequences primarily responsible for antigen recognition (VL and VH, respectively). The carboxy-terminal portion of each chain defines a constant region primarily responsible for effector function. Within light and heavy chains, the variable and constant regions are joined by a “J” region of about 12 or more amino acid sequences, with the heavy chain also including a “D” region of about 10 more amino acid sequences. Intact antibodies are properly cross-linked via disulfide bonds, as is known in the art.

The variable domains of the heavy chain and light chain of an antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Framework region” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence: FR1-HVR1-FR2-HVR2-FR3-HVR3-FR4. The FR domains of a heavy chain and a light chain may differ, as is known in the art.

The term “hypervariable region” or “HVR” as used herein refers to each of the regions of a variable domain which are hypervariable in sequence (also commonly referred to as “complementarity determining regions” or “CDR”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). As used herein, “an HVR derived from a variable region” refers to an HVR that has no more than two amino acid substitutions, as compared to the corresponding HVR from the original variable region. Exemplary HVRs herein include: (a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)); (b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); (c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and (d) combinations of (a), (b), and/or (c), as defined below for various antibodies of this disclosure. Unless otherwise indicated, HVR residues and other residues in the variable domain (e.g., FR residues) are numbered herein according to Kabat et al., supra.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “variant Fc region” comprises an amino acid sequence that can differ from that of a native Fc region by virtue of one or more amino acid substitution(s) and/or by virtue of a modified glycosylation pattern, as compared to a native Fc region or to the Fc region of a parent polypeptide. In an example, a variant Fc region can have from about one to about ten amino acid substitutions, or from about one to about five amino acid substitutions in a native sequence Fc region or in the Fc region of the parent polypeptide. The variant Fc region herein may possess at least about 80% homology, at least about 90% homology, or at least about 95% homology with a native sequence Fc region and/or with an Fc region of a parent polypeptide.

An “antibody fragment” refers to a molecule other than an intact antibody that comprises a portion of an intact antibody that binds the antigen to which the intact antibody binds. Non-limiting examples of antibody fragments include but are not limited to Fv, Fab, Fab′, Fab′-SH, F(ab′)₂; single-chain forms of antibodies and higher order variants thereof; single-domain antibodies, and multispecific antibodies formed from antibody fragments.

Single-chain forms of antibodies, and their higher order forms, may include, but are not limited to, single-domain antibodies, single chain variant fragments (scFvs), divalent scFvs (di-scFvs), trivalent scFvs (tri-scFvs), tetravalent scFvs (tetra-scFvs), diabodies, and triabodies and tetrabodies. ScFv's are comprised of heavy and light chain variable regions connected by a linker. In most instances, but not all, the linker may be a peptide. A linker peptide is preferably from about 5 to 30 amino acids in length, or from about 10 to 25 amino acids in length. Typically, the linker allows for stabilization of the variable domains without interfering with the proper folding and creation of an active binding site. In preferred embodiments, a linker peptide is rich in glycine, as well as serine or threonine. ScFvs can be used to facilitate phage display or can be used for flow cytometry, immunohistochemistry, or as targeting domains. Methods of making and using scFvs are known in the art. ScFvs may also be conjugated to a human constant domain (e.g. a heavy constant domain is derived from an IgG domain, such as IgG1, IgG2, IgG3, or IgG4, or a heavy chain constant domain derived from IgA, IgM, or IgE). Diabodies, triabodies, and tetrabodies and higher order variants are typically created by varying the length of the linker peptide from zero to several amino acids. Alternatively, it is also well known in the art that multivalent binding antibody variants can be generated using self-assembling units linked to the variable domain.

An antibody of the disclosure may be a Dual-affinity Re-targeting Antibody (DART). The DART format is based on the diabody format that separates cognate variable domains of heavy and light chains of the 2 antigen binding specificities on 2 separate polypeptide chains. Whereas the 2 polypeptide chains associate noncovalently in the diabody format, the DART format provides additional stabilization through a C-terminal disulfide bridge. DARTs can be produced in high quantity and quality and reveal exceptional stability in both formulation buffer and human serum.

A “single-domain antibody” refers to an antibody fragment consisting of a single, monomeric variable antibody domain.

Multispecific antibodies include bi-specific antibodies, tri-specific, or antibodies of four or more specificities. Multispecific antibodies may be created by combining the heavy and light chains of one antibody with the heavy and light chains of one or more other antibodies. These chains can be covalently linked.

“Monoclonal antibody” refers to an antibody that is derived from a single copy or clone, including e.g., any eukaryotic, prokaryotic, or phage clone. “Monoclonal antibody” is not limited to antibodies produced through hybridoma technology. Monoclonal antibodies can be produced using hybridoma techniques well known in the art, as well as recombinant technologies, phage display technologies, synthetic technologies or combinations of such technologies and other technologies readily known in the art. Furthermore, the monoclonal antibody may be labeled with a detectable label, immobilized on a solid phase and/or conjugated with a heterologous compound (e.g., an enzyme or toxin) according to methods known in the art.

A “heavy chain antibody” refers to an antibody that consists of two heavy chains. A heavy chain antibody may be an IgG-like antibody from camels, llamas, alpacas, sharks, etc., or an IgNAR from a cartiliaginous fish.

A “humanized antibody” refers to a non-human antibody that has been modified to reduce the risk of the non-human antibody eliciting an immune response in humans following administration but retains similar binding specificity and affinity as the starting non-human antibody. A humanized antibody binds to the same or similar epitope as the non-human antibody. The term “humanized antibody” includes an antibody that is composed partially or fully of amino acid sequences derived from a human antibody germline by altering the sequence of an antibody having non-human hypervariable regions (“HVR”). The simplest such alteration may consist simply of substituting the constant region of a human antibody for the murine constant region, thus resulting in a human/murine chimera which may have sufficiently low immunogenicity to be acceptable for pharmaceutical use. Preferably, the variable region of the antibody is also humanized by techniques that are by now well known in the art. For example, the framework regions of a variable region can be substituted by the corresponding human framework regions, while retaining one, several, or all six non-human HVRs. Some framework residues can be substituted with corresponding residues from a non-human VL domain or VH domain (e.g., the non-human antibody from which the HVR residues are derived), e.g., to restore or improve specificity or affinity of the humanized antibody. Substantially human framework regions have at least about 75% homology with a known human framework sequence (i.e. at least about 75%, at least about 80%, at least about 85%, at least about 90%, at least about 95%, or at least about 99% sequence identity). HVRs may also be randomly mutated such that binding activity and affinity for the antigen is maintained or enhanced in the context of fully human germline framework regions or framework regions that are substantially human. As mentioned above, it is sufficient for use in the methods of this discovery to employ an antibody fragment. Further, as used herein, the term “humanized antibody” refers to an antibody comprising a substantially human framework region, at least one HVR from a nonhuman antibody, and in which any constant region present is substantially human. Substantially human constant regions have at least about 90% with a known human constant sequence (i.e. about 90%, about 95%, or about 99% sequence identity). Hence, all parts of a humanized antibody, except possibly the HVRs, are substantially identical to corresponding pairs of one or more germline human immunoglobulin sequences.

If desired, the design of humanized immunoglobulins may be carried out as follows, or using similar methods familiar to those with skill in the art (for example, see Almagro, et al. Front. Biosci. 2008, 13(5):1619-33). A murine antibody variable region is aligned to the most similar human germline sequences (e.g. by using BLAST or similar algorithm). The CDR residues from the murine antibody sequence are grafted into the similar human “acceptor” germline. Subsequently, one or more positions near the CDRs or within the framework (e.g., Vernier positions) may be reverted to the original murine amino acid in order to achieve a humanized antibody with similar binding affinity to the original murine antibody. Typically, several versions of humanized antibodies with different reversion mutations are generated and empirically tested for activity. The humanized antibody variant with properties most similar to the parent murine antibody and the fewest murine framework reversions is selected as the final humanized antibody candidate.

### II. Composition

Applicant has discovered highly active antibodies that show high specificity for human coronaviruses (e.g., SARS-CoV-2). Accordingly, in various embodiments, the antibody or antigen-binding fragment thereof can selectively bind to a coronavirus. The antibodies and antigen-binding fragments described herein can have important applications, for both therapeutic and prophylactic treatment of coronavirus infections (e.g., COVID-19).

In summary, mAbs were synthesized that are clonally related and bind coronaviruses (e.g., SARS CoV-2). These antibodies are highly active neutralizers of coronavirus (e.g., SARS CoV-2) in vitro and provide broad protection from mortality and morbidity in vivo. The discovery of these mAbs raises the hope that similar antibodies can be induced in the population if the right vaccination regimen is given. Knowledge about the binding mode and epitope of these mAbs may then guide the development of universal COVID-19 vaccines.

### a) Anti-Coronavirus Spike Antibodies

The antibodies disclosed herein can be described or specified in terms of the epitope(s) that they recognize or bind. The portion of a target polypeptide that specifically interacts with the antigen binding domain of an antibody is an “epitope.” Furthermore, it should be noted that an “epitope” on can be a linear epitope or a conformational epitope, and in both instances can include non-polypeptide elements, e.g., an epitope can include a carbohydrate or lipid side chain. The term “affinity” refers to a measure of the strength of the binding of an individual epitope with an antibody's antigen binding site. In some embodiments, the epitope is an epitope in a coronavirus spike protein. In one aspect, the epitope is within the receptor binding domain (RBD). In a particular aspect, an epitope within the RBD is an epitope within amino acids 319-541 of a coronavirus spike protein. In other aspect, an epitope is within the N-terminal domain of a coronavirus spike protein.

An “anti-coronavirus spike antibody,” as used herein, refers to an isolated antibody that binds to recombinant human coronavirus spike protein or human coronavirus spike protein isolated from biological sample with an affinity constant or affinity of interaction (KD) between about 0.1 pM to about 10 μM, preferably about 0.1 pM to about 1 μM, more preferably about 0.1 pM to about 100 nM. Methods for determining the affinity of an antibody for an antigen are known in the art. Anti-coronavirus spike antibodies useful herein include those which are suitable for administration to a subject in a therapeutic amount.

Anti-coronavirus spike antibodies disclosed herein can also be described or specified in terms of their cross-reactivity. The term “cross-reactivity” refers to the ability of an antibody, specific for one antigen, to react with a second antigen; a measure of relatedness between two different antigenic substances. Thus, an antibody is cross-reactive if it binds to an epitope other than the one that induced its formation. The cross-reactive epitope generally contains many of the same complementary structural features as the inducing epitope, and in some cases, can actually fit better than the original. For example, certain antibodies have some degree of cross-reactivity, in that they bind related, but non-identical epitopes, e.g., epitopes with at least about 85%, at least about 90%, or at least about 95% identity (as calculated using methods known in the art) to a reference epitope. An antibody can be said to have little or no cross-reactivity if it does not bind epitopes with less than about 95%, less than about 90%, or less than about 85% identity to a reference epitope. An antibody can be deemed “highly specific” for a certain epitope, if it does not bind any other analog, ortholog, or homolog of that epitope.

Other aspects of anti-coronavirus spike antibodies of this disclosure are described more thoroughly below.

i) Anti-Coronavirus Spike Antibody

In an exemplary embodiment, an anti-coronavirus spike antibody comprises a VL that has one or more HVRs derived from SEQ ID NO: 6 or a VH that has one or more HVRs derived from SEQ ID NO: 7. The HVR derived from SEQ ID NO: 6 may be L1, L2, L3, or any combination thereof. In certain embodiments, the VL may comprise an L1 of SEQ ID NO: 1, an L2 of DAS, an L3 of SEQ ID NO: 2, or any combination thereof (e.g. antibodies 1-7 in Table A). The HVR derived from SEQ ID NO: 7 may be H1, H2, H3, or any combination thereof. In certain embodiments, the VH may comprise an H1 of SEQ ID NO: 3, an H2 of SEQ ID NO: 4, an H3 of SEQ ID NO: 5, or any combination thereof (e.g. antibodies 8-14 in Table A). The antibody comprising one or more HVRs derived from SEQ ID NO: 7 may further comprise a light chain variable region (VL) comprising one or more HVRs derived from SEQ ID NO: 6. The HVR may be L1, L2, L3, or any combination thereof. In a preferred embodiment, the VL may comprise an L1 of SEQ ID NO: 1, an L2 of DAS, an L3 of SEQ ID NO: 2, or any combination thereof (e.g. antibodies 15-63 in Table A). In various embodiments above, the antibody may be a humanized antibody, or the antibody may have a VL with 90, 91, 92, 93, 94, 95, 96, 97, 98, 99, or 100% identity to SEQ ID NO: 6 and/or a VH with 90, 91, 92, 93, 94, 95, 96, 97, 98, 99 or 100% identity to SEQ ID NO: 7. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 1, 2, 3, 4, 5, 6, 7, and the amino acid sequence DAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 13 or a VH that has one or more HVRs derived from SEQ ID NO: 14. The HVR derived from SEQ ID NO: 13 may be L1, L2, L3, or any combination thereof. In certain embodiments, the VL may comprise an L1 of SEQ ID NO: 8, an L2 of AAS, an L3 of SEQ ID NO: 9, or any combination thereof (e.g. antibodies 64-70 in Table A). The HVR derived from SEQ ID NO: 14 may be H1, H2, H3, or any combination thereof. In certain embodiments, the VH may comprise an H1 of SEQ ID NO: 10, an H2 of SEQ ID NO: 11, an H3 of SEQ ID NO: 12, or any combination thereof (e.g. antibodies 71-77 in Table A). The antibody comprising one or more HVRs derived from SEQ ID NO: 14 may further comprise a light chain variable region (VL) comprising one or more HVRs derived from SEQ ID NO: 13. The HVR may be L1, L2, L3, or any combination thereof. In a preferred embodiment, the VL may comprise an L1 of SEQ ID NO: 8, an L2 of AAS, an L3 of SEQ ID NO: 9, or any combination thereof (e.g. antibodies 78-126 in Table A). In various embodiments above, the antibody may be a humanized antibody, or the antibody may have a VL with 90, 91, 92, 93, 94, 95, 96, 97, 98, 99, or 100% identity to SEQ ID NO: 13 and/or a VH with 90, 91, 92, 93, 94, 95, 96, 97, 98, 99 or 100% identity to SEQ ID NO: 14. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 8, 9, 10, 11, 12, 13, 14, and the amino acid sequence AAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 20 or a VH that has one or more HVRs derived from SEQ ID NO: 21. The HVR derived from SEQ ID NO: 20 may be L1, L2, L3, or any combination thereof. In certain embodiments, the VL may comprise an L1 of SEQ ID NO: 15, an L2 of QDN, an L3 of SEQ ID NO: 16, or any combination thereof (e.g. antibodies 127-133 in Table A). The HVR derived from SEQ ID NO: 21 may be H1, H2, H3, or any combination thereof. In certain embodiments, the VH may comprise an H1 of SEQ ID NO: 17, an H2 of SEQ ID NO: 18, an H3 of SEQ ID NO: 19, or any combination thereof (e.g. antibodies 134-140 in Table A). The antibody comprising one or more HVRs derived from SEQ ID NO: 21 may further comprise a light chain variable region (VL) comprising one or more HVRs derived from SEQ ID NO: 20. The HVR may be L1, L2, L3, or any combination thereof. In a preferred embodiment, the VL may comprise an L1 of SEQ ID NO: 15, an L2 of QDN, an L3 of SEQ ID NO: 16, or any combination thereof (e.g. antibodies 141-189 in Table A). In various embodiments above, the antibody may be a humanized antibody, or the antibody may have a VL with 90, 91, 92, 93, 94, 95, 96, 97, 98, 99, or 100% identity to SEQ ID NO: 20 and/or a VH with 90, 91, 92, 93, 94, 95, 96, 97, 98, 99 or 100% identity to SEQ ID NO: 21. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 15, 16, 17, 18, 19, 20, 21, and the amino acid sequence QDN, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 27 or a VH that has one or more HVRs derived from SEQ ID NO: 28. The HVR derived from SEQ ID NO: 27 may be L1, L2, L3, or any combination thereof. In certain embodiments, the VL may comprise an L1 of SEQ ID NO: 22, an L2 of DAS, an L3 of SEQ ID NO: 23, or any combination thereof (e.g. antibodies 190-196 in Table A). The HVR derived from SEQ ID NO: 28 may be H1, H2, H3, or any combination thereof. In certain embodiments, the VH may comprise an H1 of SEQ ID NO: 24, an H2 of SEQ ID NO: 25, an H3 of SEQ ID NO: 26, or any combination thereof (e.g. antibodies 197-203 in Table A). The antibody comprising one or more HVRs derived from SEQ ID NO: 28 may further comprise a light chain variable region (VL) comprising one or more HVRs derived from SEQ ID NO: 27. The HVR may be L1, L2, L3, or any combination thereof. In a preferred embodiment, the VL may comprise an L1 of SEQ ID NO: 22, an L2 of DAS, an L3 of SEQ ID NO: 23, or any combination thereof (e.g. antibodies 204-253 in Table A). In various embodiments above, the antibody may be a humanized antibody, or the antibody may have a VL with 90, 91, 92, 93, 94, 95, 96, 97, 98, 99, or 100% identity to SEQ ID NO: 27 and/or a VH with 90, 91, 92, 93, 94, 95, 96, 97, 98, 99 or 100% identity to SEQ ID NO: 28. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 22, 23, 24, 25, 26, 27, 28, and the amino acid sequence DAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 34 or a VH that has one or more HVRs derived from SEQ ID NO: 35. The HVR derived from SEQ ID NO: 34 may be L1, L2, L3, or any combination thereof. In certain embodiments, the VL may comprise an L1 of SEQ ID NO: 29, an L2 of ATS, an L3 of SEQ ID NO: 30, or any combination thereof (e.g. antibodies 254-260 in Table A). The HVR derived from SEQ ID NO: 35 may be H1, H2, H3, or any combination thereof. In certain embodiments, the VH may comprise an H1 of SEQ ID NO: 31, an H2 of SEQ ID NO: 32, an H3 of SEQ ID NO: 33, or any combination thereof (e.g. antibodies 261-267 in Table A). The antibody comprising one or more HVRs derived from SEQ ID NO: 35 may further comprise a light chain variable region (VL) comprising one or more HVRs derived from SEQ ID NO: 34. The HVR may be L1, L2, L3, or any combination thereof. In a preferred embodiment, the VL may comprise an L1 of SEQ ID NO: 29, an L2 of ATS, an L3 of SEQ ID NO: 30, or any combination thereof (e.g. antibodies 268-316 in Table A). In various embodiments above, the antibody may be a humanized antibody, or the antibody may have a VL with 90, 91, 92, 93, 94, 95, 96, 97, 98, 99, or 100% identity to SEQ ID NO: 34 and/or a VH with 90, 91, 92, 93, 94, 95, 96, 97, 98, 99 or 100% identity to SEQ ID NO: 35. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 29, 30, 31, 32, 33, 34, 35, and the amino acid sequence ATS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 41 or a VH that has one or more HVRs derived from SEQ ID NO: 42. The HVR derived from SEQ ID NO: 41 may be L1, L2, L3, or any combination thereof. In certain embodiments, the VL may comprise an L1 of SEQ ID NO: 36, an L2 of EDN, an L3 of SEQ ID NO: 37, or any combination thereof (e.g. antibodies 317-323 in Table A). The HVR derived from SEQ ID NO: 42 may be H1, H2, H3, or any combination thereof. In certain embodiments, the VH may comprise an H1 of SEQ ID NO: 38, an H2 of SEQ ID NO: 39, an H3 of SEQ ID NO: 40, or any combination thereof (e.g. antibodies 324-330 in Table A). The antibody comprising one or more HVRs derived from SEQ ID NO: 42 may further comprise a light chain variable region (VL) comprising one or more HVRs derived from SEQ ID NO: 41. The HVR may be L1, L2, L3, or any combination thereof. In a preferred embodiment, the VL may comprise an L1 of SEQ ID NO: 36, an L2 of EDN, an L3 of SEQ ID NO: 37, or any combination thereof (e.g. antibodies 331-379 in Table A). In various embodiments above, the antibody may be a humanized antibody, or the antibody may have a VL with 90, 91, 92, 93, 94, 95, 96, 97, 98, 99, or 100% identity to SEQ ID NO: 41 and/or a VH with 90, 91, 92, 93, 94, 95, 96, 97, 98, 99 or 100% identity to SEQ ID NO: 42. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 36, 37, 38, 39, 40, 41, 42, and the amino acid sequence EDN, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 48 or a VH that has one or more HVRs derived from SEQ ID NO: 49. The HVR derived from SEQ ID NO: 48 may be L1, L2, L3, or any combination thereof. In certain embodiments, the VL may comprise an L1 of SEQ ID NO: 43, an L2 of DAS, an L3 of SEQ ID NO: 44, or any combination thereof (e.g. antibodies 380-386 in Table A). The HVR derived from SEQ ID NO: 49 may be H1, H2, H3, or any combination thereof. In certain embodiments, the VH may comprise an H1 of SEQ ID NO: 45, an H2 of SEQ ID NO: 46, an H3 of SEQ ID NO: 47, or any combination thereof (e.g. antibodies 387-393 in Table A). The antibody comprising one or more HVRs derived from SEQ ID NO: 49 may further comprise a light chain variable region (VL) comprising one or more HVRs derived from SEQ ID NO: 48. The HVR may be L1, L2, L3, or any combination thereof. In a preferred embodiment, the VL may comprise an L1 of SEQ ID NO: 43, an L2 of DAS, an L3 of SEQ ID NO: 44, or any combination thereof (e.g. antibodies 394-442 in Table A). In various embodiments above, the antibody may be a humanized antibody, or the antibody may have a VL with 90, 91, 92, 93, 94, 95, 96, 97, 98, 99, or 100% identity to SEQ ID NO: 48 and/or a VH with 90, 91, 92, 93, 94, 95, 96, 97, 98, 99 or 100% identity to SEQ ID NO: 49. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 43, 44, 45, 46, 47, 48, 49, and the amino acid sequence DAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 55 or a VH that has one or more HVRs derived from SEQ ID NO: 56. The HVR derived from SEQ ID NO: 55 may be L1, L2, L3, or any combination thereof. In certain embodiments, the VL may comprise an L1 of SEQ ID NO: 50, an L2 of WAS, an L3 of SEQ ID NO: 51, or any combination thereof (e.g. antibodies 443-449 in Table A). The HVR derived from SEQ ID NO: 56 may be H1, H2, H3, or any combination thereof. In certain embodiments, the VH may comprise an H1 of SEQ ID NO: 52, an H2 of SEQ ID NO: 53, an H3 of SEQ ID NO: 54, or any combination thereof (e.g. antibodies 450-456 in Table A). The antibody comprising one or more HVRs derived from SEQ ID NO: 56 may further comprise a light chain variable region (VL) comprising one or more HVRs derived from SEQ ID NO: 55. The HVR may be L1, L2, L3, or any combination thereof. In a preferred embodiment, the VL may comprise an L1 of SEQ ID NO: 50, an L2 of WAS, an L3 of SEQ ID NO: 51, or any combination thereof (e.g. antibodies 457-505 in Table A). In various embodiments above, the antibody may be a humanized antibody, or the antibody may have a VL with 90, 91, 92, 93, 94, 95, 96, 97, 98, 99, or 100% identity to SEQ ID NO: 55 and/or a VH with 90, 91, 92, 93, 94, 95, 96, 97, 98, 99 or 100% identity to SEQ ID NO: 56. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 50, 51, 52, 53, 54, 55, 56, and the amino acid sequence WAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 62 or a VH that has one or more HVRs derived from SEQ ID NO: 63. The HVR derived from SEQ ID NO: 62 may be L1, L2, L3, or any combination thereof. In certain embodiments, the VL may comprise an L1 of SEQ ID NO: 57, an L2 of EVS, an L3 of SEQ ID NO: 58, or any combination thereof (e.g. antibodies 506-512 in Table A). The HVR derived from SEQ ID NO: 63 may be H1, H2, H3, or any combination thereof. In certain embodiments, the VH may comprise an H1 of SEQ ID NO: 58, an H2 of SEQ ID NO: 59, an H3 of SEQ ID NO: 60, or any combination thereof (e.g. antibodies 513-519 in Table A). The antibody comprising one or more HVRs derived from SEQ ID NO: 63 may further comprise a light chain variable region (VL) comprising one or more HVRs derived from SEQ ID NO: 62. The HVR may be L1, L2, L3, or any combination thereof. In a preferred embodiment, the VL may comprise an L1 of SEQ ID NO: 57, an L2 of EVS, an L3 of SEQ ID NO: 58, or any combination thereof (e.g. antibodies 520-568 in Table A). In various embodiments above, the antibody may be a humanized antibody, or the antibody may have a VL with 90, 91, 92, 93, 94, 95, 96, 97, 98, 99, or 100% identity to SEQ ID NO: 62 and/or a VH with 90, 91, 92, 93, 94, 95, 96, 97, 98, 99 or 100% identity to SEQ ID NO: 63. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 57, 58, 59, 60, 61, 62, 63, and the amino acid sequence EVS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 69 or a VH that has one or more HVRs derived from SEQ ID NO: 70. The HVR derived from SEQ ID NO: 69 may be L1, L2, L3, or any combination thereof. In certain embodiments, the VL may comprise an L1 of SEQ ID NO: 64, an L2 of GAS, an L3 of SEQ ID NO: 65, or any combination thereof (e.g. antibodies 567-575 in Table A). The HVR derived from SEQ ID NO: 70 may be H1, H2, H3, or any combination thereof. In certain embodiments, the VH may comprise an H1 of SEQ ID NO: 66, an H2 of SEQ ID NO: 67, an H3 of SEQ ID NO: 68, or any combination thereof (e.g. antibodies 578-582 in Table A). The antibody comprising one or more HVRs derived from SEQ ID NO: 70 may further comprise a light chain variable region (VL) comprising one or more HVRs derived from SEQ ID NO: 69. The HVR may be L1, L2, L3, or any combination thereof. In a preferred embodiment, the VL may comprise an L1 of SEQ ID NO: 64, an L2 of GAS, an L3 of SEQ ID NO: 65, or any combination thereof (e.g. antibodies 583-631 in Table A). In various embodiments above, the antibody may be a humanized antibody, or the antibody may have a VL with 90, 91, 92, 93, 94, 95, 96, 97, 98, 99, or 100% identity to SEQ ID NO: 69 and/or a VH with 90, 91, 92, 93, 94, 95, 96, 97, 98, 99 or 100% identity to SEQ ID NO: 70. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 64, 65, 66, 67, 68, 69, 70, and the amino acid sequence GAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 76 or a VH that has one or more HVRs derived from SEQ ID NO: 77. The HVR derived from SEQ ID NO: 76 may be L1, L2, L3, or any combination thereof. In certain embodiments, the VL may comprise an L1 of SEQ ID NO: 71, an L2 of EDS, an L3 of SEQ ID NO: 72, or any combination thereof (e.g. antibodies 632-638 in Table A). The HVR derived from SEQ ID NO: 77 may be H1, H2, H3, or any combination thereof. In certain embodiments, the VH may comprise an H1 of SEQ ID NO: 73, an H2 of SEQ ID NO: 74, an H3 of SEQ ID NO: 75, or any combination thereof (e.g. antibodies 639-645 in Table A). The antibody comprising one or more HVRs derived from SEQ ID NO: 77 may further comprise a light chain variable region (VL) comprising one or more HVRs derived from SEQ ID NO: 76. The HVR may be L1, L2, L3, or any combination thereof. In a preferred embodiment, the VL may comprise an L1 of SEQ ID NO: 71, an L2 of EDS, an L3 of SEQ ID NO: 72, or any combination thereof (e.g. antibodies 646-694 in Table A). In various embodiments above, the antibody may be a humanized antibody, or the antibody may have a VL with 90, 91, 92, 93, 94, 95, 96, 97, 98, 99, or 100% identity to SEQ ID NO: 76 and/or a VH with 90, 91, 92, 93, 94, 95, 96, 97, 98, 99 or 100% identity to SEQ ID NO: 77. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 71, 72, 73, 74, 75, 76, 77, and the amino acid sequence EDS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 83 or a VH that has one or more HVRs derived from SEQ ID NO: 84. The HVR derived from SEQ ID NO: 83 may be L1, L2, L3, or any combination thereof. In certain embodiments, the VL may comprise an L1 of SEQ ID NO: 78, an L2 of EDS, an L3 of SEQ ID NO: 79, or any combination thereof (e.g. antibodies 695-701 in Table A). The HVR derived from SEQ ID NO: 84 may be H1, H2, H3, or any combination thereof. In certain embodiments, the VH may comprise an H1 of SEQ ID NO: 80, an H2 of SEQ ID NO: 81, an H3 of SEQ ID NO: 82, or any combination thereof (e.g. antibodies 702-708 in Table A). The antibody comprising one or more HVRs derived from SEQ ID NO: 84 may further comprise a light chain variable region (VL) comprising one or more HVRs derived from SEQ ID NO: 85. The HVR may be L1, L2, L3, or any combination thereof. In a preferred embodiment, the VL may comprise an L1 of SEQ ID NO: 78, an L2 of SEQ ID NO: EDS, an L3 of SEQ ID NO: 79, or any combination thereof (e.g. antibodies 709-757 in Table A). In various embodiments above, the antibody may be a humanized antibody, or the antibody may have a VL with 90, 91, 92, 93, 94, 95, 96, 97, 98, 99, or 100% identity to SEQ ID NO: 83 and/or a VH with 90, 91, 92, 93, 94, 95, 96, 97, 98, 99 or 100% identity to SEQ ID NO: 84. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 78, 79, 80, 81, 82, 83, 84, and the amino acid sequence EDS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 90 or a VH that has one or more HVRs derived from SEQ ID NO: 91. The HVR derived from SEQ ID NO: 90 may be L1, L2, L3, or any combination thereof. In certain embodiments, the VL may comprise an L1 of SEQ ID NO: 85, an L2 of DAS, an L3 of SEQ ID NO: 86, or any combination thereof (e.g. antibodies 758-764 in Table A). The HVR derived from SEQ ID NO: 91 may be H1, H2, H3, or any combination thereof. In certain embodiments, the VH may comprise an H1 of SEQ ID NO: 87, an H2 of SEQ ID NO: 88, an H3 of SEQ ID NO: 89, or any combination thereof (e.g. antibodies 765-771 in Table A). The antibody comprising one or more HVRs derived from SEQ ID NO: 91 may further comprise a light chain variable region (VL) comprising one or more HVRs derived from SEQ ID NO: 90. The HVR may be L1, L2, L3, or any combination thereof. In a preferred embodiment, the VL may comprise an L1 of SEQ ID NO: 85, an L2 of DAS, an L3 of SEQ ID NO: 86, or any combination thereof (e.g. antibodies 772-820 in Table A). In various embodiments above, the antibody may be a humanized antibody, or the antibody may have a VL with 90, 91, 92, 93, 94, 95, 96, 97, 98, 99, or 100% identity to SEQ ID NO: 90 and/or a VH with 90, 91, 92, 93, 94, 95, 96, 97, 98, 99 or 100% identity to SEQ ID NO: 91. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 85, 86, 87, 88, 89, 90, 91, and the amino acid sequence DAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 97 or a VH that has one or more HVRs derived from SEQ ID NO: 98. The HVR derived from SEQ ID NO: 97 may be L1, L2, L3, or any combination thereof. In certain embodiments, the VL may comprise an L1 of SEQ ID NO: 92, an L2 of NAS, an L3 of SEQ ID NO: 93, or any combination thereof (e.g. antibodies 758-764 in Table A). The HVR derived from SEQ ID NO: 98 may be H1, H2, H3, or any combination thereof. In certain embodiments, the VH may comprise an H1 of SEQ ID NO: 94, an H2 of SEQ ID NO: 95, an H3 of SEQ ID NO: 96, or any combination thereof (e.g. antibodies 765-771 in Table A). The antibody comprising one or more HVRs derived from SEQ ID NO: 98 may further comprise a light chain variable region (VL) comprising one or more HVRs derived from SEQ ID NO: 97. The HVR may be L1, L2, L3, or any combination thereof. In a preferred embodiment, the VL may comprise an L1 of SEQ ID NO: 92, an L2 of NAS, an L3 of SEQ ID NO: 93, or any combination thereof (e.g. antibodies 772-820 in Table A). In various embodiments above, the antibody may be a humanized antibody, or the antibody may have a VL with 90, 91, 92, 93, 94, 95, 96, 97, 98, 99, or 100% identity to SEQ ID NO: 97 and/or a VH with 90, 91, 92, 93, 94, 95, 96, 97, 98, 99 or 100% identity to SEQ ID NO: 98. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 92, 93, 94, 95, 96, 97, 98, and the amino acid sequence NAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 104 or a VH that has one or more HVRs derived from SEQ ID NO: 105. The HVR derived from SEQ ID NO: 104 may be L1, L2, L3, or any combination thereof. In certain embodiments, the VL may comprise an L1 of SEQ ID NO: 99, an L2 of WAS, an L3 of SEQ ID NO: 100, or any combination thereof (e.g. antibodies 821-827 in Table A). The HVR derived from SEQ ID NO: 105 may be H1, H2, H3, or any combination thereof. In certain embodiments, the VH may comprise an H1 of SEQ ID NO: 101, an H2 of SEQ ID NO: 102, an H3 of SEQ ID NO: 103, or any combination thereof (e.g. antibodies 828-834 in Table A). The antibody comprising one or more HVRs derived from SEQ ID NO: 105 may further comprise a light chain variable region (VL) comprising one or more HVRs derived from SEQ ID NO: 104. The HVR may be L1, L2, L3, or any combination thereof. In a preferred embodiment, the VL may comprise an L1 of SEQ ID NO: 99, an L2 of WAS, an L3 of SEQ ID NO: 100, or any combination thereof (e.g. antibodies 835-883 in Table A). In various embodiments above, the antibody may be a humanized antibody, or the antibody may have a VL with 90, 91, 92, 93, 94, 95, 96, 97, 98, 99, or 100% identity to SEQ ID NO: 104 and/or a VH with 90, 91, 92, 93, 94, 95, 96, 97, 98, 99 or 100% identity to SEQ ID NO: 105. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 99, 100, 101, 102, 103, 104, 105, and the amino acid sequence WAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 111 or a VH that has one or more HVRs derived from SEQ ID NO: 112. The HVR derived from SEQ ID NO: 111 may be L1, L2, L3, or any combination thereof. In certain embodiments, the VL may comprise an L1 of SEQ ID NO: 106, an L2 of EDS, an L3 of SEQ ID NO: 107, or any combination thereof (e.g. antibodies 884-890 in Table A). The HVR derived from SEQ ID NO: 112 may be H1, H2, H3, or any combination thereof. In certain embodiments, the VH may comprise an H1 of SEQ ID NO: 107, an H2 of SEQ ID NO: 108, an H3 of SEQ ID NO: 109, or any combination thereof (e.g. antibodies 891-897 in Table A). The antibody comprising one or more HVRs derived from SEQ ID NO: 112 may further comprise a light chain variable region (VL) comprising one or more HVRs derived from SEQ ID NO: 111. The HVR may be L1, L2, L3, or any combination thereof. In a preferred embodiment, the VL may comprise an L1 of SEQ ID NO: 107, an L2 of EDS, an L3 of SEQ ID NO: 107, or any combination thereof (e.g. antibodies 898-946 in Table A). In various embodiments above, the antibody may be a humanized antibody, or the antibody may have a VL with 90, 91, 92, 93, 94, 95, 96, 97, 98, 99, or 100% identity to SEQ ID NO: 111 and/or a VH with 90, 91, 92, 93, 94, 95, 96, 97, 98, 99 or 100% identity to SEQ ID NO: 112. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 106, 107, 108, 109, 110, 111, 112, and the amino acid sequence EDS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 118 or a VH that has one or more HVRs derived from SEQ ID NO: 119. The HVR derived from SEQ ID NO: 118 may be L1, L2, L3, or any combination thereof. In certain embodiments, the VL may comprise an L1 of SEQ ID NO: 113, an L2 of DAS, an L3 of SEQ ID NO: 114, or any combination thereof (e.g. antibodies 947-953 in Table A). The HVR derived from SEQ ID NO: 119 may be H1, H2, H3, or any combination thereof. In certain embodiments, the VH may comprise an H1 of SEQ ID NO: 115, an H2 of SEQ ID NO: 116, an H3 of SEQ ID NO: 117, or any combination thereof (e.g. antibodies 954-960 in Table A). The antibody comprising one or more HVRs derived from SEQ ID NO: 119 may further comprise a light chain variable region (VL) comprising one or more HVRs derived from SEQ ID NO: 118. The HVR may be L1, L2, L3, or any combination thereof. In a preferred embodiment, the VL may comprise an L1 of SEQ ID NO: 113, an L2 of DAS, an L3 of SEQ ID NO: 114, or any combination thereof (e.g. antibodies 961-1009 in Table A). In various embodiments above, the antibody may be a humanized antibody, or the antibody may have a VL with 90, 91, 92, 93, 94, 95, 96, 97, 98, 99, or 100% identity to SEQ ID NO: 118 and/or a VH with 90, 91, 92, 93, 94, 95, 96, 97, 98, 99 or 100% identity to SEQ ID NO: 119. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 113, 114, 115, 116, 117, 118, 119, and the amino acid sequence DAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 125 or a VH that has one or more HVRs derived from SEQ ID NO: 126. The HVR derived from SEQ ID NO: 125 may be L1, L2, L3, or any combination thereof. In certain embodiments, the VL may comprise an L1 of SEQ ID NO: 120, an L2 of WAS, an L3 of SEQ ID NO: 121, or any combination thereof (e.g. antibodies 1010-1016 in Table A). The HVR derived from SEQ ID NO: 126 may be H1, H2, H3, or any combination thereof. In certain embodiments, the VH may comprise an H1 of SEQ ID NO: 122, an H2 of SEQ ID NO: 123, an H3 of SEQ ID NO: 124, or any combination thereof (e.g. antibodies 1017-1023 in Table A). The antibody comprising one or more HVRs derived from SEQ ID NO: 126 may further comprise a light chain variable region (VL) comprising one or more HVRs derived from SEQ ID NO: 125. The HVR may be L1, L2, L3, or any combination thereof. In a preferred embodiment, the VL may comprise an L1 of SEQ ID NO: 120, an L2 of WAS, an L3 of SEQ ID NO: 121, or any combination thereof (e.g. antibodies 1024-1072 in Table A). In various embodiments above, the antibody may be a humanized antibody, or the antibody may have a VL with 90, 91, 92, 93, 94, 95, 96, 97, 98, 99, or 100% identity to SEQ ID NO: 125 and/or a VH with 90, 91, 92, 93, 94, 95, 96, 97, 98, 99 or 100% identity to SEQ ID NO: 126. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 120, 121, 122, 123, 124, 125, 126, and the amino acid sequence WAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 132 or a VH that has one or more HVRs derived from SEQ ID NO: 133. The HVR derived from SEQ ID NO: 132 may be L1, L2, L3, or any combination thereof. In certain embodiments, the VL may comprise an L1 of SEQ ID NO: 127, an L2 of EDN, an L3 of SEQ ID NO: 128, or any combination thereof (e.g. antibodies 1073-1079 in Table A). The HVR derived from SEQ ID NO: 133 may be H1, H2, H3, or any combination thereof. In certain embodiments, the VH may comprise an H1 of SEQ ID NO: 129, an H2 of SEQ ID NO: 130, an H3 of SEQ ID NO: 131, or any combination thereof (e.g. antibodies 1080-1086 in Table A). The antibody comprising one or more HVRs derived from SEQ ID NO: 133 may further comprise a light chain variable region (VL) comprising one or more HVRs derived from SEQ ID NO: 132. The HVR may be L1, L2, L3, or any combination thereof. In a preferred embodiment, the VL may comprise an L1 of SEQ ID NO: 127, an L2 of EDN, an L3 of SEQ ID NO: 128, or any combination thereof (e.g. antibodies 1087-1135 in Table A). In various embodiments above, the antibody may be a humanized antibody, or the antibody may have a VL with 90, 91, 92, 93, 94, 95, 96, 97, 98, 99, or 100% identity to SEQ ID NO: 132 and/or a VH with 90, 91, 92, 93, 94, 95, 96, 97, 98, 99 or 100% identity to SEQ ID NO: 133. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 127, 128, 129, 130, 131, 132, 133, and the amino acid sequence EDN, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 139 or a VH that has one or more HVRs derived from SEQ ID NO: 140. The HVR derived from SEQ ID NO: 139 may be L1, L2, L3, or any combination thereof. In certain embodiments, the VL may comprise an L1 of SEQ ID NO: 134, an L2 of DDS, an L3 of SEQ ID NO: 135, or any combination thereof (e.g. antibodies 1136-1142 in Table A). The HVR derived from SEQ ID NO: 140 may be H1, H2, H3, or any combination thereof. In certain embodiments, the VH may comprise an H1 of SEQ ID NO: 136, an H2 of SEQ ID NO: 137, an H3 of SEQ ID NO: 138, or any combination thereof (e.g. antibodies 1143-1149 in Table A). The antibody comprising one or more HVRs derived from SEQ ID NO: 140 may further comprise a light chain variable region (VL) comprising one or more HVRs derived from SEQ ID NO: 139. The HVR may be L1, L2, L3, or any combination thereof. In a preferred embodiment, the VL may comprise an L1 of SEQ ID NO: 134, an L2 of DDS, an L3 of SEQ ID NO: 135, or any combination thereof (e.g. antibodies 1150-1198 in Table A). In various embodiments above, the antibody may be a humanized antibody, or the antibody may have a VL with 90, 91, 92, 93, 94, 95, 96, 97, 98, 99, or 100% identity to SEQ ID NO: 139 and/or a VH with 90, 91, 92, 93, 94, 95, 96, 97, 98, 99 or 100% identity to SEQ ID NO: 140. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 134, 135, 136, 137, 138, 139, 140, and the amino acid sequence DDS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 146 or a VH that has one or more HVRs derived from SEQ ID NO: 147. The HVR derived from SEQ ID NO: 146 may be L1, L2, L3, or any combination thereof. In certain embodiments, the VL may comprise an L1 of SEQ ID NO: 141, an L2 of KDS, an L3 of SEQ ID NO: 142, or any combination thereof (e.g. antibodies 1199-1205 in Table A). The HVR derived from SEQ ID NO: 147 may be H1, H2, H3, or any combination thereof. In certain embodiments, the VH may comprise an H1 of SEQ ID NO: 143, an H2 of SEQ ID NO: 144, an H3 of SEQ ID NO: 145, or any combination thereof (e.g. antibodies 1206-1212 in Table A). The antibody comprising one or more HVRs derived from SEQ ID NO: 147 may further comprise a light chain variable region (VL) comprising one or more HVRs derived from SEQ ID NO: 146. The HVR may be L1, L2, L3, or any combination thereof. In a preferred embodiment, the VL may comprise an L1 of SEQ ID NO: 141, an L2 of KDS, an L3 of SEQ ID NO: 142, or any combination thereof (e.g. antibodies 1213-1261 in Table A). In various embodiments above, the antibody may be a humanized antibody, or the antibody may have a VL with 90, 91, 92, 93, 94, 95, 96, 97, 98, 99, or 100% identity to SEQ ID NO: 146 and/or a VH with 90, 91, 92, 93, 94, 95, 96, 97, 98, 99 or 100% identity to SEQ ID NO: 147. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 141, 142, 143, 144, 145, 146, 147, and the amino acid sequence KDS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 153 or a VH that has one or more HVRs derived from SEQ ID NO: 154. The HVR derived from SEQ ID NO: 153 may be L1, L2, L3, or any combination thereof. In certain embodiments, the VL may comprise an L1 of SEQ ID NO: 148, an L2 of DAS, an L3 of SEQ ID NO: 149, or any combination thereof (e.g. antibodies 1262-1268 in Table A). The HVR derived from SEQ ID NO: 154 may be H1, H2, H3, or any combination thereof. In certain embodiments, the VH may comprise an H1 of SEQ ID NO: 150, an H2 of SEQ ID NO: 151, an H3 of SEQ ID NO: 152, or any combination thereof (e.g. antibodies 1269-1275 in Table A). The antibody comprising one or more HVRs derived from SEQ ID NO: 154 may further comprise a light chain variable region (VL) comprising one or more HVRs derived from SEQ ID NO: 153. The HVR may be L1, L2, L3, or any combination thereof. In a preferred embodiment, the VL may comprise an L1 of SEQ ID NO: 148, an L2 of DAS, an L3 of SEQ ID NO: 149, or any combination thereof (e.g. antibodies 1276-1324 in Table A). In various embodiments above, the antibody may be a humanized antibody, or the antibody may have a VL with 90, 91, 92, 93, 94, 95, 96, 97, 98, 99, or 100% identity to SEQ ID NO: 153 and/or a VH with 90, 91, 92, 93, 94, 95, 96, 97, 98, 99 or 100% identity to SEQ ID NO: 154. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 148, 149, 150, 151, 152, 153, 154, and the amino acid sequence DAS, which can readily be determined by one of skill in the art, and may be incorporated into a vector or other large DNA molecule, such as a chromosome, in order to express an antibody of the disclosure.

In another exemplary embodiment, an antibody of the disclosure comprises a VL that has one or more HVRs derived from SEQ ID NO: 160 or a VH that has one or more HVRs derived from SEQ ID NO: 161. The HVR derived from SEQ ID NO: 160 may be L1, L2, L3, or any combination thereof. In certain embodiments, the VL may comprise an L1 of SEQ ID NO: 155, an L2 of DDS, an L3 of SEQ ID NO: 156, or any combination thereof (e.g. antibodies 1325-1331 in Table A). The HVR derived from SEQ ID NO: 161 may be H1, H2, H3, or any combination thereof. In certain embodiments, the VH may comprise an H1 of SEQ ID NO: 157, an H2 of SEQ ID NO: 158, an H3 of SEQ ID NO: 159, or any combination thereof (e.g. antibodies 1332-1338 in Table A). The antibody comprising one or more HVRs derived from SEQ ID NO: 161 may further comprise a light chain variable region (VL) comprising one or more HVRs derived from SEQ ID NO: 160. The HVR may be L1, L2, L3, or any combination thereof. In a preferred embodiment, the VL may comprise an L1 of SEQ ID NO: 155, an L2 of DDS, an L3 of SEQ ID NO: 156, or any combination thereof (e.g. antibodies 1339-1387 in Table A). In various embodiments above, the antibody may be a humanized antibody, or the antibody may have a VL with 90, 91, 92, 93, 94, 95, 96, 97, 98, 99, or 100% identity to SEQ ID NO: 160 and/or a VH with 90, 91, 92, 93, 94, 95, 96, 97, 98, 99 or 100% identity to SEQ ID NO: 161. In each of the above embodiments, the anti-coronavirus spike antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 155, 156, 157, 158, 159, 160, 161, and the amino acid sequence DDS, which can readily be determined by one of skill in the art, and may be incorporated into a vector or other large DNA molecule, such as a chromosome, in order to express an antibody of the disclosure.

TABLE A
Exemplary Antibodies

| | Light Chain HVR | | | Heavy Chain HVR | | |
|---|---|---|---|---|---|---|
| Antibody | L1 | L2 | L3 | H1 | H2 | H3 |
| 1 | SEQ ID NO: 1 | | | | | |
| 2 | SEQ ID NO: 1 | DAS | | | | |
| 3 | SEQ ID NO: 1 | DAS | SEQ ID NO: 2 | | | |
| 4 | | DAS | | | | |
| 5 | | DAS | SEQ ID NO: 2 | | | |
| 6 | | | SEQ ID NO: 2 | | | |
| 7 | SEQ ID NO: 1 | | SEQ ID NO: 2 | | | |
| 8 | | | | SEQ ID NO: 3 | | |
| 9 | | | | SEQ ID NO: 3 | SEQ ID NO: 4 | |
| 10 | | | | SEQ ID NO: 3 | SEQ ID NO: 4 | SEQ ID NO: 5 |
| 11 | | | | | SEQ ID NO: 4 | |
| 12 | | | | | SEQ ID NO: 4 | SEQ ID NO: 5 |
| 13 | | | | | | SEQ ID NO: 5 |
| 14 | | | | SEQ ID NO: 3 | | SEQ ID NO: 5 |
| 15 | SEQ ID NO: 1 | | | SEQ ID NO: 3 | | |
| 16 | SEQ ID NO: 1 | | | SEQ ID NO: 3 | SEQ ID NO: 4 | |
| 17 | SEQ ID NO: 1 | | | SEQ ID NO: 3 | SEQ ID NO: 4 | SEQ ID NO: 5 |
| 18 | SEQ ID NO: 1 | | | | SEQ ID NO: 4 | |
| 19 | SEQ ID NO: 1 | | | | SEQ ID NO: 4 | SEQ ID NO: 5 |
| 20 | SEQ ID NO: 1 | | | | | SEQ ID NO: 5 |
| 21 | SEQ ID NO: 1 | | | SEQ ID NO: 3 | | SEQ ID NO: 5 |
| 22 | SEQ ID NO: 1 | DAS | | SEQ ID NO: 3 | | |
| 23 | SEQ ID NO: 1 | DAS | | SEQ ID NO: 3 | SEQ ID NO: 4 | |
| 24 | SEQ ID NO: 1 | DAS | | SEQ ID NO: 3 | SEQ ID NO: 4 | SEQ ID NO: 5 |
| 25 | SEQ ID NO: 1 | DAS | | | SEQ ID NO: 4 | |
| 26 | SEQ ID NO: 1 | DAS | | | SEQ ID NO: 4 | SEQ ID NO: 5 |
| 27 | SEQ ID NO: 1 | DAS | | | | SEQ ID NO: 5 |
| 28 | SEQ ID NO: 1 | DAS | | SEQ ID NO: 3 | | SEQ ID NO: 5 |
| 29 | SEQ ID NO: 1 | DAS | SEQ ID NO: 2 | SEQ ID NO: 3 | | |
| 30 | SEQ ID NO: 1 | DAS | SEQ ID NO: 2 | SEQ ID NO: 3 | SEQ ID NO: 4 | |
| 31 | SEQ ID NO: 1 | DAS | SEQ ID NO: 2 | SEQ ID NO: 3 | SEQ ID NO: 4 | SEQ ID NO: 5 |
| 32 | SEQ ID NO: 1 | DAS | SEQ ID NO: 2 | | SEQ ID NO: 4 | |
| 33 | SEQ ID NO: 1 | DAS | SEQ ID NO: 2 | | SEQ ID NO: 4 | SEQ ID NO: 5 |
| 34 | SEQ ID NO: 1 | DAS | SEQ ID NO: 2 | SEQ ID NO: 3 | | SEQ ID NO: 5 |
| 35 | SEQ ID NO: 1 | DAS | SEQ ID NO: 2 | | | SEQ ID NO: 5 |
| 36 | | DAS | | SEQ ID NO: 3 | | |
| 37 | | DAS | | SEQ ID NO: 3 | SEQ ID NO: 4 | |
| 38 | | DAS | | SEQ ID NO: 3 | SEQ ID NO: 4 | SEQ ID NO: 5 |
| 39 | | DAS | | | SEQ ID NO: 4 | |
| 40 | | DAS | | | SEQ ID NO: 4 | SEQ ID NO: 5 |
| 41 | | DAS | | | | SEQ ID NO: 5 |
| 42 | | DAS | | SEQ ID NO: 3 | | SEQ ID NO: 5 |
| 43 | | DAS | SEQ ID NO: 2 | SEQ ID NO: 3 | | |
| 44 | | DAS | SEQ ID NO: 2 | SEQ ID NO: 3 | SEQ ID NO: 4 | |
| 45 | | DAS | SEQ ID NO: 2 | SEQ ID NO: 3 | SEQ ID NO: 4 | SEQ ID NO: 5 |
| 46 | | DAS | SEQ ID NO: 2 | | SEQ ID NO: 4 | |
| 47 | | DAS | SEQ ID NO: 2 | | SEQ ID NO: 4 | SEQ ID NO: 5 |
| 48 | | DAS | SEQ ID NO: 2 | | | SEQ ID NO: 5 |
| 49 | | DAS | SEQ ID NO: 2 | SEQ ID NO: 3 | | SEQ ID NO: 5 |
| 50 | | | SEQ ID NO: 2 | SEQ ID NO: 3 | | |
| 51 | | | SEQ ID NO: 2 | SEQ ID NO: 3 | SEQ ID NO: 4 | |
| 52 | | | SEQ ID NO: 2 | SEQ ID NO: 3 | SEQ ID NO: 4 | SEQ ID NO: 5 |
| 53 | | | SEQ ID NO: 2 | | SEQ ID NO: 4 | |
| 54 | | | SEQ ID NO: 2 | | SEQ ID NO: 4 | SEQ ID NO: 5 |
| 55 | | | SEQ ID NO: 2 | | | SEQ ID NO: 5 |
| 56 | | | SEQ ID NO: 2 | SEQ ID NO: 3 | | SEQ ID NO: 5 |
| 57 | SEQ ID NO: 1 | | SEQ ID NO: 2 | SEQ ID NO: 3 | | |
| 58 | SEQ ID NO: 1 | | SEQ ID NO: 2 | SEQ ID NO: 3 | SEQ ID NO: 4 | |
| 59 | SEQ ID NO: 1 | | SEQ ID NO: 2 | SEQ ID NO: 3 | SEQ ID NO: 4 | SEQ ID NO: 5 |
| 60 | SEQ ID NO: 1 | | SEQ ID NO: 2 | | SEQ ID NO: 4 | |
| 61 | SEQ ID NO: 1 | | SEQ ID NO: 2 | | SEQ ID NO: 4 | SEQ ID NO: 5 |
| 62 | SEQ ID NO: 1 | | SEQ ID NO: 2 | | | SEQ ID NO: 5 |
| 63 | SEQ ID NO: 1 | | SEQ ID NO: 2 | SEQ ID NO: 3 | | SEQ ID NO: 5 |
| 64 | SEQ ID NO: 8 | | | | | |
| 65 | SEQ ID NO: 8 | AAS | | | | |
| 66 | SEQ ID NO: 8 | AAS | SEQ ID NO: 9 | | | |
| 67 | | AAS | | | | |
| 68 | | AAS | SEQ ID NO: 9 | | | |
| 69 | | | SEQ ID NO: 9 | | | |
| 70 | SEQ ID NO: 8 | | SEQ ID NO: 9 | | | |
| 71 | | | | SEQ ID NO: 10 | | |
| 72 | | | | SEQ ID NO: 10 | SEQ ID NO: 11 | |
| 73 | | | | SEQ ID NO: 10 | SEQ ID NO: 11 | SEQ ID NO: 12 |
| 74 | | | | | SEQ ID NO: 11 | |
| 75 | | | | | SEQ ID NO: 11 | SEQ ID NO: 12 |
| 76 | | | | | | SEQ ID NO: 12 |
| 77 | | | | SEQ ID NO: 10 | | SEQ ID NO: 12 |
| 78 | SEQ ID NO: 8 | | | SEQ ID NO: 10 | | |
| 79 | SEQ ID NO: 8 | | | SEQ ID NO: 10 | SEQ ID NO: 11 | |
| 80 | SEQ ID NO: 8 | | | SEQ ID NO: 10 | SEQ ID NO: 11 | SEQ ID NO: 12 |
| 81 | SEQ ID NO: 8 | | | | SEQ ID NO: 11 | |
| 82 | SEQ ID NO: 8 | | | | SEQ ID NO: 11 | SEQ ID NO: 12 |
| 83 | SEQ ID NO: 8 | | | | | SEQ ID NO: 12 |
| 84 | SEQ ID NO: 8 | | | SEQ ID NO: 10 | | SEQ ID NO: 12 |
| 85 | SEQ ID NO: 8 | AAS | | SEQ ID NO: 10 | | |
| 86 | SEQ ID NO: 8 | AAS | | SEQ ID NO: 10 | SEQ ID NO: 11 | |
| 87 | SEQ ID NO: 8 | AAS | | SEQ ID NO: 10 | SEQ ID NO: 11 | SEQ ID NO: 12 |
| 88 | SEQ ID NO: 8 | AAS | | | SEQ ID NO: 11 | |
| 89 | SEQ ID NO: 8 | AAS | | | SEQ ID NO: 11 | SEQ ID NO: 12 |
| 90 | SEQ ID NO: 8 | AAS | | | | SEQ ID NO: 12 |
| 91 | SEQ ID NO: 8 | AAS | | SEQ ID NO: 10 | | SEQ ID NO: 12 |
| 92 | SEQ ID NO: 8 | AAS | SEQ ID NO: 9 | SEQ ID NO: 10 | | |
| 93 | SEQ ID NO: 8 | AAS | SEQ ID NO: 9 | SEQ ID NO: 10 | SEQ ID NO: 11 | |
| 94 | SEQ ID NO: 8 | AAS | SEQ ID NO: 9 | SEQ ID NO: 10 | SEQ ID NO: 11 | SEQ ID NO: 12 |
| 95 | SEQ ID NO: 8 | AAS | SEQ ID NO: 9 | | SEQ ID NO: 11 | |
| 96 | SEQ ID NO: 8 | AAS | SEQ ID NO: 9 | | SEQ ID NO: 11 | SEQ ID NO: 12 |
| 97 | SEQ ID NO: 8 | AAS | SEQ ID NO: 9 | | | SEQ ID NO: 12 |
| 98 | SEQ ID NO: 8 | AAS | SEQ ID NO: 9 | SEQ ID NO: 10 | | SEQ ID NO: 12 |
| 99 | | AAS | | SEQ ID NO: 10 | | |
| 100 | | AAS | | SEQ ID NO: 10 | SEQ ID NO: 11 | |
| 101 | | AAS | | SEQ ID NO: 10 | SEQ ID NO: 11 | SEQ ID NO: 12 |
| 102 | | AAS | | | SEQ ID NO: 11 | |
| 103 | | AAS | | | SEQ ID NO: 11 | SEQ ID NO: 12 |
| 104 | | AAS | | | | SEQ ID NO: 12 |
| 105 | | AAS | | SEQ ID NO: 10 | | SEQ ID NO: 12 |
| 106 | | AAS | SEQ ID NO: 9 | SEQ ID NO: 10 | | |
| 107 | | AAS | SEQ ID NO: 9 | SEQ ID NO: 10 | SEQ ID NO: 11 | |
| 108 | | AAS | SEQ ID NO: 9 | SEQ ID NO: 10 | SEQ ID NO: 11 | SEQ ID NO: 12 |
| 109 | | AAS | SEQ ID NO: 9 | | SEQ ID NO: 11 | |
| 110 | | AAS | SEQ ID NO: 9 | | SEQ ID NO: 11 | SEQ ID NO: 12 |
| 111 | | AAS | SEQ ID NO: 9 | | | SEQ ID NO: 12 |
| 112 | | AAS | SEQ ID NO: 9 | SEQ ID NO: 10 | | SEQ ID NO: 12 |
| 113 | | | SEQ ID NO: 9 | SEQ ID NO: 10 | | |
| 114 | | | SEQ ID NO: 9 | SEQ ID NO: 10 | SEQ ID NO: 11 | |
| 115 | | | SEQ ID NO: 9 | SEQ ID NO: 10 | SEQ ID NO: 11 | SEQ ID NO: 12 |
| 116 | | | SEQ ID NO: 9 | | SEQ ID NO: 11 | |
| 117 | | | SEQ ID NO: 9 | | SEQ ID NO: 11 | SEQ ID NO: 12 |
| 118 | | | SEQ ID NO: 9 | | | SEQ ID NO: 12 |
| 119 | | | SEQ ID NO: 9 | SEQ ID NO: 10 | | SEQ ID NO: 12 |
| 120 | SEQ ID NO: 8 | | SEQ ID NO: 9 | SEQ ID NO: 10 | | |
| 121 | SEQ ID NO: 8 | | SEQ ID NO: 9 | SEQ ID NO: 10 | SEQ ID NO: 11 | |
| 122 | SEQ ID NO: 8 | | SEQ ID NO: 9 | SEQ ID NO: 10 | SEQ ID NO: 11 | SEQ ID NO: 12 |
| 123 | SEQ ID NO: 8 | | SEQ ID NO: 9 | | SEQ ID NO: 11 | |
| 124 | SEQ ID NO: 8 | | SEQ ID NO: 9 | | SEQ ID NO: 11 | SEQ ID NO: 12 |
| 125 | SEQ ID NO: 8 | | SEQ ID NO: 9 | | | SEQ ID NO: 12 |
| 126 | SEQ ID NO: 8 | | SEQ ID NO: 9 | SEQ ID NO: 10 | | SEQ ID NO: 12 |
| 127 | SEQ ID NO: 15 | | | | | |
| 128 | SEQ ID NO: 15 | QDN | | | | |
| 219 | SEQ ID NO: 15 | QDN | SEQ ID NO: 16 | | | |
| 130 | | QDN | | | | |
| 131 | | QDN | SEQ ID NO: 16 | | | |
| 132 | | | SEQ ID NO: 16 | | | |
| 133 | SEQ ID NO: 15 | | SEQ ID NO: 16 | | | |
| 134 | | | | SEQ ID NO: 17 | | |
| 135 | | | | SEQ ID NO: 17 | SEQ ID NO: 18 | |
| 136 | | | | SEQ ID NO: 17 | SEQ ID NO: 18 | SEQ ID NO: 19 |
| 137 | | | | | SEQ ID NO: 18 | |
| 138 | | | | | SEQ ID NO: 18 | SEQ ID NO: 19 |
| 139 | | | | | | SEQ ID NO: 19 |
| 140 | | | | SEQ ID NO: 17 | | SEQ ID NO: 19 |
| 141 | SEQ ID NO: 15 | | | SEQ ID NO: 17 | | |
| 142 | SEQ ID NO: 15 | | | SEQ ID NO: 17 | SEQ ID NO: 18 | |
| 143 | SEQ ID NO: 15 | | | SEQ ID NO: 17 | SEQ ID NO: 18 | SEQ ID NO: 19 |
| 144 | SEQ ID NO: 15 | | | | SEQ ID NO: 18 | |
| 145 | SEQ ID NO: 15 | | | | SEQ ID NO: 18 | SEQ ID NO: 19 |
| 146 | SEQ ID NO: 15 | | | | | SEQ ID NO: 19 |
| 147 | SEQ ID NO: 15 | | | SEQ ID NO: 17 | | SEQ ID NO: 19 |
| 148 | SEQ ID NO: 15 | QDN | | SEQ ID NO: 17 | | |
| 149 | SEQ ID NO: 15 | QDN | | SEQ ID NO: 17 | SEQ ID NO: 18 | |
| 150 | SEQ ID NO: 15 | QDN | | SEQ ID NO: 17 | SEQ ID NO: 18 | SEQ ID NO: 19 |
| 151 | SEQ ID NO: 15 | QDN | | | SEQ ID NO: 18 | |
| 152 | SEQ ID NO: 15 | QDN | | | SEQ ID NO: 18 | SEQ ID NO: 19 |
| 153 | SEQ ID NO: 15 | QDN | | | | SEQ ID NO: 19 |
| 154 | SEQ ID NO: 15 | QDN | | SEQ ID NO: 17 | | SEQ ID NO: 19 |
| 155 | SEQ ID NO: 15 | QDN | SEQ ID NO: 16 | SEQ ID NO: 17 | | |
| 156 | SEQ ID NO: 15 | QDN | SEQ ID NO: 16 | SEQ ID NO: 17 | SEQ ID NO: 18 | |
| 157 | SEQ ID NO: 15 | QDN | SEQ ID NO: 16 | SEQ ID NO: 17 | SEQ ID NO: 18 | SEQ ID NO: 19 |
| 158 | SEQ ID NO: 15 | QDN | SEQ ID NO: 16 | | SEQ ID NO: 18 | |
| 159 | SEQ ID NO: 15 | QDN | SEQ ID NO: 16 | | SEQ ID NO: 18 | SEQ ID NO: 19 |
| 160 | SEQ ID NO: 15 | QDN | SEQ ID NO: 16 | SEQ ID NO: 17 | | SEQ ID NO: 19 |
| 161 | SEQ ID NO: 15 | QDN | SEQ ID NO: 16 | | | SEQ ID NO: 19 |
| 162 | | QDN | | SEQ ID NO: 17 | | |
| 163 | | QDN | | SEQ ID NO: 17 | SEQ ID NO: 18 | |
| 164 | | QDN | | SEQ ID NO: 17 | SEQ ID NO: 18 | SEQ ID NO: 19 |
| 165 | | QDN | | | SEQ ID NO: 18 | |
| 166 | | QDN | | | SEQ ID NO: 18 | SEQ ID NO: 19 |
| 167 | | QDN | | | | SEQ ID NO: 19 |
| 168 | | QDN | | SEQ ID NO: 17 | | SEQ ID NO: 19 |
| 169 | | QDN | SEQ ID NO: 16 | SEQ ID NO: 17 | | |
| 170 | | QDN | SEQ ID NO: 16 | SEQ ID NO: 17 | SEQ ID NO: 18 | |
| 171 | | QDN | SEQ ID NO: 16 | SEQ ID NO: 17 | SEQ ID NO: 18 | SEQ ID NO: 19 |
| 172 | | QDN | SEQ ID NO: 16 | | SEQ ID NO: 18 | |
| 173 | | QDN | SEQ ID NO: 16 | | SEQ ID NO: 18 | SEQ ID NO: 19 |
| 174 | | QDN | SEQ ID NO: 16 | | | SEQ ID NO: 19 |
| 175 | | QDN | SEQ ID NO: 16 | SEQ ID NO: 17 | | SEQ ID NO: 19 |
| 176 | | | SEQ ID NO: 16 | SEQ ID NO: 17 | | |
| 177 | | | SEQ ID NO: 16 | SEQ ID NO: 17 | SEQ ID NO: 18 | |
| 178 | | | SEQ ID NO: 16 | SEQ ID NO: 17 | SEQ ID NO: 18 | SEQ ID NO: 19 |
| 179 | | | SEQ ID NO: 16 | | SEQ ID NO: 18 | |
| 180 | | | SEQ ID NO: 16 | | SEQ ID NO: 18 | SEQ ID NO: 19 |
| 181 | | | SEQ ID NO: 16 | | | SEQ ID NO: 19 |
| 182 | | | SEQ ID NO: 16 | SEQ ID NO: 17 | | SEQ ID NO: 19 |
| 183 | SEQ ID NO: 15 | | SEQ ID NO: 16 | SEQ ID NO: 17 | | |
| 184 | SEQ ID NO: 15 | | SEQ ID NO: 16 | SEQ ID NO: 17 | SEQ ID NO: 18 | |
| 185 | SEQ ID NO: 15 | | SEQ ID NO: 16 | SEQ ID NO: 17 | SEQ ID NO: 18 | SEQ ID NO: 19 |
| 186 | SEQ ID NO: 15 | | SEQ ID NO: 16 | | SEQ ID NO: 18 | |
| 187 | SEQ ID NO: 15 | | SEQ ID NO: 16 | | SEQ ID NO: 18 | SEQ ID NO: 19 |
| 188 | SEQ ID NO: 15 | | SEQ ID NO: 16 | | | SEQ ID NO: 19 |
| 189 | SEQ ID NO: 15 | | SEQ ID NO: 16 | SEQ ID NO: 17 | | SEQ ID NO: 19 |
| 190 | SEQ ID NO: 22 | | | | | |
| 191 | SEQ ID NO: 22 | DAS | | | | |
| 192 | SEQ ID NO: 22 | DAS | SEQ ID NO: 23 | | | |
| 193 | | DAS | | | | |
| 194 | | DAS | SEQ ID NO: 23 | | | |
| 195 | | | SEQ ID NO: 23 | | | |
| 196 | SEQ ID NO: 22 | | SEQ ID NO: 23 | | | |
| 197 | | | | SEQ ID NO: 24 | | |
| 198 | | | | SEQ ID NO: 24 | SEQ ID NO: 25 | |
| 199 | | | | SEQ ID NO: 24 | SEQ ID NO: 25 | SEQ ID NO: 26 |
| 200 | | | | | SEQ ID NO: 25 | |
| 201 | | | | | SEQ ID NO: 25 | SEQ ID NO: 26 |
| 202 | | | | | | SEQ ID NO: 26 |
| 203 | | | | SEQ ID NO: 24 | | SEQ ID NO: 26 |
| 204 | SEQ ID NO: 22 | | | SEQ ID NO: 24 | | |
| 205 | SEQ ID NO: 22 | | | SEQ ID NO: 24 | SEQ ID NO: 25 | |
| 206 | SEQ ID NO: 22 | | | SEQ ID NO: 24 | SEQ ID NO: 25 | SEQ ID NO: 26 |
| 207 | SEQ ID NO: 22 | | | | SEQ ID NO: 25 | |
| 208 | SEQ ID NO: 22 | | | | SEQ ID NO: 25 | SEQ ID NO: 26 |
| 209 | SEQ ID NO: 22 | | | | | SEQ ID NO: 26 |
| 210 | SEQ ID NO: 22 | | | SEQ ID NO: 24 | | SEQ ID NO: 26 |
| 211 | SEQ ID NO: 22 | DAS | | SEQ ID NO: 24 | | |
| 212 | SEQ ID NO: 22 | DAS | | SEQ ID NO: 24 | SEQ ID NO: 25 | |
| 213 | SEQ ID NO: 22 | DAS | | SEQ ID NO: 24 | SEQ ID NO: 25 | SEQ ID NO: 26 |
| 214 | SEQ ID NO: 22 | DAS | | | SEQ ID NO: 25 | |
| 215 | SEQ ID NO: 22 | DAS | | | SEQ ID NO: 25 | SEQ ID NO: 26 |
| 216 | SEQ ID NO: 22 | DAS | | | | SEQ ID NO: 26 |
| 217 | SEQ ID NO: 22 | DAS | | SEQ ID NO: 24 | | SEQ ID NO: 26 |
| 218 | SEQ ID NO: 22 | DAS | SEQ ID NO: 23 | SEQ ID NO: 24 | | |
| 219 | SEQ ID NO: 22 | DAS | SEQ ID NO: 23 | SEQ ID NO: 24 | SEQ ID NO: 25 | |
| 220 | SEQ ID NO: 22 | DAS | SEQ ID NO: 23 | SEQ ID NO: 24 | SEQ ID NO: 25 | SEQ ID NO: 26 |
| 221 | SEQ ID NO: 22 | DAS | SEQ ID NO: 23 | | SEQ ID NO: 25 | |
| 222 | SEQ ID NO: 22 | DAS | SEQ ID NO: 23 | | SEQ ID NO: 25 | SEQ ID NO: 26 |
| 223 | SEQ ID NO: 22 | DAS | SEQ ID NO: 23 | SEQ ID NO: 24 | | SEQ ID NO: 26 |
| 224 | SEQ ID NO: 22 | DAS | SEQ ID NO: 23 | | | SEQ ID NO: 26 |
| 225 | | DAS | | SEQ ID NO: 24 | | |
| 226 | | DAS | | SEQ ID NO: 24 | SEQ ID NO: 25 | |
| 227 | | DAS | | SEQ ID NO: 24 | SEQ ID NO: 25 | SEQ ID NO: 26 |
| 228 | | DAS | | | SEQ ID NO: 25 | |
| 229 | | DAS | | | SEQ ID NO: 25 | SEQ ID NO: 26 |
| 230 | | DAS | | | | SEQ ID NO: 26 |
| 231 | | DAS | | SEQ ID NO: 24 | | SEQ ID NO: 26 |
| 232 | | DAS | SEQ ID NO: 23 | SEQ ID NO: 24 | | |
| 233 | | DAS | SEQ ID NO: 23 | SEQ ID NO: 24 | SEQ ID NO: 25 | |
| 234 | | DAS | SEQ ID NO: 23 | SEQ ID NO: 24 | SEQ ID NO: 25 | SEQ ID NO: 26 |
| 235 | | DAS | SEQ ID NO: 23 | | SEQ ID NO: 25 | |
| 236 | | DAS | SEQ ID NO: 23 | | SEQ ID NO: 25 | SEQ ID NO: 26 |
| 237 | | DAS | SEQ ID NO: 23 | | | SEQ ID NO: 26 |
| 238 | | DAS | SEQ ID NO: 23 | SEQ ID NO: 24 | | SEQ ID NO: 26 |
| 239 | | | SEQ ID NO: 23 | SEQ ID NO: 24 | | |
| 240 | | | SEQ ID NO: 23 | SEQ ID NO: 24 | SEQ ID NO: 25 | |
| 240 | | | SEQ ID NO: 23 | SEQ ID NO: 24 | SEQ ID NO: 25 | SEQ ID NO: 26 |
| 242 | | | SEQ ID NO: 23 | | SEQ ID NO: 25 | |
| 423 | | | SEQ ID NO: 23 | | SEQ ID NO: 25 | SEQ ID NO: 26 |
| 244 | | | SEQ ID NO: 23 | | | SEQ ID NO: 26 |
| 245 | | | SEQ ID NO: 23 | SEQ ID NO: 24 | | SEQ ID NO: 26 |
| 246 | SEQ ID NO: 22 | | SEQ ID NO: 23 | SEQ ID NO: 24 | | |
| 247 | SEQ ID NO: 22 | | SEQ ID NO: 23 | SEQ ID NO: 24 | SEQ ID NO: 25 | |
| 248 | SEQ ID NO: 22 | | SEQ ID NO: 23 | SEQ ID NO: 24 | SEQ ID NO: 25 | SEQ ID NO: 26 |
| 249 | SEQ ID NO: 22 | | SEQ ID NO: 23 | | SEQ ID NO: 25 | |
| 250 | SEQ ID NO: 22 | | SEQ ID NO: 23 | | SEQ ID NO: 25 | SEQ ID NO: 26 |
| 251 | SEQ ID NO: 22 | | SEQ ID NO: 23 | | | SEQ ID NO: 26 |
| 252 | SEQ ID NO: 22 | | SEQ ID NO: 23 | SEQ ID NO: 24 | | SEQ ID NO: 26 |
| 253 | SEQ ID NO: 29 | | | | | |
| 254 | SEQ ID NO: 29 | ATS | | | | |
| 255 | SEQ ID NO: 29 | ATS | SEQ ID NO: 30 | | | |
| 256 | | ATS | | | | |
| 257 | | ATS | SEQ ID NO: 30 | | | |
| 258 | | | SEQ ID NO: 30 | | | |
| 259 | SEQ ID NO: 29 | | SEQ ID NO: 30 | | | |
| 260 | | | | SEQ ID NO: 31 | | |
| 261 | | | | SEQ ID NO: 31 | SEQ ID NO: 32 | |
| 262 | | | | SEQ ID NO: 31 | SEQ ID NO: 32 | SEQ ID NO: 33 |
| 263 | | | | | SEQ ID NO: 32 | |
| 264 | | | | | SEQ ID NO: 32 | SEQ ID NO: 33 |
| 265 | | | | | | SEQ ID NO: 33 |
| 266 | | | | SEQ ID NO: 31 | | SEQ ID NO: 33 |
| 267 | SEQ ID NO: 29 | | | SEQ ID NO: 31 | | |
| 268 | SEQ ID NO: 29 | | | SEQ ID NO: 31 | SEQ ID NO: 32 | |
| 269 | SEQ ID NO: 29 | | | SEQ ID NO: 31 | SEQ ID NO: 32 | SEQ ID NO: 33 |
| 270 | SEQ ID NO: 29 | | | | SEQ ID NO: 32 | |
| 271 | SEQ ID NO: 29 | | | | SEQ ID NO: 32 | SEQ ID NO: 33 |
| 272 | SEQ ID NO: 29 | | | | | SEQ ID NO: 33 |
| 273 | SEQ ID NO: 29 | | | SEQ ID NO: 31 | | SEQ ID NO: 33 |
| 274 | SEQ ID NO: 29 | ATS | | SEQ ID NO: 31 | | |
| 275 | SEQ ID NO: 29 | ATS | | SEQ ID NO: 31 | SEQ ID NO: 32 | |
| 276 | SEQ ID NO: 29 | ATS | | SEQ ID NO: 31 | SEQ ID NO: 32 | SEQ ID NO: 33 |
| 277 | SEQ ID NO: 29 | ATS | | | SEQ ID NO: 32 | |
| 278 | SEQ ID NO: 29 | ATS | | | SEQ ID NO: 32 | SEQ ID NO: 33 |
| 279 | SEQ ID NO: 29 | ATS | | | | SEQ ID NO: 33 |
| 280 | SEQ ID NO: 29 | ATS | | SEQ ID NO: 31 | | SEQ ID NO: 33 |
| 281 | SEQ ID NO: 29 | ATS | SEQ ID NO: 30 | SEQ ID NO: 31 | | |
| 282 | SEQ ID NO: 29 | ATS | SEQ ID NO: 30 | SEQ ID NO: 31 | SEQ ID NO: 32 | |
| 283 | SEQ ID NO: 29 | ATS | SEQ ID NO: 30 | SEQ ID NO: 31 | SEQ ID NO: 32 | SEQ ID NO: 33 |
| 284 | SEQ ID NO: 29 | ATS | SEQ ID NO: 30 | | SEQ ID NO: 32 | |
| 285 | SEQ ID NO: 29 | ATS | SEQ ID NO: 30 | | SEQ ID NO: 32 | SEQ ID NO: 33 |
| 286 | SEQ ID NO: 29 | ATS | SEQ ID NO: 30 | SEQ ID NO: 31 | | SEQ ID NO: 33 |
| 287 | SEQ ID NO: 29 | ATS | SEQ ID NO: 30 | | | SEQ ID NO: 33 |
| 288 | | ATS | | SEQ ID NO: 31 | | |
| 289 | | ATS | | SEQ ID NO: 31 | SEQ ID NO: 32 | |
| 290 | | ATS | | SEQ ID NO: 31 | SEQ ID NO: 32 | SEQ ID NO: 33 |
| 291 | | ATS | | | SEQ ID NO: 32 | |
| 292 | | ATS | | | SEQ ID NO: 32 | SEQ ID NO: 33 |
| 293 | | ATS | | | | SEQ ID NO: 33 |
| 294 | | ATS | | SEQ ID NO: 31 | | SEQ ID NO: 33 |
| 295 | | ATS | SEQ ID NO: 30 | SEQ ID NO: 31 | | |
| 296 | | ATS | SEQ ID NO: 30 | SEQ ID NO: 31 | SEQ ID NO: 32 | |
| 297 | | ATS | SEQ ID NO: 30 | SEQ ID NO: 31 | SEQ ID NO: 32 | SEQ ID NO: 33 |
| 298 | | ATS | SEQ ID NO: 30 | | SEQ ID NO: 32 | |
| 299 | | ATS | SEQ ID NO: 30 | | SEQ ID NO: 32 | SEQ ID NO: 33 |
| 300 | | ATS | SEQ ID NO: 30 | | | SEQ ID NO: 33 |
| 301 | | ATS | SEQ ID NO: 30 | SEQ ID NO: 31 | | SEQ ID NO: 33 |
| 302 | | | SEQ ID NO: 30 | SEQ ID NO: 31 | | |
| 303 | | | SEQ ID NO: 30 | SEQ ID NO: 31 | SEQ ID NO: 32 | |
| 304 | | | SEQ ID NO: 30 | SEQ ID NO: 31 | SEQ ID NO: 32 | SEQ ID NO: 33 |
| 305 | | | SEQ ID NO: 30 | | SEQ ID NO: 32 | |
| 306 | | | SEQ ID NO: 30 | | SEQ ID NO: 32 | SEQ ID NO: 33 |
| 307 | | | SEQ ID NO: 30 | | | SEQ ID NO: 33 |
| 308 | | | SEQ ID NO: 30 | SEQ ID NO: 31 | | SEQ ID NO: 33 |
| 309 | SEQ ID NO: 29 | | SEQ ID NO: 30 | SEQ ID NO: 31 | | |
| 310 | SEQ ID NO: 29 | | SEQ ID NO: 30 | SEQ ID NO: 31 | SEQ ID NO: 32 | |
| 311 | SEQ ID NO: 29 | | SEQ ID NO: 30 | SEQ ID NO: 31 | SEQ ID NO: 32 | SEQ ID NO: 33 |
| 312 | SEQ ID NO: 29 | | SEQ ID NO: 30 | | SEQ ID NO: 32 | |
| 313 | SEQ ID NO: 29 | | SEQ ID NO: 30 | | SEQ ID NO: 32 | SEQ ID NO: 33 |
| 314 | SEQ ID NO: 29 | | SEQ ID NO: 30 | | | SEQ ID NO: 33 |
| 315 | SEQ ID NO: 29 | | SEQ ID NO: 30 | SEQ ID NO: 31 | | SEQ ID NO: 33 |
| 316 | SEQ ID NO: 36 | | | | | |
| 317 | SEQ ID NO: 36 | EDN | | | | |
| 318 | SEQ ID NO: 36 | EDN | SEQ ID NO: 37 | | | |
| 319 | | EDN | | | | |
| 320 | | EDN | SEQ ID NO: 37 | | | |
| 321 | | | SEQ ID NO: 37 | | | |
| 322 | SEQ ID NO: 36 | | SEQ ID NO: 37 | | | |
| 323 | | | | SEQ ID NO: 38 | | |
| 324 | | | | SEQ ID NO: 38 | SEQ ID NO: 39 | |
| 325 | | | | SEQ ID NO: 38 | SEQ ID NO: 39 | SEQ ID NO: 40 |
| 326 | | | | | SEQ ID NO: 39 | |
| 327 | | | | | SEQ ID NO: 39 | SEQ ID NO: 40 |
| 328 | | | | | | SEQ ID NO: 40 |
| 319 | | | | SEQ ID NO: 38 | | SEQ ID NO: 40 |
| 330 | SEQ ID NO: 36 | | | SEQ ID NO: 38 | | |
| 331 | SEQ ID NO: 36 | | | SEQ ID NO: 38 | SEQ ID NO: 39 | |
| 332 | SEQ ID NO: 36 | | | SEQ ID NO: 38 | SEQ ID NO: 39 | SEQ ID NO: 40 |
| 333 | SEQ ID NO: 36 | | | | SEQ ID NO: 39 | |
| 334 | SEQ ID NO: 36 | | | | SEQ ID NO: 39 | SEQ ID NO: 40 |
| 335 | SEQ ID NO: 36 | | | | | SEQ ID NO: 40 |
| 336 | SEQ ID NO: 36 | | | SEQ ID NO: 38 | | SEQ ID NO: 40 |
| 337 | SEQ ID NO: 36 | EDN | | SEQ ID NO: 38 | | |
| 338 | SEQ ID NO: 36 | EDN | | SEQ ID NO: 38 | SEQ ID NO: 39 | |
| 339 | SEQ ID NO: 36 | EDN | | SEQ ID NO: 38 | SEQ ID NO: 39 | SEQ ID NO: 40 |
| 340 | SEQ ID NO: 36 | EDN | | | SEQ ID NO: 39 | |
| 341 | SEQ ID NO: 36 | EDN | | | SEQ ID NO: 39 | SEQ ID NO: 40 |
| 342 | SEQ ID NO: 36 | EDN | | | | SEQ ID NO: 40 |
| 343 | SEQ ID NO: 36 | EDN | | SEQ ID NO: 38 | | SEQ ID NO: 40 |
| 344 | SEQ ID NO: 36 | EDN | SEQ ID NO: 37 | SEQ ID NO: 38 | | |
| 345 | SEQ ID NO: 36 | EDN | SEQ ID NO: 37 | SEQ ID NO: 38 | SEQ ID NO: 39 | |
| 346 | SEQ ID NO: 36 | EDN | SEQ ID NO: 37 | SEQ ID NO: 38 | SEQ ID NO: 39 | SEQ ID NO: 40 |
| 347 | SEQ ID NO: 36 | EDN | SEQ ID NO: 37 | | SEQ ID NO: 39 | |
| 348 | SEQ ID NO: 36 | EDN | SEQ ID NO: 37 | | SEQ ID NO: 39 | SEQ ID NO: 40 |
| 349 | SEQ ID NO: 36 | EDN | SEQ ID NO: 37 | SEQ ID NO: 38 | | SEQ ID NO: 40 |
| 350 | SEQ ID NO: 36 | EDN | SEQ ID NO: 37 | | | SEQ ID NO: 40 |
| 351 | | EDN | | SEQ ID NO: 38 | | |
| 352 | | EDN | | SEQ ID NO: 38 | SEQ ID NO: 39 | |
| 353 | | EDN | | SEQ ID NO: 38 | SEQ ID NO: 39 | SEQ ID NO: 40 |
| 354 | | EDN | | | SEQ ID NO: 39 | |
| 355 | | EDN | | | SEQ ID NO: 39 | SEQ ID NO: 40 |
| 356 | | EDN | | | | SEQ ID NO: 40 |
| 357 | | EDN | | SEQ ID NO: 38 | | SEQ ID NO: 40 |
| 358 | | EDN | SEQ ID NO: 37 | SEQ ID NO: 38 | | |
| 359 | | EDN | SEQ ID NO: 37 | SEQ ID NO: 38 | SEQ ID NO: 39 | |
| 360 | | EDN | SEQ ID NO: 37 | SEQ ID NO: 38 | SEQ ID NO: 39 | SEQ ID NO: 40 |
| 361 | | EDN | SEQ ID NO: 37 | | SEQ ID NO: 39 | |
| 362 | | EDN | SEQ ID NO: 37 | | SEQ ID NO: 39 | SEQ ID NO: 40 |
| 363 | | EDN | SEQ ID NO: 37 | | | SEQ ID NO: 40 |
| 364 | | EDN | SEQ ID NO: 37 | SEQ ID NO: 38 | | SEQ ID NO: 40 |
| 365 | | | SEQ ID NO: 37 | SEQ ID NO: 38 | | |
| 366 | | | SEQ ID NO: 37 | SEQ ID NO: 38 | SEQ ID NO: 39 | |
| 367 | | | SEQ ID NO: 37 | SEQ ID NO: 38 | SEQ ID NO: 39 | SEQ ID NO: 40 |
| 368 | | | SEQ ID NO: 37 | | SEQ ID NO: 39 | |
| 369 | | | SEQ ID NO: 37 | | SEQ ID NO: 39 | SEQ ID NO: 40 |
| 370 | | | SEQ ID NO: 37 | | | SEQ ID NO: 40 |
| 371 | | | SEQ ID NO: 37 | SEQ ID NO: 38 | | SEQ ID NO: 40 |
| 372 | SEQ ID NO: 36 | | SEQ ID NO: 37 | SEQ ID NO: 38 | | |
| 373 | SEQ ID NO: 36 | | SEQ ID NO: 37 | SEQ ID NO: 38 | SEQ ID NO: 39 | |
| 374 | SEQ ID NO: 36 | | SEQ ID NO: 37 | SEQ ID NO: 38 | SEQ ID NO: 39 | SEQ ID NO: 40 |
| 375 | SEQ ID NO: 36 | | SEQ ID NO: 37 | | SEQ ID NO: 39 | |
| 376 | SEQ ID NO: 36 | | SEQ ID NO: 37 | | SEQ ID NO: 39 | SEQ ID NO: 40 |
| 377 | SEQ ID NO: 36 | | SEQ ID NO: 37 | | | SEQ ID NO: 40 |
| 378 | SEQ ID NO: 36 | | SEQ ID NO: 37 | SEQ ID NO: 38 | | SEQ ID NO: 40 |
| 379 | SEQ ID NO: 43 | | | | | |
| 380 | SEQ ID NO: 43 | DAS | | | | |
| 381 | SEQ ID NO: 43 | DAS | SEQ ID NO: 44 | | | |
| 382 | | DAS | | | | |
| 383 | | DAS | SEQ ID NO: 44 | | | |
| 384 | | | SEQ ID NO: 44 | | | |
| 385 | SEQ ID NO: 43 | | SEQ ID NO: 44 | | | |
| 386 | | | | SEQ ID NO: 45 | | |
| 387 | | | | SEQ ID NO: 45 | SEQ ID NO: 46 | |
| 388 | | | | SEQ ID NO: 45 | SEQ ID NO: 46 | SEQ ID NO: 47 |
| 389 | | | | | SEQ ID NO: 46 | |
| 390 | | | | | SEQ ID NO: 46 | SEQ ID NO: 47 |
| 391 | | | | | | SEQ ID NO: 47 |
| 392 | | | | SEQ ID NO: 45 | | SEQ ID NO: 47 |
| 393 | SEQ ID NO: 43 | | | SEQ ID NO: 45 | | |
| 394 | SEQ ID NO: 43 | | | SEQ ID NO: 45 | SEQ ID NO: 46 | |
| 395 | SEQ ID NO: 43 | | | SEQ ID NO: 45 | SEQ ID NO: 46 | SEQ ID NO: 47 |
| 396 | SEQ ID NO: 43 | | | | SEQ ID NO: 46 | |
| 397 | SEQ ID NO: 43 | | | | SEQ ID NO: 46 | SEQ ID NO: 47 |
| 398 | SEQ ID NO: 43 | | | | | SEQ ID NO: 47 |
| 399 | SEQ ID NO: 43 | | | SEQ ID NO: 45 | | SEQ ID NO: 47 |
| 400 | SEQ ID NO: 43 | DAS | | SEQ ID NO: 45 | | |
| 401 | SEQ ID NO: 43 | DAS | | SEQ ID NO: 45 | SEQ ID NO: 46 | |
| 402 | SEQ ID NO: 43 | DAS | | SEQ ID NO: 45 | SEQ ID NO: 46 | SEQ ID NO: 47 |
| 403 | SEQ ID NO: 43 | DAS | | | SEQ ID NO: 46 | |
| 404 | SEQ ID NO: 43 | DAS | | | SEQ ID NO: 46 | SEQ ID NO: 47 |
| 405 | SEQ ID NO: 43 | DAS | | | | SEQ ID NO: 47 |
| 406 | SEQ ID NO: 43 | DAS | | SEQ ID NO: 45 | | SEQ ID NO: 47 |
| 407 | SEQ ID NO: 43 | DAS | SEQ ID NO: 44 | SEQ ID NO: 45 | | |
| 408 | SEQ ID NO: 43 | DAS | SEQ ID NO: 44 | SEQ ID NO: 45 | SEQ ID NO: 46 | |
| 409 | SEQ ID NO: 43 | DAS | SEQ ID NO: 44 | SEQ ID NO: 45 | SEQ ID NO: 46 | SEQ ID NO: 47 |
| 410 | SEQ ID NO: 43 | DAS | SEQ ID NO: 44 | | SEQ ID NO: 46 | |
| 411 | SEQ ID NO: 43 | DAS | SEQ ID NO: 44 | | SEQ ID NO: 46 | SEQ ID NO: 47 |
| 412 | SEQ ID NO: 43 | DAS | SEQ ID NO: 44 | | | SEQ ID NO: 47 |
| 413 | SEQ ID NO: 43 | DAS | SEQ ID NO: 44 | SEQ ID NO: 45 | | SEQ ID NO: 47 |
| 414 | | DAS | | SEQ ID NO: 45 | | |
| 415 | | DAS | | SEQ ID NO: 45 | SEQ ID NO: 46 | |
| 416 | | DAS | | SEQ ID NO: 45 | SEQ ID NO: 46 | SEQ ID NO: 47 |
| 417 | | DAS | | | SEQ ID NO: 46 | |
| 418 | | DAS | | | SEQ ID NO: 46 | SEQ ID NO: 47 |
| 419 | | DAS | | | | SEQ ID NO: 47 |
| 420 | | DAS | | SEQ ID NO: 45 | | SEQ ID NO: 47 |
| 421 | | DAS | SEQ ID NO: 44 | SEQ ID NO: 45 | | |
| 422 | | DAS | SEQ ID NO: 44 | SEQ ID NO: 45 | SEQ ID NO: 46 | |
| 423 | | DAS | SEQ ID NO: 44 | SEQ ID NO: 45 | SEQ ID NO: 46 | SEQ ID NO: 47 |
| 424 | | DAS | SEQ ID NO: 44 | | SEQ ID NO: 46 | |
| 425 | | DAS | SEQ ID NO: 44 | | SEQ ID NO: 46 | SEQ ID NO: 47 |
| 426 | | DAS | SEQ ID NO: 44 | | | SEQ ID NO: 47 |
| 427 | | DAS | SEQ ID NO: 44 | SEQ ID NO: 45 | | SEQ ID NO: 47 |
| 428 | | | SEQ ID NO: 44 | SEQ ID NO: 45 | | |
| 429 | | | SEQ ID NO: 44 | SEQ ID NO: 45 | SEQ ID NO: 46 | |
| 430 | | | SEQ ID NO: 44 | SEQ ID NO: 45 | SEQ ID NO: 46 | SEQ ID NO: 47 |
| 431 | | | SEQ ID NO: 44 | | SEQ ID NO: 46 | |
| 432 | | | SEQ ID NO: 44 | | SEQ ID NO: 46 | SEQ ID NO: 47 |
| 433 | | | SEQ ID NO: 44 | | | SEQ ID NO: 47 |
| 434 | | | SEQ ID NO: 44 | SEQ ID NO: 45 | | SEQ ID NO: 47 |
| 435 | SEQ ID NO: 43 | | SEQ ID NO: 44 | SEQ ID NO: 45 | | |
| 436 | SEQ ID NO: 43 | | SEQ ID NO: 44 | SEQ ID NO: 45 | SEQ ID NO: 46 | |
| 437 | SEQ ID NO: 43 | | SEQ ID NO: 44 | SEQ ID NO: 45 | SEQ ID NO: 46 | SEQ ID NO: 47 |
| 438 | SEQ ID NO: 43 | | SEQ ID NO: 44 | | SEQ ID NO: 46 | |
| 439 | SEQ ID NO: 43 | | SEQ ID NO: 44 | | SEQ ID NO: 46 | SEQ ID NO: 47 |
| 440 | SEQ ID NO: 43 | | SEQ ID NO: 44 | | | SEQ ID NO: 47 |
| 441 | SEQ ID NO: 43 | | SEQ ID NO: 44 | SEQ ID NO: 45 | | SEQ ID NO: 47 |
| 442 | SEQ ID NO: 50 | | | | | |
| 443 | SEQ ID NO: 50 | WAS | | | | |
| 444 | SEQ ID NO: 50 | WAS | SEQ ID NO: 51 | | | |
| 445 | | WAS | | | | |
| 446 | | WAS | SEQ ID NO: 51 | | | |
| 447 | | | SEQ ID NO: 51 | | | |
| 448 | SEQ ID NO: 50 | | SEQ ID NO: 51 | | | |
| 449 | | | | SEQ ID NO: 52 | | |
| 450 | | | | SEQ ID NO: 52 | SEQ ID NO: 53 | |
| 451 | | | | SEQ ID NO: 52 | SEQ ID NO: 53 | SEQ ID NO: 54 |
| 452 | | | | | SEQ ID NO: 53 | |
| 453 | | | | | SEQ ID NO: 53 | SEQ ID NO: 54 |
| 454 | | | | | | SEQ ID NO: 54 |
| 455 | | | | SEQ ID NO: 52 | | SEQ ID NO: 54 |
| 456 | SEQ ID NO: 50 | | | SEQ ID NO: 52 | | |
| 457 | SEQ ID NO: 50 | | | SEQ ID NO: 52 | SEQ ID NO: 53 | |
| 458 | SEQ ID NO: 50 | | | SEQ ID NO: 52 | SEQ ID NO: 53 | SEQ ID NO: 54 |
| 459 | SEQ ID NO: 50 | | | | SEQ ID NO: 53 | |
| 460 | SEQ ID NO: 50 | | | | SEQ ID NO: 53 | SEQ ID NO: 54 |
| 461 | SEQ ID NO: 50 | | | | | SEQ ID NO: 54 |
| 462 | SEQ ID NO: 50 | | | SEQ ID NO: 52 | | SEQ ID NO: 54 |
| 463 | SEQ ID NO: 50 | WAS | | SEQ ID NO: 52 | | |
| 464 | SEQ ID NO: 50 | WAS | | SEQ ID NO: 52 | SEQ ID NO: 53 | |
| 465 | SEQ ID NO: 50 | WAS | | SEQ ID NO: 52 | SEQ ID NO: 53 | SEQ ID NO: 54 |
| 466 | SEQ ID NO: 50 | WAS | | | SEQ ID NO: 53 | |
| 467 | SEQ ID NO: 50 | WAS | | | SEQ ID NO: 53 | SEQ ID NO: 54 |
| 468 | SEQ ID NO: 50 | WAS | | | | SEQ ID NO: 54 |
| 469 | SEQ ID NO: 50 | WAS | | SEQ ID NO: 52 | | SEQ ID NO: 54 |
| 470 | SEQ ID NO: 50 | WAS | SEQ ID NO: 51 | SEQ ID NO: 52 | | |
| 471 | SEQ ID NO: 50 | WAS | SEQ ID NO: 51 | SEQ ID NO: 52 | SEQ ID NO: 53 | |
| 472 | SEQ ID NO: 50 | WAS | SEQ ID NO: 51 | SEQ ID NO: 52 | SEQ ID NO: 53 | SEQ ID NO: 54 |
| 473 | SEQ ID NO: 50 | WAS | SEQ ID NO: 51 | | SEQ ID NO: 53 | |
| 474 | SEQ ID NO: 50 | WAS | SEQ ID NO: 51 | | SEQ ID NO: 53 | SEQ ID NO: 54 |
| 475 | SEQ ID NO: 50 | WAS | SEQ ID NO: 51 | | | SEQ ID NO: 54 |
| 476 | SEQ ID NO: 50 | WAS | SEQ ID NO: 51 | SEQ ID NO: 52 | | SEQ ID NO: 54 |
| 477 | | WAS | | SEQ ID NO: 52 | | |
| 478 | | WAS | | SEQ ID NO: 52 | SEQ ID NO: 53 | |
| 479 | | WAS | | SEQ ID NO: 52 | SEQ ID NO: 53 | SEQ ID NO: 54 |
| 480 | | WAS | | | SEQ ID NO: 53 | |
| 481 | | WAS | | | SEQ ID NO: 53 | SEQ ID NO: 54 |
| 482 | | WAS | | | | SEQ ID NO: 54 |
| 483 | | WAS | | SEQ ID NO: 52 | | SEQ ID NO: 54 |
| 484 | | WAS | SEQ ID NO: 51 | SEQ ID NO: 52 | | |
| 485 | | WAS | SEQ ID NO: 51 | SEQ ID NO: 52 | SEQ ID NO: 53 | |
| 486 | | WAS | SEQ ID NO: 51 | SEQ ID NO: 52 | SEQ ID NO: 53 | SEQ ID NO: 54 |
| 487 | | WAS | SEQ ID NO: 51 | | SEQ ID NO: 53 | |
| 488 | | WAS | SEQ ID NO: 51 | | SEQ ID NO: 53 | SEQ ID NO: 54 |
| 489 | | WAS | SEQ ID NO: 51 | | | SEQ ID NO: 54 |
| 490 | | WAS | SEQ ID NO: 51 | SEQ ID NO: 52 | | SEQ ID NO: 54 |
| 491 | | | SEQ ID NO: 51 | SEQ ID NO: 52 | | |
| 492 | | | SEQ ID NO: 51 | SEQ ID NO: 52 | SEQ ID NO: 53 | |
| 493 | | | SEQ ID NO: 51 | SEQ ID NO: 52 | SEQ ID NO: 53 | SEQ ID NO: 54 |
| 494 | | | SEQ ID NO: 51 | | SEQ ID NO: 53 | |
| 495 | | | SEQ ID NO: 51 | | SEQ ID NO: 53 | SEQ ID NO: 54 |
| 496 | | | SEQ ID NO: 51 | | | SEQ ID NO: 54 |
| 497 | | | SEQ ID NO: 51 | SEQ ID NO: 52 | | SEQ ID NO: 54 |
| 498 | SEQ ID NO: 50 | | SEQ ID NO: 51 | SEQ ID NO: 52 | | |
| 499 | SEQ ID NO: 50 | | SEQ ID NO: 51 | SEQ ID NO: 52 | SEQ ID NO: 53 | |
| 500 | SEQ ID NO: 50 | | SEQ ID NO: 51 | SEQ ID NO: 52 | SEQ ID NO: 53 | SEQ ID NO: 54 |
| 501 | SEQ ID NO: 50 | | SEQ ID NO: 51 | | SEQ ID NO: 53 | |
| 502 | SEQ ID NO: 50 | | SEQ ID NO: 51 | | SEQ ID NO: 53 | SEQ ID NO: 54 |
| 503 | SEQ ID NO: 50 | | SEQ ID NO: 51 | | | SEQ ID NO: 54 |
| 504 | SEQ ID NO: 50 | | SEQ ID NO: 51 | SEQ ID NO: 52 | | SEQ ID NO: 54 |
| 505 | SEQ ID NO: 57 | | | | | |
| 506 | SEQ ID NO: 57 | EVS | | | | |
| 507 | SEQ ID NO: 57 | EVS | SEQ ID NO: 58 | | | |
| 508 | | EVS | | | | |
| 509 | | EVS | SEQ ID NO: 58 | | | |
| 510 | | | SEQ ID NO: 58 | | | |
| 511 | SEQ ID NO: 57 | | SEQ ID NO: 58 | | | |
| 512 | | | | SEQ ID NO: 59 | | |
| 513 | | | | SEQ ID NO: 59 | SEQ ID NO: 60 | |
| 514 | | | | SEQ ID NO: 59 | SEQ ID NO: 60 | SEQ ID NO: 61 |
| 515 | | | | | SEQ ID NO: 60 | |
| 516 | | | | | SEQ ID NO: 60 | SEQ ID NO: 61 |
| 517 | | | | | | SEQ ID NO: 61 |
| 518 | | | | SEQ ID NO: 59 | | SEQ ID NO: 61 |
| 519 | SEQ ID NO: 57 | | | SEQ ID NO: 59 | | |
| 520 | SEQ ID NO: 57 | | | SEQ ID NO: 59 | SEQ ID NO: 60 | |
| 521 | SEQ ID NO: 57 | | | SEQ ID NO: 59 | SEQ ID NO: 60 | SEQ ID NO: 61 |
| 522 | SEQ ID NO: 57 | | | | SEQ ID NO: 60 | |
| 523 | SEQ ID NO: 57 | | | | SEQ ID NO: 60 | SEQ ID NO: 61 |
| 524 | SEQ ID NO: 57 | | | | | SEQ ID NO: 61 |
| 525 | SEQ ID NO: 57 | | | SEQ ID NO: 59 | | SEQ ID NO: 61 |
| 526 | SEQ ID NO: 57 | EVS | | SEQ ID NO: 59 | | |
| 527 | SEQ ID NO: 57 | EVS | | SEQ ID NO: 59 | SEQ ID NO: 60 | |
| 528 | SEQ ID NO: 57 | EVS | | SEQ ID NO: 59 | SEQ ID NO: 60 | SEQ ID NO: 61 |
| 529 | SEQ ID NO: 57 | EVS | | | SEQ ID NO: 60 | |
| 530 | SEQ ID NO: 57 | EVS | | | SEQ ID NO: 60 | SEQ ID NO: 61 |
| 531 | SEQ ID NO: 57 | EVS | | | | SEQ ID NO: 61 |
| 532 | SEQ ID NO: 57 | EVS | | SEQ ID NO: 59 | | SEQ ID NO: 61 |
| 533 | SEQ ID NO: 57 | EVS | SEQ ID NO: 58 | SEQ ID NO: 59 | | |
| 534 | SEQ ID NO: 57 | EVS | SEQ ID NO: 58 | SEQ ID NO: 59 | SEQ ID NO: 60 | |
| 535 | SEQ ID NO: 57 | EVS | SEQ ID NO: 58 | SEQ ID NO: 59 | SEQ ID NO: 60 | SEQ ID NO: 61 |
| 536 | SEQ ID NO: 57 | EVS | SEQ ID NO: 58 | | SEQ ID NO: 60 | |
| 537 | SEQ ID NO: 57 | EVS | SEQ ID NO: 58 | | SEQ ID NO: 60 | SEQ ID NO: 61 |
| 538 | SEQ ID NO: 57 | EVS | SEQ ID NO: 58 | SEQ ID NO: 59 | | SEQ ID NO: 61 |
| 539 | SEQ ID NO: 57 | EVS | SEQ ID NO: 58 | | | SEQ ID NO: 61 |
| 540 | | EVS | | SEQ ID NO: 59 | | |
| 541 | | EVS | | SEQ ID NO: 59 | SEQ ID NO: 60 | |
| 542 | | EVS | | SEQ ID NO: 59 | SEQ ID NO: 60 | SEQ ID NO: 61 |
| 543 | | EVS | | | SEQ ID NO: 60 | |
| 544 | | EVS | | | SEQ ID NO: 60 | SEQ ID NO: 61 |
| 545 | | EVS | | | | SEQ ID NO: 61 |
| 546 | | EVS | | SEQ ID NO: 59 | | SEQ ID NO: 61 |
| 547 | | EVS | SEQ ID NO: 58 | SEQ ID NO: 59 | | |
| 548 | | EVS | SEQ ID NO: 58 | SEQ ID NO: 59 | SEQ ID NO: 60 | |
| 549 | | EVS | SEQ ID NO: 58 | SEQ ID NO: 59 | SEQ ID NO: 60 | SEQ ID NO: 61 |
| 550 | | EVS | SEQ ID NO: 58 | | SEQ ID NO: 60 | |
| 551 | | EVS | SEQ ID NO: 58 | | SEQ ID NO: 60 | SEQ ID NO: 61 |
| 552 | | EVS | SEQ ID NO: 58 | | | SEQ ID NO: 61 |
| 553 | | EVS | SEQ ID NO: 58 | SEQ ID NO: 59 | | SEQ ID NO: 61 |
| 554 | | | SEQ ID NO: 58 | SEQ ID NO: 59 | | |
| 555 | | | SEQ ID NO: 58 | SEQ ID NO: 59 | SEQ ID NO: 60 | |
| 556 | | | SEQ ID NO: 58 | SEQ ID NO: 59 | SEQ ID NO: 60 | SEQ ID NO: 61 |
| 557 | | | SEQ ID NO: 58 | | SEQ ID NO: 60 | |
| 558 | | | SEQ ID NO: 58 | | SEQ ID NO: 60 | SEQ ID NO: 61 |
| 559 | | | SEQ ID NO: 58 | | | SEQ ID NO: 61 |
| 560 | | | SEQ ID NO: 58 | SEQ ID NO: 59 | | SEQ ID NO: 61 |
| 561 | SEQ ID NO: 57 | | SEQ ID NO: 58 | SEQ ID NO: 59 | | |
| 562 | SEQ ID NO: 57 | | SEQ ID NO: 58 | SEQ ID NO: 59 | SEQ ID NO: 60 | |
| 563 | SEQ ID NO: 57 | | SEQ ID NO: 58 | SEQ ID NO: 59 | SEQ ID NO: 60 | SEQ ID NO: 61 |
| 564 | SEQ ID NO: 57 | | SEQ ID NO: 58 | | SEQ ID NO: 60 | |
| 565 | SEQ ID NO: 57 | | SEQ ID NO: 58 | | SEQ ID NO: 60 | SEQ ID NO: 61 |
| 566 | SEQ ID NO: 57 | | SEQ ID NO: 58 | | | SEQ ID NO: 61 |
| 567 | SEQ ID NO: 57 | | SEQ ID NO: 58 | SEQ ID NO: 59 | | SEQ ID NO: 61 |
| 568 | SEQ ID NO: 64 | | | | | |
| 569 | SEQ ID NO: 64 | EDS | | | | |
| 570 | SEQ ID NO: 64 | EDS | SEQ ID NO: 65 | | | |
| 571 | | EDS | | | | |
| 572 | | EDS | SEQ ID NO: 65 | | | |
| 573 | | | SEQ ID NO: 65 | | | |
| 574 | SEQ ID NO: 64 | | SEQ ID NO: 65 | | | |
| 575 | | | | SEQ ID NO: 66 | | |
| 576 | | | | SEQ ID NO: 66 | SEQ ID NO: 67 | |
| 577 | | | | SEQ ID NO: 66 | SEQ ID NO: 67 | SEQ ID NO: 68 |
| 578 | | | | | SEQ ID NO: 67 | |
| 579 | | | | | SEQ ID NO: 67 | SEQ ID NO: 68 |
| 580 | | | | | | SEQ ID NO: 68 |
| 581 | | | | SEQ ID NO: 66 | | SEQ ID NO: 68 |
| 582 | SEQ ID NO: 64 | | | SEQ ID NO: 66 | | |
| 583 | SEQ ID NO: 64 | | | SEQ ID NO: 66 | SEQ ID NO: 67 | |
| 584 | SEQ ID NO: 64 | | | SEQ ID NO: 66 | SEQ ID NO: 67 | SEQ ID NO: 68 |
| 585 | SEQ ID NO: 64 | | | | SEQ ID NO: 67 | |
| 586 | SEQ ID NO: 64 | | | | SEQ ID NO: 67 | SEQ ID NO: 68 |
| 587 | SEQ ID NO: 64 | | | | | SEQ ID NO: 68 |
| 588 | SEQ ID NO: 64 | | | SEQ ID NO: 66 | | SEQ ID NO: 68 |
| 589 | SEQ ID NO: 64 | EDS | | SEQ ID NO: 66 | | |
| 590 | SEQ ID NO: 64 | EDS | | SEQ ID NO: 66 | SEQ ID NO: 67 | |
| 591 | SEQ ID NO: 64 | EDS | | SEQ ID NO: 66 | SEQ ID NO: 67 | SEQ ID NO: 68 |
| 592 | SEQ ID NO: 64 | EDS | | | SEQ ID NO: 67 | |
| 593 | SEQ ID NO: 64 | EDS | | | SEQ ID NO: 67 | SEQ ID NO: 68 |
| 594 | SEQ ID NO: 64 | EDS | | | | SEQ ID NO: 68 |
| 595 | SEQ ID NO: 64 | EDS | | SEQ ID NO: 66 | | SEQ ID NO: 68 |
| 596 | SEQ ID NO: 64 | EDS | SEQ ID NO: 65 | SEQ ID NO: 66 | | |
| 597 | SEQ ID NO: 64 | EDS | SEQ ID NO: 65 | SEQ ID NO: 66 | SEQ ID NO: 67 | |
| 598 | SEQ ID NO: 64 | EDS | SEQ ID NO: 65 | SEQ ID NO: 66 | SEQ ID NO: 67 | SEQ ID NO: 68 |
| 599 | SEQ ID NO: 64 | EDS | SEQ ID NO: 65 | | SEQ ID NO: 67 | |
| 600 | SEQ ID NO: 64 | EDS | SEQ ID NO: 65 | | SEQ ID NO: 67 | SEQ ID NO: 68 |
| 601 | SEQ ID NO: 64 | EDS | SEQ ID NO: 65 | SEQ ID NO: 66 | | SEQ ID NO: 68 |
| 602 | SEQ ID NO: 64 | EDS | SEQ ID NO: 65 | | | SEQ ID NO: 68 |
| 603 | | EDS | | SEQ ID NO: 66 | | |
| 604 | | EDS | | SEQ ID NO: 66 | SEQ ID NO: 67 | |
| 605 | | EDS | | SEQ ID NO: 66 | SEQ ID NO: 67 | SEQ ID NO: 68 |
| 606 | | EDS | | | SEQ ID NO: 67 | |
| 607 | | EDS | | | SEQ ID NO: 67 | SEQ ID NO: 68 |
| 608 | | EDS | | | | SEQ ID NO: 68 |
| 609 | | EDS | | SEQ ID NO: 66 | | SEQ ID NO: 68 |
| 610 | | EDS | SEQ ID NO: 65 | SEQ ID NO: 66 | | |
| 611 | | EDS | SEQ ID NO: 65 | SEQ ID NO: 66 | SEQ ID NO: 67 | |
| 612 | | EDS | SEQ ID NO: 65 | SEQ ID NO: 66 | SEQ ID NO: 67 | SEQ ID NO: 68 |
| 613 | | EDS | SEQ ID NO: 65 | | SEQ ID NO: 67 | |
| 614 | | EDS | SEQ ID NO: 65 | | SEQ ID NO: 67 | SEQ ID NO: 68 |
| 615 | | EDS | SEQ ID NO: 65 | | | SEQ ID NO: 68 |
| 616 | | EDS | SEQ ID NO: 65 | SEQ ID NO: 66 | | SEQ ID NO: 68 |
| 617 | | | SEQ ID NO: 65 | SEQ ID NO: 66 | | |
| 618 | | | SEQ ID NO: 65 | SEQ ID NO: 66 | SEQ ID NO: 67 | |
| 619 | | | SEQ ID NO: 65 | SEQ ID NO: 66 | SEQ ID NO: 67 | SEQ ID NO: 68 |
| 620 | | | SEQ ID NO: 65 | | SEQ ID NO: 67 | |
| 621 | | | SEQ ID NO: 65 | | SEQ ID NO: 67 | SEQ ID NO: 68 |
| 622 | | | SEQ ID NO: 65 | | | SEQ ID NO: 68 |
| 623 | | | SEQ ID NO: 65 | SEQ ID NO: 66 | | SEQ ID NO: 68 |
| 624 | SEQ ID NO: 64 | | SEQ ID NO: 65 | SEQ ID NO: 66 | | |
| 625 | SEQ ID NO: 64 | | SEQ ID NO: 65 | SEQ ID NO: 66 | SEQ ID NO: 67 | |
| 626 | SEQ ID NO: 64 | | SEQ ID NO: 65 | SEQ ID NO: 66 | SEQ ID NO: 67 | SEQ ID NO: 68 |
| 627 | SEQ ID NO: 64 | | SEQ ID NO: 65 | | SEQ ID NO: 67 | |
| 628 | SEQ ID NO: 64 | | SEQ ID NO: 65 | | SEQ ID NO: 67 | SEQ ID NO: 68 |
| 629 | SEQ ID NO: 64 | | SEQ ID NO: 65 | | | SEQ ID NO: 68 |
| 630 | SEQ ID NO: 64 | | SEQ ID NO: 65 | SEQ ID NO: 66 | | SEQ ID NO: 68 |
| 631 | SEQ ID NO: 71 | | | | | |
| 632 | SEQ ID NO: 71 | EDS | | | | |
| 633 | SEQ ID NO: 71 | EDS | SEQ ID NO: 72 | | | |
| 634 | | EDS | | | | |
| 635 | | EDS | SEQ ID NO: 72 | | | |
| 636 | | | SEQ ID NO: 72 | | | |
| 637 | SEQ ID NO: 71 | | SEQ ID NO: 72 | | | |
| 638 | | | | SEQ ID NO: 73 | | |
| 639 | | | | SEQ ID NO: 73 | SEQ ID NO: 74 | |
| 640 | | | | SEQ ID NO: 73 | SEQ ID NO: 74 | SEQ ID NO: 75 |
| 641 | | | | | SEQ ID NO: 74 | |
| 642 | | | | | SEQ ID NO: 74 | SEQ ID NO: 75 |
| 643 | | | | | | SEQ ID NO: 75 |
| 644 | | | | SEQ ID NO: 73 | | SEQ ID NO: 75 |
| 645 | SEQ ID NO: 71 | | | SEQ ID NO: 73 | | |
| 646 | SEQ ID NO: 71 | | | SEQ ID NO: 73 | SEQ ID NO: 74 | |
| 647 | SEQ ID NO: 71 | | | SEQ ID NO: 73 | SEQ ID NO: 74 | SEQ ID NO: 75 |
| 648 | SEQ ID NO: 71 | | | | SEQ ID NO: 74 | |
| 649 | SEQ ID NO: 71 | | | | SEQ ID NO: 74 | SEQ ID NO: 75 |
| 650 | SEQ ID NO: 71 | | | | | SEQ ID NO: 75 |
| 651 | SEQ ID NO: 71 | | | SEQ ID NO: 73 | | SEQ ID NO: 75 |
| 652 | SEQ ID NO: 71 | EDS | | SEQ ID NO: 73 | | |
| 653 | SEQ ID NO: 71 | EDS | | SEQ ID NO: 73 | SEQ ID NO: 74 | |
| 654 | SEQ ID NO: 71 | EDS | | SEQ ID NO: 73 | SEQ ID NO: 74 | SEQ ID NO: 75 |
| 655 | SEQ ID NO: 71 | EDS | | | SEQ ID NO: 74 | |
| 656 | SEQ ID NO: 71 | EDS | | | SEQ ID NO: 74 | SEQ ID NO: 75 |
| 657 | SEQ ID NO: 71 | EDS | | | | SEQ ID NO: 75 |
| 658 | SEQ ID NO: 71 | EDS | | SEQ ID NO: 73 | | SEQ ID NO: 75 |
| 659 | SEQ ID NO: 71 | EDS | SEQ ID NO: 72 | SEQ ID NO: 73 | | |
| 660 | SEQ ID NO: 71 | EDS | SEQ ID NO: 72 | SEQ ID NO: 73 | SEQ ID NO: 74 | |
| 661 | SEQ ID NO: 71 | EDS | SEQ ID NO: 72 | SEQ ID NO: 73 | SEQ ID NO: 74 | SEQ ID NO: 75 |
| 662 | SEQ ID NO: 71 | EDS | SEQ ID NO: 72 | | SEQ ID NO: 74 | |
| 663 | SEQ ID NO: 71 | EDS | SEQ ID NO: 72 | | SEQ ID NO: 74 | SEQ ID NO: 75 |
| 664 | SEQ ID NO: 71 | EDS | SEQ ID NO: 72 | SEQ ID NO: 73 | | SEQ ID NO: 75 |
| 665 | SEQ ID NO: 71 | EDS | SEQ ID NO: 72 | | | SEQ ID NO: 75 |
| 666 | | EDS | | SEQ ID NO: 73 | | |
| 667 | | EDS | | SEQ ID NO: 73 | SEQ ID NO: 74 | |
| 668 | | EDS | | SEQ ID NO: 73 | SEQ ID NO: 74 | SEQ ID NO: 75 |
| 669 | | EDS | | | SEQ ID NO: 74 | |
| 670 | | EDS | | | SEQ ID NO: 74 | SEQ ID NO: 75 |
| 671 | | EDS | | | | SEQ ID NO: 75 |
| 672 | | EDS | | SEQ ID NO: 73 | | SEQ ID NO: 75 |
| 673 | | EDS | SEQ ID NO: 72 | SEQ ID NO: 73 | | |
| 674 | | EDS | SEQ ID NO: 72 | SEQ ID NO: 73 | SEQ ID NO: 74 | |
| 675 | | EDS | SEQ ID NO: 72 | SEQ ID NO: 73 | SEQ ID NO: 74 | SEQ ID NO: 75 |
| 676 | | EDS | SEQ ID NO: 72 | | SEQ ID NO: 74 | |
| 677 | | EDS | SEQ ID NO: 72 | | SEQ ID NO: 74 | SEQ ID NO: 75 |
| 678 | | EDS | SEQ ID NO: 72 | | | SEQ ID NO: 75 |
| 679 | | EDS | SEQ ID NO: 72 | SEQ ID NO: 73 | | SEQ ID NO: 75 |
| 680 | | | SEQ ID NO: 72 | SEQ ID NO: 73 | | |
| 681 | | | SEQ ID NO: 72 | SEQ ID NO: 73 | SEQ ID NO: 74 | |
| 682 | | | SEQ ID NO: 72 | SEQ ID NO: 73 | SEQ ID NO: 74 | SEQ ID NO: 75 |
| 683 | | | SEQ ID NO: 72 | | SEQ ID NO: 74 | |
| 684 | | | SEQ ID NO: 72 | | SEQ ID NO: 74 | SEQ ID NO: 75 |
| 685 | | | SEQ ID NO: 72 | | | SEQ ID NO: 75 |
| 686 | | | SEQ ID NO: 72 | SEQ ID NO: 73 | | SEQ ID NO: 75 |
| 687 | SEQ ID NO: 71 | | SEQ ID NO: 72 | SEQ ID NO: 73 | | |
| 688 | SEQ ID NO: 71 | | SEQ ID NO: 72 | SEQ ID NO: 73 | SEQ ID NO: 74 | |
| 689 | SEQ ID NO: 71 | | SEQ ID NO: 72 | SEQ ID NO: 73 | SEQ ID NO: 74 | SEQ ID NO: 75 |
| 690 | SEQ ID NO: 71 | | SEQ ID NO: 72 | | SEQ ID NO: 74 | |
| 691 | SEQ ID NO: 71 | | SEQ ID NO: 72 | | SEQ ID NO: 74 | SEQ ID NO: 75 |
| 692 | SEQ ID NO: 71 | | SEQ ID NO: 72 | | | SEQ ID NO: 75 |
| 693 | SEQ ID NO: 71 | | SEQ ID NO: 72 | SEQ ID NO: 73 | | SEQ ID NO: 75 |
| 694 | SEQ ID NO: 78 | | | | | |
| 695 | SEQ ID NO: 78 | EDS | | | | |
| 696 | SEQ ID NO: 78 | EDS | SEQ ID NO: 79 | | | |
| 697 | | EDS | | | | |
| 698 | | EDS | SEQ ID NO: 79 | | | |
| 699 | | | SEQ ID NO: 79 | | | |
| 700 | SEQ ID NO: 78 | | SEQ ID NO: 79 | | | |
| 701 | | | | SEQ ID NO: 80 | | |
| 702 | | | | SEQ ID NO: 80 | SEQ ID NO: 81 | |
| 703 | | | | SEQ ID NO: 80 | SEQ ID NO: 81 | SEQ ID NO: 82 |
| 704 | | | | | SEQ ID NO: 81 | |
| 705 | | | | | SEQ ID NO: 81 | SEQ ID NO: 82 |
| 706 | | | | | | SEQ ID NO: 82 |
| 707 | | | | SEQ ID NO: 80 | | SEQ ID NO: 82 |
| 708 | SEQ ID NO: 78 | | | SEQ ID NO: 80 | | |
| 709 | SEQ ID NO: 78 | | | SEQ ID NO: 80 | SEQ ID NO: 81 | |
| 710 | SEQ ID NO: 78 | | | SEQ ID NO: 80 | SEQ ID NO: 81 | SEQ ID NO: 82 |
| 711 | SEQ ID NO: 78 | | | | SEQ ID NO: 81 | |
| 712 | SEQ ID NO: 78 | | | | SEQ ID NO: 81 | SEQ ID NO: 82 |
| 713 | SEQ ID NO: 78 | | | | | SEQ ID NO: 82 |
| 714 | SEQ ID NO: 78 | | | SEQ ID NO: 80 | | SEQ ID NO: 82 |
| 715 | SEQ ID NO: 78 | EDS | | SEQ ID NO: 80 | | |
| 716 | SEQ ID NO: 78 | EDS | | SEQ ID NO: 80 | SEQ ID NO: 81 | |
| 717 | SEQ ID NO: 78 | EDS | | SEQ ID NO: 80 | SEQ ID NO: 81 | SEQ ID NO: 82 |
| 718 | SEQ ID NO: 78 | EDS | | | SEQ ID NO: 81 | |
| 719 | SEQ ID NO: 78 | EDS | | | SEQ ID NO: 81 | SEQ ID NO: 82 |
| 720 | SEQ ID NO: 78 | EDS | | | | SEQ ID NO: 82 |
| 721 | SEQ ID NO: 78 | EDS | | SEQ ID NO: 80 | | SEQ ID NO: 82 |
| 722 | SEQ ID NO: 78 | EDS | SEQ ID NO: 79 | SEQ ID NO: 80 | | |
| 723 | SEQ ID NO: 78 | EDS | SEQ ID NO: 79 | SEQ ID NO: 80 | SEQ ID NO: 81 | |
| 724 | SEQ ID NO: 78 | EDS | SEQ ID NO: 79 | SEQ ID NO: 80 | SEQ ID NO: 81 | SEQ ID NO: 82 |
| 725 | SEQ ID NO: 78 | EDS | SEQ ID NO: 79 | | SEQ ID NO: 81 | |
| 726 | SEQ ID NO: 78 | EDS | SEQ ID NO: 79 | | SEQ ID NO: 81 | SEQ ID NO: 82 |
| 727 | SEQ ID NO: 78 | EDS | SEQ ID NO: 79 | | | SEQ ID NO: 82 |
| 728 | SEQ ID NO: 78 | EDS | SEQ ID NO: 79 | SEQ ID NO: 80 | | SEQ ID NO: 82 |
| 729 | | EDS | | SEQ ID NO: 80 | | |
| 730 | | EDS | | SEQ ID NO: 80 | SEQ ID NO: 81 | |
| 731 | | EDS | | SEQ ID NO: 80 | SEQ ID NO: 81 | SEQ ID NO: 82 |
| 732 | | EDS | | | SEQ ID NO: 81 | |
| 733 | | EDS | | | SEQ ID NO: 81 | SEQ ID NO: 82 |
| 734 | | EDS | | | | SEQ ID NO: 82 |
| 735 | | EDS | | SEQ ID NO: 80 | | SEQ ID NO: 82 |
| 736 | | EDS | SEQ ID NO: 79 | SEQ ID NO: 80 | | |
| 737 | | EDS | SEQ ID NO: 79 | SEQ ID NO: 80 | SEQ ID NO: 81 | |
| 738 | | EDS | SEQ ID NO: 79 | SEQ ID NO: 80 | SEQ ID NO: 81 | SEQ ID NO: 82 |
| 739 | | EDS | SEQ ID NO: 79 | | SEQ ID NO: 81 | |
| 740 | | EDS | SEQ ID NO: 79 | | SEQ ID NO: 81 | SEQ ID NO: 82 |
| 741 | | EDS | SEQ ID NO: 79 | | | SEQ ID NO: 82 |
| 742 | | EDS | SEQ ID NO: 79 | SEQ ID NO: 80 | | SEQ ID NO: 82 |
| 743 | | | SEQ ID NO: 79 | SEQ ID NO: 80 | | |
| 744 | | | SEQ ID NO: 79 | SEQ ID NO: 80 | SEQ ID NO: 81 | |
| 745 | | | SEQ ID NO: 79 | SEQ ID NO: 80 | SEQ ID NO: 81 | SEQ ID NO: 82 |
| 746 | | | SEQ ID NO: 79 | | SEQ ID NO: 81 | |
| 747 | | | SEQ ID NO: 79 | | SEQ ID NO: 81 | SEQ ID NO: 82 |
| 748 | | | SEQ ID NO: 79 | | | SEQ ID NO: 82 |
| 749 | | | SEQ ID NO: 79 | SEQ ID NO: 80 | | SEQ ID NO: 82 |
| 750 | SEQ ID NO: 78 | | SEQ ID NO: 79 | SEQ ID NO: 80 | | |
| 751 | SEQ ID NO: 78 | | SEQ ID NO: 79 | SEQ ID NO: 80 | SEQ ID NO: 81 | |
| 752 | SEQ ID NO: 78 | | SEQ ID NO: 79 | SEQ ID NO: 80 | SEQ ID NO: 81 | SEQ ID NO: 82 |
| 753 | SEQ ID NO: 78 | | SEQ ID NO: 79 | | SEQ ID NO: 81 | |
| 754 | SEQ ID NO: 78 | | SEQ ID NO: 79 | | SEQ ID NO: 81 | SEQ ID NO: 82 |
| 755 | SEQ ID NO: 78 | | SEQ ID NO: 79 | | | SEQ ID NO: 82 |
| 756 | SEQ ID NO: 78 | | SEQ ID NO: 79 | SEQ ID NO: 80 | | SEQ ID NO: 82 |
| 757 | SEQ ID NO: 85 | | | | | |
| 758 | SEQ ID NO: 85 | DAS | | | | |
| 759 | SEQ ID NO: 85 | DAS | SEQ ID NO: 86 | | | |
| 760 | | DAS | | | | |
| 761 | | DAS | SEQ ID NO: 86 | | | |
| 762 | | | SEQ ID NO: 86 | | | |
| 763 | SEQ ID NO: 85 | | SEQ ID NO: 86 | | | |
| 764 | | | | SEQ ID NO: 87 | | |
| 765 | | | | SEQ ID NO: 87 | SEQ ID NO: 88 | |
| 766 | | | | SEQ ID NO: 87 | SEQ ID NO: 88 | SEQ ID NO: 89 |
| 767 | | | | | SEQ ID NO: 88 | |
| 768 | | | | | SEQ ID NO: 88 | SEQ ID NO: 89 |
| 769 | | | | | | SEQ ID NO: 89 |
| 770 | | | | SEQ ID NO: 87 | | SEQ ID NO: 89 |
| 771 | SEQ ID NO: 85 | | | SEQ ID NO: 87 | | |
| 772 | SEQ ID NO: 85 | | | SEQ ID NO: 87 | SEQ ID NO: 88 | |
| 773 | SEQ ID NO: 85 | | | SEQ ID NO: 87 | SEQ ID NO: 88 | SEQ ID NO: 89 |
| 774 | SEQ ID NO: 85 | | | | SEQ ID NO: 88 | |
| 775 | SEQ ID NO: 85 | | | | SEQ ID NO: 88 | SEQ ID NO: 89 |
| 776 | SEQ ID NO: 85 | | | | | SEQ ID NO: 89 |
| 777 | SEQ ID NO: 85 | | | SEQ ID NO: 87 | | SEQ ID NO: 89 |
| 778 | SEQ ID NO: 85 | DAS | | SEQ ID NO: 87 | | |
| 779 | SEQ ID NO: 85 | DAS | | SEQ ID NO: 87 | SEQ ID NO: 88 | |
| 780 | SEQ ID NO: 85 | DAS | | SEQ ID NO: 87 | SEQ ID NO: 88 | SEQ ID NO: 89 |
| 781 | SEQ ID NO: 85 | DAS | | | SEQ ID NO: 88 | |
| 782 | SEQ ID NO: 85 | DAS | | | SEQ ID NO: 88 | SEQ ID NO: 89 |
| 783 | SEQ ID NO: 85 | DAS | | | | SEQ ID NO: 89 |
| 784 | SEQ ID NO: 85 | DAS | | SEQ ID NO: 87 | | SEQ ID NO: 89 |
| 785 | SEQ ID NO: 85 | DAS | SEQ ID NO: 86 | SEQ ID NO: 87 | | |
| 786 | SEQ ID NO: 85 | DAS | SEQ ID NO: 86 | SEQ ID NO: 87 | SEQ ID NO: 88 | |
| 787 | SEQ ID NO: 85 | DAS | SEQ ID NO: 86 | SEQ ID NO: 87 | SEQ ID NO: 88 | SEQ ID NO: 89 |
| 788 | SEQ ID NO: 85 | DAS | SEQ ID NO: 86 | | SEQ ID NO: 88 | |
| 789 | SEQ ID NO: 85 | DAS | SEQ ID NO: 86 | | SEQ ID NO: 88 | SEQ ID NO: 89 |
| 790 | SEQ ID NO: 85 | DAS | SEQ ID NO: 86 | SEQ ID NO: 87 | | SEQ ID NO: 89 |
| 791 | SEQ ID NO: 85 | DAS | SEQ ID NO: 86 | | | SEQ ID NO: 89 |
| 792 | | DAS | | SEQ ID NO: 87 | | |
| 793 | | DAS | | SEQ ID NO: 87 | SEQ ID NO: 88 | |
| 794 | | DAS | | SEQ ID NO: 87 | SEQ ID NO: 88 | SEQ ID NO: 89 |
| 795 | | DAS | | | SEQ ID NO: 88 | |
| 796 | | DAS | | | SEQ ID NO: 88 | SEQ ID NO: 89 |
| 797 | | DAS | | | | SEQ ID NO: 89 |
| 798 | | DAS | | SEQ ID NO: 87 | | SEQ ID NO: 89 |
| 799 | | DAS | SEQ ID NO: 86 | SEQ ID NO: 87 | | |
| 800 | | DAS | SEQ ID NO: 86 | SEQ ID NO: 87 | SEQ ID NO: 88 | |
| 801 | | DAS | SEQ ID NO: 86 | SEQ ID NO: 87 | SEQ ID NO: 88 | SEQ ID NO: 89 |
| 802 | | DAS | SEQ ID NO: 86 | | SEQ ID NO: 88 | |
| 803 | | DAS | SEQ ID NO: 86 | | SEQ ID NO: 88 | SEQ ID NO: 89 |
| 804 | | DAS | SEQ ID NO: 86 | | | SEQ ID NO: 89 |
| 805 | | DAS | SEQ ID NO: 86 | SEQ ID NO: 87 | | SEQ ID NO: 89 |
| 806 | | | SEQ ID NO: 86 | SEQ ID NO: 87 | | |
| 807 | | | SEQ ID NO: 86 | SEQ ID NO: 87 | SEQ ID NO: 88 | |
| 808 | | | SEQ ID NO: 86 | SEQ ID NO: 87 | SEQ ID NO: 88 | SEQ ID NO: 89 |
| 809 | | | SEQ ID NO: 86 | | SEQ ID NO: 88 | |
| 810 | | | SEQ ID NO: 86 | | SEQ ID NO: 88 | SEQ ID NO: 89 |
| 811 | | | SEQ ID NO: 86 | | | SEQ ID NO: 89 |
| 812 | | | SEQ ID NO: 86 | SEQ ID NO: 87 | | SEQ ID NO: 89 |
| 813 | SEQ ID NO: 85 | | SEQ ID NO: 86 | SEQ ID NO: 87 | | |
| 814 | SEQ ID NO: 85 | | SEQ ID NO: 86 | SEQ ID NO: 87 | SEQ ID NO: 88 | |
| 815 | SEQ ID NO: 85 | | SEQ ID NO: 86 | SEQ ID NO: 87 | SEQ ID NO: 88 | SEQ ID NO: 89 |
| 816 | SEQ ID NO: 85 | | SEQ ID NO: 86 | | SEQ ID NO: 88 | |
| 817 | SEQ ID NO: 85 | | SEQ ID NO: 86 | | SEQ ID NO: 88 | SEQ ID NO: 89 |
| 818 | SEQ ID NO: 85 | | SEQ ID NO: 86 | | | SEQ ID NO: 89 |
| 819 | SEQ ID NO: 85 | | SEQ ID NO: 86 | SEQ ID NO: 87 | | SEQ ID NO: 89 |
| 820 | SEQ ID NO: 92 | | | | | |
| 821 | SEQ ID NO: 92 | NAS | | | | |
| 822 | SEQ ID NO: 92 | NAS | SEQ ID NO: 93 | | | |
| 823 | | NAS | | | | |
| 824 | | NAS | SEQ ID NO: 93 | | | |
| 825 | | | SEQ ID NO: 93 | | | |
| 826 | SEQ ID NO: 92 | | SEQ ID NO: 93 | | | |
| 827 | | | | SEQ ID NO: 94 | | |
| 828 | | | | SEQ ID NO: 94 | SEQ ID NO: 95 | |
| 829 | | | | SEQ ID NO: 94 | SEQ ID NO: 95 | SEQ ID NO: 96 |
| 830 | | | | | SEQ ID NO: 95 | |
| 831 | | | | | SEQ ID NO: 95 | SEQ ID NO: 96 |
| 832 | | | | | | SEQ ID NO: 96 |
| 833 | | | | SEQ ID NO: 94 | | SEQ ID NO: 96 |
| 834 | SEQ ID NO: 92 | | | SEQ ID NO: 94 | | |
| 835 | SEQ ID NO: 92 | | | SEQ ID NO: 94 | SEQ ID NO: 95 | |
| 836 | SEQ ID NO: 92 | | | SEQ ID NO: 94 | SEQ ID NO: 95 | SEQ ID NO: 96 |
| 837 | SEQ ID NO: 92 | | | | SEQ ID NO: 95 | |
| 838 | SEQ ID NO: 92 | | | | SEQ ID NO: 95 | SEQ ID NO: 96 |
| 839 | SEQ ID NO: 92 | | | | | SEQ ID NO: 96 |
| 840 | SEQ ID NO: 92 | | | SEQ ID NO: 94 | | SEQ ID NO: 96 |
| 841 | SEQ ID NO: 92 | NAS | | SEQ ID NO: 94 | | |
| 842 | SEQ ID NO: 92 | NAS | | SEQ ID NO: 94 | SEQ ID NO: 95 | |
| 843 | SEQ ID NO: 92 | NAS | | SEQ ID NO: 94 | SEQ ID NO: 95 | SEQ ID NO: 96 |
| 844 | SEQ ID NO: 92 | NAS | | | SEQ ID NO: 95 | |
| 845 | SEQ ID NO: 92 | NAS | | | SEQ ID NO: 95 | SEQ ID NO: 96 |
| 846 | SEQ ID NO: 92 | NAS | | | | SEQ ID NO: 96 |
| 847 | SEQ ID NO: 92 | NAS | | SEQ ID NO: 94 | | SEQ ID NO: 96 |
| 848 | SEQ ID NO: 92 | NAS | SEQ ID NO: 93 | SEQ ID NO: 94 | | |
| 849 | SEQ ID NO: 92 | NAS | SEQ ID NO: 93 | SEQ ID NO: 94 | SEQ ID NO: 95 | |
| 850 | SEQ ID NO: 92 | NAS | SEQ ID NO: 93 | SEQ ID NO: 94 | SEQ ID NO: 95 | SEQ ID NO: 96 |
| 851 | SEQ ID NO: 92 | NAS | SEQ ID NO: 93 | | SEQ ID NO: 95 | |
| 852 | SEQ ID NO: 92 | NAS | SEQ ID NO: 93 | | SEQ ID NO: 95 | SEQ ID NO: 96 |
| 853 | SEQ ID NO: 92 | NAS | SEQ ID NO: 93 | SEQ ID NO: 94 | | SEQ ID NO: 96 |
| 854 | SEQ ID NO: 92 | NAS | SEQ ID NO: 93 | | | SEQ ID NO: 96 |
| 855 | | NAS | | SEQ ID NO: 94 | | |
| 856 | | NAS | | SEQ ID NO: 94 | SEQ ID NO: 95 | |
| 857 | | NAS | | SEQ ID NO: 94 | SEQ ID NO: 95 | SEQ ID NO: 96 |
| 858 | | NAS | | | SEQ ID NO: 95 | |
| 859 | | NAS | | | SEQ ID NO: 95 | SEQ ID NO: 96 |
| 860 | | NAS | | | | SEQ ID NO: 96 |
| 861 | | NAS | | SEQ ID NO: 94 | | SEQ ID NO: 96 |
| 862 | | NAS | SEQ ID NO: 93 | SEQ ID NO: 94 | | |
| 863 | | NAS | SEQ ID NO: 93 | SEQ ID NO: 94 | SEQ ID NO: 95 | |
| 864 | | NAS | SEQ ID NO: 93 | SEQ ID NO: 94 | SEQ ID NO: 95 | SEQ ID NO: 96 |
| 865 | | NAS | SEQ ID NO: 93 | | SEQ ID NO: 95 | |
| 866 | | NAS | SEQ ID NO: 93 | | SEQ ID NO: 95 | SEQ ID NO: 96 |
| 867 | | NAS | SEQ ID NO: 93 | | | SEQ ID NO: 96 |
| 868 | | NAS | SEQ ID NO: 93 | SEQ ID NO: 94 | | SEQ ID NO: 96 |
| 869 | | | SEQ ID NO: 93 | SEQ ID NO: 94 | | |
| 870 | | | SEQ ID NO: 93 | SEQ ID NO: 94 | SEQ ID NO: 95 | |
| 871 | | | SEQ ID NO: 93 | SEQ ID NO: 94 | SEQ ID NO: 95 | SEQ ID NO: 96 |
| 872 | | | SEQ ID NO: 93 | | SEQ ID NO: 95 | |
| 873 | | | SEQ ID NO: 93 | | SEQ ID NO: 95 | SEQ ID NO: 96 |
| 874 | | | SEQ ID NO: 93 | | | SEQ ID NO: 96 |
| 875 | | | SEQ ID NO: 93 | SEQ ID NO: 94 | | SEQ ID NO: 96 |
| 876 | SEQ ID NO: 92 | | SEQ ID NO: 93 | SEQ ID NO: 94 | | |
| 877 | SEQ ID NO: 92 | | SEQ ID NO: 93 | SEQ ID NO: 94 | SEQ ID NO: 95 | |
| 878 | SEQ ID NO: 92 | | SEQ ID NO: 93 | SEQ ID NO: 94 | SEQ ID NO: 95 | SEQ ID NO: 96 |
| 879 | SEQ ID NO: 92 | | SEQ ID NO: 93 | | SEQ ID NO: 95 | |
| 880 | SEQ ID NO: 92 | | SEQ ID NO: 93 | | SEQ ID NO: 95 | SEQ ID NO: 96 |
| 881 | SEQ ID NO: 92 | | SEQ ID NO: 93 | | | SEQ ID NO: 96 |
| 882 | SEQ ID NO: 92 | | SEQ ID NO: 93 | SEQ ID NO: 94 | | SEQ ID NO: 96 |
| 883 | SEQ ID NO: 99 | | | | | |
| 884 | SEQ ID NO: 99 | WAS | | | | |
| 885 | SEQ ID NO: 99 | WAS | SEQ ID NO: 100 | | | |
| 886 | | WAS | | | | |
| 887 | | WAS | SEQ ID NO: 100 | | | |
| 888 | | | SEQ ID NO: 100 | | | |
| 889 | SEQ ID NO: 99 | | SEQ ID NO: 100 | | | |
| 890 | | | | SEQ ID NO: 101 | | |
| 891 | | | | SEQ ID NO: 101 | SEQ ID NO: 102 | |
| 892 | | | | SEQ ID NO: 101 | SEQ ID NO: 102 | SEQ ID NO: 103 |
| 893 | | | | | SEQ ID NO: 102 | |
| 894 | | | | | SEQ ID NO: 102 | SEQ ID NO: 103 |
| 895 | | | | | | SEQ ID NO: 103 |
| 896 | | | | SEQ ID NO: 101 | | SEQ ID NO: 103 |
| 897 | SEQ ID NO: 99 | | | SEQ ID NO: 101 | | |
| 898 | SEQ ID NO: 99 | | | SEQ ID NO: 101 | SEQ ID NO: 102 | |
| 899 | SEQ ID NO: 99 | | | SEQ ID NO: 101 | SEQ ID NO: 102 | SEQ ID NO: 103 |
| 900 | SEQ ID NO: 99 | | | | SEQ ID NO: 102 | |
| 901 | SEQ ID NO: 99 | | | | SEQ ID NO: 102 | SEQ ID NO: 103 |
| 902 | SEQ ID NO: 99 | | | | | SEQ ID NO: 103 |
| 903 | SEQ ID NO: 99 | | | SEQ ID NO: 101 | | SEQ ID NO: 103 |
| 904 | SEQ ID NO: 99 | WAS | | SEQ ID NO: 101 | | |
| 905 | SEQ ID NO: 99 | WAS | | SEQ ID NO: 101 | SEQ ID NO: 102 | |
| 906 | SEQ ID NO: 99 | WAS | | SEQ ID NO: 101 | SEQ ID NO: 102 | SEQ ID NO: 103 |
| 907 | SEQ ID NO: 99 | WAS | | | SEQ ID NO: 102 | |
| 908 | SEQ ID NO: 99 | WAS | | | SEQ ID NO: 102 | SEQ ID NO: 103 |
| 909 | SEQ ID NO: 99 | WAS | | | | SEQ ID NO: 103 |
| 910 | SEQ ID NO: 99 | WAS | | SEQ ID NO: 101 | | SEQ ID NO: 103 |
| 911 | SEQ ID NO: 99 | WAS | SEQ ID NO: 100 | SEQ ID NO: 101 | | |
| 912 | SEQ ID NO: 99 | WAS | SEQ ID NO: 100 | SEQ ID NO: 101 | SEQ ID NO: 102 | |
| 913 | SEQ ID NO: 99 | WAS | SEQ ID NO: 100 | SEQ ID NO: 101 | SEQ ID NO: 102 | SEQ ID NO: 103 |
| 914 | SEQ ID NO: 99 | WAS | SEQ ID NO: 100 | | SEQ ID NO: 102 | |
| 915 | SEQ ID NO: 99 | WAS | SEQ ID NO: 100 | | SEQ ID NO: 102 | SEQ ID NO: 103 |
| 916 | SEQ ID NO: 99 | WAS | SEQ ID NO: 100 | SEQ ID NO: 101 | | SEQ ID NO: 103 |
| 917 | SEQ ID NO: 99 | WAS | SEQ ID NO: 100 | | | SEQ ID NO: 103 |
| 918 | | WAS | | SEQ ID NO: 101 | | |
| 919 | | WAS | | SEQ ID NO: 101 | SEQ ID NO: 102 | |
| 920 | | WAS | | SEQ ID NO: 101 | SEQ ID NO: 102 | SEQ ID NO: 103 |
| 921 | | WAS | | | SEQ ID NO: 102 | |
| 922 | | WAS | | | SEQ ID NO: 102 | SEQ ID NO: 103 |
| 923 | | WAS | | | | SEQ ID NO: 103 |
| 924 | | WAS | | SEQ ID NO: 101 | | SEQ ID NO: 103 |
| 925 | | WAS | SEQ ID NO: 100 | SEQ ID NO: 101 | | |
| 926 | | WAS | SEQ ID NO: 100 | SEQ ID NO: 101 | SEQ ID NO: 102 | |
| 927 | | WAS | SEQ ID NO: 100 | SEQ ID NO: 101 | SEQ ID NO: 102 | SEQ ID NO: 103 |
| 928 | | WAS | SEQ ID NO: 100 | | SEQ ID NO: 102 | |
| 929 | | WAS | SEQ ID NO: 100 | | SEQ ID NO: 102 | SEQ ID NO: 103 |
| 930 | | WAS | SEQ ID NO: 100 | | | SEQ ID NO: 103 |
| 931 | | WAS | SEQ ID NO: 100 | SEQ ID NO: 101 | | SEQ ID NO: 103 |
| 932 | | | SEQ ID NO: 100 | SEQ ID NO: 101 | | |
| 933 | | | SEQ ID NO: 100 | SEQ ID NO: 101 | SEQ ID NO: 102 | |
| 934 | | | SEQ ID NO: 100 | SEQ ID NO: 101 | SEQ ID NO: 102 | SEQ ID NO: 103 |
| 935 | | | SEQ ID NO: 100 | | SEQ ID NO: 102 | |
| 936 | | | SEQ ID NO: 100 | | SEQ ID NO: 102 | SEQ ID NO: 103 |
| 937 | | | SEQ ID NO: 100 | | | SEQ ID NO: 103 |
| 938 | | | SEQ ID NO: 100 | SEQ ID NO: 101 | | SEQ ID NO: 103 |
| 939 | SEQ ID NO: 99 | | SEQ ID NO: 100 | SEQ ID NO: 101 | | |
| 940 | SEQ ID NO: 99 | | SEQ ID NO: 100 | SEQ ID NO: 101 | SEQ ID NO: 102 | |
| 941 | SEQ ID NO: 99 | | SEQ ID NO: 100 | SEQ ID NO: 101 | SEQ ID NO: 102 | SEQ ID NO: 103 |
| 942 | SEQ ID NO: 99 | | SEQ ID NO: 100 | | SEQ ID NO: 102 | |
| 943 | SEQ ID NO: 99 | | SEQ ID NO: 100 | | SEQ ID NO: 102 | SEQ ID NO: 103 |
| 944 | SEQ ID NO: 99 | | SEQ ID NO: 100 | | | SEQ ID NO: 103 |
| 945 | SEQ ID NO: 99 | | SEQ ID NO: 100 | SEQ ID NO: 101 | | SEQ ID NO: 103 |
| 946 | SEQ ID NO: 106 | | | | | |
| 947 | SEQ ID NO: 106 | EDS | | | | |
| 948 | SEQ ID NO: 106 | EDS | SEQ ID NO: 107 | | | |
| 949 | | EDS | | | | |
| 950 | | EDS | SEQ ID NO: 107 | | | |
| 951 | | | SEQ ID NO: 107 | | | |
| 952 | SEQ ID NO: 106 | | SEQ ID NO: 107 | | | |
| 953 | | | | SEQ ID NO: 108 | | |
| 954 | | | | SEQ ID NO: 108 | SEQ ID NO: 109 | |
| 955 | | | | SEQ ID NO: 108 | SEQ ID NO: 109 | SEQ ID NO: 110 |
| 956 | | | | | SEQ ID NO: 109 | |
| 957 | | | | | SEQ ID NO: 109 | SEQ ID NO: 110 |
| 958 | | | | | | SEQ ID NO: 110 |
| 959 | | | | SEQ ID NO: 108 | | SEQ ID NO: 110 |
| 960 | SEQ ID NO: 106 | | | SEQ ID NO: 108 | | |
| 961 | SEQ ID NO: 106 | | | SEQ ID NO: 108 | SEQ ID NO: 109 | |
| 962 | SEQ ID NO: 106 | | | SEQ ID NO: 108 | SEQ ID NO: 109 | SEQ ID NO: 110 |
| 963 | SEQ ID NO: 106 | | | | SEQ ID NO: 109 | |
| 964 | SEQ ID NO: 106 | | | | SEQ ID NO: 109 | SEQ ID NO: 110 |
| 965 | SEQ ID NO: 106 | | | | | SEQ ID NO: 110 |
| 966 | SEQ ID NO: 106 | | | SEQ ID NO: 108 | | SEQ ID NO: 110 |
| 967 | SEQ ID NO: 106 | EDS | | SEQ ID NO: 108 | | |
| 968 | SEQ ID NO: 106 | EDS | | SEQ ID NO: 108 | SEQ ID NO: 109 | |
| 969 | SEQ ID NO: 106 | EDS | | SEQ ID NO: 108 | SEQ ID NO: 109 | SEQ ID NO: 110 |
| 970 | SEQ ID NO: 106 | EDS | | | SEQ ID NO: 109 | |
| 971 | SEQ ID NO: 106 | EDS | | | SEQ ID NO: 109 | SEQ ID NO: 110 |
| 972 | SEQ ID NO: 106 | EDS | | | | SEQ ID NO: 110 |
| 973 | SEQ ID NO: 106 | EDS | | SEQ ID NO: 108 | | SEQ ID NO: 110 |
| 974 | SEQ ID NO: 106 | EDS | SEQ ID NO: 107 | SEQ ID NO: 108 | | |
| 975 | SEQ ID NO: 106 | EDS | SEQ ID NO: 107 | SEQ ID NO: 108 | SEQ ID NO: 109 | |
| 976 | SEQ ID NO: 106 | EDS | SEQ ID NO: 107 | SEQ ID NO: 108 | SEQ ID NO: 109 | SEQ ID NO: 110 |
| 977 | SEQ ID NO: 106 | EDS | SEQ ID NO: 107 | | SEQ ID NO: 109 | |
| 978 | SEQ ID NO: 106 | EDS | SEQ ID NO: 107 | | SEQ ID NO: 109 | SEQ ID NO: 110 |
| 979 | SEQ ID NO: 106 | EDS | SEQ ID NO: 107 | SEQ ID NO: 108 | | SEQ ID NO: 110 |
| 980 | SEQ ID NO: 106 | EDS | SEQ ID NO: 107 | | | SEQ ID NO: 110 |
| 981 | | EDS | | SEQ ID NO: 108 | | |
| 982 | | EDS | | SEQ ID NO: 108 | SEQ ID NO: 109 | |
| 983 | | EDS | | SEQ ID NO: 108 | SEQ ID NO: 109 | SEQ ID NO: 110 |
| 984 | | EDS | | | SEQ ID NO: 109 | |
| 985 | | EDS | | | SEQ ID NO: 109 | SEQ ID NO: 110 |
| 986 | | EDS | | | | SEQ ID NO: 110 |
| 987 | | EDS | | SEQ ID NO: 108 | | SEQ ID NO: 110 |
| 988 | | EDS | SEQ ID NO: 107 | SEQ ID NO: 108 | | |
| 989 | | EDS | SEQ ID NO: 107 | SEQ ID NO: 108 | SEQ ID NO: 109 | |
| 990 | | EDS | SEQ ID NO: 107 | SEQ ID NO: 108 | SEQ ID NO: 109 | SEQ ID NO: 110 |
| 991 | | EDS | SEQ ID NO: 107 | | SEQ ID NO: 109 | |
| 992 | | EDS | SEQ ID NO: 107 | | SEQ ID NO: 109 | SEQ ID NO: 110 |
| 993 | | EDS | SEQ ID NO: 107 | | | SEQ ID NO: 110 |
| 994 | | EDS | SEQ ID NO: 107 | SEQ ID NO: 108 | | SEQ ID NO: 110 |
| 995 | | | SEQ ID NO: 107 | SEQ ID NO: 108 | | |
| 996 | | | SEQ ID NO: 107 | SEQ ID NO: 108 | SEQ ID NO: 109 | |
| 997 | | | SEQ ID NO: 107 | SEQ ID NO: 108 | SEQ ID NO: 109 | SEQ ID NO: 110 |
| 998 | | | SEQ ID NO: 107 | | SEQ ID NO: 109 | |
| 999 | | | SEQ ID NO: 107 | | SEQ ID NO: 109 | SEQ ID NO: 110 |
| 1000 | | | SEQ ID NO: 107 | | | SEQ ID NO: 110 |
| 1001 | | | SEQ ID NO: 107 | SEQ ID NO: 108 | | SEQ ID NO: 110 |
| 1002 | SEQ ID NO: 106 | | SEQ ID NO: 107 | SEQ ID NO: 108 | | |
| 1003 | SEQ ID NO: 106 | | SEQ ID NO: 107 | SEQ ID NO: 108 | SEQ ID NO: 109 | |
| 1004 | SEQ ID NO: 106 | | SEQ ID NO: 107 | SEQ ID NO: 108 | SEQ ID NO: 109 | SEQ ID NO: 110 |
| 1005 | SEQ ID NO: 106 | | SEQ ID NO: 107 | | SEQ ID NO: 109 | |
| 1006 | SEQ ID NO: 106 | | SEQ ID NO: 107 | | SEQ ID NO: 109 | SEQ ID NO: 110 |
| 1007 | SEQ ID NO: 106 | | SEQ ID NO: 107 | | | SEQ ID NO: 110 |
| 1008 | SEQ ID NO: 106 | | SEQ ID NO: 107 | SEQ ID NO: 108 | | SEQ ID NO: 110 |
| 1009 | SEQ ID NO: 113 | | | | | |
| 1010 | SEQ ID NO: 113 | WAS | | | | |
| 1011 | SEQ ID NO: 113 | WAS | SEQ ID NO: 121 | | | |
| 1012 | | WAS | | | | |
| 1013 | | WAS | SEQ ID NO: 121 | | | |
| 1014 | | | SEQ ID NO: 121 | | | |
| 1015 | SEQ ID NO: 113 | | SEQ ID NO: 121 | | | |
| 1016 | | | | SEQ ID NO: 115 | | |
| 1017 | | | | SEQ ID NO: 115 | SEQ ID NO: 116 | |
| 1018 | | | | SEQ ID NO: 115 | SEQ ID NO: 116 | SEQ ID NO: 117 |
| 1019 | | | | | SEQ ID NO: 116 | |
| 1020 | | | | | SEQ ID NO: 116 | SEQ ID NO: 117 |
| 1021 | | | | | | SEQ ID NO: 117 |
| 1022 | | | | SEQ ID NO: 115 | | SEQ ID NO: 117 |
| 1023 | SEQ ID NO: 113 | | | SEQ ID NO: 115 | | |
| 1024 | SEQ ID NO: 113 | | | SEQ ID NO: 115 | SEQ ID NO: 116 | |
| 1025 | SEQ ID NO: 113 | | | SEQ ID NO: 115 | SEQ ID NO: 116 | SEQ ID NO: 117 |
| 1026 | SEQ ID NO: 113 | | | | SEQ ID NO: 116 | |
| 1027 | SEQ ID NO: 113 | | | | SEQ ID NO: 116 | SEQ ID NO: 117 |
| 1028 | SEQ ID NO: 113 | | | | | SEQ ID NO: 117 |
| 1029 | SEQ ID NO: 113 | | | SEQ ID NO: 115 | | SEQ ID NO: 117 |
| 1030 | SEQ ID NO: 113 | WAS | | SEQ ID NO: 115 | | |
| 1031 | SEQ ID NO: 113 | WAS | | SEQ ID NO: 115 | SEQ ID NO: 116 | |
| 1032 | SEQ ID NO: 113 | WAS | | SEQ ID NO: 115 | SEQ ID NO: 116 | SEQ ID NO: 117 |
| 1033 | SEQ ID NO: 113 | WAS | | | SEQ ID NO: 116 | |
| 1034 | SEQ ID NO: 113 | WAS | | | SEQ ID NO: 116 | SEQ ID NO: 117 |
| 1035 | SEQ ID NO: 113 | WAS | | | | SEQ ID NO: 117 |
| 1036 | SEQ ID NO: 113 | WAS | | SEQ ID NO: 115 | | SEQ ID NO: 117 |
| 1037 | SEQ ID NO: 113 | WAS | SEQ ID NO: 121 | SEQ ID NO: 115 | | |
| 1038 | SEQ ID NO: 113 | WAS | SEQ ID NO: 121 | SEQ ID NO: 115 | SEQ ID NO: 116 | |
| 1039 | SEQ ID NO: 113 | WAS | SEQ ID NO: 121 | SEQ ID NO: 115 | SEQ ID NO: 116 | SEQ ID NO: 117 |
| 1040 | SEQ ID NO: 113 | WAS | SEQ ID NO: 121 | | SEQ ID NO: 116 | |
| 1041 | SEQ ID NO: 113 | WAS | SEQ ID NO: 121 | | SEQ ID NO: 116 | SEQ ID NO: 117 |
| 1042 | SEQ ID NO: 113 | WAS | SEQ ID NO: 121 | | | SEQ ID NO: 117 |
| 1043 | SEQ ID NO: 113 | WAS | SEQ ID NO: 121 | SEQ ID NO: 115 | | SEQ ID NO: 117 |
| 1044 | | WAS | | SEQ ID NO: 115 | | |
| 1045 | | WAS | | SEQ ID NO: 115 | SEQ ID NO: 116 | |
| 1046 | | WAS | | SEQ ID NO: 115 | SEQ ID NO: 116 | SEQ ID NO: 117 |
| 1047 | | WAS | | | SEQ ID NO: 116 | |
| 1048 | | WAS | | | SEQ ID NO: 116 | SEQ ID NO: 117 |
| 1049 | | WAS | | | | SEQ ID NO: 117 |
| 1050 | | WAS | | SEQ ID NO: 115 | | SEQ ID NO: 117 |
| 1051 | | WAS | SEQ ID NO: 121 | SEQ ID NO: 115 | | |
| 1052 | | WAS | SEQ ID NO: 121 | SEQ ID NO: 115 | SEQ ID NO: 116 | |
| 1053 | | WAS | SEQ ID NO: 121 | SEQ ID NO: 115 | SEQ ID NO: 116 | SEQ ID NO: 117 |
| 1054 | | WAS | SEQ ID NO: 121 | | SEQ ID NO: 116 | |
| 1055 | | WAS | SEQ ID NO: 121 | | SEQ ID NO: 116 | SEQ ID NO: 117 |
| 1056 | | WAS | SEQ ID NO: 121 | | | SEQ ID NO: 117 |
| 1057 | | WAS | SEQ ID NO: 121 | SEQ ID NO: 115 | | SEQ ID NO: 117 |
| 1058 | | | SEQ ID NO: 121 | SEQ ID NO: 115 | | |
| 1059 | | | SEQ ID NO: 121 | SEQ ID NO: 115 | SEQ ID NO: 116 | |
| 1060 | | | SEQ ID NO: 121 | SEQ ID NO: 115 | SEQ ID NO: 116 | SEQ ID NO: 117 |
| 1061 | | | SEQ ID NO: 121 | | SEQ ID NO: 116 | |
| 1062 | | | SEQ ID NO: 121 | | SEQ ID NO: 116 | SEQ ID NO: 117 |
| 1063 | | | SEQ ID NO: 121 | | | SEQ ID NO: 117 |
| 1064 | | | SEQ ID NO: 121 | SEQ ID NO: 115 | | SEQ ID NO: 117 |
| 1065 | SEQ ID NO: 113 | | SEQ ID NO: 121 | SEQ ID NO: 115 | | |
| 1066 | SEQ ID NO: 113 | | SEQ ID NO: 121 | SEQ ID NO: 115 | SEQ ID NO: 116 | |
| 1067 | SEQ ID NO: 113 | | SEQ ID NO: 121 | SEQ ID NO: 115 | SEQ ID NO: 116 | SEQ ID NO: 117 |
| 1068 | SEQ ID NO: 113 | | SEQ ID NO: 121 | | SEQ ID NO: 116 | |
| 1069 | SEQ ID NO: 113 | | SEQ ID NO: 121 | | SEQ ID NO: 116 | SEQ ID NO: 117 |
| 1070 | SEQ ID NO: 113 | | SEQ ID NO: 121 | | | SEQ ID NO: 117 |
| 1071 | SEQ ID NO: 113 | | SEQ ID NO: 121 | SEQ ID NO: 115 | | SEQ ID NO: 117 |
| 1072 | SEQ ID NO: 120 | | | | | |
| 1073 | SEQ ID NO: 120 | WAS | | | | |
| 1074 | SEQ ID NO: 120 | WAS | SEQ ID NO: 121 | | | |
| 1075 | | WAS | | | | |
| 1076 | | WAS | SEQ ID NO: 121 | | | |
| 1077 | | | SEQ ID NO: 121 | | | |
| 1078 | SEQ ID NO: 120 | | SEQ ID NO: 121 | | | |
| 1079 | | | | SEQ ID NO: 122 | | |
| 1080 | | | | SEQ ID NO: 122 | SEQ ID NO: 123 | |
| 1081 | | | | SEQ ID NO: 122 | SEQ ID NO: 123 | SEQ ID NO: 124 |
| 1082 | | | | | SEQ ID NO: 123 | |
| 1083 | | | | | SEQ ID NO: 123 | SEQ ID NO: 124 |
| 1084 | | | | | | SEQ ID NO: 124 |
| 1085 | | | | SEQ ID NO: 122 | | SEQ ID NO: 124 |
| 1086 | SEQ ID NO: 120 | | | SEQ ID NO: 122 | | |
| 1087 | SEQ ID NO: 120 | | | SEQ ID NO: 122 | SEQ ID NO: 123 | |
| 1088 | SEQ ID NO: 120 | | | SEQ ID NO: 122 | SEQ ID NO: 123 | SEQ ID NO: 124 |
| 1089 | SEQ ID NO: 120 | | | | SEQ ID NO: 123 | |
| 1090 | SEQ ID NO: 120 | | | | SEQ ID NO: 123 | SEQ ID NO: 124 |
| 1091 | SEQ ID NO: 120 | | | | | SEQ ID NO: 124 |
| 1092 | SEQ ID NO: 120 | | | SEQ ID NO: 122 | | SEQ ID NO: 124 |
| 1093 | SEQ ID NO: 120 | WAS | | SEQ ID NO: 122 | | |
| 1094 | SEQ ID NO: 120 | WAS | | SEQ ID NO: 122 | SEQ ID NO: 123 | |
| 1095 | SEQ ID NO: 120 | WAS | | SEQ ID NO: 122 | SEQ ID NO: 123 | SEQ ID NO: 124 |
| 1096 | SEQ ID NO: 120 | WAS | | | SEQ ID NO: 123 | |
| 1097 | SEQ ID NO: 120 | WAS | | | SEQ ID NO: 123 | SEQ ID NO: 124 |
| 1098 | SEQ ID NO: 120 | WAS | | | | SEQ ID NO: 124 |
| 1099 | SEQ ID NO: 120 | WAS | | SEQ ID NO: 122 | | SEQ ID NO: 124 |
| 1100 | SEQ ID NO: 120 | WAS | SEQ ID NO: 121 | SEQ ID NO: 122 | | |
| 1101 | SEQ ID NO: 120 | WAS | SEQ ID NO: 121 | SEQ ID NO: 122 | SEQ ID NO: 123 | |
| 1102 | SEQ ID NO: 120 | WAS | SEQ ID NO: 121 | SEQ ID NO: 122 | SEQ ID NO: 123 | SEQ ID NO: 124 |
| 1103 | SEQ ID NO: 120 | WAS | SEQ ID NO: 121 | | SEQ ID NO: 123 | |
| 1104 | SEQ ID NO: 120 | WAS | SEQ ID NO: 121 | | SEQ ID NO: 123 | SEQ ID NO: 124 |
| 1105 | SEQ ID NO: 120 | WAS | SEQ ID NO: 121 | | | SEQ ID NO: 124 |
| 1106 | SEQ ID NO: 120 | WAS | SEQ ID NO: 121 | SEQ ID NO: 122 | | SEQ ID NO: 124 |
| 1107 | | WAS | | SEQ ID NO: 122 | | |
| 1108 | | WAS | | SEQ ID NO: 122 | SEQ ID NO: 123 | |
| 1109 | | WAS | | SEQ ID NO: 122 | SEQ ID NO: 123 | SEQ ID NO: 124 |
| 1110 | | WAS | | | SEQ ID NO: 123 | |
| 1111 | | WAS | | | SEQ ID NO: 123 | SEQ ID NO: 124 |
| 1112 | | WAS | | | | SEQ ID NO: 124 |
| 1113 | | WAS | | SEQ ID NO: 122 | | SEQ ID NO: 124 |
| 1114 | | WAS | SEQ ID NO: 121 | SEQ ID NO: 122 | | |
| 1115 | | WAS | SEQ ID NO: 121 | SEQ ID NO: 122 | SEQ ID NO: 123 | |
| 1116 | | WAS | SEQ ID NO: 121 | SEQ ID NO: 122 | SEQ ID NO: 123 | SEQ ID NO: 124 |
| 1117 | | WAS | SEQ ID NO: 121 | | SEQ ID NO: 123 | |
| 1118 | | WAS | SEQ ID NO: 121 | | SEQ ID NO: 123 | SEQ ID NO: 124 |
| 1119 | | WAS | SEQ ID NO: 121 | | | SEQ ID NO: 124 |
| 1120 | | WAS | SEQ ID NO: 121 | SEQ ID NO: 122 | | SEQ ID NO: 124 |
| 1121 | | | SEQ ID NO: 121 | SEQ ID NO: 122 | | |
| 1122 | | | SEQ ID NO: 121 | SEQ ID NO: 122 | SEQ ID NO: 123 | |
| 1123 | | | SEQ ID NO: 121 | SEQ ID NO: 122 | SEQ ID NO: 123 | SEQ ID NO: 124 |
| 1124 | | | SEQ ID NO: 121 | | SEQ ID NO: 123 | |
| 1125 | | | SEQ ID NO: 121 | | SEQ ID NO: 123 | SEQ ID NO: 124 |
| 1126 | | | SEQ ID NO: 121 | | | SEQ ID NO: 124 |
| 1127 | | | SEQ ID NO: 121 | SEQ ID NO: 122 | | SEQ ID NO: 124 |
| 1128 | SEQ ID NO: 120 | | SEQ ID NO: 121 | SEQ ID NO: 122 | | |
| 1129 | SEQ ID NO: 120 | | SEQ ID NO: 121 | SEQ ID NO: 122 | SEQ ID NO: 123 | |
| 1130 | SEQ ID NO: 120 | | SEQ ID NO: 121 | SEQ ID NO: 122 | SEQ ID NO: 123 | SEQ ID NO: 124 |
| 1131 | SEQ ID NO: 120 | | SEQ ID NO: 121 | | SEQ ID NO: 123 | |
| 1132 | SEQ ID NO: 120 | | SEQ ID NO: 121 | | SEQ ID NO: 123 | SEQ ID NO: 124 |
| 1133 | SEQ ID NO: 120 | | SEQ ID NO: 121 | | | SEQ ID NO: 124 |
| 1134 | SEQ ID NO: 120 | | SEQ ID NO: 121 | SEQ ID NO: 122 | | SEQ ID NO: 124 |
| 1135 | SEQ ID NO: 127 | | | | | |
| 1136 | SEQ ID NO: 127 | EDN | | | | |
| 1137 | SEQ ID NO: 127 | EDN | SEQ ID NO: 128 | | | |
| 1138 | | EDN | | | | |
| 1139 | | EDN | SEQ ID NO: 128 | | | |
| 1140 | | | SEQ ID NO: 128 | | | |
| 1141 | SEQ ID NO: 127 | | SEQ ID NO: 128 | | | |
| 1142 | | | | SEQ ID NO: 129 | | |
| 1143 | | | | SEQ ID NO: 129 | SEQ ID NO: 130 | |
| 1144 | | | | SEQ ID NO: 129 | SEQ ID NO: 130 | SEQ ID NO: 131 |
| 1145 | | | | | SEQ ID NO: 130 | |
| 1146 | | | | | SEQ ID NO: 130 | SEQ ID NO: 131 |
| 1147 | | | | | | SEQ ID NO: 131 |
| 1148 | | | | SEQ ID NO: 129 | | SEQ ID NO: 131 |
| 1149 | SEQ ID NO: 127 | | | SEQ ID NO: 129 | | |
| 1150 | SEQ ID NO: 127 | | | SEQ ID NO: 129 | SEQ ID NO: 130 | |
| 1151 | SEQ ID NO: 127 | | | SEQ ID NO: 129 | SEQ ID NO: 130 | SEQ ID NO: 131 |
| 1152 | SEQ ID NO: 127 | | | | SEQ ID NO: 130 | |
| 1153 | SEQ ID NO: 127 | | | | SEQ ID NO: 130 | SEQ ID NO: 131 |
| 1154 | SEQ ID NO: 127 | | | | | SEQ ID NO: 131 |
| 1155 | SEQ ID NO: 127 | | | SEQ ID NO: 129 | | SEQ ID NO: 131 |
| 1156 | SEQ ID NO: 127 | EDN | | SEQ ID NO: 129 | | |
| 1157 | SEQ ID NO: 127 | EDN | | SEQ ID NO: 129 | SEQ ID NO: 130 | |
| 1158 | SEQ ID NO: 127 | EDN | | SEQ ID NO: 129 | SEQ ID NO: 130 | SEQ ID NO: 131 |
| 1159 | SEQ ID NO: 127 | EDN | | | SEQ ID NO: 130 | |
| 1160 | SEQ ID NO: 127 | EDN | | | SEQ ID NO: 130 | SEQ ID NO: 131 |
| 1161 | SEQ ID NO: 127 | EDN | | | | SEQ ID NO: 131 |
| 1162 | SEQ ID NO: 127 | EDN | | SEQ ID NO: 129 | | SEQ ID NO: 131 |
| 1163 | SEQ ID NO: 127 | EDN | SEQ ID NO: 128 | SEQ ID NO: 129 | | |
| 1164 | SEQ ID NO: 127 | EDN | SEQ ID NO: 128 | SEQ ID NO: 129 | SEQ ID NO: 130 | |
| 1165 | SEQ ID NO: 127 | EDN | SEQ ID NO: 128 | SEQ ID NO: 129 | SEQ ID NO: 130 | SEQ ID NO: 131 |
| 1166 | SEQ ID NO: 127 | EDN | SEQ ID NO: 128 | | SEQ ID NO: 130 | |
| 1167 | SEQ ID NO: 127 | EDN | SEQ ID NO: 128 | | SEQ ID NO: 130 | SEQ ID NO: 131 |
| 1168 | SEQ ID NO: 127 | EDN | SEQ ID NO: 128 | | | SEQ ID NO: 131 |
| 1169 | SEQ ID NO: 127 | EDN | SEQ ID NO: 128 | SEQ ID NO: 129 | | SEQ ID NO: 131 |
| 1170 | | EDN | | SEQ ID NO: 129 | | |
| 1171 | | EDN | | SEQ ID NO: 129 | SEQ ID NO: 130 | |
| 1172 | | EDN | | SEQ ID NO: 129 | SEQ ID NO: 130 | SEQ ID NO: 131 |
| 1173 | | EDN | | | SEQ ID NO: 130 | |
| 1174 | | EDN | | | SEQ ID NO: 130 | SEQ ID NO: 131 |
| 1175 | | EDN | | | | SEQ ID NO: 131 |
| 1176 | | EDN | | SEQ ID NO: 129 | | SEQ ID NO: 131 |
| 1177 | | EDN | SEQ ID NO: 128 | SEQ ID NO: 129 | | |
| 1178 | | EDN | SEQ ID NO: 128 | SEQ ID NO: 129 | SEQ ID NO: 130 | |
| 1179 | | EDN | SEQ ID NO: 128 | SEQ ID NO: 129 | SEQ ID NO: 130 | SEQ ID NO: 131 |
| 1180 | | EDN | SEQ ID NO: 128 | | SEQ ID NO: 130 | |
| 1181 | | EDN | SEQ ID NO: 128 | | SEQ ID NO: 130 | SEQ ID NO: 131 |
| 1182 | | EDN | SEQ ID NO: 128 | | | SEQ ID NO: 131 |
| 1183 | | EDN | SEQ ID NO: 128 | SEQ ID NO: 129 | | SEQ ID NO: 131 |
| 1184 | | | SEQ ID NO: 128 | SEQ ID NO: 129 | | |
| 1185 | | | SEQ ID NO: 128 | SEQ ID NO: 129 | SEQ ID NO: 130 | |
| 1186 | | | SEQ ID NO: 128 | SEQ ID NO: 129 | SEQ ID NO: 130 | SEQ ID NO: 131 |
| 1187 | | | SEQ ID NO: 128 | | SEQ ID NO: 130 | |
| 1188 | | | SEQ ID NO: 128 | | SEQ ID NO: 130 | SEQ ID NO: 131 |
| 1189 | | | SEQ ID NO: 128 | | | SEQ ID NO: 131 |
| 1190 | | | SEQ ID NO: 128 | SEQ ID NO: 129 | | SEQ ID NO: 131 |
| 1191 | SEQ ID NO: 127 | | SEQ ID NO: 128 | SEQ ID NO: 129 | | |
| 1192 | SEQ ID NO: 127 | | SEQ ID NO: 128 | SEQ ID NO: 129 | SEQ ID NO: 130 | |
| 1193 | SEQ ID NO: 127 | | SEQ ID NO: 128 | SEQ ID NO: 129 | SEQ ID NO: 130 | SEQ ID NO: 131 |
| 1194 | SEQ ID NO: 127 | | SEQ ID NO: 128 | | SEQ ID NO: 130 | |
| 1195 | SEQ ID NO: 127 | | SEQ ID NO: 128 | | SEQ ID NO: 130 | SEQ ID NO: 131 |
| 1196 | SEQ ID NO: 127 | | SEQ ID NO: 128 | | | SEQ ID NO: 131 |
| 1197 | SEQ ID NO: 127 | | SEQ ID NO: 128 | SEQ ID NO: 129 | | SEQ ID NO: 131 |
| 1198 | SEQ ID NO: 134 | | | | | |
| 1199 | SEQ ID NO: 134 | DDS | | | | |
| 1200 | SEQ ID NO: 134 | DDS | SEQ ID NO: 135 | | | |
| 1201 | | DDS | | | | |
| 1202 | | DDS | SEQ ID NO: 135 | | | |
| 1203 | | | SEQ ID NO: 135 | | | |
| 1204 | SEQ ID NO: 134 | | SEQ ID NO: 135 | | | |
| 1205 | | | | SEQ ID NO: 136 | | |
| 1206 | | | | SEQ ID NO: 136 | SEQ ID NO: 137 | |
| 1207 | | | | SEQ ID NO: 136 | SEQ ID NO: 137 | SEQ ID NO: 138 |
| 1208 | | | | | SEQ ID NO: 137 | |
| 1209 | | | | | SEQ ID NO: 137 | SEQ ID NO: 138 |
| 1210 | | | | | | SEQ ID NO: 138 |
| 1211 | | | | SEQ ID NO: 136 | | SEQ ID NO: 138 |
| 1212 | SEQ ID NO: 134 | | | SEQ ID NO: 136 | | |
| 1213 | SEQ ID NO: 134 | | | SEQ ID NO: 136 | SEQ ID NO: 137 | |
| 1214 | SEQ ID NO: 134 | | | SEQ ID NO: 136 | SEQ ID NO: 137 | SEQ ID NO: 138 |
| 1215 | SEQ ID NO: 134 | | | | SEQ ID NO: 137 | |
| 1216 | SEQ ID NO: 134 | | | | SEQ ID NO: 137 | SEQ ID NO: 138 |
| 1217 | SEQ ID NO: 134 | | | | | SEQ ID NO: 138 |
| 1218 | SEQ ID NO: 134 | | | SEQ ID NO: 136 | | SEQ ID NO: 138 |
| 1219 | SEQ ID NO: 134 | DDS | | SEQ ID NO: 136 | | |
| 1220 | SEQ ID NO: 134 | DDS | | SEQ ID NO: 136 | SEQ ID NO: 137 | |
| 1221 | SEQ ID NO: 134 | DDS | | SEQ ID NO: 136 | SEQ ID NO: 137 | SEQ ID NO: 138 |
| 1223 | SEQ ID NO: 134 | DDS | | | SEQ ID NO: 137 | |
| 1224 | SEQ ID NO: 134 | DDS | | | SEQ ID NO: 137 | SEQ ID NO: 138 |
| 1225 | SEQ ID NO: 134 | DDS | | | | SEQ ID NO: 138 |
| 1226 | SEQ ID NO: 134 | DDS | | SEQ ID NO: 136 | | SEQ ID NO: 138 |
| 1227 | SEQ ID NO: 134 | DDS | SEQ ID NO: 135 | SEQ ID NO: 136 | | |
| 1228 | SEQ ID NO: 134 | DDS | SEQ ID NO: 135 | SEQ ID NO: 136 | SEQ ID NO: 137 | |
| 1229 | SEQ ID NO: 134 | DDS | SEQ ID NO: 135 | SEQ ID NO: 136 | SEQ ID NO: 137 | SEQ ID NO: 138 |
| 1230 | SEQ ID NO: 134 | DDS | SEQ ID NO: 135 | | SEQ ID NO: 137 | |
| 1231 | SEQ ID NO: 134 | DDS | SEQ ID NO: 135 | | SEQ ID NO: 137 | SEQ ID NO: 138 |
| 1232 | SEQ ID NO: 134 | DDS | SEQ ID NO: 135 | | | SEQ ID NO: 138 |
| 1233 | SEQ ID NO: 134 | DDS | SEQ ID NO: 135 | SEQ ID NO: 136 | | SEQ ID NO: 138 |
| 1234 | | DDS | | SEQ ID NO: 136 | | |
| 1235 | | DDS | | SEQ ID NO: 136 | SEQ ID NO: 137 | |
| 1236 | | DDS | | SEQ ID NO: 136 | SEQ ID NO: 137 | SEQ ID NO: 138 |
| 1237 | | DDS | | | SEQ ID NO: 137 | |
| 1238 | | DDS | | | SEQ ID NO: 137 | SEQ ID NO: 138 |
| 1239 | | DDS | | | | SEQ ID NO: 138 |
| 1240 | | DDS | | SEQ ID NO: 136 | | SEQ ID NO: 138 |
| 1241 | | DDS | SEQ ID NO: 135 | SEQ ID NO: 136 | | |
| 1242 | | DDS | SEQ ID NO: 135 | SEQ ID NO: 136 | SEQ ID NO: 137 | |
| 1243 | | DDS | SEQ ID NO: 135 | SEQ ID NO: 136 | SEQ ID NO: 137 | SEQ ID NO: 138 |
| 1244 | | DDS | SEQ ID NO: 135 | | SEQ ID NO: 137 | |
| 1245 | | DDS | SEQ ID NO: 135 | | SEQ ID NO: 137 | SEQ ID NO: 138 |
| 1246 | | DDS | SEQ ID NO: 135 | | | SEQ ID NO: 138 |
| 1247 | | DDS | SEQ ID NO: 135 | SEQ ID NO: 136 | | SEQ ID NO: 138 |
| 1248 | | | SEQ ID NO: 135 | SEQ ID NO: 136 | | |
| 1249 | | | SEQ ID NO: 135 | SEQ ID NO: 136 | SEQ ID NO: 137 | |
| 1250 | | | SEQ ID NO: 135 | SEQ ID NO: 136 | SEQ ID NO: 137 | SEQ ID NO: 138 |
| 1251 | | | SEQ ID NO: 135 | | SEQ ID NO: 137 | |
| 1252 | | | SEQ ID NO: 135 | | SEQ ID NO: 137 | SEQ ID NO: 138 |
| 1253 | | | SEQ ID NO: 135 | | | SEQ ID NO: 138 |
| 1254 | | | SEQ ID NO: 135 | SEQ ID NO: 136 | | SEQ ID NO: 138 |
| 1255 | SEQ ID NO: 134 | | SEQ ID NO: 135 | SEQ ID NO: 136 | | |
| 1256 | SEQ ID NO: 134 | | SEQ ID NO: 135 | SEQ ID NO: 136 | SEQ ID NO: 137 | |
| 1257 | SEQ ID NO: 134 | | SEQ ID NO: 135 | SEQ ID NO: 136 | SEQ ID NO: 137 | SEQ ID NO: 138 |
| 1258 | SEQ ID NO: 134 | | SEQ ID NO: 135 | | SEQ ID NO: 137 | |
| 1259 | SEQ ID NO: 134 | | SEQ ID NO: 135 | | SEQ ID NO: 137 | SEQ ID NO: 138 |
| 1260 | SEQ ID NO: 134 | | SEQ ID NO: 135 | | | SEQ ID NO: 138 |
| 1261 | SEQ ID NO: 134 | | SEQ ID NO: 135 | SEQ ID NO: 136 | | SEQ ID NO: 138 |
| 1262 | SEQ ID NO: 141 | | | | | |
| 1263 | SEQ ID NO: 141 | KDS | | | | |
| 1264 | SEQ ID NO: 141 | KDS | SEQ ID NO: 142 | | | |
| 1265 | | KDS | | | | |
| 1266 | | KDS | SEQ ID NO: 142 | | | |
| 1267 | | | SEQ ID NO: 142 | | | |
| 1268 | SEQ ID NO: 141 | | SEQ ID NO: 142 | | | |
| 1269 | | | | SEQ ID NO: 143 | | |
| 1270 | | | | SEQ ID NO: 143 | SEQ ID NO: 144 | |
| 1271 | | | | SEQ ID NO: 143 | SEQ ID NO: 144 | SEQ ID NO: 145 |
| 1272 | | | | | SEQ ID NO: 144 | |
| 1273 | | | | | SEQ ID NO: 144 | SEQ ID NO: 145 |
| 1274 | | | | | | SEQ ID NO: 145 |
| 1275 | | | | SEQ ID NO: 143 | | SEQ ID NO: 145 |
| 1276 | SEQ ID NO: 141 | | | SEQ ID NO: 143 | | |
| 1277 | SEQ ID NO: 141 | | | SEQ ID NO: 143 | SEQ ID NO: 144 | |
| 1278 | SEQ ID NO: 141 | | | SEQ ID NO: 143 | SEQ ID NO: 144 | SEQ ID NO: 145 |
| 1279 | SEQ ID NO: 141 | | | | SEQ ID NO: 144 | |
| 1280 | SEQ ID NO: 141 | | | | SEQ ID NO: 144 | SEQ ID NO: 145 |
| 1281 | SEQ ID NO: 141 | | | | | SEQ ID NO: 145 |
| 1282 | SEQ ID NO: 141 | | | SEQ ID NO: 143 | | SEQ ID NO: 145 |
| 1283 | SEQ ID NO: 141 | KDS | | SEQ ID NO: 143 | | |
| 1284 | SEQ ID NO: 141 | KDS | | SEQ ID NO: 143 | SEQ ID NO: 144 | |
| 1285 | SEQ ID NO: 141 | KDS | | SEQ ID NO: 143 | SEQ ID NO: 144 | SEQ ID NO: 145 |
| 1286 | SEQ ID NO: 141 | KDS | | | SEQ ID NO: 144 | |
| 1287 | SEQ ID NO: 141 | KDS | | | SEQ ID NO: 144 | SEQ ID NO: 145 |
| 1288 | SEQ ID NO: 141 | KDS | | | | SEQ ID NO: 145 |
| 1289 | SEQ ID NO: 141 | KDS | | SEQ ID NO: 143 | | SEQ ID NO: 145 |
| 1290 | SEQ ID NO: 141 | KDS | SEQ ID NO: 142 | SEQ ID NO: 143 | | |
| 1291 | SEQ ID NO: 141 | KDS | SEQ ID NO: 142 | SEQ ID NO: 143 | SEQ ID NO: 144 | |
| 1292 | SEQ ID NO: 141 | KDS | SEQ ID NO: 142 | SEQ ID NO: 143 | SEQ ID NO: 144 | SEQ ID NO: 145 |
| 1293 | SEQ ID NO: 141 | KDS | SEQ ID NO: 142 | | SEQ ID NO: 144 | |
| 1294 | SEQ ID NO: 141 | KDS | SEQ ID NO: 142 | | SEQ ID NO: 144 | SEQ ID NO: 145 |
| 1295 | SEQ ID NO: 141 | KDS | SEQ ID NO: 142 | | | SEQ ID NO: 145 |
| 1296 | SEQ ID NO: 141 | KDS | SEQ ID NO: 142 | SEQ ID NO: 143 | | SEQ ID NO: 145 |
| 1297 | | KDS | | SEQ ID NO: 143 | | |
| 1298 | | KDS | | SEQ ID NO: 143 | SEQ ID NO: 144 | |
| 1299 | | KDS | | SEQ ID NO: 143 | SEQ ID NO: 144 | SEQ ID NO: 145 |
| 1300 | | KDS | | | SEQ ID NO: 144 | |
| 1301 | | KDS | | | SEQ ID NO: 144 | SEQ ID NO: 145 |
| 1302 | | KDS | | | | SEQ ID NO: 145 |
| 1303 | | KDS | | SEQ ID NO: 143 | | SEQ ID NO: 145 |
| 1304 | | KDS | SEQ ID NO: 142 | SEQ ID NO: 143 | | |
| 1305 | | KDS | SEQ ID NO: 142 | SEQ ID NO: 143 | SEQ ID NO: 144 | |
| 1306 | | KDS | SEQ ID NO: 142 | SEQ ID NO: 143 | SEQ ID NO: 144 | SEQ ID NO: 145 |
| 1307 | | KDS | SEQ ID NO: 142 | | SEQ ID NO: 144 | |
| 1308 | | KDS | SEQ ID NO: 142 | | SEQ ID NO: 144 | SEQ ID NO: 145 |
| 1309 | | KDS | SEQ ID NO: 142 | | | SEQ ID NO: 145 |
| 1310 | | KDS | SEQ ID NO: 142 | SEQ ID NO: 143 | | SEQ ID NO: 145 |
| 1311 | | | SEQ ID NO: 142 | SEQ ID NO: 143 | | |
| 1312 | | | SEQ ID NO: 142 | SEQ ID NO: 143 | SEQ ID NO: 144 | |
| 1313 | | | SEQ ID NO: 142 | SEQ ID NO: 143 | SEQ ID NO: 144 | SEQ ID NO: 145 |
| 1314 | | | SEQ ID NO: 142 | | SEQ ID NO: 144 | |
| 1315 | | | SEQ ID NO: 142 | | SEQ ID NO: 144 | SEQ ID NO: 145 |
| 1316 | | | SEQ ID NO: 142 | | | SEQ ID NO: 145 |
| 1317 | | | SEQ ID NO: 142 | SEQ ID NO: 143 | | SEQ ID NO: 145 |
| 1318 | SEQ ID NO: 141 | | SEQ ID NO: 142 | SEQ ID NO: 143 | | |
| 1319 | SEQ ID NO: 141 | | SEQ ID NO: 142 | SEQ ID NO: 143 | SEQ ID NO: 144 | |
| 1320 | SEQ ID NO: 141 | | SEQ ID NO: 142 | SEQ ID NO: 143 | SEQ ID NO: 144 | SEQ ID NO: 145 |
| 1321 | SEQ ID NO: 141 | | SEQ ID NO: 142 | | SEQ ID NO: 144 | |
| 1323 | SEQ ID NO: 141 | | SEQ ID NO: 142 | | SEQ ID NO: 144 | SEQ ID NO: 145 |
| 1324 | SEQ ID NO: 141 | | SEQ ID NO: 142 | | | SEQ ID NO: 145 |
| 1325 | SEQ ID NO: 141 | | SEQ ID NO: 142 | SEQ ID NO: 143 | | SEQ ID NO: 145 |
| 1326 | SEQ ID NO: 148 | | | | | |
| 1327 | SEQ ID NO: 148 | DAS | | | | |
| 1328 | SEQ ID NO: 148 | DAS | SEQ ID NO: 149 | | | |
| 1329 | | DAS | | | | |
| 1330 | | DAS | SEQ ID NO: 149 | | | |
| 1331 | | | SEQ ID NO: 149 | | | |
| 1332 | SEQ ID NO: 148 | | SEQ ID NO: 149 | | | |
| 1333 | | | | SEQ ID NO: 150 | | |
| 1334 | | | | SEQ ID NO: 150 | SEQ ID NO: 151 | |
| 1335 | | | | SEQ ID NO: 150 | SEQ ID NO: 151 | SEQ ID NO: 152 |
| 1336 | | | | | SEQ ID NO: 151 | |
| 1337 | | | | | SEQ ID NO: 151 | SEQ ID NO: 152 |
| 1338 | | | | | | SEQ ID NO: 152 |
| 1339 | | | | SEQ ID NO: 150 | | SEQ ID NO: 152 |
| 1340 | SEQ ID NO: 148 | | | SEQ ID NO: 150 | | |
| 1341 | SEQ ID NO: 148 | | | SEQ ID NO: 150 | SEQ ID NO: 151 | |
| 1342 | SEQ ID NO: 148 | | | SEQ ID NO: 150 | SEQ ID NO: 151 | SEQ ID NO: 152 |
| 1343 | SEQ ID NO: 148 | | | | SEQ ID NO: 151 | |
| 1344 | SEQ ID NO: 148 | | | | SEQ ID NO: 151 | SEQ ID NO: 152 |
| 1345 | SEQ ID NO: 148 | | | | | SEQ ID NO: 152 |
| 1346 | SEQ ID NO: 148 | | | SEQ ID NO: 150 | | SEQ ID NO: 152 |
| 1347 | SEQ ID NO: 148 | DAS | | SEQ ID NO: 150 | | |
| 1348 | SEQ ID NO: 148 | DAS | | SEQ ID NO: 150 | SEQ ID NO: 151 | |
| 1349 | SEQ ID NO: 148 | DAS | | SEQ ID NO: 150 | SEQ ID NO: 151 | SEQ ID NO: 152 |
| 1350 | SEQ ID NO: 148 | DAS | | | SEQ ID NO: 151 | |
| 1351 | SEQ ID NO: 148 | DAS | | | SEQ ID NO: 151 | SEQ ID NO: 152 |
| 1352 | SEQ ID NO: 148 | DAS | | | | SEQ ID NO: 152 |
| 1353 | SEQ ID NO: 148 | DAS | | SEQ ID NO: 150 | | SEQ ID NO: 152 |
| 1354 | SEQ ID NO: 148 | DAS | SEQ ID NO: 149 | SEQ ID NO: 150 | | |
| 1355 | SEQ ID NO: 148 | DAS | SEQ ID NO: 149 | SEQ ID NO: 150 | SEQ ID NO: 151 | |
| 1356 | SEQ ID NO: 148 | DAS | SEQ ID NO: 149 | SEQ ID NO: 150 | SEQ ID NO: 151 | SEQ ID NO: 152 |
| 1357 | SEQ ID NO: 148 | DAS | SEQ ID NO: 149 | | SEQ ID NO: 151 | |
| 1358 | SEQ ID NO: 148 | DAS | SEQ ID NO: 149 | | SEQ ID NO: 151 | SEQ ID NO: 152 |
| 1359 | SEQ ID NO: 148 | DAS | SEQ ID NO: 149 | | | SEQ ID NO: 152 |
| 1360 | SEQ ID NO: 148 | DAS | SEQ ID NO: 149 | SEQ ID NO: 150 | | SEQ ID NO: 152 |
| 1361 | | DAS | | SEQ ID NO: 150 | | |
| 1362 | | DAS | | SEQ ID NO: 150 | SEQ ID NO: 151 | |
| 1363 | | DAS | | SEQ ID NO: 150 | SEQ ID NO: 151 | SEQ ID NO: 152 |
| 1364 | | DAS | | | SEQ ID NO: 151 | |
| 1365 | | DAS | | | SEQ ID NO: 151 | SEQ ID NO: 152 |
| 1366 | | DAS | | | | SEQ ID NO: 152 |
| 1367 | | DAS | | SEQ ID NO: 150 | | SEQ ID NO: 152 |
| 1368 | | DAS | SEQ ID NO: 149 | SEQ ID NO: 150 | | |
| 1369 | | DAS | SEQ ID NO: 149 | SEQ ID NO: 150 | SEQ ID NO: 151 | |
| 1370 | | DAS | SEQ ID NO: 149 | SEQ ID NO: 150 | SEQ ID NO: 151 | SEQ ID NO: 152 |
| 1371 | | DAS | SEQ ID NO: 149 | | SEQ ID NO: 151 | |
| 1372 | | DAS | SEQ ID NO: 149 | | SEQ ID NO: 151 | SEQ ID NO: 152 |
| 1373 | | DAS | SEQ ID NO: 149 | | | SEQ ID NO: 152 |
| 1374 | | DAS | SEQ ID NO: 149 | SEQ ID NO: 150 | | SEQ ID NO: 152 |
| 1375 | | | SEQ ID NO: 149 | SEQ ID NO: 150 | | |
| 1376 | | | SEQ ID NO: 149 | SEQ ID NO: 150 | SEQ ID NO: 151 | |
| 1377 | | | SEQ ID NO: 149 | SEQ ID NO: 150 | SEQ ID NO: 151 | SEQ ID NO: 152 |
| 1378 | | | SEQ ID NO: 149 | | SEQ ID NO: 151 | |
| 1379 | | | SEQ ID NO: 149 | | SEQ ID NO: 151 | SEQ ID NO: 152 |
| 1380 | | | SEQ ID NO: 149 | | | SEQ ID NO: 152 |
| 1381 | | | SEQ ID NO: 149 | SEQ ID NO: 150 | | SEQ ID NO: 152 |
| 1382 | SEQ ID NO: 148 | | SEQ ID NO: 149 | SEQ ID NO: 150 | | |
| 1383 | SEQ ID NO: 148 | | SEQ ID NO: 149 | SEQ ID NO: 150 | SEQ ID NO: 151 | |
| 1384 | SEQ ID NO: 148 | | SEQ ID NO: 149 | SEQ ID NO: 150 | SEQ ID NO: 151 | SEQ ID NO: 152 |
| 1385 | SEQ ID NO: 148 | | SEQ ID NO: 149 | | SEQ ID NO: 151 | |
| 1386 | SEQ ID NO: 148 | | SEQ ID NO: 149 | | SEQ ID NO: 151 | SEQ ID NO: 152 |
| 1387 | SEQ ID NO: 148 | | SEQ ID NO: 149 | | | SEQ ID NO: 152 |
| 1388 | SEQ ID NO: 148 | | SEQ ID NO: 149 | SEQ ID NO: 150 | | SEQ ID NO: 152 |
| 1389 | SEQ ID NO: 155 | | | | | |
| 1390 | SEQ ID NO: 155 | DDS | | | | |
| 1391 | SEQ ID NO: 155 | DDS | SEQ ID NO: 156 | | | |
| 1392 | | DDS | | | | |
| 1393 | | DDS | SEQ ID NO: 156 | | | |
| 1394 | | | SEQ ID NO: 156 | | | |
| 1395 | SEQ ID NO: 155 | | SEQ ID NO: 156 | | | |
| 1396 | | | | SEQ ID NO: 157 | | |
| 1397 | | | | SEQ ID NO: 157 | SEQ ID NO: 158 | |
| 1398 | | | | SEQ ID NO: 157 | SEQ ID NO: 158 | SEQ ID NO: 159 |
| 1399 | | | | | SEQ ID NO: 158 | |
| 1400 | | | | | SEQ ID NO: 158 | SEQ ID NO: 159 |
| 1401 | | | | | | SEQ ID NO: 159 |
| 1402 | | | | SEQ ID NO: 157 | | SEQ ID NO: 159 |
| 1403 | SEQ ID NO: 155 | | | SEQ ID NO: 157 | | |
| 1404 | SEQ ID NO: 155 | | | SEQ ID NO: 157 | SEQ ID NO: 158 | |
| 1405 | SEQ ID NO: 155 | | | SEQ ID NO: 157 | SEQ ID NO: 158 | SEQ ID NO: 159 |
| 1406 | SEQ ID NO: 155 | | | | SEQ ID NO: 158 | |
| 1407 | SEQ ID NO: 155 | | | | SEQ ID NO: 158 | SEQ ID NO: 159 |
| 1408 | SEQ ID NO: 155 | | | | | SEQ ID NO: 159 |
| 1409 | SEQ ID NO: 155 | | | SEQ ID NO: 157 | | SEQ ID NO: 159 |
| 1410 | SEQ ID NO: 155 | DDS | | SEQ ID NO: 157 | | |
| 1411 | SEQ ID NO: 155 | DDS | | SEQ ID NO: 157 | SEQ ID NO: 158 | |
| 1412 | SEQ ID NO: 155 | DDS | | SEQ ID NO: 157 | SEQ ID NO: 158 | SEQ ID NO: 159 |
| 1413 | SEQ ID NO: 155 | DDS | | | SEQ ID NO: 158 | |
| 1414 | SEQ ID NO: 155 | DDS | | | SEQ ID NO: 158 | SEQ ID NO: 159 |
| 1415 | SEQ ID NO: 155 | DDS | | | | SEQ ID NO: 159 |
| 1416 | SEQ ID NO: 155 | DDS | | SEQ ID NO: 157 | | SEQ ID NO: 159 |
| 1417 | SEQ ID NO: 155 | DDS | SEQ ID NO: 156 | SEQ ID NO: 157 | | |
| 1418 | SEQ ID NO: 155 | DDS | SEQ ID NO: 156 | SEQ ID NO: 157 | SEQ ID NO: 158 | |
| 1419 | SEQ ID NO: 155 | DDS | SEQ ID NO: 156 | SEQ ID NO: 157 | SEQ ID NO: 158 | SEQ ID NO: 159 |
| 1420 | SEQ ID NO: 155 | DDS | SEQ ID NO: 156 | | SEQ ID NO: 158 | |
| 1421 | SEQ ID NO: 155 | DDS | SEQ ID NO: 156 | | SEQ ID NO: 158 | SEQ ID NO: 159 |
| 1422 | SEQ ID NO: 155 | DDS | SEQ ID NO: 156 | | | SEQ ID NO: 159 |
| 1423 | SEQ ID NO: 155 | DDS | SEQ ID NO: 156 | SEQ ID NO: 157 | | SEQ ID NO: 159 |
| 1424 | | DDS | | SEQ ID NO: 157 | | |
| 1425 | | DDS | | SEQ ID NO: 157 | SEQ ID NO: 158 | |
| 1426 | | DDS | | SEQ ID NO: 157 | SEQ ID NO: 158 | SEQ ID NO: 159 |
| 1427 | | DDS | | | SEQ ID NO: 158 | |
| 1428 | | DDS | | | SEQ ID NO: 158 | SEQ ID NO: 159 |
| 1429 | | DDS | | | | SEQ ID NO: 159 |
| 1430 | | DDS | | SEQ ID NO: 157 | | SEQ ID NO: 159 |
| 1431 | | DDS | SEQ ID NO: 156 | SEQ ID NO: 157 | | |
| 1432 | | DDS | SEQ ID NO: 156 | SEQ ID NO: 157 | SEQ ID NO: 158 | |
| 1433 | | DDS | SEQ ID NO: 156 | SEQ ID NO: 157 | SEQ ID NO: 158 | SEQ ID NO: 159 |
| 1434 | | DDS | SEQ ID NO: 156 | | SEQ ID NO: 158 | |
| 1435 | | DDS | SEQ ID NO: 156 | | SEQ ID NO: 158 | SEQ ID NO: 159 |
| 1436 | | DDS | SEQ ID NO: 156 | | | SEQ ID NO: 159 |
| 1437 | | DDS | SEQ ID NO: 156 | SEQ ID NO: 157 | | SEQ ID NO: 159 |
| 1438 | | | SEQ ID NO: 156 | SEQ ID NO: 157 | | |
| 1439 | | | SEQ ID NO: 156 | SEQ ID NO: 157 | SEQ ID NO: 158 | |
| 1440 | | | SEQ ID NO: 156 | SEQ ID NO: 157 | SEQ ID NO: 158 | SEQ ID NO: 159 |
| 1441 | | | SEQ ID NO: 156 | | SEQ ID NO: 158 | |
| 1442 | | | SEQ ID NO: 156 | | SEQ ID NO: 158 | SEQ ID NO: 159 |
| 1443 | | | SEQ ID NO: 156 | | | SEQ ID NO: 159 |
| 1444 | | | SEQ ID NO: 156 | SEQ ID NO: 157 | | SEQ ID NO: 159 |
| 1445 | SEQ ID NO: 155 | | SEQ ID NO: 156 | SEQ ID NO: 157 | | |
| 1446 | SEQ ID NO: 155 | | SEQ ID NO: 156 | SEQ ID NO: 157 | SEQ ID NO: 158 | |
| 1447 | SEQ ID NO: 155 | | SEQ ID NO: 156 | SEQ ID NO: 157 | SEQ ID NO: 158 | SEQ ID NO: 159 |
| 1448 | SEQ ID NO: 155 | | SEQ ID NO: 156 | | SEQ ID NO: 158 | |
| 1449 | SEQ ID NO: 155 | | SEQ ID NO: 156 | | SEQ ID NO: 158 | SEQ ID NO: 159 |
| 1450 | SEQ ID NO: 155 | | SEQ ID NO: 156 | | | SEQ ID NO: 159 |
| 1451 | SEQ ID NO: 155 | | SEQ ID NO: 156 | SEQ ID NO: 157 | | SEQ ID NO: 159 |

TABLE B
Illustrative Sequences for
Anti-SARS-CoV-2 antibodies

| | | | SEQ ID |
|---|---|---|---|
| Antibody | Description | Amino Acids | NO: |
| 07.1A11 | L1 | QDISNY | 1 |
| 07.1A11 | L2 | DAS | |
| 07.1A11 | L3 | QQYDNLPPT | 2 |
| 07.1A11 | H1 | GFTFSYAW | 3 |
| 07.1A11 | H2 | IKSKTDGGTT | 4 |
| 07.1A11 | H3 | TTGWFTGTYGDYFDY | 5 |
| 07.1A11 | VL | DIQMTQSPSSLSASVGDRVTIT | 6 |
| | | CQASQDISNYLNWYQQKPGKA | |
| | | PKLLIYDASNLQTGVPSRFSGS | |
| | | GSGTDFTFTISSLQPEDIATYYC | |
| | | QQYDNLPPTFGGGTKVEIK | |
| 07.1A11 | VH | EVQLVESGGGLVKPGGSLRLS | 7 |
| | | CAASGFTFSYAWMTWVRQAP | |
| | | GKGLEWVGRIKSKTDGGTTDY | |
| | | AAPVKGRFTISRDDSKNTLFLQ | |
| | | MNSLKTEDTAVYFCTTGWFTG | |
| | | TYGDYFDYWGQGTLVTVSS | |
| 07.1H09 | L1 | QGISSY | 8 |
| 07.1H09 | L2 | AAS | |
| 07.1H09 | L3 | QQLNSYPPT | 9 |
| 07.1H09 | H1 | GIIVSSNY | 10 |
| 07.1H09 | H2 | IYSGGST | 11 |
| 07.1H09 | H3 | ARDFREGAFDI | 12 |
| 07.1H09 | VL | DIQLTQSPSFLSASVGDRVTITC | 13 |
| | | RASQGISSYLAVVYQQKPGKAP | |
| | | KLLIYAASTLQSGVPSRFSGSG | |
| | | SGTEFTLTISSLQPEDFATYYCQ | |
| | | QLNSYPPTFGGGTKVEIK | |
| 07.1H09 | VH | EVQLVESGGGLVQPGGSLRLS | 14 |
| | | CAASGIIVSSNYMSWVRQAPGK | |
| | | GLEWVSVIYSGGSTYYADSVK | |
| | | GRFTISRDNSKNTLYLQMSSLR | |
| | | AEDTAVYYCARDFREGAFDIW | |
| | | GQGTMVTVSS | |
| 07.2A08 | L1 | KLGNKY | 15 |
| 07.2A08 | L2 | QDN | |
| 07.2A08 | L3 | QAWGSSTVV | 16 |
| 07.2A08 | H1 | GGSISSYY | 17 |
| 07.2A08 | H2 | IYTSGST | 18 |
| 07.2A08 | H3 | ATDGGWYTFDH | 19 |
| 07.2A08 | VL | SYELTQPPSVSVSPGQTASITC | 20 |
| | | SGDKLGNKYACWYQQKPGQS | |
| | | PVLVIYQDNKRPSGIPERFSGS | |
| | | NSGNTATLTISGTQAMDEADYY | |
| | | CQAWGSSTVVFGGGTKLTVL | |
| 07.2A08 | VH | QVQLQESGPGLVKPSETLSLTC | 21 |
| | | TVSGGSISSYYWNWIRQPAGK | |
| | | GLEWIGRIYTSGSTNYNPSLKS | |
| | | RVTMSVDTSKNQFSLKLSSVTA | |
| | | ADTAVYYCATDGGWYTFDHW | |
| | | GQGTLVTVSS | |
| 07.2A10 | L1 | QDISNY | 22 |
| 07.2A10 | L2 | DAS | |
| 07.2A10 | L3 | QHYDNLPPT | 23 |
| 07.2A10 | H1 | GGSISSGGYY | 24 |
| 07.2A10 | H2 | IYYSGST | 25 |
| 07.2A10 | H3 | ARYPVWGAFDI | 26 |
| 07.2A10 | VL | DIQMTQSPSSLSASVGDRVTIT | 27 |
| | | CQASQDISNYLNWYQQKPGKA | |
| | | PNLLIYDASNLETGVPSRFSGS | |
| | | GSGTDFTFTISSLQPEDFATYY | |
| | | CQHYDNLPPTFGPGTKVDIK | |
| 07.2A10 | VH | QVQLQESGPGLAKPSQTLSLTC | 28 |
| | | TVSGGSISSGGYYWSWIRQHP | |
| | | GKGLEWIGYIYYSGSTYYNPSL | |
| | | KSRVTISVDTSKNQFSLKLSSVT | |
| | | AADTAVYYCARYPVWGAFDIW | |
| | | GQGTMVTVSS | |
| 07.2C08 | L1 | QSVSSSY | 29 |
| 07.2C08 | L2 | ATS | |
| 07.2C08 | L3 | QQYGSSPWT | 30 |
| 07.2C08 | H1 | GFTFSSSA | 31 |
| 07.2C08 | H2 | IVVGSGNT | 32 |
| 07.2C08 | H3 | AAAYCSGGSCSDGFDI | 33 |
| 07.2C08 | VL | EIVLTQSPGTLSLSPGERATLSC | 34 |
| | | RASQSVSSSYLAVVYQQKPGQA | |
| | | PRLLICATSSRATGIPDRFSGSG | |
| | | SGTDFTLTIRRLEPEDFAIYYCQ | |
| | | QYGSSPVVTFGQGTKVEIK | |
| 07.2C08 | VH | EVQLVQSGPEVKKPGTSVKVS | 35 |
| | | CKASGFTFSSSAVQWVRQARG | |
| | | QRLEWIGWIVVGSGNTNYAQK | |
| | | FQERVTITRDMSTNTAYMELSS | |
| | | LRSEDTAVYYCAAAYCSGGSC | |
| | | SDGFDIWGQGTMVTVSS | |
| 07.3D07 | L1 | SGSIASNY | 36 |
| 07.3D07 | L2 | EDN | |
| 07.3D07 | L3 | QSYDISNHWV | 37 |
| 07.3D07 | H1 | GFTFSRYT | 38 |
| 07.3D07 | H2 | ISYDGSNK | 39 |
| 07.3D07 | H3 | ARVLWLRGMFDY | 40 |
| 07.3D07 | VL | NFMLTQPHSVSESPGKTVTISC | 41 |
| | | TGSSGSIASNYVQWYQQRPGS | |
| | | APTTVIYEDNQRPSGVPDRFSG | |
| | | SIDSSSNSASLTISGLKTEDEAD | |
| | | YYCQSYDISNHVVVFGGGTKLT | |
| | | VL | |
| 07.3D07 | VH | EVQLVESGGGVVQPGRSLRLS | 42 |
| | | CAASGFTFSRYTMHWVRQAPG | |
| | | KGLEWVAFISYDGSNKYYADSV | |
| | | KGRFTISRDNSKNTLYLQMNSL | |
| | | RAEDTAVYYCARVLWLRGMFD | |
| | | YWGQGTLVTVSS | |
| 07.4A07 | L1 | QDITNY | 43 |
| 07.4A07 | L2 | DAS | |
| 07.4A07 | L3 | QQYDNLPLT | 44 |
| 07.4A07 | H1 | GFTFSSYA | 45 |
| 07.4A07 | H2 | ISYDGSNE | 46 |
| 07.4A07 | H3 | ARGDYYGSGSYPGKTFDY | 47 |
| 07.4A07 | VL | DIQMTQSPSSLSASVGDRVTIT | 48 |
| | | CQASQDITNYLNWYQQKPGKA | |
| | | PKLLIYDASNLETGVPSRFSGS | |
| | | GSGTDFTFTISSLQPEDIATYYC | |
| | | QQYDNLPLTFGGGTKVEIK | |
| 07.4A07 | VH | EVQLVESGGGVVQPGRSLRLS | 49 |
| | | CAASGFTFSSYAMFVVVRQAPG | |
| | | KGLEWVAVISYDGSNEYYADSV | |
| | | KGRFTISRDNSKNTLYLQMNSL | |
| | | RAEDTAVYYCARGDYYGSGSY | |
| | | PGKTFDYWGQGTLVTVSS | |
| 07.4B05 | L1 | QSVLYSSNNKDY | 50 |
| 07.4B05 | L2 | WAS | |
| 07.4B05 | L3 | QQYYSTPYT | 51 |
| 07.4B05 | H1 | GGTFSSYA | 52 |
| 07.4B05 | H2 | IIPILGIA | 53 |
| 07.4B05 | H3 | ARGRLDSYSGSYYSWFDP | 54 |
| 07.4B05 | VL | DIVMTQSPDSLAVSLGERATIN | 55 |
| | | CKSSQSVLYSSNNKDYLAWYQ | |
| | | QKPGQPPNLLIYWASTRESGVP | |
| | | DRFSGSGSGTDFTLTISSLQAE | |
| | | DVAVYYCQQYYSTPYTFGQGT | |
| | | KVEIK | |
| 07.4B05 | VH | EVQLVQSGAEVKKPGSSVKVS | 56 |
| | | CKASGGTFSSYAINWVRQAPG | |
| | | QGLEWMGRIIPILGIANYAQKFQ | |
| | | GRVTITADKSTSTAYMELSSLR | |
| | | SEDTAVYYCARGRLDSYSGSY | |
| | | YSWFDPWGQGTLVTVSS | |
| 07.4D09 | L1 | SSDVGSYNL | 57 |
| 07.4D09 | L2 | EVS | |
| 07.4D09 | L3 | CSYAGSSTWV | 58 |
| 07.4D09 | H1 | GGSISSSNW | 59 |
| 07.4D09 | H2 | IYHSGNT | 60 |
| 07.4D09 | H3 | ATKYCSGGSCSYFGY | 61 |
| 07.4D09 | VL | QSAITQPASVSGSPGQSITISC | 62 |
| | | TGTSSDVGSYNLVSWYQQHPG | |
| | | KAPKLMIYEVSKRPSGVSNRFS | |
| | | GSKSGNTASLTISGLQAEDEAD | |
| | | YYCCSYAGSST- | |
| | | WVFGGGTKLTVL | |
| 07.4D09 | VH | QVQLQESGPGLVKPSGTLSLTC | 63 |
| | | AVSGGSISSSNWWSWVRQPP | |
| | | GKGLEWIGEIYHSGNTNYNPSL | |
| | | KSRVTISVDKSKNQFSLKLSSVT | |
| | | AADTAVYYCATKYCSGGSCSY | |
| | | FGYWGQGTLVTVSS | |
| 20.1A12 | L1 | QSVSSSY | 64 |
| 20.1A12 | L2 | GAS | |
| 20.1A12 | L3 | QQYGSSYT | 65 |
| 20.1A12 | H1 | GFTFSSCG | 66 |
| 20.1A12 | H2 | ISYDGSNK | 67 |
| 20.1A12 | H3 | AKGHSGSYRAPFDY | 68 |
| 20.1A12 | VL | EIVLTQSPGTLSLSPGERATLSC | 69 |
| | | RASQSVSSSYLAWYQQKPGQA | |
| | | PRLLIYGASSRATGIPDRFSGS | |
| | | GSGTDFTLTISRLEPEDFAVYY | |
| | | CQQYGSSYTFGQGTKVEIK | |
| 20.1A12 | VH | EVQLVESGGGVVQPGRSLRLS | 70 |
| | | CAASGFTFSSCGMHWVRQAP | |
| | | GKGLEWVAVISYDGSNKYYAD | |
| | | SVKGRFTISRDNSKNTLYLQMN | |
| | | SLRAEDTAVYYCAKGHSGSYR | |
| | | APFDYWGQGTLVTVSS | |
| 20.2A03 | L1 | ALPKKY | 71 |
| 20.2A03 | L2 | EDS | |
| 20.2A03 | L3 | YSTDSSDNHRRV | 72 |
| 20.2A03 | H1 | GFTFSTYG | 73 |
| 20.2A03 | H2 | IWYDGSNK | 74 |
| 20.2A03 | H3 | AREAYFGSGSSPDY | 75 |
| 20.2A03 | VL | SYELTQPPSVSVSPGQTARITC | 76 |
| | | SGDALPKKYAYWYQQKSGQAP | |
| | | VLVIYEDSKRPSGIPERFSGSSS | |
| | | GTMATLTISGAQVGDEADYYCY | |
| | | STDSSDNHRRVFGGGTKLTVL | |
| 20.2A03 | VH | EVQLVESGGGVVQPGRSLRLS | 77 |
| | | CAASGFTFSTYGMHWVRQAPG | |
| | | KGLEWVAVIWYDGSNKYYADS | |
| | | VKGRFTISRDNSKNTLYLQMNS | |
| | | LRAEDTAVYYCAREAYFGSGS | |
| | | SPDYWGQGTLVTVSS | |
| 20.3C08 | L1 | ALPKKY | 78 |
| 20.3C08 | L2 | EDS | |
| 20.3C08 | L3 | YSTDSGGNPQGV | 79 |
| 20.3C08 | H1 | GFTFSSYW | 80 |
| 20.3C08 | H2 | IKEDGSEK | 81 |
| 20.3C08 | H3 | AREGTYYYDSSAYYNGGLDY | 82 |
| 20.3C08 | VL | SYELTQPPSVSVSPGQTARITC | 83 |
| | | SGDALPKKYAYWFQQKSGQAP | |
| | | VLVIYEDSKRPSGIPERFSGSSS | |
| | | GTMATLTISGAQVEDEADYYCY | |
| | | STDSSGNHRRLFGTGTKVTVL | |
| 20.3C08 | VH | EVQLVESGGGLVQPGGSLRLS | 84 |
| | | CAASGFTFSSYWMSWVRQAP | |
| | | GKGLEWVANIKEDGSEKYYVD | |
| | | SVKGRFTISRDNAKNSLYLQMN | |
| | | SLRAEDTAVYYCAREGTYYYDS | |
| | | SAYYNGGLDYWGQGTLVTVSS | |
| 22.1A12 | L1 | QDISNY | 85 |
| 22.1A12 | L2 | DAS | |
| 22.1A12 | L3 | QQYDNIPLT | 86 |
| 22.1A12 | H1 | GFTFYNYG | 87 |
| 22.1A12 | H2 | ISYDGSNK | 88 |
| 22.1A12 | H3 | AKQGGGTYCGGGSCYRGYFD | 89 |
| | | Y | |
| 22.1A12 | VL | DIQMTQSPSSLSASVGDRVTIT | 90 |
| | | CQASQDISNYLNWYQQKPGKA | |
| | | PKLLIYDASNLETGVPSRFSGS | |
| | | GSGTDFTFIISSLQPEDIATYYC | |
| | | QQYDNIPLTFGGGTKVEIK | |
| 22.1A12 | VH | EVQLVESGGVVVQPGRSLRLS | 91 |
| | | CAASGFTFYNYGMHWVRQAP | |
| | | GKGLEWVAVISYDGSNKYYAD | |
| | | SVKGRFTISRDNSKNTLYLQMN | |
| | | SLRAEDTAVYYCAKQGGGTYC | |
| | | GGGSCYRGYFDYWGQGTLVT | |
| | | VSS | |
| 22.1B08 | L1 | QSVSSY | 92 |
| 22.1B08 | L2 | NAS | |
| 22.1B08 | L3 | QQRSNRPPRWT | 93 |
| 22.1B08 | H1 | GYTFSNYY | 94 |
| 22.1B08 | H2 | FNPSGGGT | 95 |
| 22.1B08 | H3 | ARDPRVPAVTNVNDAFDL | 96 |
| 22.1B08 | VL | EIVLTQSPATLSLSPGERATLSC | 97 |
| | | RASQSVSSYLAWYQHKPGQAP | |
| | | RLIIYNASNRATGIPARFSGSRS | |
| | | GTDFTLTISSLEPEDFAVYYCQ | |
| | | QRSNRPPRVVTFGQGTKVEIK | |
| 22.1B08 | VH | EVQLVQSGAEAKKPGASVNISC | 98 |
| | | RTSGYTFSNYYIHWVRQAPGQ | |
| | | GLEWMGIFNPSGGGTSYAQNF | |
| | | QGRLTMTSDTSTSTVFMELSSL | |
| | | GSEDTAVYYCARDPRVPAVTN | |
| | | VNDAFDLWGQGTMVTVSS | |
| 22.1B12 | L1 | QSVLYSSNNKNY | 99 |
| 22.1B12 | L2 | WAS | |
| 22.1B12 | L3 | QQYYSTPCS | 100 |
| 22.1B12 | H1 | EFTVSSNY | 101 |
| 22.1B12 | H2 | IYLGGST | 102 |
| 22.1B12 | H3 | ARSHLEVRGVFDN | 103 |
| 22.1B12 | VL | DIVMTQSPDSLAVSLGERATVN | 104 |
| | | CKSSQSVLYSSNNKNYLAWYQ | |
| | | QKPGQPPKLLIYWASTRESGVP | |
| | | DRFSGSGSGTDFTLTISSLQAE | |
| | | DVAVYYCQQYYSTPCSFGQGT | |
| | | KVEIK | |
| 22.1B12 | VH | EVQLVETGGGLIQPGGSLRLSC | 105 |
| | | AVSEFTVSSNYMSWVRQAPGE | |
| | | GLEWVSVIYLGGSTDYADSVKG | |
| | | RFTISRDNSKNTLYLQMNSLRA | |
| | | EDTAVYYCARSHLEVRGVFDN | |
| | | WGQGTLVTVSS | |
| 22.1E07 | L1 | ALPKKY | 106 |
| 22.1E07 | L2 | EDS | |
| 22.1E07 | L3 | YSTDSSVNGRV | 107 |
| 22.1E07 | H1 | GFTFSSYG | 108 |
| 22.1E07 | H2 | IWYDGGNK | 109 |
| 22.1E07 | H3 | AREGVYGDIGGAGLDY | 110 |
| 22.1E07 | VL | SYELTQPPSVSVSPGQTARITC | 111 |
| | | SGDALPKKYAYWYQQKSGQAP | |
| | | VLVIYEDSKRPSGIPERFSGSSS | |
| | | GTMATLTISGAQVEDEADYYCY | |
| | | STDSSVNGRVFGTGTKVTVL | |
| 22.1E07 | VH | EVQLVESGGGVVQPGRSLRLS | 112 |
| | | CAASGFTFSSYGMHWVRQAP | |
| | | GKGLEWVAVIWYDGGNKHYAD | |
| | | SVKGRFTISRDNSKNTLYLQMD | |
| | | SLRAEDTAVYYCAREGVYGDIG | |
| | | GAGLDYWGQGTLVTVSS | |
| 22.1E11 | L1 | QDISNY | 113 |
| 22.1E11 | L2 | DAS | |
| 22.1E11 | L3 | QQYDNLLT | 114 |
| 22.1E11 | H1 | GFTFSSYG | 115 |
| 22.1E11 | H2 | ISYDGSNK | 116 |
| 22.1E11 | H3 | AKMGGVYCSAGNCYSGRLEY | 117 |
| 22.1E11 | VL | DIQMTQSPSSLSASVGDRVTIT | 118 |
| | | CQASQDISNYLNWYQQKPGKA | |
| | | PKLLIYDASNLETGVPSRFSGS | |
| | | GSGTDFTFTISSLQPEDIATYYC | |
| | | QQYDNLLTFGPGTKVDIK | |
| 22.1E11 | VH | EVQLVESGGGVVQPGRSLRLS | 119 |
| | | CAASGFTFSSYGMHWVRQAP | |
| | | GKGLEWVAVISYDGSNKYYAD | |
| | | SVKGRFTISRDNSKNTLFLQMS | |
| | | SLRAEDTAVYYCAKMGGVYCS | |
| | | AGNCYSGRLEYWGLGTLVTVS | |
| | | S | |
| 22.1G10 | L1 | QSISYFSNNKNY | 120 |
| 22.1G10 | L2 | WAS | |
| 22.1G10 | L3 | QQYFTTPWT | 121 |
| 22.1G10 | H1 | GGSMNSNY | 122 |
| 22.1G10 | H2 | IYYRGST | 123 |
| 22.1G10 | H3 | ARETVNNWVDP | 124 |
| 22.1G10 | VL | DIVMTQSPDSLTVSLGERATINC | 125 |
| | | KSSQSISYFSNNKNYLAWYQQ | |
| | | KPGQPPKLLIYWASTRESGVPD | |
| | | RFGGSGSGADFTLTISSLQAED | |
| | | VAVYYCQQYFTTPVVTFGQGTK | |
| | | VEIK | |
| 22.1G10 | VH | QVQLQESGPRLVRPLETLSLTC | 126 |
| | | TVSGGSMNSNYWSWIRQPPG | |
| | | KRLEWIGYIYYRGSTNYNPSLK | |
| | | SRVTISVDTSKNQFSLNLTSVTA | |
| | | ADTAIYYCARETVNNWVDPWG | |
| | | QGTLVTVSS | |
| 22.2A06 | L1 | RGSIAGNY | 127 |
| 22.2A06 | L2 | EDN | |
| 22.2A06 | L3 | QSFDSSNVV | 128 |
| 22.2A06 | H1 | GYSFTSYW | 129 |
| 22.2A06 | H2 | IYPGDSDT | 130 |
| 22.2A06 | H3 | ARREWGGSLGHIDY | 131 |
| 22.2A06 | VL | NFMLTQPHSVSESPGKTVTISC | 132 |
| | | TRSRGSIAGNYVQWYQQRPGS | |
| | | APTTVIYEDNQRPSGVPDRFSG | |
| | | SIDSSSNSASLTISGLKTEDEAE | |
| | | YYCQSFDSSNVVFGGGTKVTV | |
| | | L | |
| 22.2A06 | VH | EVQLVQSGAEVKKPGESLKISC | 133 |
| | | KGSGYSFTSYWIGWVRQMPG | |
| | | RGLEWMGIIYPGDSDTRYSPSF | |
| | | QGQVTISADKSISTAYLQWSSL | |
| | | KASDTAMYYCARREWGGSLG | |
| | | HIDYWGQGTLVTVSS | |
| 22.2B06 | L1 | NIGSNS | 134 |
| 22.2B06 | L2 | DDS | |
| 22.2B06 | L3 | QVWDSSSDPVV | 135 |
| 22.2B06 | H1 | GFTVSSNY | 136 |
| 22.2B06 | H2 | IYSGGST | 137 |
| 22.2B06 | H3 | ARDLQLYGMDV | 138 |
| 22.2B06 | VL | SYELTQPPSVSVAPGQTARITC | 139 |
| | | GGNNIGSNSVHVVYQQKPGQA | |
| | | PVLVVYDDSDRPSGIPERFSGS | |
| | | NSGNTATLTISRVEAGDEADYH | |
| | | CQVWDSSSDPVVFGGGTKLTV | |
| | | L | |
| 22.2B06 | VH | EVQLVETGGGLIQPGGSLRLSC | 140 |
| | | AASGFTVSSNYMTWVRQAPGK | |
| | | GLEWVSLIYSGGSTYYADSVKG | |
| | | RFTISRDNSKNTLYLQMNSLRA | |
| | | EDTAVYYCARDLQLYGMDVWG | |
| | | QGTTVTVSS | |
| 22.2F03 | L1 | ALPKQY | 141 |
| 22.2F03 | L2 | KDS | |
| 22.2F03 | L3 | QSADSSGTYV | 142 |
| 22.2F03 | H1 | GYIFTSYG | 143 |
| 22.2F03 | H2 | ISAYNGNT | 144 |
| 22.2F03 | H3 | ARVPGLVGYSSSVVYDNEKNYY | 145 |
| | | YYYYGMDV | |
| 22.2F03 | VL | SYELTQPPSVSVSPGQTARITC | 146 |
| | | SGDALPKQYAYWYQQKPGQA | |
| | | PVLVIYKDSERPSGIPERFSGSS | |
| | | TGTTVTLTISGVQAEDEADYYC | |
| | | QSADSSGTYVFGTGTKVTVL | |
| 22.2F03 | VH | EVQLVQSGAEVKKPGASVKVS | 147 |
| | | CKASGYIFTSYGISWVRQAPGQ | |
| | | GLEWMGWISAYNGNTNYAQKL | |
| | | QGRVTMTTDTSTSTAYMELRSL | |
| | | RSDDTAVYYCARVPGLVGYSS | |
| | | SWYDNEKNYYYYYYGMDVWG | |
| | | QGTTVTVSS | |
| 22.3A06 | L1 | QSVSTY | 148 |
| 22.3A06 | L2 | DAS | |
| 22.3A06 | L3 | QHRSNWPLT | 149 |
| 22.3A06 | H1 | GFTFSSYA | 150 |
| 22.3A06 | H2 | ISGSGGST | 151 |
| 22.3A06 | H3 | AKADTAMAWYNWFDP | 152 |
| 22.3A06 | VL | EIVLTQSPATLSLSPGERATLSC | 153 |
| | | RASQSVSTYLAWYQQKPGQAL | |
| | | RLLIYDASNRATGIPARFSGSGS | |
| | | GTDFTLTISSLEPEDFAVYYCQ | |
| | | HRSNWPLTFGGGTKVEIK | |
| 22.3A06 | VH | EVQLLESGGGLVQPGGSLRLS | 154 |
| | | CAASGFTFSSYAMSWVRQAPG | |
| | | KGLEWVSAISGSGGSTYYADS | |
| | | VKGRLTISRDNSKNTLYMQMNS | |
| | | LRAEDTAVYYCAKADTAMAWY | |
| | | NWFDPWGQGTLVTVSS | |
| 22.3A11 | L1 | NIGRKS | 155 |
| 22.3A11 | L2 | DDS | |
| 22.3A11 | L3 | QVWDNSSDQPNYV | 156 |
| 22.3A11 | H1 | GGSFSGYY | 157 |
| 22.3A11 | H2 | INHSGST | 158 |
| 22.3A11 | H3 | ARVWVRWWYFDL | 159 |
| 22.3A11 | VL | SYELTQPPSVSVAPGQTARITC | 160 |
| | | GGNNIGRKSVHWYQQKPGQA | |
| | | PVLVVYDDSDRPSGIPERFSGS | |
| | | NSGNTATLTLSRVEAGDEADYY | |
| | | CQVWDNSSDQPNYVFGTGTKV | |
| | | TVL | |
| 22.3A11 | VH | QVQLQQWGAGLLKPSETLSLT | 161 |
| | | CAVYGGSFSGYYWSWIRQPPG | |
| | | KGLEVVLGEINHSGSTNYNPSLK | |
| | | SRVTISVDTSKNQFSLKLSSVTA | |
| | | ADTAVYYCARVWVRWWYFDL | |
| | | WGRGTLVTVSS | |

Also provided are peptides, polypeptides and/or proteins derived from any of the antibodies or antibody binding fragments described herein. Generally, as used herein, the derivatives provided here are substantially similar to the antibodies or antibody binding fragments described herein. For example, they may contain one or more conservative substitutions in their amino acid sequences or may contain a chemical modification. The derivatives and modified peptides/polypeptides/proteins all are considered “structurally similar” which means they retain the structure (e.g., the secondary, tertiary or quaternary structure) of the parent molecule and are ex-pected to interact with the antigen in the same way as the parent molecule.

A class of synthetically derived antibodies or antigen-binding moieties can be generated by conservatively mutating resides on the parent molecule to generate a peptide, polypeptide or protein maintaining the same activity as the parent molecule. Representative conservative substitutions are known in the art and are also summarized here.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

A second way to generate a functional peptide/polypeptide or protein based on the sequences provided herein is through the use of computational, “in-silico” design. For example, computationally designed antibodies or antigen-binding fragments may be designed using standard methods of the art. For example, see Strauch E M et al., (Nat Biotechnol. 2017 July; 35(7):667-671), Fleishman S J et al., (Science. 2011 May 13; 332(6031):816-21), and Koday M T et al., (PLoS Pathog. 2016 Feb. 4; 12(2):e1005409), each incorporated by reference in their entirety.

In various embodiments, an antibody or antibody binding fragment thereof is provided that binds a coronavirus (e.g., SARS-CoV-2) and is structurally similar to any of the antibodies described herein. That is it has the same secondary, tertiary or quaternary structure as the antibodies or antigen-binding fragments described herein. For example, the antibody or antigen-binding fragment can have a tertiary structure that is structurally similar to a single CDR loop. For example, the antibody or antigen-binding fragment can have a tertiary structure that is structurally similar to a H3 loop, e.g., a loop comprising those disclosed in Table A and/or Table B or any combination thereof. Alternatively or in addition, the antibody or antigen-binding fragment can have a tertiary structure that is structurally similar to a CDR loop comprising any one of those disclosed in Table A and/or Table B.

In various embodiments, the antibody can comprise at least one amino acid substitution, deletion, or insertion in a variable region, a hinge region or an Fc region relative to the sequence of a wild-type variable region, hinge region or a wild-type Fc region.

For example, the antibody can comprise an Fc region that contains at least one amino acid substitution, deletion, or insertion relative to the sequence of a wild-type Fc region. In various embodiments, this substitution, deletion or insertion can prevent or reduce recycling of the antibody (e.g., in vivo).

In various embodiments, the antibody or antigen-binding fragment can comprise a heavy chain variable region and/or light chain variable region comprising at least one amino acid substitution, deletion, or insertion as compared to any one of the antibodies disclosed in Table A or Table B.

Further, as described further below, the antibodies or antigen-binding fragments described herein can be expressed recombinantly (e.g., using a recombinant cell line or recombinant organism). Accordingly, the antibodies or antigen-binding fragments may comprise post-translational modifications (e.g., glycosylation profiles, methylation) that differs from naturally occurring antibodies.

The antibodies and antigen-binding fragments thereof described herein have some measure of binding affinity to a coronavirus. Most preferably, the antibody or antigen-binding fragment binds SARS-CoV-2 (that is, the coronavirus comprises SARS-CoV-2). In various embodiments, the antibodies and antigen-binding fragments thereof described herein can bind a receptor binding domain (RBD) expressed by the coronavirus (e.g., SARS-CoV-2).

Further, the antibodies and antigen-binding fragments herein may have a certain affinity for a specific epitope on the coronavirus (e.g., an epitope on the receptor binding domain, RBD).

The binding of the antibody or antigen-binding fragment can neutralize the coronavirus (e.g., SARS-CoV-2). In various embodiments, the antibodies and/or binding fragment neutralize the coronavirus with an IC₅₀ of about 0.0001 μg/ml to about 30 μg/ml. For example, the antibody or antigen-binding fragment can have an IC₅₀ of about 0.001 μg/ml to about 30 μg/ml. The neutralizing ability of the antibody or antigen-binding fragment can be determined by measuring, for example, the ability of the virus to replicate in the presence or absence of the antibody or antigen-binding fragment.

In various embodiments, the antibody or antigen-binding fragment described herein is humanized. “Humanized” antibodies are generally chimeric or mutant monoclonal antibodies from mouse, rat, hamster, rabbit or other species, bearing human constant and/or variable region domains or specific changes.

In various embodiments, the antibody or antigen-binding fragment described herein is a monoclonal antibody. As used herein, the term “monoclonal antibodies” refer to antibodies or antigen-binding fragments that are expressed from the same genetic sequence or sequences and consist of identical antibody molecules.

In various embodiments, the antibody or antigen-binding fragment described herein is an IgG type antibody. For example, the antibody or antigen-binding fragment can be an IgG1, IgG2, IgG3, or an IgG4 type antibody.

DNA molecules encoding light chain variable regions and/or heavy chain variable regions can be chemically synthesized. Synthetic DNA molecules can be ligated to other appropriate nucleotide sequences, including, e.g., constant region coding sequences, and expression control sequences, to produce conventional gene expression constructs encoding the desired antibody. Production of defined gene constructs is within routine skill in the art.

Nucleic acids encoding desired antibodies can be incorporated (ligated) into expression vectors, which can be introduced into host cells through conventional transfection or transformation techniques. Illustrative host cells are E. coli cells, Chinese hamster ovary (CHO) cells, HeLa cells, baby hamster kidney (BHK) cells, monkey kidney cells (COS), human hepatocellular carcinoma cells (e.g., Hep G2), human embryonal kidney (HEK) cells and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. If the engineered gene is to be expressed in eukaryotic host cells, e.g., CHO cells, it is first inserted into an expression vector containing a suitable eukaryotic promoter, a secretion signal, a poly A sequence, and a stop codon, and, optionally, may contain enhancers, and various introns. This expression vector optionally contains sequences encoding all or part of a constant region, enabling an entire, or a part of, a heavy or light chain to be expressed. The gene construct can be introduced into eukaryotic host cells using conventional techniques. The host cells express VL or VH fragments, VL-VH heterodimers, VH-VL or VL-VH single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector encoding a polypeptide comprising an entire, or part of, a heavy chain or heavy chain variable region, and another expression vector encoding a polypeptide comprising an entire, or part of, a light chain or light chain variable region).

A polypeptide comprising an immunoglobulin heavy chain variable region or light chain variable region can be produced by growing (culturing) a host cell transfected with an expression vector encoding such variable region, under conditions that permit expression of the polypeptide. Following expression, the polypeptide can be harvested and purified or isolated using techniques, e.g., using affinity tags such as glutathione-S-transferase (GST) and histidine tags.

A monoclonal antibody, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit ex-pression of both chains. The intact antibody (or antigen-binding fragment of the antibody) can be harvested and purified or isolated using other techniques, e.g., Protein A, Protein G, affinity tags such as glutathione-S-transferase (GST) and histidine tags. The heavy chain and the light chain can be expressed from a single expression vector or from two separate expression vectors.

Therefore, in various embodiments, a nucleic acid is provided, the nucleic acid comprising a nucleotide sequence encoding the antibody or antigen-binding fragment described herein. The skilled man will appreciate that functional variants of these nucleic acid molecules are also intended to be a part of the present invention. Functional variants are nucleic acid sequences that can be directly translated, using the standard genetic code, to provide an amino acid sequence identical to that translated from the parental nucleic acid molecules.

Suitable nucleic acids that can encode portions of the inventive antibodies can be determined using standard techniques. In various embodiments, the nucleic acid comprises a nucleotide sequence encoding an immunoglobulin heavy chain variable region of the antibody or antigen-binding fragment described herein. In various embodiments, the nucleic acid comprises a nucleotide sequence encoding an immunoglobulin light chain variable region of the antibody or antigen-binding fragment described herein. In some embodiments, the nucleic acids encode one or more complementary determining regions (CDR) having the amino acid sequences described herein. As described above, a single nucleic acid may be provided that encodes more than one protein product (e.g., the immunoglobulin light chain and the immunoglobulin heavy chain). Alternatively, two or more separate nucleic acids may be provided each encoding one component of the antibody and/or antigen-binding fragment (e.g., the light chain or the heavy chain).

In various embodiments, an expression vector is provided comprising one or more of the nucleic acids described herein. Vectors can be derived from plasmids such as: F, F1, RP1, Col, pBR322, TOL, Ti, etc; cosmids; phages such as lambda, lambdoid, M13, Mu, P1, P22, Qβ, T-even, T-odd, T2, T4, T7 etc; or plant viruses. Vectors can be used for cloning and/or expression of the binding molecules of the invention and might even be used for gene therapy purposes. Vectors comprising one or more nucleic acid molecules according to the invention operably linked to one or more expression-regulating nucleic acid molecules are also covered by the present invention. The choice of the vector is dependent on the recombinant procedures followed and the host used. Introduction of vectors in host cells can be affected by inter alia calcium phosphate transfection, virus infection, DEAE-dextran mediated transfection, lipofectamine transfection or electroporation. Vectors may be autonomously replicating or may replicate together with the chromosome into which they have been integrated. Preferably, the vectors contain one or more selection markers. The choice of the markers may depend on the host cells of choice. They include, but are not limited to, kanamycin, neomycin, puromycin, hygromycin, zeocin, thymidine kinase gene from Herpes simplex virus (HSV-TK), dihydrofolate reductase gene from mouse (dhfr). Vectors comprising one or more nucleic acid molecules encoding the human binding molecules as described above operably linked to one or more nucleic acid molecules encoding proteins or peptides that can be used to isolate the human binding molecules are also covered by the invention. These proteins or peptides include, but are not limited to, glutathione-S-transferase, maltose binding protein, metal-binding polyhistidine, green fluorescent protein, luciferase and beta-galactosidase.

The expression vector may be transfected into a host cell to induce the translation and expression of the nucleic acid into the heavy chain variable region and/or the light chain variable region. Therefore, a host cell is provided comprising any expression vector described herein. Host cells include, but are not limited to, cells of mammalian, plant, insect, fungal or bacterial origin. Bacterial cells include, but are not limited to, cells from Gram-positive bacteria or Gram-negative bacteria such as several species of the genera Escherichia, such as E. coli, and Pseudomonas. In the group of fungal cells preferably yeast cells are used. Expression in yeast can be achieved by using yeast strains such as inter alia Pichia pastoris, Saccharomyces cerevisiae and Hansenula polymorpha. Furthermore, insect cells such as cells from Drosophila and Sf9 can be used as host cells. Besides that, the host cells can be plant cells such as inter alia cells from crop plants such as forestry plants, or cells from plants providing food and raw materials such as cereal plants, or medicinal plants, or cells from ornamentals, or cells from flower bulb crops. Transformed (transgenic) plants or plant cells are produced by methods such as Agrobacterium-mediated gene trans-fer, transformation of leaf discs, protoplast transformation by polyethylene glycol-induced DNA transfer, electroporation, sonication, microinjection or bolistic gene transfer. Additionally, a suitable expression system can be a baculovirus system. Expression systems using mammalian cells, such as Chinese Hamster Ovary (CHO) cells, COS cells, BHK cells, NSO cells or Bowes melanoma cells are preferred in the present invention. Since the present invention deals with molecules that may have to be administered to humans, a completely human expression system would be particularly preferred. Therefore, even more preferably, the host cells are human cells. Examples of human cells are, inter alia, HeLa, 911, AT1080, A549, HEK293, 293F and HEK293T cells.

Accordingly, the antibody or antigen-binding fragment can be expressed using a recombinant cell line or recombinant organism.

Further a method is provided for producing an antibody or antigen-binding fragment that binds a coronavirus, the method comprising growing a host cell as described herein under conditions so that the host cell expresses a polypeptide or polypeptides comprising the immunoglobulin heavy chain variable region and the immunoglobulin light chain variable region, thereby producing the antibody or antigen-binding fragment and purifying the antibody or antigen-binding fragment.

Also provided are pharmaceutical compositions comprising at least one antibody or antigen-binding fragment described herein.

Pharmaceutical compositions containing one or more of the antibodies or antigen-binding fragments described herein can be formulated in any conventional manner. Proper formulation is dependent in part upon the route of administration selected. Routes of administration include, but are not limited to parenteral (e.g., intravenous, intraarterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration. Preferably, the composition is administered parenterally or is inhaled (e.g., intranasal). For example, the composition can be administered by intravenous infusion.

The pharmaceutical compositions can be formulated for parenteral administration, e.g., formulated for injection via intravenous, intra-arterial, subcutaneous, rectal, subcutaneous, intramuscular, intraorbital, intracapsular, intraspinal, intraperitoneal, or intrasternal routes. Dosage forms suitable for parenteral administration include solutions, suspensions, dispersions, emulsions or any other dosage form that can be administered parenterally.

The pharmaceutical composition can be formulated without blood, plasma or a major component of blood or plasma (e.g., blood cells, fibrin, hemoglobin, albumin, etc.).

The pharmaceutical composition can comprise from about 0.001 to about 99.99 wt. % of the antibody or antigen-binding fragment according to the total weight of the composition. For example, the pharmaceutical composition can comprise from about 0.001 to about 1%, about 0.001 to about 5%, about 0.001 to about 10%, about 0.001 to about 15%, about 0.001 to about 20%, about 0.001 to about 25%, about 0.001 to about 30%, about 1 to about 10%, about 1 to about 20%, about 1 to about 30%, about 10 to about 20%, about 10 to about 30%, about 10 to about 40%, about 10 to about 50%, about 20 to about 30%, about 20 to about 40%, about 20 to about 50%, about 20 to about 60%, about 20 to about 70%, about 20 to about 80%, about 20 to about 90%, about 30 to about 40%, about 30 to about 50%, about 30 to about 60%, about 30 to about 70%, about 30 to about 80%, about 30 to about 90%, about 40 to about 50%, about 40 to about 60%, about 40 to about 70%, about 40 to about 80%, about 40 to about 90%, about 50 to about 99.99%, about 50 to about 99%, about 60 to about 99%, about 70 to about 99%, about 80 to about 99%, about 90 to about 99%, about 50 to about 95%, about 60 to about 95%, about 70 to about 95%, about 80 to about 95%, about 90 to about 95%, about 50 to about 90%, about 60 to about 90%, about 70 to about 90%, about 80 to about 90%, about 85 to about 90%, about 50 to about 80%, about 60 to about 80%, about 70 to about 80%, about 75 to about 80%, about 50 to about 70%, about 60 to about 70%, or from about 50 to about 60% of the antibody or antigen-binding fragment by weight according to the total weight of the composition.

The compositions described herein can also comprise one or more pharmaceutically acceptable excipients and/or carriers. The pharmaceutically acceptable excipients and/or carriers for use in the compositions of the present invention can be selected based upon a number of factors including the particular compound used, and its concentration, stability and intended bioavailability; the subject, its age, size and general condition; and the route of administration.

Some examples of materials which can serve as pharmaceutically acceptable carriers in the compositions described herein are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

Pharmaceutically acceptable excipients are identified, for example, in The Handbook of Pharmaceutical Excipients, (American Pharmaceutical Association, Washington, D.C., and The Pharmaceutical Society of Great Britain, London, England, 1968). Additional excipients can be included in the pharmaceutical compositions of the invention for a variety of purposes. These excipients can impart properties which enhance retention of the compound at the site of administration, protect the stability of the composition, control the pH, facilitate processing of the compound into pharmaceutical compositions, and so on. Other excipients include, for example, fillers or diluents, surface active, wetting or emulsifying agents, preservatives, agents for adjusting pH or buffering agents, thickeners, colorants, dyes, flow aids, nonvolatile silicones, adhesives, bulking agents, flavorings, sweeteners, adsorbents, binders, disintegrating agents, lubricants, coating agents, and antioxidants.

In some embodiments, the composition further comprises at least one other therapeutic, prophylactic and/or diagnostic agent. Preferably, the therapeutic and/or prophylactic agents are capable of preventing and/or treating a coronavirus infection and/or a condition/symptom resulting from such an infection. Therapeutic and/or prophylactic agents include, but are not limited to, antiviral agents. Such agents can be binding molecules, small molecules, organic or inorganic compounds, enzymes, polynucleotide sequences, antiviral peptides, etc. The therapeutic and/or prophylactic agent can comprise an M2 inhibitor (e.g., amantadine, rimantadine) and/or a neuraminidase inhibitor (e.g., zanamivir, oseltamivir). In various embodiments, the anti-viral agent can comprise baloxavir, oseltamivir, zanamivir, peramivir, remdesivir, or any combination thereof. The therapeutic and/or prophylactic agent can also include various anti-malarial such as chloroquine, hydroxychloroquine, and analogues thereof.

The additional antibodies or therapeutic/prophylactic and/or diagnostic agents may be used in combination with the antibodies and antigen-binding fragments of the present invention. “In combination” herein, means simultaneously, as separate formulations (e.g., co-administered), or as one single combined formulation or according to a sequential administration regiment as separate formulations, in any order. Agents capable of preventing and/or treating an infection with coronavirus (e.g., SARS-CoV-2) and/or a condition resulting from such an infection that are in the experimental phase might also be used as other therapeutic and/or prophylactic agents useful in the present invention.

### II. Treatment Methods

The present disclosure encompasses methods to treat, prevent, or reduce the infectivity of a virus in a subject in need thereof. In some embodiments, the methods prevent or reduce the infectivity of a viral infection by preventing internalization of a virus into a cell of the subject or by preventing internalization of a viral genome into a cell of the subject. In some embodiments, administration of a composition provided herein, for instance those described in Section I, may disrupt or prevent an interaction between a viral surface protein (e.g., a spike protein) and a host receptor protein (e.g., an epithelial angiotensin converting enzyme (ACE)). For example, administration of a composition of the disclosure may block internalization of a coronavirus into a cell of a subject by blocking or disrupting interactions between a coronavirus spike protein and a host receptor protein and/or by sequestering the virus in vivo allowing for the virus bound to the composition to be eliminated by the subject's immune cells. Administering a composition of the disclosure to a subject at risk for a viral infection may reduce the risk of coronavirus infection in the subject.

In other embodiments, the present disclosure provides methods to treat, prevent, or reduce the infectivity of a respiratory viral infection. In some embodiments, the viral infection may be a coronavirus infection. The coronavirus may be SARS-CoV, SARS-CoV-2, MERS-CoV, HKU1, OC43, or 229E. The coronavirus may be a beta-coronavirus. A subject at risk for a coronavirus infection may come in contact with an asymptomatic carrier of the coronavirus infection, thereby unknowingly contracting the coronavirus infection.

In some embodiments, the compositions, methods, or treatment regiments disclosed herein may treat or prevent a SARS-CoV-2 infection (e.g., COVID-19). A SARS-CoV-2 infection may depend on host cell ACE-2 enzyme. In some embodiments, a SARS-CoV-2 infection may be blocked (e.g., prevented, treated, or slowed) by a composition of the disclosure. In various embodiments, a method of preventing or treating a coronavirus infection (e.g., COVID-19 caused by SARS-CoV-2) in a subject in need thereof is provided. The method can comprise administering any antibody or antigen-binding fragment (including any nucleic acid or expression vector that encodes the antibody or antigen-binding fragment), any vaccine, or any composition as described herein to the subject.

In various embodiments, the composition is administered parentally (e.g., systemically). In other embodiments, the composition is inhaled orally (e.g., intranasally). In both cases the composition is formulated (e.g., with carriers/excipients) according to its mode of administration as described above.

In various embodiments the composition is administered via intranasal, intramuscular, intravenous, and/or intradermal routes. In some embodiments, the composition is provided as an aerosol (e.g., for nasal administration).

Dosing regiments can be adjusted to provide the optimum desired response (e.g., a prophylactic or therapeutic response). Therefore, the dose used in the methods herein can vary depended on the intended use (e.g., for prophylactic vs. therapeutic use). Nevertheless, the com-positions described herein may be administered at a dose of about 1 to about 100 mg/kg body weight, or from about 1 to about 70 mg/kg body weight. Furthermore, a single bolus may be administered, several divided doses may be administered over time, or the dose may be proportionally reduced or increased as indicated by the exigencies of the therapeutic of the therapeutic situation.

In various embodiments, the antibody or antigen-binding fragment is delivered using a gene therapy technique. Such techniques generally comprise administering a viral vector comprising a nucleic acid that codes for a gene product of interest to a subject in need thereof. Therefore, in certain embodiments, the antibody or antigen-binding fragment described herein is delivered to a subject in need thereof by administering a viral vector or vectors (e.g., an adenovirus) containing one or more of the necessary nucleic acids (such as, for example, the nucleic acids provided herein) for expressing the antibody or antibody binding fragment in vivo. Similar delivery methods have successfully lead to the expression of protective antibodies in other disease con-texts. For example, see Sofer-Podesta C. et al., “Adenovirus-mediated delivery of an Anti-V Antigen Monoclonal Antibody Protects Mice against a Lethal Yersinia pestis Challenge” Infection and Immunity March 2009, 77 (4) 1561-1568, the entire disclosure of which is incorporated herein by reference.

In various embodiments, the coronavirus infection to be treated is a SARS infection (e.g., severe acute respiratory syndrome caused by the coronavirus). In various embodiments, the coronavirus infection comprises COVID-19.

Generally, the methods as described herein comprise administration of a therapeutically effective amount of a composition of the disclosure to a subject. The methods described herein are generally performed on a subject in need thereof. A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion ani-mal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

The concentration of antibody in formulations to be administered is an effective amount and ranges from as low as about 0.1% by weight to as much as about 15 or about 20% by weight and will be selected primarily based on fluid volumes, viscosities, and so forth, in accordance with the particular mode of administration selected if desired. A typical composition for injection to a living subject could be made up to contain 1 mL sterile buffered water of phosphate buffered saline and about 1-1000 mg of any one of or a combination of the antibodies disclosed herein. The formulation could be sterile filtered after making the formulation, or otherwise made microbiologically acceptable. A typical composition for intravenous infusion could have volumes between 1-250 mL of fluid, such as sterile Ringer's solution, and 1-100 mg per ml, or more in antibody of the disclosure concentration. Antibodies disclosed herein can be frozen or lyophilized for storage and reconstituted in a suitable sterile carrier prior to use. Lyophilization and reconstitution may lead to varying degrees of antibody activity loss (e.g. with conventional immune globulins, IgM antibodies tend to have greater activity loss than IgG antibodies). Dosages administered are effective dosages and may have to be adjusted to compensate. The pH of the formulations generally pharmaceutical grade quality, will be selected to balance antibody stability (chemical and physical) and comfort to the subject when administered. Generally, a pH between 4 and 8 is tolerated. Doses will vary from individual to individual based on size, weight, and other physiobiological characteristics of the individual receiving the successful administration.

As used herein, the term “therapeutically effective amount” means an amount of a substance (e.g. an antibody of the disclosure) that leads to measurable and beneficial effects for the subject administered the substance, i.e., significant efficacy. The therapeutically effective amount or dose of compound administered according to this discovery will be determined using standard clinical techniques and may be by influenced by the circumstances surrounding the case, including the antibody administered, the route of administration, and the status of the symptoms being treated, among other considerations. A typical dose may contain from about 0.01 mg/kg to about 100 mg/kg of an antibody of the disclosure described herein. Doses can range from about 0.05 mg/kg to about 50 mg/kg, more preferably from about 0.1 mg/kg to about 25 mg/kg. The frequency of dosing may be daily or once, twice, three times or more per week or per month, as needed as to effectively treat the symptoms.

The timing of administration of the treatment relative to the disease itself and duration of treatment will be determined by the circumstances surrounding the case. Duration of treatment could range from a single dose administered on a one-time basis to a life-long course of therapeutic treatments.

Although the foregoing methods appear the most convenient and most appropriate and effective for administration of proteins such as humanized antibodies, by suitable adaptation, other effective techniques for administration, such as intraventricular administration, transdermal administration and oral administration may be employed provided proper formulation is utilized herein. In addition, a person skilled in the art can use a polynucleotide of the invention encoding any one of the above-described antibodies instead of the proteinaceous material itself. For example,

In addition, it may be desirable to employ controlled release formulations using biodegradable films and matrices, or osmotic mini-pumps, or delivery systems based on dextran beads, alginate, or collagen.

### EXAMPLES

The following non-limiting examples are provided to further illustrate various iterations of the invention.

### Example 1—SARS-CoV-2 mRNA Vaccines Induce Persistent Human Germinal Centre Responses

SARS-CoV-2 mRNA-based vaccines are about 95% effective in preventing COVID-19. The dynamics of antibody-secreting plasmablasts and germinal centre B cells induced by these vaccines in humans remain unclear. The present example examined antigen-specific B cell responses in peripheral blood (n=41) and draining lymph nodes in 14 individuals who had received 2 doses of BNT162b2, an mRNA-based vaccine that encodes the full-length SARS-CoV-2 spike (S) gene. Circulating IgG- and IgA-secreting plasmablasts that target the S protein peaked one week after the second immunization and then declined, becoming undetectable three weeks later. These plasmablast responses preceded maximal levels of serum anti-S binding and neutralizing antibodies to an early circulating SARS-CoV-2 strain as well as emerging variants, especially in individuals who had previously been infected with SARS-CoV-2 (who produced the most robust serological responses). By examining fine needle aspirates of draining axillary lymph nodes, germinal centre B cells that bound S protein in all participants were identified who were sampled after primary immunization. High frequencies of S-binding germinal centre B cells and plasmablasts were sustained in these draining lymph nodes for at least 12 weeks after the booster immunization. S-binding monoclonal antibodies derived from germinal centre B cells predominantly targeted the receptor-binding domain of the S protein, and fewer clones bound to the N-terminal domain or to epitopes shared with the S proteins of the human betacoronaviruses OC43 and HKU1. These latter cross-reactive B cell clones had higher levels of somatic hypermutation as compared to those that recognized only the SARS-CoV-2 S protein, which suggests a memory B cell origin. The present example demonstrates that SARS-CoV-2 mRNA-based vaccination of humans induces a persistent germinal centre B cell response, which enables the generation of robust humoral immunity.

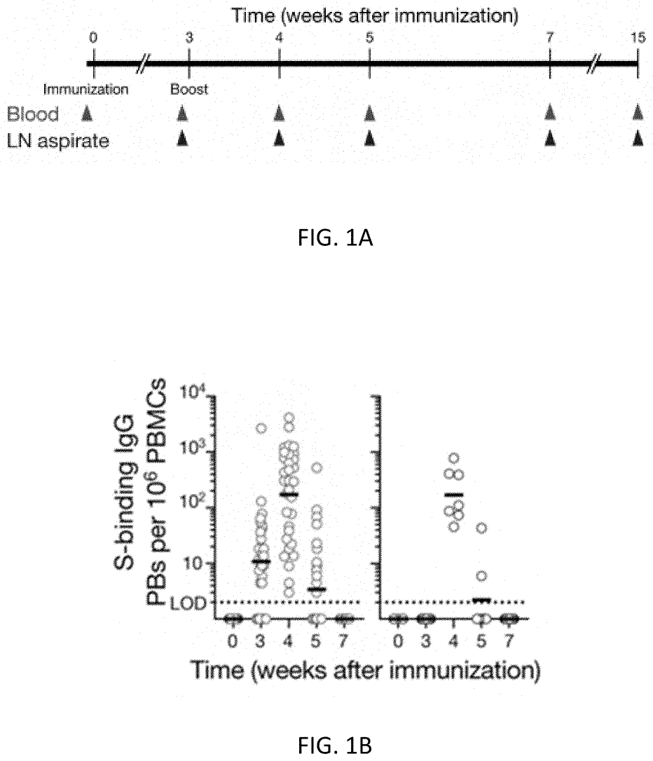

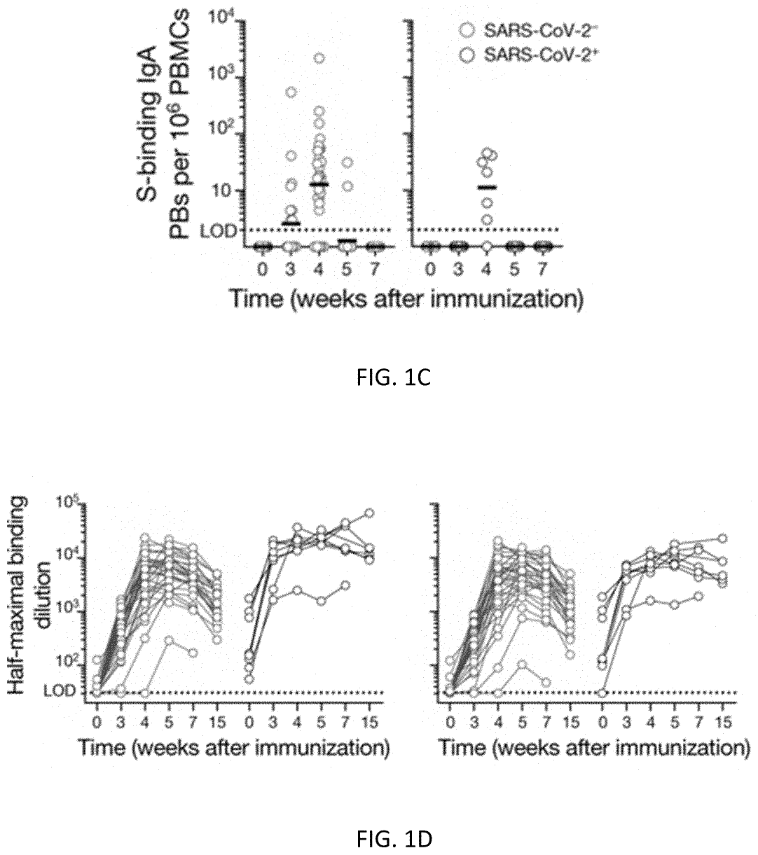

The concept of using mRNAs as vaccines was introduced over 30 years ago. Key refinements that improved the biological stability and translation capacity of exogenous mRNA enabled development of these molecules as vaccines. The emergence of SARS-CoV-2 in December 2019, and the ensuing pandemic, has revealed the potential of this platform. Hundreds of millions of people have received one of the two SARS-CoV-2 mRNA-based vaccines that were granted emergency use authorization by the US Food and Drug Administration in December 2020. Both of these vaccines demonstrated notable immunogenicity in phase-I/II studies and efficacy in phase-III studies. Whether these vaccines induce the robust and persistent germinal centre reactions that are critical for generating high-affinity and durable antibody responses has not been examined in humans. To address this question, an observational study was conducted of 41 healthy adults (8 of whom had a history of confirmed SARS-CoV-2 infection) who received the Pfizer-BioNTech SARS-CoV-2 mRNA vaccine BNT162b2 (Table 1 and 2). Blood samples were collected at baseline, and at weeks 3 (pre-boost), 4, 5, 7 and 15 after the first immunization. Fine needle aspirates (FNAs) of the draining axillary lymph nodes were collected from 14 participants (none with history of SARS-CoV-2 infection) at weeks 3 (pre-boost), 4, 5, 7, and 15 after the first immunization (FIG. 1A).

TABLE 1
Participant Demographics

| | Total N = 32 | | Lymph node N = 12 | |
|---|---|---|---|---|
| Variable | N (%) | | N (%) | |
| Age (median [range]) | 37 | (28-73) | 36.5 | (28-52) |
| Sex | | | | |
| Female | 16 | (50) | 7 | (58.3) |
| Male | 16 | (50) | 5 | (41.7) |
| Race | | | | |
| White | 25 | (78.1) | 10 | (83.3) |
| Asian | 5 | (15.6) | 1 | (8.3) |
| Black | 1 | (3.1) | 1 | (8.3) |
| Other | 1 | (3.1) | 0 | (0) |
| Ethnicity | | | | |
| Not of Hispanic, Latinx, or | 30 | (93.8) | 11 | (91.7) |
| Spanish origin | | | | |
| Hispanic, Latinx, Spanish origin | 2 | (6.3) | 1 | (8.3) |
| BMI (median [range]) | 25.3 | (21.4-40) | 23.5 | (21.4-40) |
| Comorbidities | | | | |
| Lung disease | 2 | (6.3) | 1 | (8.3) |
| Diabetes mellitus | 0 | (0) | 0 | (0) |
| Hypertension | 5 | (15.6) | 2 | (16.7) |
| Cardiovascular | 0 | (0) | 0 | (0) |
| Liver disease | 0 | (0) | 0 | (0) |
| Chronic kidney disease | 0 | (0) | 0 | (0) |
| Cancer on chemotherapy | 0 | (0) | 0 | (0) |
| Hematological malignancy | 0 | (0) | 0 | (0) |
| Pregnancy | 0 | (0) | 0 | (0) |
| Neurological | 0 | (0) | 0 | (0) |
| HIV | 0 | (0) | 0 | (0) |
| Solid organ transplant recipient | 0 | (0) | 0 | (0) |
| Bone marrow transplant | 0 | (0) | 0 | (0) |
| recipient | 1 | (3.1) | 0 | (0) |
| Hyperlipidemia | | | | |
| Confirmed SARS-CoV-2 | 7 | (21.9) | 0 | (0) |

| | | | |
|---|---|---|---|
| infection | 106 | (50-230) | — |

Time from SARS-CoV-2
infection to baseline visit in
days (median [range])

TABLE 2
Vaccine Side-Effects

| | Total | | | Lymph node | |
|---|---|---|---|---|---|
| Variable | N = 32 | | | N = 12 | |
| First dose | N (%) | | Second dose | N (%) | |
| None | 4 | (12.5) | None | 2 | (6.2) |
| Chills | 5 | (15.6) | Chills | 10 | (31.3) |
| Fever | 2 | (6.3) | Fever | 5 | (15.6) |
| Headache | 5 | (15.6) | Headache | 9 | (28.1) |
| Injection | 25 | (78.1) | Injection | 27 | (84.4) |
| site pain | | | site pain | | |
| Muscle or | 7 | (21.9) | Muscle or | 16 | (50) |
| joint pain | | | joint pain | | |
| Fatigue | 7 | (21.9) | Fatigue | 14 | (43.8) |
| Sweating | 0 | (0) | Sweating | 2 | (6.3) |

Duration of side effects in hours (median [range])

| | | | | | |
|---|---|---|---|---|---|
| Chills | 48 | (6-72) | Chills | 21 | (4-48) |
| Fever | 9 | (6-12) | Fever | 24 | (1-48) |
| Headache | 12 | (5-48) | Headache | 24 | (4-48) |
| Injection | 36 | (2-120) | Injection | 36 | (2-96) |
| site pain | | | site pain | | |
| Muscle or | 36 | (0-48) | Muscle or | 31.5 | (1-48) |
| joint pain | | | joint pain | | |
| Fatigue | 36 | (5-48) | Fatigue | 25.5 | (2-144) |
| Sweating | 0 | (0) | Sweating | 18 | (18) |

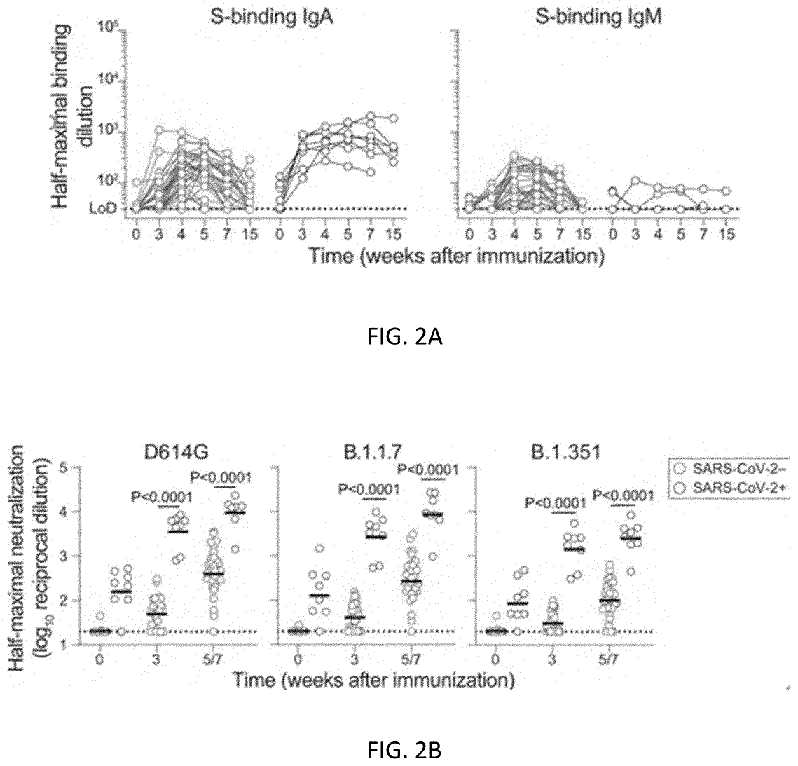

An enzyme-linked immune absorbent spot (ELISpot) assay was used to measure antibody-secreting plasmablasts in blood that bound SARS-CoV-2 S protein. SARS-CoV-2-S-specific IgG- and IgA-secreting plasmablasts were detected 3 weeks after primary immunization in 24 of 33 participants with no history of SARS-CoV-2 infection, but in 0 of 8 participants who had previously been infected with SARS-CoV-2. Plasmablasts peaked in blood during the first week after boosting (week 4 after primary immunization), with frequencies that varied widely from 3 to 4,100 S-binding plasmablasts per 106 peripheral blood mononuclear cells (PBMCs) (FIG. 1B and FIG. 1C). It was found that plasma IgG antibody titres against S, measured by enzyme-linked immunosorbent assay (ELISA), increased in all participants over time, and reached peak geometric mean half-maximal binding titres of 5,567 and 15,850 at 5 weeks after immunization among participants without and with history of SARS-CoV-2 infection, respectively, with a subsequent decline by 15 weeks after immunization. Anti-S IgA titres and IgG titres against the receptor-binding domain (RBD) of S showed similar kinetics, and reached peak geometric mean half-maximal binding titres of 172 and 739 for anti-S IgA and 4,501 and 7,965 for anti-RBD IgG among participants without and with history of SARS-CoV-2 infection, respectively, before declining. IgM responses were weaker and more transient, peaking 4 weeks after immunization among participants without history of SARS-CoV-2 infection with a geometric mean half-maximal binding titre of 78 and were undetectable in all but 2 previously infected participants (FIG. 1D, FIG. 2A).

The functional quality of serum antibody was measured using high-throughput focus reduction neutralization tests on Vero cells expressing TMPRSS2 against three authentic infectious SARS-CoV-2 strains with sequence variations in the S gene: (1) a Washington strain (2019n-CoV/USA) with a prevailing D614G substitution (WA1/2020 D614G); (2) a B.1.1.7 isolate with signature changes in the S gene, including mutations resulting in the deletion of residues 69, 70, 144 and 145 as well as N501Y, A570D, D614G and P681H substitutions; and (3) a chimeric SARS-CoV-2 with a B.1.351 S gene in the Washington strain background (Wash-B.1.351) that contained the following changes: D80A, deletion of residues 242-244, R246I, K417N, E484K, N501Y, D614G and A701V. Serum neutralizing titres increased markedly in participants without a history of SARS-CoV-2 infection after boosting, with geometric mean neutralization titres against WA1/2020 D614G of 58 at 3 weeks after primary immunization and 572 at 2 or 4 weeks after boost (5 or 7 weeks after primary immunization). Neutralizing titres against the B.1.1.7 and B.1.351 variants were lower, with geometric mean neutralization titres of 49 and 373 against B.1.1.7 and 36 and 137 against B.1.351 after primary and secondary immunization, respectively. In participants with a history of previous SARS-CoV-2 infection, neutralizing titres against all three viruses were detected at baseline (geometric mean neutralization titres of 241.8, 201.8 and 136.7 against WA1/2020 D614G, B.1.1.7 and B.1.351, respectively). In these participants, neutralizing titres increased more rapidly and to higher levels after immunization, with geometric mean neutralization titres of 4,544, 3,584 and 1,897 against WA1/2020 D614G, B.1.1.7 and B.1.351, respectively, after primary immunization, and 9,381, 9,351 and 2,749 against WA1/2020 D614G, B.1.1.7 and B.1.351, respectively, after secondary immunization. These geometric mean neutralization titres were 78-, 73- and 53-fold higher after primary immunization and 16-, 25- and 20-fold higher after boosting against WA1/2020 D614G, B.1.1.7 and B.1.351, respectively, than in participants without a history of SARS-CoV-2 infection (FIG. 2B).

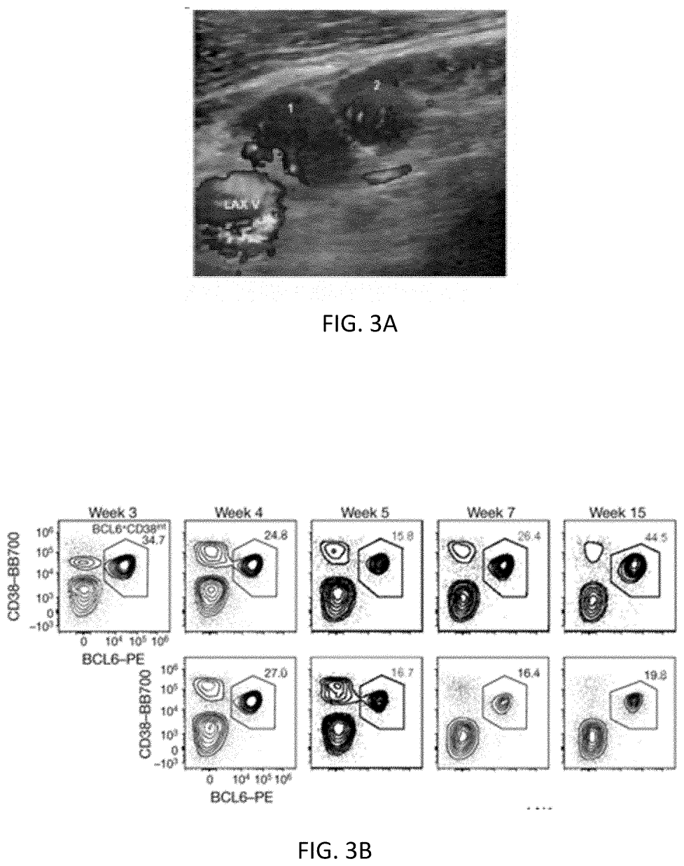

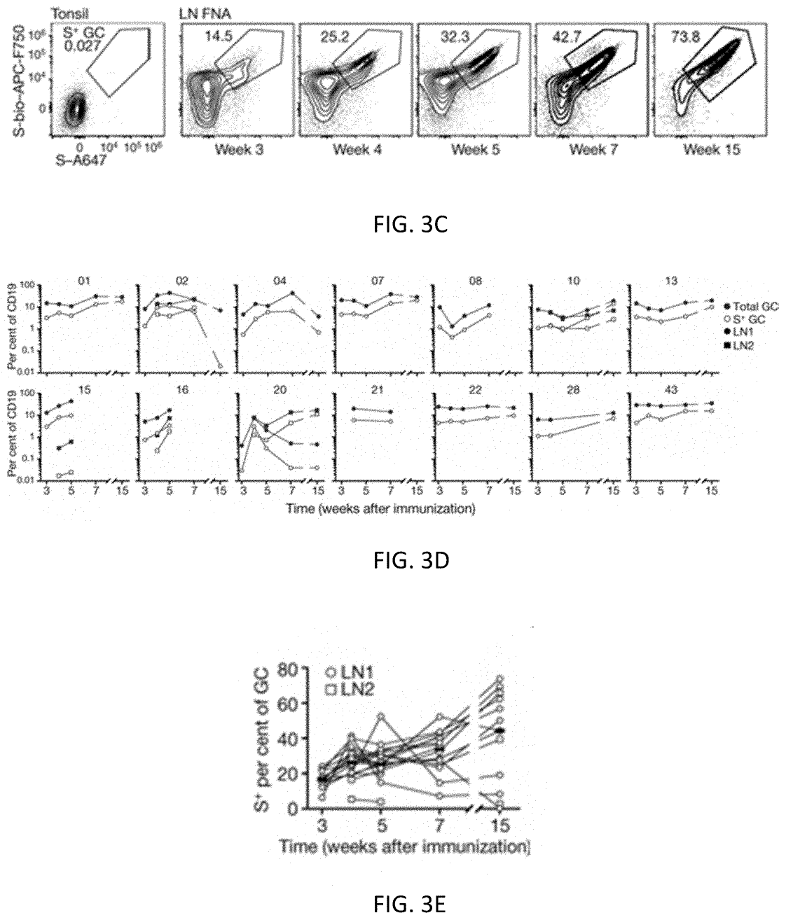

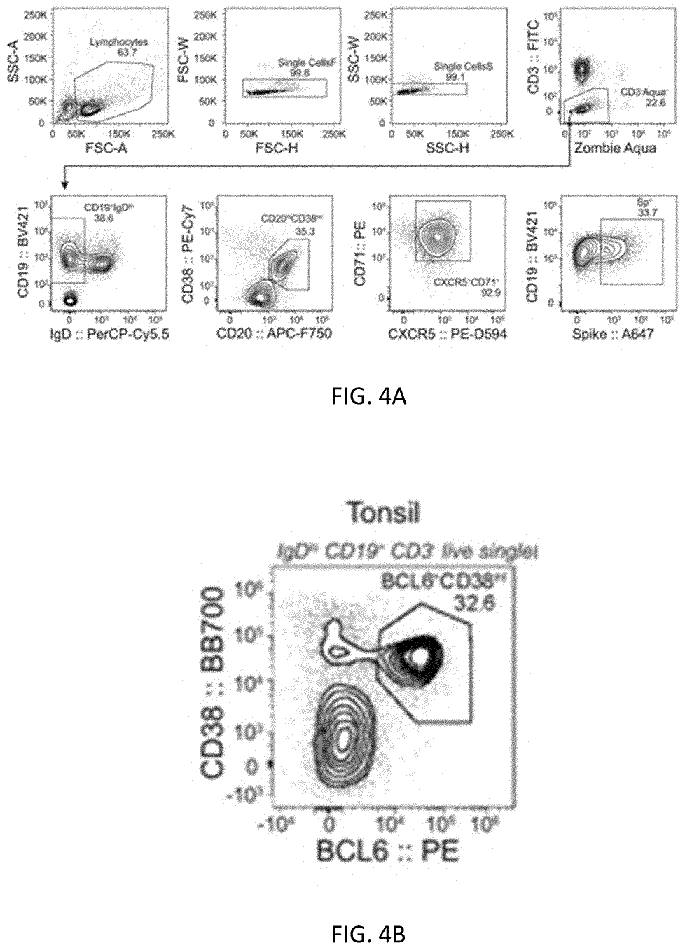

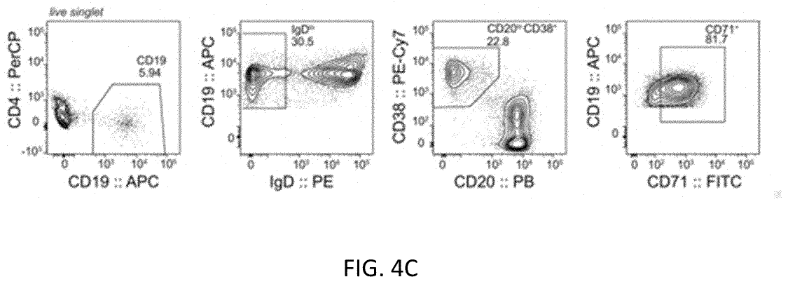

The BNT162b2 vaccine is injected into the deltoid muscle, which drains primarily to the lateral axillary lymph nodes. Ultrasonography was used to identify and guide FNA of accessible axillary nodes on the side of immunization approximately 3 weeks after primary immunization. In 5 of the 14 participants, a second draining lymph node was identified and sampled after secondary immunization (FIG. 3A). Germinal centre B cells (defined as CD19⁺CD3⁻IgD^{low}BCL6⁺CD38^{int} lymphocytes) were detected in all lymph nodes (FIG. 3B, FIG. 3D, FIG. 4A). FNA samples were co-stained with two fluorescently labelled S probes to detect S-binding germinal centre B cells. A control tonsillectomy sample with a high frequency of germinal centre B cells that was collected before the COVID-19 pandemic from an unrelated donor was stained as a negative control. S-binding germinal centre B cells were detected in FNAs from all 14 participants following primary immunization. The kinetics of the germinal centre response varied among participants, but S-binding germinal centre B cell frequencies increased at least transiently in all participants after boosting and persisted at high frequency in most individuals for at least 7 weeks. Notably, S-binding germinal centre B cells remained at or near their peak frequency 15 weeks after immunization in 8 of the 10 participants sampled at that time point, and these prolonged germinal centre responses had high proportions of S-binding cells (FIG. 3C-3E, FIG. 4B).

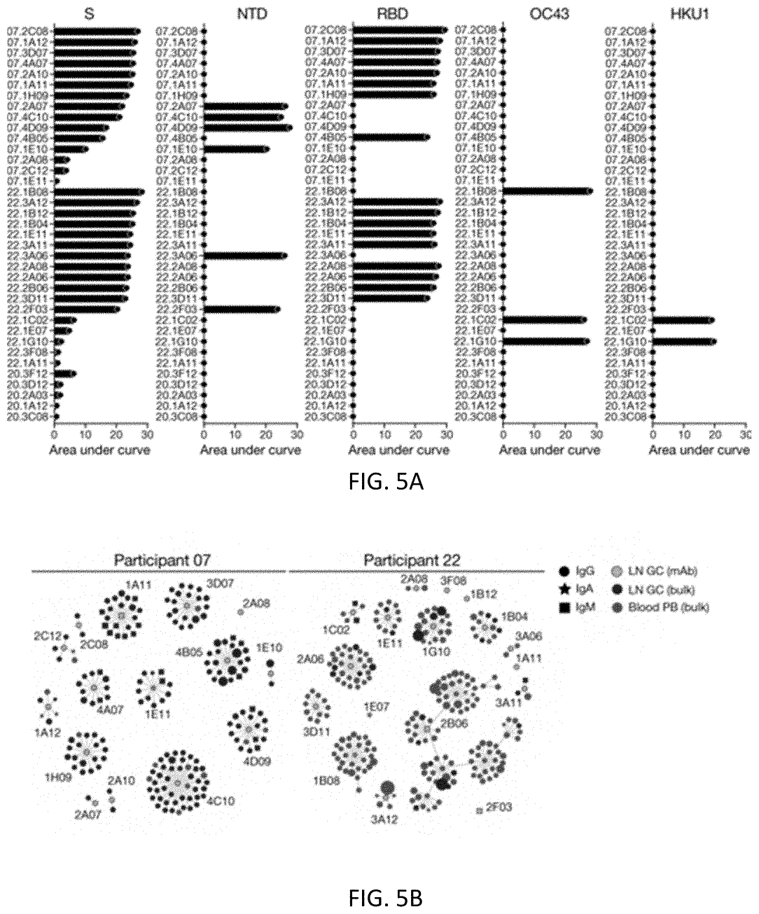

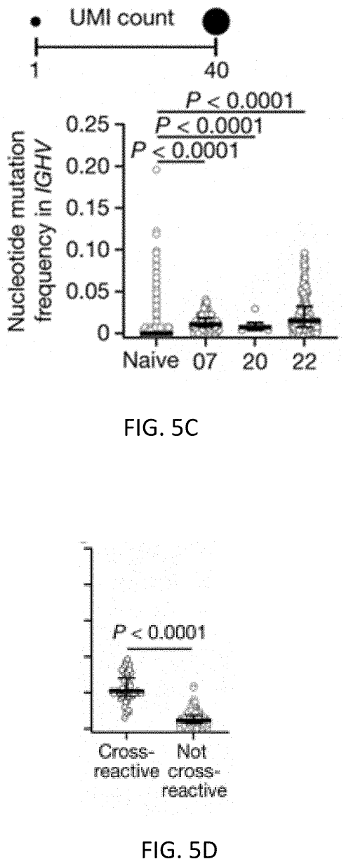

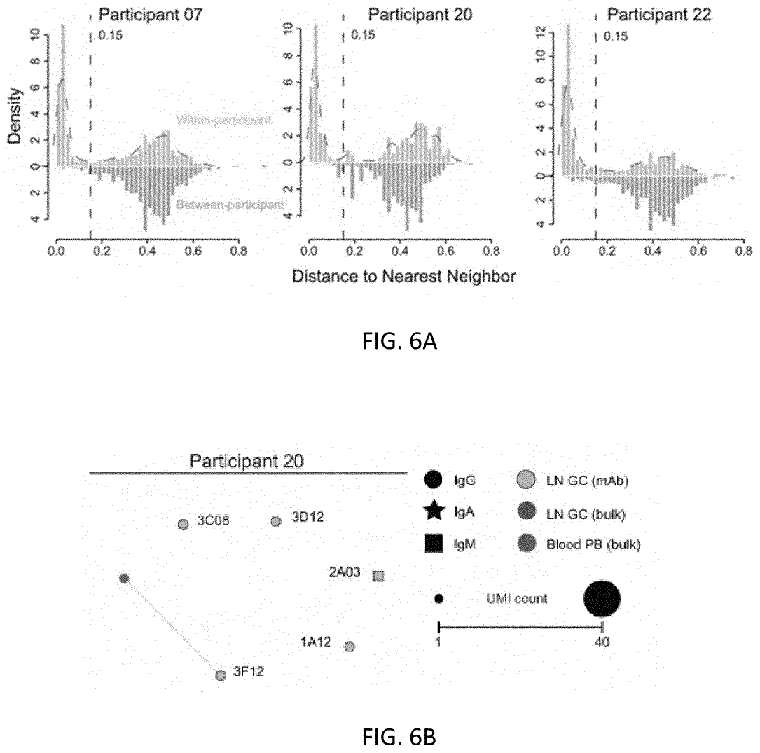

To evaluate the domains targeted by the S-protein-specific germinal centre response after vaccination, recombinant monoclonal antibodies were generated from single-cell-sorted S-binding germinal centre B cells (defined by the surface-marker phenotype CD19⁺CD3⁻IgD^{low}CD20^{high}CD38^{int}CD71⁺CXCR5⁺ lymphocytes) from three of the participants one week after boosting (FIG. 4A). Fifteen, five and seventeen S-binding, clonally distinct monoclonal antibodies were generated from participants 07, 20 (lymph node 1) and 22, respectively (Table 3). Of the 37 S-binding monoclonal antibodies, 17 bound the RBD, 6 recognized the N-terminal domain and 3 were cross-reactive with S proteins from seasonal betacoronavirus OC43; 2 of these monoclonal antibodies also bound S from seasonal betacoronavirus HKU1 (FIG. 5A). Clonal relatives of 14 out of 15, 1 out of 5 and 12 out of 17 of the S-binding monoclonal antibodies were identified among bulk-sorted total plasmablasts from PBMCs and germinal centre B cells at 4 weeks after immunization from participants 07, 20 and 22, respectively (FIG. 5B, FIG. 4C, FIG. 6A, FIG. 6B). Clones related to S-binding monoclonal antibodies had significantly increased mutation frequencies in their immunoglobulin heavy chain variable region (IGHV) genes compared to previously published naive B cells, particularly those related to monoclonal antibodies that cross-reacted with seasonal betacoronaviruses (FIG. 5C, FIG. 5D).

TABLE 3
Immunoglobulin gene usage of S-binding mAbs

| | | | | | | HCDR3 or |
|---|---|---|---|---|---|---|
| Chain | | Clone | Native | | | LCDR3 AA |
| Type | Name | Size | Isotype | Gene Usage | Mutations | Sequence |
| Heavy | 07.1A11 | 1/21 | IgM | VH3-15 DH1- | 4/283 = 0.0141 | SEQ ID NO: 172 |
| Chain | | | | 7 JH4 | | CTTGWFTGTYG |
| | | | | | | DYFDYW |
| | 07.1H09 | 1/21 | IgG1 | VH3-66 DH3- | 3/275 = 0.0109 | SEQ ID NO: 173 |
| | | | | 10 JH3 | | CARDFREGAFDI |
| | | | | | | W |
| | 07.2A08 | 1/21 | IgG1 | VH4-4 DH6- | 2/275 = 0.0073 | SEQ ID NO: 174 |
| | | | | 19 JH4 | | CATDGGWYTFD |
| | | | | | | HW |
| | 07.2A10 | 1/21 | IgG1 | VH4-31 DH3- | 1/278 = 0.0036 | SEQ ID NO: 175 |
| | | | | 16 JH3 | | CARYPVWGAFDI |
| | | | | | | W |
| | 07.2C08 | 1/21 | IgG1 | VH1-58 DH2- | 2/275 = 0.0073 | SEQ ID NO: 176 |
| | | | | 15 JH3 | | CAAAYCSGGSC |
| | | | | | | SDGFDIW |
| | 07.3D07 | 2/21 | IgG1 | VH3-30 DHS- | 3/277 = 0.0108 | SEQ ID NO: 177 |
| | | | | 18 JH4 | | CARVLWLRGMF |
| | | | | | | DYW |
| | 07.4A07 | 1/21 | IgG1 | VH3-30 DH3- | 3/277 = 0.0108 | SEQ ID NO: 178 |
| | | | | 10 JH4 | | CARGDYYGSGS |
| | | | | | | YPGKTFDYW |
| | 07.4B05 | 1/21 | IgG1 | VH1-69 DH1- | 1/277 = 0.0036 | SEQ ID NO: 179 |
| | | | | 26 JH5 | | CARGRLDSYSG |
| | | | | | | SYYSWFDPW |
| | 07.4D09 | 1/21 | IgG1 | VH4-4 DH2- | 1/274 = 0.0036 | SEQ ID NO: 180 |
| | | | | 15 JH4 | | CATKYCSGGSC |
| | | | | | | SYFGYW |
| | 20.1A12 | 23/46 | IgG1 | VH3-30 DH1- | 2/277 = 0.0072 | SEQ ID NO: 181 |
| | | | | 26 JH4 | | CAKGHSGSYRA |
| | | | | | | PFDYW |
| | 20.2A03 | 5/46 | IgM | VH3-33 DH3- | 1/278 = 0.0036 | SEQ ID NO: 182 |
| | | | | 10 JH4 | | CAREAYFGSGSS |
| | | | | | | PDYW |
| | 20.3C08 | 2/46 | IgG1 | VH3-7 DH3- | 1/278 = 0.0036 | SEQ ID NO: 183 |
| | | | | 22 JH4 | | CAREGTYYYDSS |
| | | | | | | AYYNGGLDYW |
| | 22.1A12 | 3/55 | IgG1 | VH3-30 DH2- | 4/274 = 0.0146 | SEQ ID NO: 184 |
| | | | | 15 JH4 | | CAKQGGGTYCG |
| | | | | | | GGSCYRGYFDY |
| | | | | | | W |
| | 22.1B08 | 1/55 | IgA1 | VH1-46 DH4- | 16/278 = 0.0576 | SEQ ID NO: 185 |
| | | | | 17 JH3 | | CARDPRVPAVTN |
| | | | | | | VNDAFDLW |
| | 22.1B12 | 1/55 | IgG1 | VH3-53 DH3- | 8/273 = 0.0293 | SEQ ID NO: 186 |
| | | | | 10 JH4 | | CARSHLEVRGVF |
| | | | | | | DNW |
| | 22.1E07 | 1/55 | IgA1 | VH3-33 DH4- | 3/278 = 0.0108 | SEQ ID NO: 187 |
| | | | | 17 JH4 | | CAREGVYGDIGG |
| | | | | | | AGLDYW |
| | 22.1E11 | 1/55 | IgG1 | VH3-30 DH2- | 2/274 = 0.0073 | SEQ ID NO: 188 |
| | | | | 15 JH4 | | CAKMGGVYCSA |
| | | | | | | GNCYSGRLEYW |
| | 22.1G10 | 2/55 | IgG1 | VH4-59 DH2- | 12/275 = 0.0436 | SEQ ID NO: 189 |
| | | | | 21 JHS | | CARETVNNWVD |
| | | | | | | PW |
| | 22.2A06 | 6/55 | IgG3 | VHS-51 DH3- | 1/277 = 0.0036 | SEQ ID NO: 190 |
| | | | | 3 JH4 | | CARREWGGSLG |
| | | | | | | HIDYW |
| | 22.2B06 | 2/55 | IgM | VH3-53 DH1- | 2/275 = 0.0073 | SEQ ID NO: 191 |
| | | | | 1 JH6 | | CARDLQLYGMD |
| | | | | | | VW |
| | 22.2F03 | 1/55 | IgM | VH1-18 DH6- | 1/277 = 0.0036 | SEQ ID NO: 192 |
| | | | | 13 JH6 | | CARVPGLVGYSS |
| | | | | | | SWYDNEKNYYY |
| | | | | | | YYYGMDVW |
| | 22.3A06 | 1/55 | IgG1 | VH3-23 DHS- | 2/277 = 0.0072 | SEQ ID NO: 193 |
| | | | | 18 JH5 | | CAKADTAMAWY |
| | | | | | | NWFDPW |
| | 22.3A11 | 1/55 | IgG1 | VH4-34 DH7- | 1/270 = 0.0037 | SEQ ID NO: 194 |
| | | | | 27 JH2 | | CARVWVRWWYF |
| | | | | | | DLW |
| Light | 07.1A11 | 1/21 | IgM | VK1-33 JK4 | 1/267 = 0.0037 | SEQ ID NO: 195 |
| Chain | | | | | | CQQYDNLPPTF |
| | 07.1H09 | 1/21 | IgG1 | VK1-9 JK4 | 0/266 = 0 | SEQ ID NO: 196 |
| | | | | | | CQQLNSYPPTF |
| | 07.2A08 | 1/21 | IgG1 | VL3-1 JL2 | 3/265 = 0.0113 | SEQ ID NO: 197 |
| | | | | | | CQAWGSSTVVF |
| | 07.2A10 | 1/21 | IgG1 | VK1-33 JK3 | 3/267 = 0.0112 | SEQ ID NO: 198 |
| | | | | | | CQHYDNLPPTF |
| | 07.2C08 | 1/21 | IgG1 | VK3-20 JK1 | 5/266 = 0.0188 | SEQ ID NO: 199 |
| | | | | | | CQQYGSSPWTF |
| | 07.3D07 | 2/21 | IgG1 | VL6-57 JL3 | 2/278 = 0.0072 | SEQ ID NO: 200 |
| | | | | | | CQSYDISNHWVF |
| | 07.4A07 | 1/21 | IgG1 | VK1-33 JK4 | 1/266 = 0.0038 | SEQ ID NO: 201 |
| | | | | | | CQQYDNLPLTF |
| | 07.4B05 | 1/21 | IgG1 | VK4-1 JK2 | 2/283 = 0.0071 | SEQ ID NO: 202 |
| | | | | | | CQQYYSTPYTF |
| | 07.4D09 | 1/21 | IgG1 | VL2-23 JL3 | 0/277 = 0 | SEQ ID NO: 203 |
| | | | | | | CCSYAGSSTWV |
| | | | | | | F |
| | 20.1A12 | 23/46 | IgG1 | VK3-20 JK2 | 0/263 = 0 | SEQ ID NO: 204 |
| | | | | | | CQQYGSSYTF |
| | 20.2A03 | 5/46 | IgM | VL3-10 JL2 | 2/272 = 0.0074 | SEQ ID NO: 205 |
| | | | | | | CYSTDSSDNHR |
| | | | | | | RVF |
| | 20.3C08 | 2/46 | IgG1 | VL3-10 JL1 | 1/272 = 0.0037 | SEQ ID NO: 206 |
| | | | | | | CYSTDSSGNHR |
| | | | | | | RLF |
| | 22.1A12 | 3/55 | IgG1 | VK1-33 JK4 | 2/264 = 0.0076 | SEQ ID NO: 207 |
| | | | | | | CQQYDNIPLTF |
| | 22.1B08 | 1/55 | IgA1 | VK3-11 JK2 | 5/267 = 0.0187 | SEQ ID NO: 162 |
| | | | | | | CQQRSNRPPRW |
| | | | | | | TF |
| | 22.1B12 | 1/55 | IgG1 | VK4-1 JK2 | 1/282 = 0.0035 | SEQ IN NO: 163 |
| | | | | | | CQQYYSTPCSF |
| | 22.1E07 | 1/55 | IgA1 | VL3-10 JL1 | 4/272 = 0.0147 | SEQ ID NO: 164 |
| | | | | | | CYSTDSSVNGRV |
| | | | | | | F |
| | 22.1E11 | 1/55 | IgG1 | VK1-33 JK3 | 0/263 = 0 | SEQ ID NO: 165 |
| | | | | | | CQQYDNLLTF |
| | 22.1G10 | 2/55 | IgG1 | VK4-1 JK1 | 10/282 = 0.0355 | SEQ ID NO: 167 |
| | | | | | | CQQYFTTPWTF |
| | 22.2A06 | 6/55 | IgG3 | VL6-57 JL2 | 4/276 = 0.0145 | SEQ ID NO: 167 |
| | | | | | | CQSFDSSNVVF |
| | 22.2B06 | 2/55 | IgM | VL3-21 JL2 | 2/268 = 0.0075 | SEQ ID NO: 168 |
| | | | | | | CQVWDSSSDPV |
| | | | | | | VF |
| | 22.2F03 | 1/55 | IgM | VL3-25 JL1 | 1/270 = 0.0037 | SEQ ID NO: 169 |
| | | | | | | CQSADSSGTYVF |
| | 22.3A06 | 1/55 | IgG1 | VK3-11 JK4 | 3/264 = 0.0114 | SEQ ID NO: 170 |
| | | | | | | CQHRSNWPLTF |
| | 22.3A11 | 1/55 | IgG1 | VL3-21 JL1 | 4/272 = 0.0147 | SEQ ID NO: 171 |
| | | | | | | CQVWDNSSDQP |
| | | | | | | NYVF |

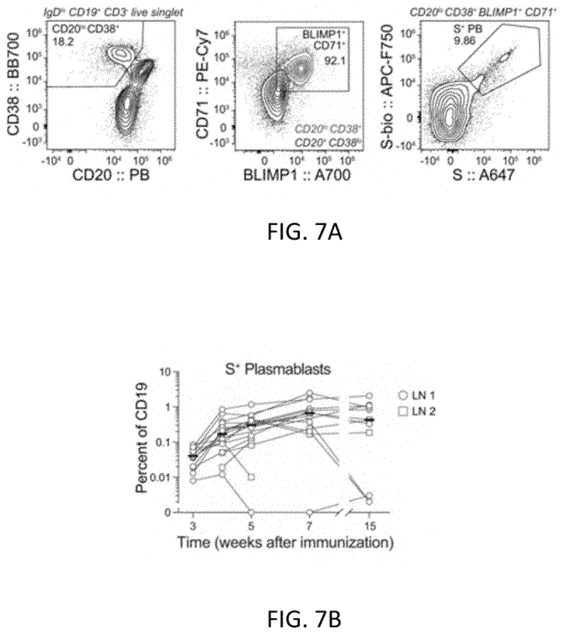

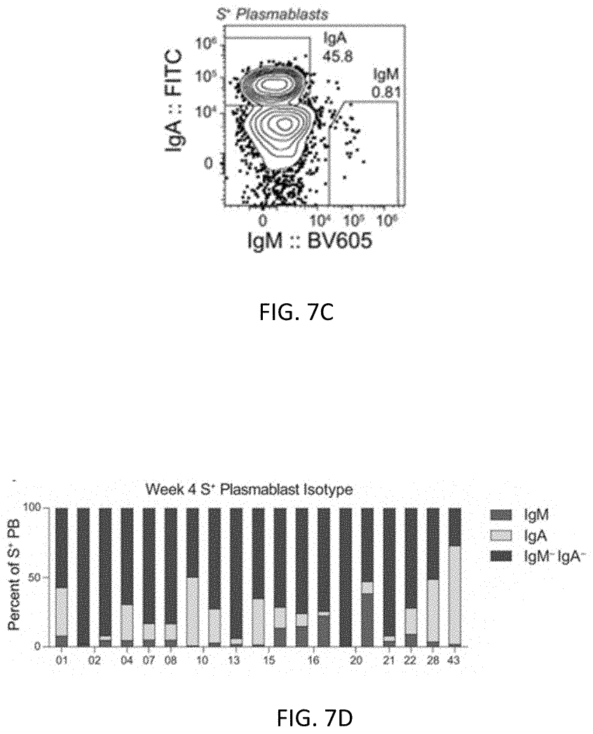

In addition to germinal centre B cells, robust plasmablast responses were detected in the draining lymph nodes of all 14 participants in the FNA cohort. S-binding plasmablasts (defined as CD19⁺CD3⁻IgD^{low}CD20^{low}CD38⁺CD71⁺BLIMP1⁺ lymphocytes) were detected in all of the lymph nodes that were sampled, and increased in frequency after boosting (FIG. 7A, FIG. 7B). The detected plasmablasts were unlikely to be a contaminant of blood, because CD14+ monocyte and/or granulocyte frequencies were below 1% in all FNA samples (well below the 10% threshold that was previously established). Moreover, S-binding plasmablasts were detected in FNA samples at 5, 7 and 15 weeks after immunization, when they had become undetectable in blood from all participants in the cohort. The vast majority of S-binding lymph node plasmablasts were isotype-switched at 4 weeks after primary immunization, and IgA-switched cells accounted for 25% or more of the plasmablasts in 6 out of 14 participants (FIG. 7C, FIG. 7D).

This example evaluated whether SARS-CoV-2 mRNA-based vaccines induce antigen-specific plasmablast and germinal centre B cell responses in humans. The vaccine induced a strong IgG-dominated plasmablast response in blood that peaked one week after the booster immunization. In the draining lymph nodes, robust SARS-CoV-2 S-binding germinal centre B cell and plasmablast responses were detected in aspirates from all 14 of the participants. These responses were detectable after the first immunization but greatly expanded after the booster injection. Notably, S-binding germinal centre B cells and plasmablasts persisted for at least 15 weeks after the first immunization (12 weeks after secondary immunization) in 8 of the 10 participants who were sampled at that time point. These responses to mRNA vaccination are superior to those seen after seasonal influenza virus vaccination in humans, in whom haemagluttinin-binding germinal centre B cells were detected in only three out of eight participants. More robust germinal centre responses are consistent with antigen dissemination to multiple lymph nodes and the self-adjuvating characteristics of the mRNA-lipid nanoparticle vaccine platform compared to nonadjuvanted inactivated vaccines used for seasonal influenza virus vaccination. These data in humans corroborate reports that demonstrate the induction of potent germinal centre responses by SARS-CoV-2 mRNA-based vaccines in mice.

It is believed that this is the first study to provide direct evidence for the induction of a persistent antigen-specific germinal centre B cell response after vaccination in humans. Dynamics of germinal centre B cell responses vary widely depending on the model system in which they are studied, although the most active period of the response usually occurs over the course of a few weeks. Primary alum-adjuvanted protein immunization of mice typically leads to germinal centre responses that peak 1-2 weeks after immunization and contract at least 10-fold within 5-7 weeks. Germinal centre responses induced by immunization with more robust adjuvants such as sheep red blood cells, complete Freund's adjuvant or saponin-based adjuvants tend to peak slightly later, at 2-4 weeks after vaccination, and can persist at low frequencies for several months. Although studies of extended durability are rare, antigen-specific germinal centre B cells have been found to persist for at least one year, albeit at very low levels. In this example, it is shown SARS-CoV-2 mRNA vaccine-induced germinal centre B cells are maintained at or near peak frequencies for at least 12 weeks after secondary immunization.

A preliminary observation from this example is the dominance of RBD-targeting clones among responding germinal centre B cells. A more detailed analysis of these RBD-binding monoclonal antibodies assessed their in vitro inhibitory capacity against the WA1/2020 D614G strain using an authentic SARS-CoV-2 neutralization assay: five showed high neutralization potency, with 80% neutralization values of less than 100 ng ml-1. For the most part, RBD-binding clones contained few (<3) nonsynonymous nucleotide substitutions in their IGHV genes, which indicates that they originated from recently engaged naive B cells. This contrasts with the three cross-reactive germinal centre B cell clones that recognized conserved epitopes within the S proteins of betacoronaviruses. These cross-reactive clones had significantly higher mutation frequencies, which suggests a memory B cell origin. These data are consistent with previous findings from seasonal influenza virus vaccination in humans that show that the germinal centre reaction can engage pre-existing memory B cells directed against conserved epitopes as well as naive clones targeting novel epitopes. However, these cross-reactive clones were not identified in all individuals and comprised a small fraction of responding B cells, consistent with a similar analysis of SARS-CoV-2 mRNA vaccine-induced plasmablasts. Overall, these data demonstrate the capacity of SARS-CoV-2 mRNA-based vaccines to induce robust and prolonged germinal centre reactions. The induced germinal centre reaction recruited cross-reactive memory B cells as well as newly engaged clones that target unique epitopes within SARS-CoV-2 S protein. Elicitation of high affinity and durable protective antibody responses is a hallmark of a successful humoral immune response to vaccination. By inducing robust germinal centre reactions, SARS-CoV-2 mRNA-based vaccines are on track for achieving this outcome.

### Methods

Sample collection, preparation, and storage: All studies were approved by the Institutional Review Board of Washington University in St Louis. Written consent was obtained from all participants. Forty-one healthy volunteers were enrolled, of whom 14 provided axillary lymph node samples (Table 1). In 5 of the 14 participants, a second draining lymph node was identified and sampled following secondary immunization. One participant (15) received the second immunization in the contralateral arm; draining lymph nodes were identified and sampled on both sides. Blood samples were collected in EDTA tubes, and PBMCs were enriched by density gradient centrifugation over Ficoll 1077 (GE) or Lymphopure (BioLegend). The residual red blood cells were lysed with ammonium chloride lysis buffer, and cells were immediately used or cryopreserved in 10% dimethylsulfoxide in fetal bovine serum (FBS). Ultrasound-guided FNA of axillary lymph nodes was performed by a radiologist or a qualified physician's assistant under the supervision of a radiologist. Lymph node dimensions and cortical thickness were measured, and the presence and degree of cortical vascularity and location of the lymph node relative to the axillary vein were determined before each FNA. For each FNA sample, six passes were made under continuous real-time ultrasound guidance using 25-gauge needles, each of which was flushed with 3 ml of RPMI 1640 supplemented with 10% FBS and 100 U ml-1 penicillin-streptomycin, followed by three 1-ml rinses. Red blood cells were lysed with ammonium chloride buffer (Lonza), washed with phosphate-buffered saline (PBS) supplemented with 2% FBS and 2 mM EDTA, and immediately used or cryopreserved in 10% dimethylsulfoxide in FBS. Participants reported no adverse effects from phlebotomies or serial FNAs.

Cell lines: Expi293F cells were cultured in Expi293 Expression Medium (Gibco). Vero E6 (CRL-1586, American Type Culture Collection), Vero cells expressing TMPRSS2 (Vero-TMPRSS2 cells) (a gift from S. Ding), and Vero cells expressing human ACE2 and TMPRSS2 (Vero-hACE2-TMPRSS2) (a gift of A. Creanga and B. Graham) cells were cultured at 37° C. in Dulbecco's modified Eagle medium (DMEM) supplemented with 10% FBS, 10 mM HEPES (pH 7.3), 1 mM sodium pyruvate, 1× nonessential amino acids and 100 U ml⁻¹ of penicillin-streptomycin. Vero-TMPRSS2 cell cultures were supplemented with 5 μg ml⁻¹ of blasticidin. Vero-hACE2-TMPRSS2 cell cultures were supplemented with 10 μg ml⁻¹ of puromycin.

Viruses: The 2019n-CoV/USA_WA1/2020 isolate of SARS-CoV-2 was obtained from the US Centers for Disease Control. The B.1.1.7 isolate from the UK was obtained from an infected individual. The point mutation D614G in the S gene was introduced into an infectious complementary DNA clone of the 2019n-CoV/USA_WA1/2020 strain as previously described. Nucleotide substitutions were introduced into a subclone puc57-CoV-2-F5-7 containing the S gene of the SARS-CoV-2 wild-type infectious clone. The S gene of the B.1.351 variant (first identified in South Africa) was produced synthetically by Gibson assembly. The full-length infectious cDNA clones of the variant SARS-CoV-2 viruses were assembled by in vitro ligation of seven contiguous cDNA fragments following a previously described protocol. In vitro transcription was then performed to synthesize full-length genomic RNA. To recover the mutant viruses, the RNA transcripts were electroporated into Vero E6 cells. The viruses from the supernatant of cells were collected 40 h later and served as p0 stocks. All viruses were passaged once in Vero-hACE2-TMPRSS2 cells and subjected to deep sequencing after RNA extraction to confirm the introduction and stability of substitutions. All virus preparation and experiments were performed in an approved biosafety level 3 facility.

Antigens: Recombinant soluble SARS-CoV-2 S protein, recombinant RBD of S, human coronavirus OC43 S, and human coronavirus HKU1 S were expressed as previously described. In brief, mammalian cell codon-optimized nucleotide sequences coding for the soluble ectodomain of the S protein of SARS-CoV-2 (GenBank: MN908947.3, amino acids 1-1213) including a C-terminal thrombin cleavage site, T4 foldon trimerization domain and hexahistidine tag, and for the RBD (amino acids 319-541) along with the signal peptide (amino acids 1-14) plus a hexahistidine tag were cloned into mammalian expression vector pCAGGS. The S protein sequence was modified to remove the polybasic cleavage site (RRAR to A), and two pre-fusion stabilizing proline mutations were introduced (K986P and V987P, wild-type numbering). Expression plasmids encoding for the S of common human coronaviruses OC43 and HKU1 were provided by B. Graham. Recombinant proteins were produced in Expi293F cells (ThermoFisher) by transfection with purified DNA using the ExpiFectamine 293 Transfection Kit (ThermoFisher). Supernatants from transfected cells were collected 3 days after transfection, and recombinant proteins were purified using Ni-NTA agarose (ThermoFisher), then buffer-exchanged into PBS and concentrated using Amicon Ultracel centrifugal filters (EMD Millipore). For flow cytometry staining, recombinant S was labelled with Alexa Fluor 647-NHS ester or biotinylated using the EZ-Link Micro NHS-PEG4-Biotinylation Kit (Thermo Fisher); excess Alexa Fluor 647 and biotin were removed using 7-kDa Zeba desalting columns (Pierce).

EL/Spot assay: Plates were coated with Flucelvax Quadrivalent 2019/2020 seasonal influenza virus vaccine (Sequiris), S or RBD. A direct ex vivo ELISpot assay was performed to determine the number of total, vaccine-binding or recombinant S-binding IgG- and IgA-secreting cells present in PBMC samples using IgG/IgA double-colour ELISpot Kits (Cellular Technology) according to the manufacturer's instructions. ELISpot plates were analysed using an ELISpot counter (Cellular Technology).

ELISAs: Assays were performed in 96-well plates (MaxiSorp; Thermo) coated with 100 μl of recombinant S, RBD, N-terminal domain of S (SinoBiological), OC43 S, HKU1 S or bovine serum albumin diluted to 1 μg ml⁻¹ in PBS, and plates were incubated at 4° C. overnight. Plates then were blocked with 10% FBS and 0.05% Tween 20 in PBS. Plasma or purified monoclonal antibodies were serially diluted in blocking buffer and added to the plates. Plates were incubated for 90 min at room temperature and then washed 3 times with 0.05% Tween 20 in PBS. Goat anti-human IgG-HRP (goat polyclonal, Jackson ImmunoResearch, 1:2,500), IgA (goat polyclonal, Jackson ImmuoResearch, 1:2,500) or IgM (goat polyclonal, Caltag, 1:4,000) were diluted in blocking buffer before adding to wells and incubating for 60 min at room temperature. Plates were washed 3 times with 0.05% Tween 20 in PBS and 3 times with PBS before the addition of o-phenylenediamine dihydrochloride peroxidase substrate (Sigma-Aldrich). Reactions were stopped by the addition of 1 M hydrochloric acid. Optical density measurements were taken at 490 nm. The area under the curve for each monoclonal antibody and half-maximal binding dilution for each plasma sample were calculated using Graphpad Prism v.8.

Focus reduction neutralization test: Plasma samples were declotted by diluting 1:10 in DMEM supplemented with 2% FBS, 10 mM HEPES and 100 U ml⁻¹ penicillin-streptomycin and incubating for 3 h at 37° C. Serial dilutions of resulting serum were incubated with 10² focus-forming units of different strains or variants of SARS-CoV-2 for 1 h at 37° C. Antibody-virus complexes were added to Vero-TMPRSS2 cell monolayers in 96-well plates and incubated at 37° C. for 1 h. Subsequently, cells were overlaid with 1% (w/v) methylcellulose in MEM supplemented with 2% FBS. Plates were collected 30 h later by removing overlays and fixed with 4% PFA in PBS for 20 min at room temperature. Plates were washed and sequentially incubated with an oligoclonal pool of mouse anti-S monoclonal antibodies (SARS2-2, SARS2-11, SARS2-16, SARS2-31, SARS2-38, SARS2-57 and SARS2-71) and HRP-conjugated goat anti-mouse IgG (polyclonal, Sigma, 1:500) in PBS supplemented with 0.1% saponin and 0.1% bovine serum albumin. SARS-CoV-2-infected cell foci were visualized using TrueBlue peroxidase substrate (KPL) and quantified on an ImmunoSpot microanalyser (Cellular Technology).

Flow cytometry and cell sorting: Staining for flow cytometry analysis and sorting was performed using freshly isolated or cryo-preserved FNA, PBMC or tonsil samples. For analysis, cells were incubated for 30 min on ice with biotinylated and Alexa Fluor 647 conjugated recombinant soluble S and PD-1-BB515 (EH12.1, BD Horizon, 1:100) in 2% FBS and 2 mM EDTA in PBS (P2), washed twice, then stained for 30 min on ice with IgG-BV480 (goat polyclonal, Jackson ImmunoResearch, 1:100), IgA-FITC (M24A, Millipore, 1:500), CD45-A532 (H130, Thermo, 1:50), CD38-BB700 (HIT2, BD Horizon, 1:500), CD20-Pacific Blue (2H7, 1:400), CD27-BV510 (0323, 1:50), CD8-BV570 (RPA-T8, 1:200), IgM-BV605 (MHM-88, 1:100), HLA-DR-BV650 (L243, 1:100), CD19-BV750 (HIB19, 1:100), CXCR5-PE-Dazzle 594 (J252D4, 1:50), IgD-PE-Cy5 (IA6-2, 1:200), CD14-PerCP (HCD14, 1:50), CD71-PE-Cy7 (CY1G4, 1:400), CD4-Spark685 (SK3, 1:200), streptavidin-APC-Fire750, CD3-APC-Fire810 (SK7, 1:50) and Zombie NIR (all BioLegend) diluted in Brilliant Staining buffer (BD Horizon). Cells were washed twice with P2, fixed for 1 h at 25° C. using the True Nuclear fixation kit (BioLegend), washed twice with True Nuclear Permeabilization/Wash buffer, stained with FOXP3-BV421 (206D, BioLegend, 1:15), Ki-67-BV711 (Ki-67, BioLegend, 1:200), Tbet-BV785 (4B10, BioLegend, 1:400), BCL6-PE (K112-91, BD Pharmingen, 1:25), and BLIMP1-A700 (646702, R&D, 1:50) for 1 h at 25° C., washed twice with True Nuclear Permeabilization/Wash buffer, and acquired on an Aurora using SpectroFlo v.2.2 (Cytek). Flow cytometry data were analysed using FlowJo v.10 (Treestar).

For sorting germinal centre B cells, FNA single-cell suspensions were stained for 30 min on ice with CD19-BV421 (HIB19, 1:100), CD3-FITC (HIT3a, 1:200), IgD-PerCP-Cy5.5 (IA6-2, 1:200), CD71-PE (CY1G4, 1:400), CXCR5-PE-Dazzle 594 (J252D4, 1:50), CD38-PE-Cy7 (HIT2, 1:200), CD20-APC-Fire750 (2H7, 1:100), Zombie Aqua (all BioLegend), and Alexa Fluor 647 conjugated recombinant soluble S. For sorting plasmablasts, PBMCs were stained for 30 min on ice with CD20-PB (2H7, 1:400), CD71-FITC (CY1G4, 1:200), CD4-PerCP (OKT4, 1:100), IgD-PE (IA6-2, 1:200), CD38-PE-Cy7 (HIT2, 1:200), CD19-APC (HIB19, 1:200) and Zombie Aqua (all BioLegend). Cells were washed twice, and single S-binding germinal centre B cells (live singlet CD3⁻CD19⁺IgD^{low}CD20^{high}CD38^{int}CD71⁺CXCR5⁺S⁺) were sorted using a FACSAria II into 96-well plates containing 2 μl Lysis Buffer (Clontech) supplemented with 1 U μl⁻¹ RNase inhibitor (NEB), or total germinal centre B cells or plasmablasts (live singlet CD3⁻CD19⁺IgD^{low}CD20^{low}CD38⁺CD71⁺) were bulk-sorted into buffer RLT Plus (Qiagen) and immediately frozen on dry ice.

Monoclonal antibody generation: Antibodies were cloned as previously described. In brief, VH, Vκ and Vλ genes were amplified by reverse transcription PCR and nested PCR reactions from singly sorted germinal centre B cells using primer combinations specific for IgG, IgM, IgA, Igκ and Igλ from previously described primer sets45, and then sequenced. To generate recombinant antibodies, restriction sites were incorporated via PCR with primers to the corresponding heavy and light chain V and J genes. The amplified VH, Vκ and Vλ genes were cloned into IgG1 and Igκ or Igλ expression vectors, respectively, as previously described. Heavy and light chain plasmids were co-transfected into Expi293F cells (Gibco) for expression, and antibody was purified using protein A agarose chromatography (Goldbio). Sequences were obtained from PCR reaction products and annotated using the ImMunoGeneTics (IMGT)/V-QUEST database (imgt.org/IMGT_vquest/). Mutation frequency was calculated by counting the number of nonsynonymous nucleotide mismatches from the germline sequence in the heavy chain variable segment leading up to the CDR3, while excluding the 5′ primer sequences that could be error-prone.

Bulk B cell receptor sequencing: RNA was purified from sorted plasmablasts from PBMCs and germinal centre B cells from lymph nodes from participants 07, 20 (lymph node 1) and 22 using the RNeasy Plus Micro kit (Qiagen). Reverse transcription, unique molecular identifier (UMI) barcoding, cDNA amplification, and Illumina linker addition to B cell heavy chain transcripts were performed using the human NEBNext Immune Sequencing Kit (New England Biolabs) according to the manufacturer's instructions. High-throughput 2×300-bp paired-end sequencing was performed on the Illumina MiSeq platform with a 30% PhiX spike-in according to manufacturer's recommendations, except for performing 325 cycles for read 1 and 275 cycles for read 2.

Processing of B cell receptor bulk-sequencing reads: Demultiplexed pair-end reads were BLAST'ed using blastn v.2.11.0 for PhiX removal and subsequently preprocessed using pRESTO v.0.6.2 as follows. (1) Reads with a mean Phred quality score below 20 were filtered. (2) Reads were aligned against template switch sequences and constant region primers (Extended Data Table 5), with a maximum mismatch rate of 0.5 and 0.2 respectively. (3) A UMI was assigned to each read by extracting the first 17 nucleotides preceding the template switch site. (4) Sequencing and multiplexing errors in the UMI region were then corrected using a previously published approach. In brief, reads with similar UMIs were clustered using cd-hit-est v.4.8.1 on the basis of the pairwise distance of their UMIs with a similarity threshold of 0.83 that was estimated from 10,000 reads. The UMI-based read groups were further clustered within themselves on the basis of the pairwise distance of the non-UMI region of their reads with a similarity threshold of 0.8. Read clusters spanning multiple multiplexed samples were assigned to the majority sample. (5) Separate consensus sequences for the forward and reverse reads within each read cluster were constructed with a maximum error score of 0.1 and minimum constant region primer frequency of 0.6. If multiple constant region primers were associated with a particular read cluster, the majority primer was used. (6) Forward and reverse consensus sequence pairs were assembled by first attempting de novo assembly with a minimum overlap of 8 nucleotides and a maximum mismatch rate of 0.3. If unsuccessful, this was followed by reference-guided assembly using blastn v.2.11.0 with a minimum identity of 0.5 and an E-value threshold of 1×10−5. (7) Isotypes were assigned by local alignment of the 3′ end of each consensus sequence to isotype-specific internal constant region sequences with a maximum mismatch rate of 0.3. Sequences with inconsistent isotype assignment and constant region primer alignment were removed. (8) Duplicate consensus sequences, except those with different isotype assignments, were collapsed into unique sequences. Only unique consensus sequences with at least two contributing reads were used subsequently.

B cell receptor genotyping: Initial germline V(D)J gene annotation was performed using IgBLAST v.1.17.1 with IMGT/GENE-DB release 202113-2. IgBLAST output was parsed using Change-O v.1.0.2. Quality control was performed, requiring each sequence to have non-empty V and J gene annotations; exhibit chain consistency in all annotations; bear fewer than 10 non-informative (non-A/T/G/C, such as N or −) positions; and carry a CDR3 with no N and a nucleotide length that is a multiple of 3. Individualized genotypes were inferred using TIgGER v.1.0.0 and used to finalize V(D)J annotations. Sequences annotated as non-productively rearranged by IgBLAST were removed from further analysis.

Clonal lineage analysis: B cell clonal lineages were inferred on the basis of productively rearranged heavy chain sequences using hierarchical clustering with single linkage. Sequences were first partitioned based on common V and J gene annotations and CDR3 lengths. Within each partition, sequences with CDR3s that were within 0.15 normalized Hamming distance from each other were clustered as clones. This distance threshold was determined by manual inspection in conjunction with kernel density estimates to identify the local minimum between the two modes of the within-participant bimodal distance-to-nearest distribution. Following clonal clustering, full-length clonal consensus germline sequences were reconstructed for each clone with D-segment and N/P regions masked with Ns, resolving any ambiguous gene assignments by majority rule. Within each clone, duplicate IMGT-aligned V(D)J sequences from bulk sequencing were collapsed with the exception of duplicates derived from different B cell compartments or isotypes. Clones were visualized as networks using igraph v.1.2.5. First, a full network was calculated for each clone, in which an edge was drawn between every pair of sequences with CDR3s that were within 0.15 normalized Hamming distance from each other. Then, a minimum spanning tree was derived from the full network, in which only edges essential for ensuring that all sequences connected in the full network remain connected in the minimum spanning tree either directly or indirectly were retained. The minimum spanning tree was then visualized for each clone.

Calculation of somatic hypermutation frequency: Mutation frequency was calculated by counting the number of nucleotide mismatches from the germline sequence in the observed heavy chain variable segment leading up to the CDR3, while excluding the first 18 positions that could be error-prone owing to the primers used for generating the monoclonal antibody sequences. Calculation was performed using the calcObservedMutations function from SHazaM v.1.0.2.

### Example 2—a Public Vaccine-Induced Human Antibody Protects Against SARS-CoV-2 and Emerging Variants

The emergence of antigenically distinct severe acute respiratory syndrome coronavirus 2 (SARS-CoV-2) variants with increased transmissibility is a public health threat. Some of these variants show substantial resistance to neutralization by SARS-CoV-2 infection- or vaccination-induced antibodies, which principally target the receptor binding domain (RBD) on the virus spike glycoprotein. The present example describes 2C08, a SARS-CoV-2 mRNA vaccine-induced germinal center B cell-derived human monoclonal antibody that binds to the receptor binding motif within the RBD. 2C08 broadly neutralizes SARS-CoV-2 variants with remarkable potency and reduces lung inflammation, viral load, and morbidity in hamsters challenged with either an ancestral SARS-CoV-2 strain or a recent variant of concern. Clonal analysis identified 2C08-like public clonotypes among B cell clones responding to SARS-CoV-2 infection or vaccination in at least 20 out of 78 individuals. Thus, 2C08-like antibodies can be readily induced by SARS-CoV-2 vaccines and mitigate resistance by circulating variants of concern.

SARS-CoV-2 is a highly pathogenic coronavirus that first emerged in Wuhan, Hubei province of China in late 2019. The virus quickly spread to multiple continents, leading to the coronavirus disease 2019 (COVID-19) pandemic. To date, SARS-CoV-2 has caused more than 120 million confirmed infections, leading to approximately three million deaths. The damaging impact of the morbidity and mortality caused by the COVID-19 pandemic has triggered a global effort towards developing SARS-CoV-2 countermeasures. These campaigns led to the rapid development and deployment of antibody-based therapeutics (immune plasma therapy, monoclonal antibodies (mAbs)) and vaccines (lipid nanoparticle-encapsulated mRNA, virus-inactivated, and viral-vectored platforms). The high efficacy of mRNA-based vaccines in particular has raised hope for ending the pandemic. However, the emergence of multiple SARS-CoV-2 variants that are antigenically distinct from the early circulating strains used to develop the first generation of vaccines has raised concerns for compromised vaccine-induced protective immunity. Indeed, multiple studies have demonstrated that these variants show reduced neutralization in vitro by antibodies elicited in humans in response to SARS-CoV-2 infection or vaccination. This observation highlights the need for better understanding of the breadth of SARS-CoV-2 vaccine-induced antibody responses and possible adjustments of prophylactic and therapeutic reagents to combat emerging variants.

SARS-CoV-2 entry into host cells is mediated primarily by the binding of the viral spike (S) protein through its receptor-binding domain (RBD) to the cellular receptor, human angiotensin-converting enzyme 2 (ACE2). Thus, the S protein is a critical target for antibody-based therapeutics to prevent SARS-CoV-2 virus infection and limit its spread. Indeed, the RBD is recognized by many potently neutralizing monoclonal antibodies. Pfizer-BioNTech SARS-CoV-2 mRNA vaccine (BNT162b2) encodes the full-length prefusion stabilized SARS-CoV-2 S protein and induces robust serum binding and neutralizing antibody responses in humans. The S-specific plasmablast and germinal center (GC) B cell responses induced by BNT162b2 vaccination in healthy adults is described in the above example. GC B cells were analyzed in aspirates from the draining axillary lymph nodes of 12 participants after vaccination. The specificity of the GC response was verified by generating a panel of recombinant human mAbs from single cell-sorted S⁺GC B cells isolated from three participants. The majority of these vaccine-induced antibodies are directed against the RBD. The present example assess the capacity of these anti-RBD mAbs to recognize and neutralize recently emerged SARS-CoV-2 variants.

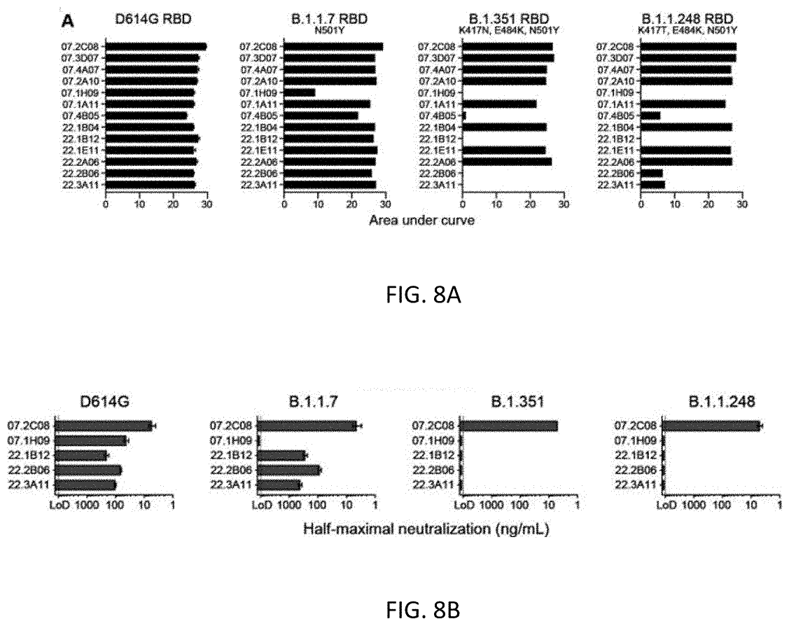

From a pool of S⁺GC B cell-derived mAbs, 13 human anti-RBD mAbs were selected that bound avidly to the predominantly circulating WA1/2020 D614G SARS-CoV-2 strain referred to hereafter as the D614G strain. mAbs binding to recombinant RBDs derived from the D614G strain were assessed and three SARS-CoV-2 variants, B.1.1.7, B.1.351 and B.1.1.248 by enzyme-linked immunosorbent assay (ELISA). Only one mAb, 1H09, showed decreased binding to the RBD derived from the B.1.1.7 variant (FIG. 8A). Four additional mAbs completely lost or showed substantially reduced binding to the B.1.351 and B.1.1.248 variant RBDs (FIG. 8A). The remaining eight mAbs showed equivalent binding to RBDs from all tested strains (FIG. 8A). Next, the in vitro neutralization capacity of the 13 mAbs were examined against the D614G SARS-CoV-2 strain using a high-throughput focus reduction neutralization test (FRNT) with authentic virus. Only five mAbs (2C08, 1H09, 1B12, 2B06, and 3A11) showed high neutralization potency against D614G with 80% neutralization values of less than 100 ng/mL. The ability of these five mAbs to neutralize the B.1.1.7, B.1.351 and B.1.1.248 variants was then assessed. Consistent with the RBD binding data, 1H09 failed to neutralize any of the emerging variants, whereas 1B12, 2B06 and 3A11 neutralized B.1.1.7 but not the B.1.351 and B.1.1.248 variants (FIG. 8B). One antibody, 2C08, neutralized the four SARS-CoV-2 strains tested with remarkable potency (half-maximal inhibitory concentration of 5 ng/mL) (FIG. 8B), indicating that it recognizes RBD residues that are not altered in these variants.

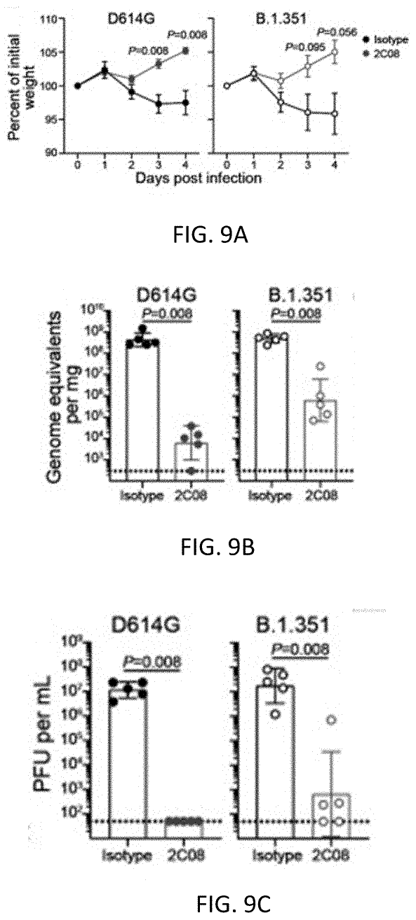

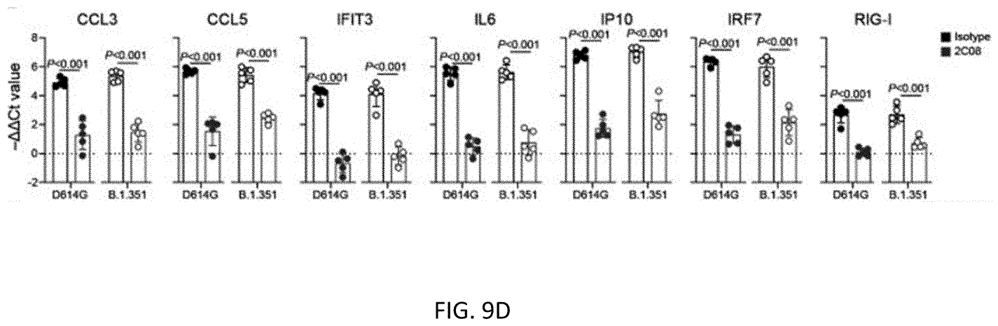

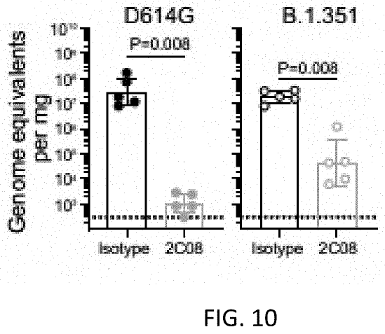

To assess the protective capacity of 2C08 in vivo, a hamster model of SARS-CoV-2 infection was utilized. The prophylactic efficacy of 2C08 against the D614G strain and against a fully infectious recombinant SARS-CoV-2 with B.1.351 spike gene (Wash SA-B.1.351; D80A, 242-244 deletion, R246I, K417N, E484K, N501Y, D614G and A701V) was evaluated in 4-6-week-old male Syrian hamsters. Animals treated with 2C08 and challenged with either virus did not lose weight during the experiment and started to gain weight (relative to starting weight) on 3 dpi. In contrast, animals treated with the isotype control mAb started losing weight on 2 dpi (FIG. 9A). The average weights between the isotype- and 2C08-treated animals differed by 5.9 percent on 3 dpi (P=0.008) and 7.7 percent 4 dpi (P=0.008) for the D614G challenge and by 6.8 percent on 3 dpi (P=0.095) and 9.1 percent 4 dpi (P=0.056) for the B.1.351 challenge. Consistent with the weight loss data, 2C08 treatment reduced viral RNA levels by more than 10,000-fold in the lungs of the D614G challenged hamsters and by approximately 1000-fold in those challenged with B.1.351 SARS-CoV-2 (P=0.008 for both) (FIG. 9B, FIG. 10) on 4 dpi compared to the isotype control mAb groups. Prophylactic treatment also significantly reduced infectious virus titers for both strains detected in the lungs on 4 dpi (P=0.008 for both) (FIG. 9C). In addition to viral load, concentrations of proinflammatory cytokines were significantly reduced in animals that received 2C08 prophylaxis (FIG. 9D). In comparison to control mAb treated animals, a significant decrease in host gene-expression was observed for Ccl3, CcL5, Ifit3, Ifit6, Ip10, Irf7 and Rig-I in lungs of 2C08-treated animals. Overall, prophylaxis with 2C08 showed substantial capacity to decrease viral infection in lower respiratory tissues upon challenge with SARS-CoV-2 strains with spike genes corresponding to ancestral and a key emerging variant.

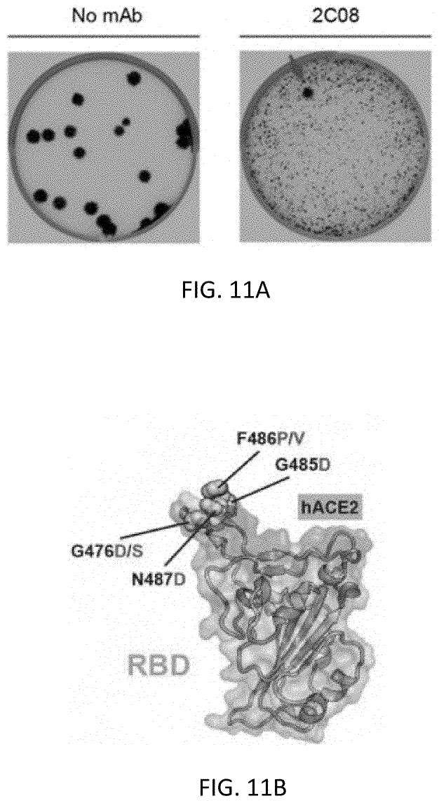

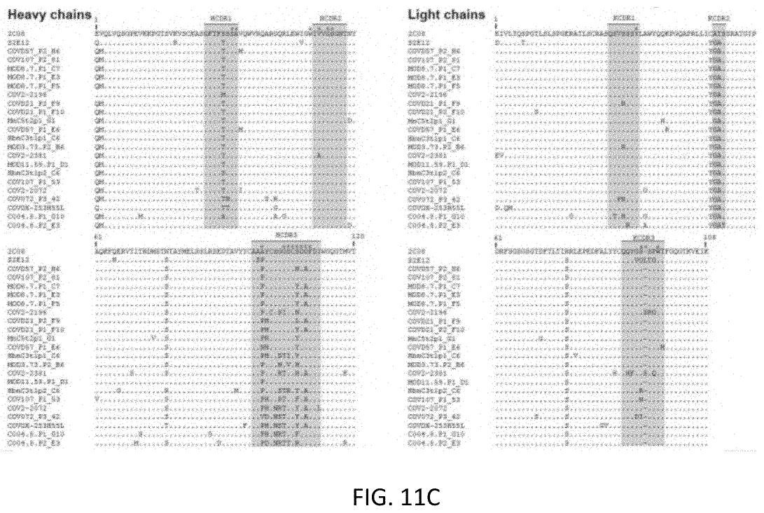

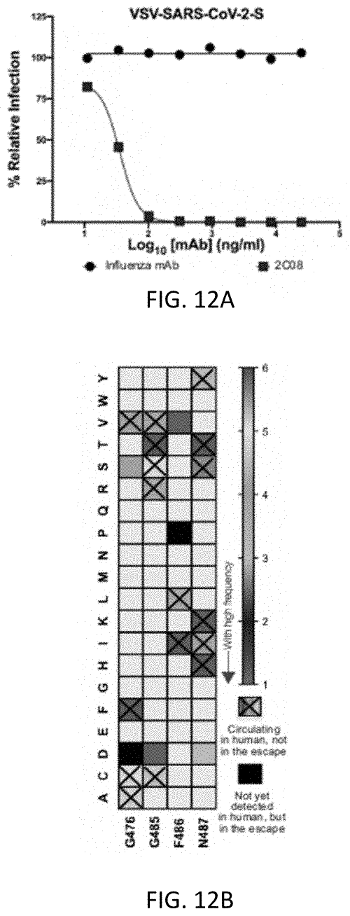

To define the RBD residues targeted by 2C08, VSV-SARS-CoV-2-S chimeric viruses (S from D614G strain) were used to select for variants that escape 2C08 neutralization as previously described. Plaque assays on Vero cells were performed with 2C08 in the overlay, purified the neutralization-resistant plaques, and sequenced the S genes (FIG. 11A, FIG. 12A). Sequence analysis identified the S escape mutations G476D, G476S, G485D, F486P, F486V and N487D, all of which are within the RBD and map to residues involved in ACE2 binding (FIG. 11B). To determine whether any of the 2C08 escape mutants isolated are represented among SARS-CoV-2 variants circulating in humans, all publicly available genome sequences of SARS-CoV-2 were screened. Using 829,162 genomes from Global Initiative on Sharing Avian Influenza Data (GISAID), each substitution frequency was calculated in the identified residues site. Of the six escape variants identified, four were detected among circulating isolates of SARS-CoV-2. The frequency of these substitutions among clinical isolates detected so far is exceedingly rare, with the escape variants representing less than 0.008% of sequenced viruses. In comparison, the D614G substitution is present in 49% of sequenced isolates (FIG. 12B).

TABLE 4
Heavy chain gene usage of mAbs referenced

| | Induced | | | | | | |
|---|---|---|---|---|---|---|---|
| | after | | V-GENE | | D-GENE | HCDR- | |
| | SARS- | | and | J-GENE | and | IMGT | |
| mAb | CoV-2 | Publication | allele | and allele | allele | lengths | HCDR3 |
| 2C08⋄ | mRNA | | IGHV1- | IGHJ3*02 | IGHD2- | 8.8.16 | SEQ ID NO: |
| | vaccine | | 58*01 | | 15*01 | | 33 |
| | | | | | | | AAAYCSGG |
| | | | | | | | SCSDGFDI |
| S2E12⋄ | Infection | (25)Tortorici | IGHV1- | IGHJ3*02 | IGHD2- | 8.8.16 | SEQ ID NO: |
| | | et al., 2020 | 58*01 | | 15*01 | | 227 |
| | | | | | | | AAPDCNRT |
| | | | | | | | TCRDGFDI |
| COVD57_P2_H6\hat{ } | Infection | (24)Robbiani | IGHV1- | IGHJ3*02 | IGHD2- | 8.8.16 | SEQ ID NO: |
| | | et al., 2020 | 58*02 | | 15*01 | | 225 |
| | | | | | | | AAPYCSGG |
| | | | | | | | SCNDAFDI |
| COV107_P2_81\hat{ } | Infection | (24) | IGHV1- | IGHJ3*02 | IGHD2- | 8.8.16 | SEQ ID NO: |
| | | | 58*01 | | 15*01 | | 226 |
| | | | | | | | AAPYCSGG |
| | | | | | | | SCSDAFDI |
| MOD8.7.P1_C7 | mRNA | (15)Wang et | IGHV1- | IGHJ3*02 | IGHD2- | 8.8.16 | SEQ ID NO: |
| | vaccine | al., 2021 | 58*01 | | 15*01 | | 224 |
| | | | | | | | AAPYCSGG |
| | | | | | | | SCYDAFDI |
| MOD8.7.P1_E3 | mRNA | (15) | IGHV1- | IGHJ3*02 | IGHD2- | 8.8.16 | SEQ ID NO: |
| | vaccine | | 58*01 | | 15*01 | | 224 |
| | | | | | | | AAPYCSGG |
| | | | | | | | SCYDAFDI |
| MOD8.7.P1_F5 | mRNA | (15) | IGHV1- | IGHJ3*02 | IGHD2- | 8.8.16 | SEQ ID NO: |
| | vaccine | | 58*01 | | 15*01 | | 224 |
| | | | | | | | AAPYCSGG |
| | | | | | | | SCYDAFDI |
| COV2-2196⋄ | Infection | (23)Zost et | IGHV1- | IGHJ3*02 | IGHD2- | 8.8.16 | SEQ ID NO: |
| | | al., 2020 | 58*01 | | 2*01 | | 223 |
| | | | | | | | AAPYCSSIS |
| | | | | | | | CNDGFDI |
| COVD21_P2_F9\hat{ } | Infection | (24) | IGHV1- | IGHJ3*02 | IGHD2- | 8.8.16 | SEQ ID NO: |
| | | | 58*01 | | 15*01 | | 222 |
| | | | | | | | AAPHCSGG |
| | | | | | | | SCLDAFDI |
| COVD21_P1_F710 | Infection | (24) | IGHV1- | IGHJ3*02 | IGHD2- | 8.8.16 | SEQ ID NO: |
| | | | 58*01 | | 15*01 | | 221 |
| | | | | | | | AAPHCSGG |
| | | | | | | | SCYDAFDI |
| MnC5t2p1_G1\hat{ } | Infection | (26)Kreer et | IGHV1- | IGHJ3*02 | IGHD2- | 8.8.16 | SEQ ID NO: |
| | | al., 2020 | 58*01 | | 15*01 | | 220 |
| | | | | | | | AAPRCSGG |
| | | | | | | | SCYDGFDI |
| COVD57_P1_E6 | Infection | (24) | IGHV1- | IGHJ3*02 | IGHD2- | 8.8.16 | SEQ ID NO: |
| | | | 58*02 | | 15*01 | | 214 |
| | | | | | | | AANHCSGG |
| | | | | | | | SCYDGFDI |
| HbnC3t1p1_C6\hat{ } | Infection | (26) | IGHV1- | IGHJ3*02 | IGHD2- | 8.8.16 | SEQ ID NO: |
| | | | 58*01 | | 2*01 | | 215 |
| | | | | | | | AAPHCSSTI |
| | | | | | | | CYDGFDI |
| MOD3.73.P2_B6 | mRNA | (15) | IGHV1- | IGHJ3*02 | IGHD2- | 8.8.16 | SEQ ID NO: |
| | vaccine | | 58*01 | | 8*01 | | 216 |
| | | | | | | | AAPYCSNG |
| | | | | | | | VCHDGFDI |
| COV2-2381⋄ | Infection | (23) | IGHV1- | IGHJ3*02 | IGHD2- | 8.8.16 | SEQ ID NO: |
| | | | 58*01 | | 2*01 | | 217 |
| | | | | | | | AAPYCSRT |
| | | | | | | | SCHDAFDI |
| MOD11.59.P1_D1 | mRNA | (15) | IGHV1- | IGHJ3*02 | IGHD2- | 8.8.16 | SEQ ID NO: |
| | vaccine | | 58*01 | | 2*01 | | 218 |
| | | | | | | | AAPYCSSTS |
| | | | | | | | CHDGFDI |
| HbnC3t1p2_C6\hat{ } | Infection | (26) | IGHV1- | IGHJ3*02 | IGHD2- | 8.8.16 | SEQ ID NO: |
| | | | 58*01 | | 2*01 | | 219 |
| | | | | | | | AAPYCSST |
| | | | | | | | RCYDAFDI |
| COV107_P1_53 | Infection | (24) | IGHV1- | IGHJ3*02 | IGHD2- | 8.8.16 | SEQ ID NO: |
| | | | 58*01 | | 2*01 | | 210 |
| | | | | | | | AAPHCSST |
| | | | | | | | SCFDAFDI |
| COV2-2072⋄ | Infection | (23) | IGHV1- | IGHJ3*01 | IGHD2- | 8.8.16 | SEQ ID NO: |
| | | | 58*02 | | 2*01 | | 211 |
| | | | | | | | AAPHCNRT |
| | | | | | | | SCYDAFDL |
| COV072_P3_42 | Infection | (24) | IGHV1- | IGHJ3*02 | IGHD2- | 8.8.16 | SEQ ID NO: |
| | | | 58*01 | | 2*01 | | 212 |
| | | | | | | | AAVDCNST |
| | | | | | | | SCYDAFDI |
| C004.8.P1_G10 | mRNA | (15) | IGHV1- | IGHJ3*02 | IGHD2- | 8.8.16 | SEQ ID NO: |
| | vaccine | | 58*01 | | 2*01 | | 213 |
| | | | | | | | AAPHCNRT |
| | | | | | | | SCFDGFDI |
| C004.8.P2_E3 | mRNA | (15) | IGHV1- | IGHJ3*02 | IGHD2- | 8.8.16 | SEQ ID NO: |
| | vaccine | | 58*01 | | 2*01 | | 227 |
| | | | | | | | AAPDCNRT |
| | | | | | | | TCRDGFDI |
| MOD6.24.P2_A7* | mRNA | (15) | IGHV1- | IGHJ3*02 | IGHD2- | 8.8.15 | SEQ ID NO: |
| | vaccine | | 58*01 | | 2*01 | | 21 |
| | | | | | | | AAVYCTTTC |
| | | | | | | | SDAFDI |
| mAb55*\hat{ } | Infection | (54)Dejnirattisai | IGHV1- | IGHJ3*02 | IGHD2- | | SEQ ID NO: |
| | | et al., | 58*01 | | 2*01 | | 208 |
| | | 2021 | | | | | AAPACGTS |
| | | | | | | | CSDAFDI |
| mAb165*\hat{ } | Infection | (54) | IGHV1- | IGHJ3*02 | IGHD2- | | SEQ ID NO: |
| | | | 58*01 | | 15*01 | | 209 |
| | | | | | | | AAPHCIGGS |
| | | | | | | | CHDAFDI |

TABLE 5
Light chain gene usage of mAbs referenced

| | Induced | | | | | |
|---|---|---|---|---|---|---|
| | after | | V-GENE | | LCDR- | |
| | SARS- | | and | J-GENE | IMGT | |
| mAb | CoV-2 | Publication | allele | and allele | lengths | LCDR3 |
| 2C08⋄ | mRNA | | IGKV3- | IGKJ1*01 | 7.3.9 | SEQ ID NO: 30 |
| | vaccine | | 20*01 | | | QQYGSSPWT |
| S2E12⋄ | Infection | (25)Tortorici et | IGKV3- | IGKJ1 | 7.3.9 | SEQ ID NO: 30 |
| | | al., 2020 | 20 | | | QQYGSSPWT |
| COVD57_P2_H6\hat{ } | Infection | (24)Robbiani et | IGKV3- | IGKJ1*01 | 7.3.9 | SEQ ID NO: 30 |
| | | al., 2020 | 20*01 | | | QQYGSSPWT |
| COV107_P2_81\hat{ } | Infection | (24) | IGKV3- | IGKJ1*01 | 7.3.9 | SEQ ID NO: 30 |
| | | | 20*01 | | | QQYGSSPWT |
| MOD8.7.P1_C7 | mRNA | (15)Wang et al., | IGKV3- | IGKJ1*01 | 7.3.9 | SEQ ID NO: 30 |
| | vaccine | 2021 | 20*01 | | | QQYGSSPWT |
| MOD8.7.P1_E3 | mRNA | (15) | IGKV3- | IGKJ1*01 | 7.3.9 | SEQ ID NO: 30 |
| | vaccine | | 20*01 | | | QQYGSSPWT |
| MOD8.7.P1_F5 | mRNA | (15) | IGKV3- | IGKJ1*01 | 7.3.9 | SEQ ID NO: 30 |
| | vaccine | | 20*01 | | | QQYGSSPWT |
| COV2-2196⋄ | Infection | (23)Zost et al., | IGKV3- | IGKJ1*01 | 7.3.10 | SEQ ID NO: |
| | | 2020 | 20*01 | | | 228 |
| | | | | | | QHYGSSRGWT |
| COVD21_P2_F9\hat{ } | Infection | (24) | IGKV3- | IGKJ1*01 | 7.3.9 | SEQ ID NO: 30 |
| | | | 20*01 | | | QQYGSSPWT |
| COVD21_P1_F10 | Infection | (24) | IGKV3- | IGKJ1*01 | 7.3.9 | SEQ ID NO: 30 |
| | | | 20*01 | | | QQYGSSPWT |
| MnC5t2p1_G1\hat{ } | Infection | (26)Kreer et al., | IGKV3- | IGKJ1*01 | 7.3.9 | SEQ ID NO: 30 |
| | | 2020 | 20*01 | | | QQYGSSPWT |
| COVD57_P1_E6 | Infection | (24) | IGKV3- | IGKJ1*01 | 7.3.9 | SEQ ID NO: |
| | | | 20*01 | | | 229 |
| | | | | | | QQYGSSPWM |
| HbnC3t1p1_C6\hat{ } | Infection | (26) | IGKV3- | IGKJ1*01 | 7.3.9 | SEQ ID NO: 30 |
| | | | 20*01 | | | QQYGSSPWT |
| MOD3.73.P2_B6 | mRNA | (15) | IGKV3- | IGKJ1*01 | 7.3.9 | SEQ ID NO: 30 |
| | vaccine | | 20*01 | | | QQYGSSPWT |
| COV2-2381⋄ | Infection | (23) | IGKV3- | IGKJ1*01 | 7.3.10 | SEQ ID NO: |
| | | | 20*01 | | | 232 |
| | | | | | | QHFGSSSQWT |
| MOD11.59.P1_D1 | mRNA | (15) | IGKV3- | IGKJ1*01 | 7.3.9 | SEQ ID NO: 30 |
| | vaccine | | 20*01 | | | QQYGSSPWT |
| HbnC3t1p2_C6\hat{ } | Infection | (26) | IGKV3- | IGKJ1*01 | 7.3.9 | SEQ ID NO: |
| | | | 20*01 | | | 230 |
| | | | | | | QQYGRSPWT |
| COV107_P1_53 | Infection | (24) | IGKV3- | IGKJ1*01 | 7.3.9 | SEQ ID NO: |
| | | | 20*01 | | | 231 |
| | | | | | | QQYGNSPWT |
| COV2-2072⋄ | Infection | (23) | IGKV3- | IGKJ1*01 | 7.3.9 | SEQ ID NO: 30 |
| | | | 20*01 | | | QQYGSSPWT |
| COV072_P3_42 | Infection | (24) | IGKV3- | IGKJ1*01 | 7.3.9 | SEQ ID NO: |
| | | | 20*01 | | | 232 |
| | | | | | | QQYDISPWT |
| C004.8.P1_G10\hat{ } | mRNA | (15) | IGKV3- | IGKJ1*01 | 7.3.9 | SEQ ID NO: 30 |
| | vaccine | | 20*01 | | | QQYGSSPWT |
| C004.8.P2_E3 | mRNA | (15) | IGKV3- | IGKJ1*01 | 7.3.9 | SEQ ID NO: 30 |
| | vaccine | | 20*01 | | | QQYGSSPWT |
| MOD6.24.P2_A7* | mRNA | (15) | IGKV3- | IGKJ1*01 | 7.3.9 | SEQ ID NO: |
| | vaccine | | 20*01 | | | 232 |
| | | | | | | QQYDISPWT |
| mAb55*\hat{ } | Infection | (54)Dejnirattisai | IGKV3- | IGKJ1*01 | | SEQ ID NO: 30 |
| | | et al., 2021 | 20*01 | | | QQYGSSPWT |
| mAb165*\hat{ } | Infection | (54) | IGKV3- | IGKJ1*01 | | SEQ ID NO: 30 |
| | | | 20*01 | | | QQYGSSPWT |

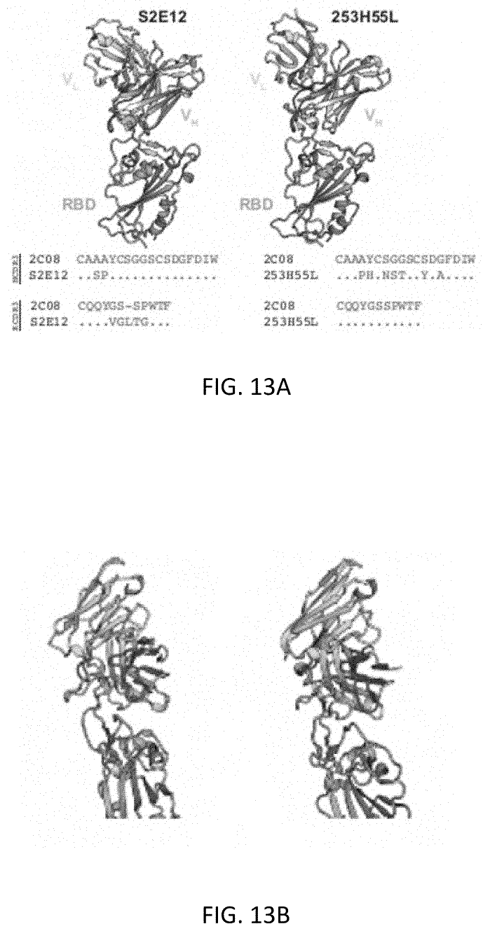

It's noted that 2008 targeted residues are similar to those recognized by a previously described human mAb, S2E12, which was isolated from an infected patient. S2E12 shares a high sequence identity with 2008 (95% amino acid identity) and is encoded by the same immunoglobulin heavy and light chain variable region genes (FIG. 11C, Table 4). Similar to 2008, S2E12 exhibits potent neutralizing activity in vitro and protective capacity in vivo. The cryo-EM structure of S2E12 in complex with S shows that the mAb recognizes an RBD epitope that partially overlaps with the ACE2 receptor footprint known as the receptor binding motif (FIG. 13). S2E12 heavy chain amino acid residues that engage the RBD are identical to those in 2C08, suggesting that 2C08 likely engages the RBD in a manner similar to that of the structurally characterized S2E12. Furthermore, we identified two additional human mAbs, 253H55L and COV2-2196, that share genetic and functional features with 2C08 and have nearly identical antibody-RBD interactions as those of S2E12 (FIG. 11C, FIG. 13). Dong et al. noted that COV2-2196 is likely part of a public B cell clone, citing S2E12 and mAbs generated by two other groups which have similar characteristics. This prompted an expanded search for 2C08-like clonotypes and mAbs. 20 additional mAbs were identified that share the same genetic attributes of 2C08, S2E12, 253H55L and COV2-2196 isolated by different groups from SARS-CoV-2 patients or vaccine recipients (FIG. 11C and Table 4). The primary contact residues described for S2E12 were largely conserved for all mAbs (FIG. 11C).

Cloning and expression of recombinant human mAbs from single cell sorted B cells is now an established method for generating potential therapeutics against a variety of human pathogens. The source cells are predominantly plasmablasts or memory B cells that are isolated from blood after infection or vaccination. The present example describes 2C08, a SARS-CoV-2 vaccine-induced mAb cloned from a GC B cell clone isolated from a draining axillary lymph node sampled from a healthy adult after receiving their second dose of mRNA-based vaccine. 2C08 is a potently neutralizing antibody that targets the receptor binding motif within the RBD of SARS-CoV-2 S protein and blocks infection by circulating SARS-CoV-2 and emerging variants of concern both in vitro and in vivo.

2C08 is a “public” mAb, meaning that it is encoded by multiple B cell clonotypes isolated from different individuals that share similar genetic features. Public antibody responses in humans have been observed after many infections, including SARS-CoV-2 infection. In the case of 2C08-like clonotypes, the mAbs not only share the immunoglobulin heavy and light chain variable region genes, but also have near identical CDRs and are functionally similar. Several have been shown to bind RBD and neutralize D614G as well as variants B.1.17 and B.1.351. 2C08-like mAbs were isolated from multiple SARS-CoV-2 infected patients independently of demographics or severity of infection. Robbiani et al. (Nature. 584, 437-442 (2020)) isolated 2C08-like mAbs from three of six infected individuals analyzed. Tortorici et al. (Science. 370, 950 (2020)) and Zost et al. (Nature. 584, 443-449 (2020)) detected a 2C08-like antibody in one or both of two infected individuals they examined, respectively, whereas Kreer et al. (Cell. 182, 843-854.e12 (2020)) detected a 2C08-like clone in two of seven patients, in one of whom it was expanded. Wang et al. (Nature (2021), doi:10.1038/s41586-021-03324-6) isolated 2C08-like mAbs from five of 14 individuals who received a SARS-CoV-2 mRNA-based vaccine. Nielsen et al. (Cell Host Microbe. 28, 516-525.e5 (2020)) identified 2C08-like rearrangements in sequences derived from four of 13 SARS-CoV-2 patients. It remains to be determined what fraction of the antibody responses induced by SARS-CoV-2 vaccines in humans are comprised of 2C08-type antibodies that are public, potently neutralizing, and so far, minimally impacted by the mutations found in the variants of concern. It is important to note that at least one 2C08-like mAb, COV2-2196, is currently being developed for clinical use.

Notably, most of SARS-CoV-2 vaccine induced anti-RBD mAbs also recognized RBDs from the recent variants. It is of some concern, however, that four of the five neutralizing anti-RBD mAbs lost their activity against the B.1.351 and B.1.1.248 SARS-CoV-2 variants. This is consistent with the data reported by Wang et al. showing that the neutralizing activity of 14 of 17 vaccine induced anti-RBD mAbs was abolished by the introduction of the mutations associated with these variants. More extensive analyses with a larger number of mAbs that target the RBD and non-RBD sites will be needed to precisely determine the fraction of vaccine-induced neutralizing antibody response that is compromised due to antigenic changes in emerging SARS-CoV-2 variants of concern. It's noted that somewhat higher levels of lung viral RNA were recovered from the 2C08-treated animals challenged with the B.1.351-like variant compared to those challenged with the D614G strain. This was unexpected given the similar in vitro potency of 2C08 against both viruses and its capacity to protect animals from both groups against weight loss equivalently. One possibility is that 2C08 more readily selected for a partial escape mutant against viruses displaying the B.1.351 variant spike than the WA1/2020 D614G spike.

Given the germinal center B cell origin of 2C08, the binding of 2C08-related clones could be further refined through somatic hypermutation, and their descendants could become part of the high affinity memory B cell and long-lived plasma cell compartments that confer durable protective immunity. Together, these data suggest that first-generation SARS-CoV-2 mRNA-based vaccines can induce public antibodies with robust neutralizing and potentially durable protective activity against ancestral circulating and key emerging SARS-CoV-2 variants.

### Methods

Cell lines: Expi293F cells were cultured in Expi293 Expression Medium (Gibco). Vero-TMPRSS2 cells (a gift from Siyuan Ding, Washington University School of Medicine) were cultured at 37° C. in Dulbecco's Modified Eagle medium (DMEM) supplemented with 10% fetal bovine serum (FBS), 10 mM HEPES pH 7.3, 1 mM sodium pyruvate, 1× non-essential amino acids, and 100 U/ml of penicillin-streptomycin.

Viruses: The 2019n-CoV/USA_WA1/2020 isolate of SARS-CoV-2 was obtained from the US Centers for Disease Control. The UK B.1.1.7 isolate was obtained from an infected individual. The point mutation D614G in the spike gene was introduced into an infectious complementary DNA clone of the 2019n-CoV/USA_WA1/2020 strain as described previously. The generation of a SARS-CoV-2 virus with the South African variant spike gene (B.1.351) in the background of 2019n-CoV/USA_WA1/2020 was described previously. All viruses were passaged once in Vero-TMPRSS2 cells and subjected to deep sequencing after RNA extraction to confirm the introduction and stability of substitutions. All virus preparation and experiments were performed in an approved Biosafety level 3 (BSL-3) facility.

Monoclonal antibody (mAb) generation: Antibodies were cloned as described previously. Briefly, VH, Vκ, and Vλ genes were amplified by reverse transcription-PCR and nested PCR reactions from singly sorted GC B cells using primer combinations specific for IgG, IgM/A, Igκ, and Igλ from previously described primer sets and then sequenced. To generate recombinant mAbs, restriction sites were incorporated via PCR with primers to the corresponding heavy and light chain V and J genes. The amplified VH, Vκ, and Vλ genes were cloned into IgG1, Igκ, and Igλ expression vectors, respectively, as described previously. Heavy and light chain plasmids were co-transfected into Expi293F cells (Gibco) for expression, and mAbs were purified with protein A agarose (GoldBio).

Antigens: Recombinant receptor binding domain of S (RBD), was expressed as previously described. Briefly, RBD, along with the signal peptide (amino acids 1-14) plus a hexahistidine tag were cloned into mammalian expression vector pCAGGS. RBD mutants were generated in the pCAGGS RBD construct by changing single residues using mutagenesis primers. Recombinant proteins were produced in Expi293F cells (ThermoFisher) by transfection with purified DNA using the ExpiFectamine 293 Transfection Kit (ThermoFisher). Supernatants from transfected cells were harvested 4 days post-transfection, and recombinant proteins were purified using Ni-NTA agarose (ThermoFisher), then buffer exchanged into phosphate buffered saline (PBS) and concentrated using Amicon Ultracel centrifugal filters (EMD Millipore).

Enzyme-linked immunosorbant assay: Assays were performed in 96-well plates (MaxiSorp; Thermo). Each well was coated with 100 μL of wild-type or variant RBD or bovine serum albumin (1 μg/mL) in PBS, and plates were incubated at 4° C. overnight. Plates were then blocked with 0.05% Tween20 and 10% FBS in PBS. mAbs were serially diluted in blocking buffer and added to the plates. Plates were incubated for 90 min at room temperature and then washed 3 times with 0.05% Tween-20 in PBS. Goat anti-human IgG-HRP (Jackson ImmunoResearch 109-035-088, 1:2,500) was diluted in blocking buffer before adding to wells and incubating for 60 min at room temperature. Plates were washed 3 times with 0.05% Tween20 in PBS, and then washed 3 times with PBS. o-Phenylenediamine dihydrochloride substrate dissolved in phosphate-citrate buffer (Sigma-Aldrich) with H₂O₂ catalyst was incubated in the wells until reactions were stopped by the addition of 1 M HCl. Optical density measurements were taken at 490 nm. Area under the curve was calculated using Graphpad Prism v8.

Focus reduction neutralization test: Serial dilutions of each mAb diluted in DMEM with 2% FBS were incubated with 102 focus-forming units (FFU) of different strains or variants of SARS-CoV-2 for 1 h at 37° C. Antibody-virus complexes were added to Vero-TMPRSS2 cell monolayers in 96-well plates and incubated at 37° C. for 1 h. Subsequently, cells were overlaid with 1% (w/v) methylcellulose in MEM supplemented with 2% FBS. Plates were harvested 24 h later by removing overlays and fixed with 4% PFA in PBS for 20 min at room temperature. Plates were washed and sequentially incubated with an oligoclonal pool of SARS2-2, SARS2-11, SARS2-16, SARS2-31, SARS2-38, SARS2-57, and SARS2-71 anti-S antibodies and HRP-conjugated goat anti-mouse IgG (Sigma 12-349) in PBS supplemented with 0.1% saponin and 0.1% bovine serum albumin. SARS-CoV-2-infected cell foci were visualized using TrueBlue peroxidase substrate (KPL) and quantitated on an ImmunoSpot microanalyzer (Cellular Technologies).

SARS-CoV-2 hamster studies: All procedures involving animals were performed in accordance with guidelines of the Institutional Animal Care and Use Committee of Washington University in Saint Louis. Four- to six-week old male Syrian hamsters were obtained from Charles River Laboratories and housed in an enhanced ABSL3 facility at Washington University in St Louis. Animals were randomized from different litters into experimental groups and were acclimatized at the BSL3 facilities for 4-6 days prior to experiments. Animals received intra-peritoneal (IP) injection of isotype control or anti-SARS-CoV-2 mAbs 24 h prior to SARS-CoV-2 challenge. Hamsters were anesthetized with ketamine (150 mg/kg) and xylazine (10 mg/kg) via IP injection and were intranasally inoculated 5×10⁵ PFU of 2019n-CoV/USA_WA1/2020-D614G or Wash SA-B.1.351 SARS-CoV-2 in 100 μL PBS. Animal weights were measured every day for the duration of experiments. Animals were euthanized 4 dpi and the lungs were collected for virological analyses. Left lung lobes were homogenized in 1 mL of PBS or DMEM, clarified by centrifugation, and used for virus titer and cytokine assays.

Virus titration assays from hamster lung homogenates: Plaque assays were performed on Vero-Creanga cells in 24-well plates. Lung tissue homogenates were serially diluted 10-fold, starting at 1:10, in cell infection medium (DMEM+2% FBS+L-glutamine+penicillin+streptomycin). Two hundred and fifty microliters of the diluted virus were added to a single well per dilution per sample. After 1 h at 37° C., the inoculum was aspirated, the cells were washed with PBS, and a 1% methylcellulose overlay in MEM supplemented with 2% FBS was added. Seventy-two hours after virus inoculation, the cells were fixed with 4% formalin, and the monolayer was stained with crystal violet (0.5% w/v in 25% methanol in water) for 1 h at 20° C. The number of plaques were counted and used to calculate the plaque forming units/mL (PFU/mL).

To quantify viral load in lung tissue homogenates, RNA was extracted from 140 μL samples using QIAamp viral RNA mini kit (Qiagen) and eluted with 50 μL of water. Four μL RNA was used for real-time qRT-PCR to detect and quantify N gene of SARS-CoV-2 using TaqMan™ Fast Virus 1-Step Master Mix as described or using the following primers and probes: Forward: SEQ ID NO: 236 GACCCCAAAATCAGCGAAAT; Reverse: SEQ ID NO: 237 TCTGGTTACTGCCAGTTGAATCTG; Probe: SEQ ID NO: 238 ACCCCGCATTACGTTTGGTGGACC; 5′Dye/3′Quencher: 6-FAM/ZEN/IBFQ. Viral RNA was expressed as (N) gene copy numbers per mg for lung tissue homogenates, based on a standard included in the assay, which was created via in vitro transcription of a synthetic DNA molecule containing the target region of the N gene.

For ease of reference, Table 4 showing details on the heavy chains of the antibodies referenced to in this paper is included here. * indicates the antibody is not present in the Figures, panel c alignment. \hat{ } indicates the antibody was previously demonstrated to neutralize D614G. ⋄ indicates the antibody was previously demonstrated to neutralize D614G and viral variants B.1.17 and B.1.351; this study for 2C08).

For ease of reference, Table 5 showing details on the light chains of the antibodies refer-enced to in this paper is included here. * indicates the antibody is not present in Figures, panel c alignment. \hat{ } indicates the antibody was previously demonstrated to neutralize D614G. ⋄ indi-cates the antibody was previously demonstrated to neutralize D614G and viral variants B.1.17 and B.1.351; this study for 2C08).

What is claimed is:

1. An isolated antibody comprising a light chain variable region comprising an L1 of SEQ ID NO: 29, an L2 of ATS, an L3 of SEQ ID NO: 30, or any combination thereof; and/or a heavy chain variable region comprising an H1 of SEQ ID NO: 31, an H2 of SEQ ID NO: 32, an H3 of SEQ ID NO: 33, or any combination thereof.

2. The isolated antibody of claim 1, wherein the amino acid sequence of the light chain variable region comprises SEQ ID NO: 34; and/or the amino acid sequence of the heavy chain variable region comprises SEQ ID NO: 35.

3. A pharmaceutical composition comprising an antibody of claim 1 and a pharmaceutically acceptable carrier or excipient.

4. The pharmaceutical composition of claim 3, further comprising a dispersing agent, buffer, surfactant, preservative, solubilizing agent, isotonicity agent, or stabilizing agent.

5. The pharmaceutical composition of claim 4, wherein said carrier comprises physiological saline, ion exchanger, alumina, aluminum stearate, lecithin, serum protein, human serum albumin, buffer, phosphate, glycine, sorbic acid, potassium sorbate, partial glyceride mixture of saturated vegetable fatty acids, water, salts or electrolytes, protamine sulfate, disodium hydrogen phosphate, potassium hydrogen phosphate, sodium chloride, zinc salt, colloidal silica, magnesium trisilicate, polyvinyl pyrrolidone, cellulose, polyethylene glycol, sodium carboxymethylcellulose, polyacrylates, wax, polyethylene-polyoxypropylene-block polymer, polyethylene glycol, wool fat, or a combination thereof.

6. The antibody of claim 1, wherein the antibody is selected from the group consisting of a humanized antibody, a single chain variable fragment (scFv) antibody, an antibody fragment, or a chimeric antibody.

7. A method of preventing or treating a coronavirus infection in a subject in need thereof, the method comprising administering to the subject a therapeutically effective amount of a composition comprising an antibody or antigen-binding fragment, wherein the antibody or antigen-binding fragment comprises a light chain variable region comprising an L1 of SEQ ID NO: 29, an L2 of ATS, an L3 of SEQ ID NO: 30, or any combination thereof; and/or a heavy chain variable region comprising an H1 of SEQ ID NO: 31, an H2 of SEQ ID NO: 32, an H3 of SEQ ID NO: 33, or any combination thereof.

8. The method of claim 7, wherein the composition is administered intramuscularly, intravenously, intradermally, or intranasally.

9. The method of claim 7, wherein the composition is administered therapeutically to treat an active coronavirus infection.

10. The method of claim 7, wherein the composition is administered prophylactically to prevent a coronavirus infection.

11. The method of claim 7, wherein the coronavirus infection is COVID-19.

12. The method of claim 7, further comprising administering an antiviral drug selected from baloxavir, oseltamivir, zanamivir, peramivir or any combination thereof.

13. An isolated antibody comprising,
(i) a light chain variable region comprising an L1 of SEQ ID NO: 1, an L2 of DAS, an L3 of SEQ ID NO: 2, or any combination thereof and/or a heavy chain variable region comprising an H1 of SEQ ID NO: 3, an H2 of SEQ ID NO: 4, an H3 of SEQ ID NO: 5, or any combination thereof;
(ii) a light chain variable region comprising an L1 of SEQ ID NO: 8, an L2 of AAS, an L3 of SEQ ID NO: 9, or any combination thereof and/or a heavy chain variable region comprising an H1 of SEQ ID NO: 10, an H2 of SEQ ID NO: 11, an H3 of SEQ ID NO: 12, or any combination thereof;
(iii) a light chain variable region comprising an L1 of SEQ ID NO: 15, an L2 of QDN, an L3 of SEQ ID NO: 16, or any combination thereof and/or a heavy chain variable region comprising an H1 of SEQ ID NO: 17, an H2 of SEQ ID NO: 18, an H3 of SEQ ID NO: 19, or any combination thereof;
(iv) a light chain variable region comprising an L1 of SEQ ID NO: 22, an L2 of DAS, an L3 of SEQ ID NO: 23, or any combination thereof and/or a heavy chain variable region comprising an H1 of SEQ ID NO: 24, an H2 of SEQ ID NO: 25, an H3 of SEQ ID NO: 26, or any combination thereof;
(v) a light chain variable region comprising an L1 of SEQ ID NO: 36, an L2 of EDN, an L3 of SEQ ID NO: 37, or any combination thereof and/or a heavy chain variable region comprising an H1 of SEQ ID NO: 38, an H2 of SEQ ID NO: 39, an H3 of SEQ ID NO: 40, or any combination thereof;
(vi) a light chain variable region comprising an L1 of SEQ ID NO: 43, an L2 of DAS, an L3 of SEQ ID NO: 44, or any combination thereof and/or a heavy chain variable region comprising an H1 of SEQ ID NO: 45, an H2 of SEQ ID NO: 46, an H3 of SEQ ID NO: 47, or any combination thereof;
(vii) a light chain variable region comprising an L1 of SEQ ID NO: 50, an L2 of WAS, an L3 of SEQ ID NO: 51, or any combination thereof and/or a heavy chain variable region comprising an H1 of SEQ ID NO: 52, an H2 of SEQ ID NO: 53, an H3 of SEQ ID NO: 54, or any combination thereof;
(viii) a light chain variable region comprising an L1 of SEQ ID NO: 57, an L2 of EVS, an L3 of SEQ ID NO: 58, or any combination thereof and/or a heavy chain variable region comprising an H1 of SEQ ID NO: 59, an H2 of SEQ ID NO: 60, an H3 of SEQ ID NO: 61, or any combination thereof;
(ix) a light chain variable region comprising an L1 of SEQ ID NO: 64, an L2 of GAS, an L3 of SEQ ID NO: 65, or any combination thereof and/or a heavy chain variable region comprising an H1 of SEQ ID NO: 66, an H2 of SEQ ID NO: 67, an H3 of SEQ ID NO: 68, or any combination thereof;
(x) a light chain variable region comprising an L1 of SEQ ID NO: 71, an L2 of EDS, an L3 of SEQ ID NO: 72, or any combination thereof and/or a heavy chain variable region comprising an H1 of SEQ ID NO: 73, an H2 of SEQ ID NO: 74, an H3 of SEQ ID NO: 75, or any combination thereof;
(xi) a light chain variable region comprising an L1 of SEQ ID NO: 78, an L2 of EDS, an L3 of SEQ ID NO: 79, or any combination thereof and/or a heavy chain variable region comprising an H1 of SEQ ID NO: 80, an H2 of SEQ ID NO: 81, an H3 of SEQ ID NO: 82, or any combination thereof;
(xii) a light chain variable region comprising an L1 of SEQ ID NO: 85, an L2 of DAS, an L3 of SEQ ID NO: 86, or any combination thereof and/or a heavy chain variable region comprising an H1 of SEQ ID NO: 87, an H2 of SEQ ID NO: 88, an H3 of SEQ ID NO: 89, or any combination thereof;
(xiii) a light chain variable region comprising an L1 of SEQ ID NO: 92, an L2 of NAS, an L3 of SEQ ID NO: 93, or any combination thereof and/or a heavy chain variable region comprising an H1 of SEQ ID NO: 94, an H2 of SEQ ID NO: 95, an H3 of SEQ ID NO: 96, or any combination thereof;
(xiv) a light chain variable region comprising an L1 of SEQ ID NO: 99, an L2 of WAS, an L3 of SEQ ID NO: 100, or any combination thereof and/or a heavy chain variable region comprising an H1 of SEQ ID NO: 101, an H2 of SEQ ID NO: 102, an H3 of SEQ ID NO: 103, or any combination thereof;
(xv) a light chain variable region comprising an L1 of SEQ ID NO: 106, an L2 of EDS, an L3 of SEQ ID NO: 107, or any combination thereof and/or a heavy chain variable region comprising an H1 of SEQ ID NO: 108, an H2 of SEQ ID NO: 109, an H3 of SEQ ID NO: 110, or any combination thereof;
(xvi) a light chain variable region comprising an L1 of SEQ ID NO: 113, an L2 of DAS, an L3 of SEQ ID NO: 114, or any combination thereof and/or a heavy chain variable region comprising an H1 of SEQ ID NO: 115, an H2 of SEQ ID NO: 116, an H3 of SEQ ID NO: 117, or any combination thereof;
(xvii) a light chain variable region comprising an L1 of SEQ ID NO: 120, an L2 of WAS, an L3 of SEQ ID NO: 121, or any combination thereof and/or a heavy chain variable region comprising an H1 of SEQ ID NO: 122, an H2 of SEQ ID NO: 123, an H3 of SEQ ID NO: 124, or any combination thereof;
(xviii) a light chain variable region comprising an L1 of SEQ ID NO: 127, an L2 of EDN, an L3 of SEQ ID NO: 128, or any combination thereof and/or a heavy chain variable region comprising an H1 of SEQ ID NO: 129, an H2 of SEQ ID NO: 130, an H3 of SEQ ID NO: 131, or any combination thereof;
(xix) a light chain variable region comprising an L1 of SEQ ID NO: 134, an L2 of DDS, an L3 of SEQ ID NO: 135, or any combination thereof and/or a heavy chain variable region comprising an H1 of SEQ ID NO: 136, an H2 of SEQ ID NO: 137, an H3 of SEQ ID NO: 138, or any combination thereof;
(xx) a light chain variable region comprising an L1 of SEQ ID NO: 141, an L2 of KDS, an L3 of SEQ ID NO: 142, or any combination thereof and/or a heavy chain variable region comprising an H1 of SEQ ID NO: 143, an H2 of SEQ ID NO: 144, an H3 of SEQ ID NO: 145, or any combination thereof;
(xxi) a light chain variable region comprising an L1 of SEQ ID NO: 148, an L2 of DAS, an L3 of SEQ ID NO: 149, or any combination thereof and/or a heavy chain variable region comprising an H1 of SEQ ID NO: 150, an H2 of SEQ ID NO: 151, an H3 of SEQ ID NO: 152, or any combination thereof; or
(xxii) a light chain variable region comprising an L1 of SEQ ID NO: 155, an L2 of DDS, an L3 of SEQ ID NO: 156, or any combination thereof and/or a heavy chain variable region comprising an H1 of SEQ ID NO: 157, an H2 of SEQ ID NO: 158, an H3 of SEQ ID NO: 159, or any combination thereof.

14. The isolated antibody of claim 13, wherein the amino acid sequence of the light chain variable region comprises SEQ ID NO: 6, and/or the amino acid sequence of the heavy chain variable region comprises SEQ ID NO: 7; the amino acid sequence of the light chain variable region comprises SEQ ID NO: 13, and/or the amino acid sequence of the heavy chain variable region comprises SEQ ID NO: 14; the amino acid sequence of the light chain variable region comprises SEQ ID NO: 20, and/or the amino acid sequence of the heavy chain variable region comprises SEQ ID NO: 21; the amino acid sequence of the light chain variable region comprises SEQ ID NO: 27, and/or the amino acid sequence of the heavy chain variable region comprises SEQ ID NO: 28; the amino acid sequence of the light chain variable region comprises SEQ ID NO:34, and/or the amino acid sequence of the heavy chain variable region comprises SEQ ID NO: 35; the amino acid sequence of the light chain variable region comprises SEQ ID NO: 41, and/or the amino acid sequence of the heavy chain variable region comprises SEQ ID NO: 42; the amino acid sequence of the light chain variable region comprises SEQ ID NO: 48, and/or the amino acid sequence of the heavy chain variable region comprises SEQ ID NO: 49; the amino acid sequence of the light chain variable region comprises SEQ ID NO: 55, and/or the amino acid sequence of the heavy chain variable region comprises SEQ ID NO: 56; the amino acid sequence of the light chain variable region comprises SEQ ID NO: 62, and/or the amino acid sequence of the heavy chain variable region comprises SEQ ID NO: 63; the amino acid sequence of the light chain variable region comprises SEQ ID NO: 69, and/or the amino acid sequence of the heavy chain variable region comprises SEQ ID NO: 70; the amino acid sequence of the light chain variable region comprises SEQ ID NO: 76, and/or the amino acid sequence of the heavy chain variable region comprises SEQ ID NO:77; the amino acid sequence of the light chain variable region comprises SEQ ID NO: 83, and/or the amino acid sequence of the heavy chain variable region comprises SEQ ID NO: 84; the amino acid sequence of the light chain variable region comprises SEQ ID NO: 90, and/or the amino acid sequence of the heavy chain variable region comprises SEQ ID NO: 91; the amino acid sequence of the light chain variable region comprises SEQ ID NO: 97, and/or the amino acid sequence of the heavy chain variable region comprises SEQ ID NO: 98; the amino acid sequence of the light chain variable region comprises SEQ ID NO: 104, and/or the amino acid sequence of the heavy chain variable region comprises SEQ ID NO: 105; the amino acid sequence of the light chain variable region comprises SEQ ID NO: 111, and/or the amino acid sequence of the heavy chain variable region comprises SEQ ID NO: 112; the amino acid sequence of the light chain variable region comprises SEQ ID NO: 118, and/or the amino acid sequence of the heavy chain variable region comprises SEQ ID NO: 119; the amino acid sequence of the light chain variable region comprises SEQ ID NO: 125, and/or the amino acid sequence of the heavy chain variable region comprises SEQ ID NO: 126; the amino acid sequence of the light chain variable region comprises SEQ ID NO: 132, and/or the amino acid sequence of the heavy chain variable region comprises SEQ ID NO: 133; the amino acid sequence of the light chain variable region comprises SEQ ID NO: 139, and/or the amino acid sequence of the heavy chain variable region comprises SEQ ID NO: 140; the amino acid sequence of the light chain variable region comprises SEQ ID NO: 146, and/or the amino acid sequence of the heavy chain variable region comprises SEQ ID NO: 147; the amino acid sequence of the light chain variable region comprises SEQ ID NO: 153, and/or the amino acid sequence of the heavy chain variable region comprises SEQ ID NO: 154; or the amino acid sequence of the light chain variable region comprises SEQ ID NO: 160, and/or the amino acid sequence of the heavy chain variable region comprises SEQ ID NO: 161.

15. A pharmaceutical composition comprising an antibody of claim 13 and a pharmaceutically acceptable carrier or excipient.

16. A method of preventing or treating a coronavirus infection in a subject in need thereof, the method comprising administering to the subject a therapeutically effective amount of a composition comprising an antibody or antigen-binding fragment of claim 13.

17. The method of claim 16, wherein the composition is administered intramuscularly, intravenously, intradermally, or intranasally.

18. The method of claim 16, wherein the composition is administered therapeutically to treat an active coronavirus infection.

19. The method of claim 16, wherein the composition is administered prophylactically to prevent a coronavirus infection.

20. The method of claim 16, wherein the coronavirus infection is COVID-19.
